SEQUENCE LISTING (58 sequences)

SEQ ID NO: 1 - DNA, 21 bp, Artificial Sequence
  Description of Artifical Sequence: Primer/Probe
actggaagcatctgcttgact

SEQ ID NO: 2 - DNA, 24 bp, Artificial Sequence
  Description of Artifical Sequence: Primer/Probe
tcacctgacagcacagaccctttg

SEQ ID NO: 3 - DNA, 22 bp, Artificial Sequence
  Description of Artifical Sequence: Primer/Probe
atcactcccttcttgtcttggt

SEQ ID NO: 4 - DNA, 21 bp, Artificial Sequence
  Description of Artifical Sequence: Primer/Probe
catcttcaacaggccatggtt

SEQ ID NO: 5 - DNA, 31 bp, Artificial Sequence
  Description of Artifical Sequence: Primer/Probe
ttgagaacaatggtcagataccgccttacca

SEQ ID NO: 6 - DNA, 20 bp, Artificial Sequence
  Description of Artifical Sequence: Primer/Probe
agcagctgtgtccactgcaa

SEQ ID NO: 7 - DNA, 30 bp, Artificial Sequence
  Description of Artifical Sequence: Primer/Probe
atacactgtgttgtaagacatttcattatc

SEQ ID NO: 8 - DNA, 27 bp, Artificial Sequence
  Description of Artifical Sequence: Primer/Probe
ttggcactgaatgacatttcaactcct

SEQ ID NO: 9 - protein, 15 aa, Artificial Sequence
  Description of Artifical Sequence:  Domain
VGHKPGRNTTQRHRC

SEQ ID NO: 10 - protein, 15 aa, Artificial Sequence
  Description of Artifical Sequence:  Domain
CRFKEQKSPDSTWTP

SEQ ID NO: 11 - protein, 20 aa, Artificial Sequence [sequence omitted]

SEQ ID NO: 12 - protein, 16 aa, Artificial Sequence
  Description of Artifical Sequence:  Domain
CSHNHQDKKGVIRESY

SEQ ID NO: 13 - DNA, 4250 bp, Homo sapiens
  CDS (250)..(3390)
gaacacatcgcgtttgcatcccagaaagtagtcgccgcgactatttcccccaaagagacaagcacacatgtaggaatgacaaaggcttgcgaaggagagagcgcagcccgcggcccggagagatcccctcgataatggattactaaatgggatacacgctgtaccagttcgctccgagccccggccgcctgtccgtcgatgcaccgaaaagggtgaagtagagaaataaagtctccccgctgaactactatgaggtcagaagccttgctgctatatttcacactgctacactttgctggggctggtttcccagaagattctgagccaatcagtatttcgcattgcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttacaacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacataatcccagggggagggggtcaggtgtcgaaccagcaggcaaggcgaggtgcccgctcagctcagcaaggttctcaactgcctcgagtacccaccagaccaagaaggcctgcggcagagccgaggacgctgggtcctcctctctgggacacaggggtactcacgaaaactgggccgcgtggtttggtgaaggtttgcaacggcggggactcaccttcattctcttccttcactttcccccacaccctacaacaggtcggacccacaaaagacttcagttatcatcacaaacatgagccaaaagcacatacctaccccatcccccacccccacacacacacacacatgcacacaacacatacacacacacgcacagaggtgaacagaaactgaaacattttgtccacaacttcacgggacgtggccagactgggtttgcgttccaacctgcaaaacacaaatacattttttaaaatcaagaaaatttaaaaagacaaaaaaaaaagaattcattgataattctaactcagactttaacaatggcagaagtttactatgcgcaaatactgtgaaatgcccgccagtgttacagctttctgttgcagcagataaatgccatgttgggcaactatgtcatagatttctgctcctcctctcttttaatgaaataacgtgaccgttaacgcaagtaactctttatttattgttcaccctttttttccttaaggaaaggactcttccaaatatcatcctatgaacagctcttcagaaagcccattgaaagttaaactatttaacgtgaaatccattaactggaataattgagtttctttatttttacaataaattcactgagtaaat

SEQ ID NO: 14 - protein, 1047 aa, Homo sapiens
MRSEALLLYFTLLHFAGAGFPEDSEPISISHCNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACT

SEQ ID NO: 15 - DNA, 1921 bp, Homo sapiens
  CDS (1)..(1920)
gccggatccagtatttcgcattgcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttacaacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccaggtcgacggtg

SEQ ID NO: 16 - protein, 640 aa, Homo sapiens
AGSSISHCNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQVDG

SEQ ID NO: 17 - DNA, 3106 bp, Homo sapiens
  CDS (2)..(3106)
caccggatccggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttacaacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacagtcgacggc

SEQ ID NO: 18 - protein, 1035 aa, Homo sapiens
TGSGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACTVDG

SEQ ID NO: 19 - DNA, 2995 bp, Homo sapiens
  CDS (2)..(2995)
caccggatccctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttataacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccacaatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacagtcgacggc

SEQ ID NO: 20 - protein, 998 aa, Homo sapiens
TGSLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACTVDG

SEQ ID NO: 21 - DNA, 1327 bp, Homo sapiens
  CDS (2)..(1327)
caccggatccctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcaggtcgacggc

SEQ ID NO: 22 - protein, 442 aa, Homo sapiens
TGSLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQVDG

SEQ ID NO: 23 - DNA, 1492 bp, Homo sapiens
  CDS (2)..(1492)
caccggatccatgaggtcagaagccttgctgctatatttcacactgctacactttgctggggctggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcaggtcgacggc

SEQ ID NO: 24 - protein, 497 aa, Homo sapiens
TGSMRSEALLLYFTLLHFAGAGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQVDG

SEQ ID NO: 25 - DNA, 1438 bp, Homo sapiens
  CDS (2)..(1438)
caccggatccggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaattgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcaggtcgacggc

SEQ ID NO: 26 - protein, 479 aa, Homo sapiens
TGSGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQVDG

SEQ ID NO: 27 - DNA, 3055 bp, Homo sapiens
  CDS (2)..(3055)
caccggatccggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacagtcgacggc

SEQ ID NO: 28 - protein, 1018 aa, Homo sapiens
TGSGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACTVDG

SEQ ID NO: 29 - DNA, 2944 bp, Homo sapiens
  CDS (2)..(2944)
caccggatccatgaggtcagaagccttgctgctatatttcacactgctacactttgctggggctggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaaggggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgacgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacagtcgacggc

SEQ ID NO: 30 - protein, 981 aa, Homo sapiens
TGSMRSEALLLYFTLLHFAGAGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRIKGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACTVDG

SEQ ID NO: 31 - DNA, 3498 bp, Homo sapiens
  CDS (214)..(3030)
  misc_feature (3047)..(3047): Wherein n may be a, c, g or t
gcgactatttcccccaaagagacaagcacacatgtaggaatgacaaaggcttgcgaaggagagagcgcagcccgcggcccggagagatcccctcgataatggattactaaatgggatacacgctgtaccagttcgctccgagccccggccgcctgtccgtcgatgcaccgaaaagggtgaagtagagaaataaagtctccccgctgaactactatgaggtcagaagccttgctgctatatttcacactgctacactttgctggggctggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcgggaacccagccgcggcacccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccacctgaccacctactctcatcagaagcaacactaaccccgacaattcanctctgacttcaaagggaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacataatcccagggggagggggtcaggtgtcgaaccagcaggcaaggcgaggtgtccgctcagctcagcaaggttctcaactgcctcgagtacccaccaaaccaaaaaggcctgcggcagaaccgagggacgctgggtcctcctctctgggacacaggggtactcacgaaaactgggccgcgtggtttggtgaaag

SEQ ID NO: 32 - protein, 939 aa, Homo sapiens
MRSEALLLYFTLLHFAGAGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKREPSRGTREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHLTTYSHQKQH

SEQ ID NO: 33 - DNA, 1878 bp, Homo sapiens
  CDS (1)..(1878)
ggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccag

SEQ ID NO: 34 - protein, 626 aa, Homo sapiens
GFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGHSSSLLPSTTTSDSTAQEGYESRGGMLDWKHLLDSPDSTDPLGAVSSHNHQDKKGVIRESYLKGHDQ

SEQ ID NO: 35 - DNA, 1908 bp, Homo sapiens
  CDS (1)..(1908)
ggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagatgcagctatgatggagtcgaagacaaaaggatcatgggcatgcagctggacagagcaagcagctctctgtatgttgcgttctctacctgtgtgataaaggtcccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaa

SEQ ID NO: 36 - protein, 636 aa, Homo sapiens
GFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQ

SEQ ID NO: 37 - DNA, 54 bp, Homo sapiens
  CDS (1)..(54)
ggagaaagccctaccctgcggaccgtcaagcacgattcaaaatggttgaaagaa

SEQ ID NO: 38 - protein, 18 aa, Homo sapiens
GESPTLRTVKHDSKWLKE

SEQ ID NO: 39 - DNA, 54 bp, Homo sapiens; identical to SEQ ID NO: 37
  CDS (1)..(54)

SEQ ID NO: 40 - protein, 18 aa, Homo sapiens
GESPTLRTVKHDSKWLKE

SEQ ID NO: 41 - DNA, 51 bp, Homo sapiens
  CDS (1)..(51)
tcatcctccttagaaagatatgcaacctccaatgagttccctgatgatacc

SEQ ID NO: 42 - protein, 17 aa, Homo sapiens
SSSLERYATSNEFPDDT

SEQ ID NO: 43 - DNA, 60 bp, Homo sapiens
  CDS (1)..(60)
gaggagatgagtgtttacaactctgaaaaatgcagctatgatggagtcgaagacaaaagg

SEQ ID NO: 44 - protein, 20 aa, Homo sapiens
EEMSVYNSEKCSYDGVEDKR

SEQ ID NO: 45 - DNA, 3983 bp, Homo sapiens
  CDS (214)..(3126)
gcgactatttcccccaaagagacaagcacacatgtaggaatgacaaaggcttgcgaaggagagagcgcagcccgcggcccggagagatcccctcgataatggattactaaatgggatacacgctgtaccagttcgctccgagccccggccgcctgtccgtcgatgcaccgaaaagggtgaagtagagaaataaagtctccccgctgaactactatgaggtcagaagccttgctgctatatttcacactgctacactttgctggggctggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacggtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacataatcccagggggagggggtcaggtgtcgaaccagcaggcaaggcgaggtgcccgctcagctcagcaaggttctcaactgcctcgagtacccaccagaccaagaaggcctgcggcagagccgaggacgctgggtcctcctctctgggacacaggggtactcacgaaaactgggccgcgtggtttggtgaaggtttgcaacggcggggactcaccttcattctcttccttcactttcccccacaccctacaacaggtcggacccacaaaagacttcagttatcatcacaaacatgagccaaaagcacatacctaccccatcccccacccccacacacacacacatgcacacaacacatacacacacacgcacagaggtgaacagaaactgaaacattttgtccacaacttcacgggacgtggccagactgggtttgcgttccaacctgcaaaacacaaatacattttttaaaatcaagaaaatttaaaaagacaaaaaaaagaattcattgataattctaactcagactttaacaatggcagaagtttactatgcgcaaatactgtgaaatgcccgccagtgttacagctttctgttgcagcagataaatgccatgttgggcagctatgtcatagatttctgctcctcctctcttgtcatagatttctgctcctcctctcttgtcatagatttctgctcctcctctcttgtcatagatttctgctcctcctctcttgtcatagatttctgctcctcctctcttgtcatagatttctgctcctcctctcttgtcatagatttctgctcctcctctcttgtcatagatttctgctcctcctctcttgtcatagatttctg

SEQ ID NO: 46 - protein, 971 aa, Homo sapiens
MRSEALLLYFTLLHFAGAGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKRWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACT

SEQ ID NO: 47 - DNA, 3165 bp, Homo sapiens
  CDS (13)..(3153)
cagcgcggatccatgaggtcagaagccttgctgctgtatttcacactgctacactttgctggggctggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttacaacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatctcagcagcaagagtcccaaccatggggtgaaccttgtggagaacctggacagcctgccccccaaagttccacagcgggaggcctccctgggtcccccgggagcctccctgtctcagaccggtctaagcaagcggctggaaatgcaccactcctcttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacagtcgacgcgctg

SEQ ID NO: 48 - protein, 1047 aa, Homo sapiens
MRSEALLLYFTLLHFAGAGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHLSSKSPNHGVNLVENLDSLPPKVPQREASLGPPGASLSQTGLSKRLEMHHSSSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACT

SEQ ID NO: 49 - DNA, 1948 bp, Homo sapiens
  CDS (2)..(1948)
caccggatccggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttataacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccacaatcaccaagacaagaagggagtgattcgggaaagttacctcaaaggccacgaccaggtcgacggc

SEQ ID NO: 50 - protein, 649 aa, Homo sapiens
TGSGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQVDG

SEQ ID NO: 51 - DNA, 2583 bp, Homo sapiens
  CDS (1)..(2583)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggcggcggcggcggcggcggcggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccag

SEQ ID NO: 52 - protein, 861 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGGGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGHSSSLLPSTTTSDSTAQEGYESRGGMLDWKHLLDSPDSTDPLGAVSSHNHQDKKGVIRESYLKGHDQ

SEQ ID NO: 53 - DNA, 2634 bp, Homo sapiens
  CDS (1)..(2634)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggcggcggcggcggcggcggcggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttataacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccacaatcaccaagacaagaagggagtgattcgggaaagttacctcaaaggccacgaccag

SEQ ID NO: 54 - protein, 878 aa, Homo sapiens [sequence omitted]

SEQ ID NO: 55 - DNA, 2113 bp, Homo sapiens
  CDS (1)..(1998)
gccaccatggagacagacacactcctgctatgggtactgctgctctgggttccaggttccactggtgacggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacggaggaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaattgcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccgtattgtggatggataaaggaaggtggtgcctgcagccatttatcacccaacagcagactgacttttgagcaggacatagagcgtggcaatacagatggtctgggggactgtcacaattcctttgtggcactgaatgacatttcaactcctctaccagataatgaaatgtcttacaacacagtgtatgggcattccagttccctcttgcccagcacaaccacatcagattcgacggctcaagaggggtatgagtctaggggaggaatgctggactggaagcatctgcttgactcacctgacagcacagaccctttgggggcagtgtcttcccataatcaccaagacaagaagggagtgattcgggaaagttacctcaaaggccacgaccagtgactcgaggactacaaggatgacgatgacaaggattacaaagacgacgatgataaggactataaggatgatgacgacaaataatagcaattcctcgacgctgcatagggttaca

SEQ ID NO: 56 - protein, 666 aa, Homo sapiens
ATMETDTLLLWVLLLWVPGSTGDGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNDISTPLPDNEMSYNTVYGHSSSLLPSTTTSDSTAQEGYESRGGMLDWKHLLDSPDSTDPLGAVSSHNHQDKKGVIRESYLKGHDQ

SEQ ID NO: 57 - skipped

SEQ ID NO: 58 - DNA, 21 bp, Artificial Sequence
  Description of Artifical Sequence: Primer/Probe
gggactgtcacaattcctttg

(19) United States
(12) Patent Application Publication
(10) Pub. No.: US 2004/0018977 A1
(43) Pub. Date: Jan. 29, 2004

(54) Semaphorin-like proteins and methods of using same

(76) Inventors: Enrique Alvarez, Clinton, CT (US); David W. Anderson, Plantsville, CT (US); Mohanraj Dhanabal, Branford, CT (US); Nikolai V. Khramtsov, Branford, CT (US); William J. LaRochelle, Madison, CT (US); Henri S. Lichenstein, Guilford, CT (US); Li Li, Branford, CT (US); Chean Eng Ooi, Branford, CT (US); Muralidhara Padigaru, Branford, CT (US); Richard A. Shimkets, Guilford, CT (US); Mei Zhong, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/449,548

(22) Filed: May 30, 2003

Related U.S. Application Data
(63) Continuation-in-part of application No. 09/520,781, filed on Mar. 8, 2000.
(63) Continuation-in-part of application No. 10/403,676, filed on Mar. 31, 2003.
(60) Provisional application No. 60/123,667, filed on Mar. 9, 1999.
(60) Provisional application No. 60/371,002, filed on Apr. 9, 2002.
(60) Provisional application No. 60/384,798, filed on May 30, 2002.
(60) Provisional application No. 60/402,407, filed on Aug. 9, 2002.
(60) Provisional application No. 60/443,062, filed on Jan. 28, 2003.

(51) Int. Cl. A61K 38/17; A61K 48/00; C12N 5/08

(52) U.S. Cl. 514/12; 435/366; 435/372; 514/44

(57) ABSTRACT

The proteins of the invention are a family used to modulate the activity of cells and their ability to attract blood vessels. The proteins are used to inhibit angiogenesis, inhibit cell migration, and inhibit actin filament formation. These proteins are used in this context to diagnose and treat proliferative disorders such as cancer.

### RELATED APPLICATIONS

This application is a continuation-in-part of U.S. Ser. No. 09/520,781 filed Mar. 3, 2000, which claims priority to U.S. Ser. No. 60/123,667, filed Mar. 9, 1999; U.S. Ser. No. 60/234,082, filed Sep. 20, 2000; U.S. Ser. No. 60/233,798, filed Sep. 19, 2000; and U.S. Ser. No. 60/174,485, filed Jan. 4, 2000; and is a continuation-in-part of U.S. Ser. No. 10/403,676, filed Mar. 31, 2003 which claims priority to U.S. Ser. No. 60/371,002, filed Apr. 9, 2002; U.S. Ser. No. 60/384,798, filed May 30, 2002; U.S. Ser. No. 60/402,407, filed Aug. 9, 2002; U.S. Ser. No. 60/443,062, filed Jan. 28, 2003 all of which are incorporated herein in their entirety.

### FIELD OF THE INVENTION

The present invention relates to semaphorin like novel polypeptides, and the nucleic acids encoding them, having anti-angiogenic properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as anti-angiogenic therapeutics including but not limited to methods of treating renal cancers, glioblastoma and pancreatic cancers.

### BACKGROUND OF THE INVENTION

During development, different cell surface molecules regulate the interaction between cells and the extracellular matrix, that are essential for the processes of cell migration, proliferation, differentiation and apoptosis (Shima, D. T., and C. Mailhos 2000, Curr Opin Genet Dev. 10:536-42). These spatially and temporally coordinated interactions are essential to ensure that progenitor cells differentiate in the appropriate environment. In this regard, the semaphorin family of proteins plays an important and crucial role in the development of the central nervous system (CNS). Semaphorins were originally characterized in the nervous system as axonal guidance molecules (Kolodkin, A. L. 1998. Prog Brain Res. 117:115-32), a finding receiving support from several studies in knock-out mice ( Behar et al, 1996 Nature 383: 525-528 ), Taniguchi et al 1997, Neuron 19: 519-530). Semaphorins have also been implicated in cardiac and skeletal development ( Behar et al, 1996 Nature 383: 525-528), in the immune response (Hall et al 1996, Proc Natl Acad Sci U S A. 93:11780-5), in the regulation of angiogenesis (Miao et al, 1999, J Cell Biol. 146: 233-242) and in tumor growth and metastasis (Christensen et al, 1998, Cancer Res. 58:1238-44). The semaphorins comprise a large family (>25 genes) of secreted and transmembrane glycoproteins categorized into eight different classes (Kolodkin, A. L. 1998, Prog Brain Res. 117:115-32). The semaphorins possess an extracellular Sema domain of ˜500 amino acids, followed by a short transmembrane domain containing 17 highly conserved cysteine residues. Recent studies have identified Sema 3B as a tumor suppressor gene (TSG) that is frequently inactivated in lung cancers (Tomizawa et al 2001, Proc Natl Acad Sci U S A. 98:13954-9). Among the most widely studied semaphorins, Sema 3A acts as a repellent for axons and has also been shown to inhibit the migration of endothelial cells. This observation lead to the hypothesis that the balance between the guidance molecule (Sema 3A) and angiogenic factors (VEGF) might modulate the migration, apoptosis and proliferation of neural progenitor cells through shared receptors (Miao et al, 1999, J Cell Biol. 146: 233-242)).

Semaphorin 6A-1 is a transmembrane semaphorin, the expression of which suggests a function in embryonic nervous system development (Xu et al, 2000 J. Neuroscie. 20:2638-2648). The murine Sema 6A-1 ortholog has also been isolated (Xu et al, 2000, J Neurosci. 20:2638-48) and the soluble ectodomain was reported to cause sympathetic neurons and dorsal root ganglion growth cone collapse. Klostermann et al indirectly linked Sema 6A-1 signaling to cytoskeletal element binding proteins such as Ena/VASP, thereby suggesting its role in retrograde signaling and cytoskeletal rearrangement (Klostermann et al 2000, J Biol Chem. 275:39647-53). Further studies (Klostermann et al 2000, J Biol Chem. 275:39647-53) suggested that Sema 6A-1 functions as a key element in targeting filament synthesis machinery to cell sites associated with motility, growth and adhesion.

Recent studies have shown that semaphorin interacts with NP-1 which signals by complexing with plexin (Kolodkin, A. L. 1998. Prog Brain Res. 117:115-32)). Depending upon the specific plexin co-expressed in the receptor complex, cells might display either repulsion or attraction (Chen et al, 1997, Neuron 19: 547-559). Previous studies have shown that NP-1 is expressed on endothelial cells and is apparently also overexpressed on some tumor (breast and prostate) cells (Soker, S. 2001, J Biochem Cell Biol. 33:433-7). Binding of NP-1 to semaphorin/collapsin inhibited the motility of axons, whereas its binding to VEGF₁₆₅ enhanced chemotaxis in the endothelial cell compartment (Soker, S. 2001, J Biochem Cell Biol. 33:433-7). Plexins are essential semaphorin receptor components in all neurons, transducing extracellular events to cytoplasmic signaling cascades. A number of semaphorins bind directly to plexin receptors, except for class 3 semaphorins, which require neuropilins as obligatory ligand-binding co-receptors for plexin-based signaling functions (Liu, B. P., and S. M. Strittmatter 2001, Curr Opin Cell Biol. 13:619-26).

Studies have implicated rho family GTPases in semaphorin signaling (Rohm et al, 2000 FEBS Lett. 486:68-72). The growth cone collapse induced by semaphorins utilizes both f-actin rearrangement and endocytosis. The small GTPase is a well-known modulator of actin dynamics that is also involved in stress fiber formation, focal adhesion assembly and cell migration (Sanders et al, 1999, Science. 283:2083-2085). Coordinated migration is controlled by the effect of rac on rho a which allows switching-over between the contractile and non-contractile state. Similarly migration induced by VEGF in endothelial cells is an essential component of angiogenesis that requires a tight regulation of the contractile and non-contractile states of the cell. Recent studies have identified major signaling pathways downstream of VEGFR2 that regulate contractile forces of the endothelial cells by modulating actin organization and dynamics (Rousseau et al 2000, J Biol. Chem. 275:10661-10672). Studies have shown that NP-1 binding to two different ligands differentially affects motility. All of these studies demonstrate comparable developmental tasks between endothelial and neuronal cells.

The present invention related to novel semaphorin -like nucleotides and polypeptides and their therapeutic uses as anti-angiogenic agent in the treatment of cancers including but not limited to renal carcinoma and glioblastoma.

### SUMMARY OF THE INVENTION

The invention is based on the discovery of proteins and nucleic acids which inhibited cell migration, angiogenesis and actin filament formation. Accordingly, the invention features methods of modulating (i.e.,preventing, inhibiting or promotong) angiogenesis, cell motility, and actin filament formation in a cell or bodily tissue.

Cell migration, angiogenesis or actin filament formation is inhibited by contacting or introducing to a cell or tissue a composition containing a NOVX polypeptide (e.g., SEQ ID NOs: 14, 16, 18, 20, 22, 24, 26, 28, 30, 32, 34, 36, 38, 40, 42, 44, 46, 48, 50, 52, 54 or 56) or a NOVX nucleic acid (e.g., SEQ ID NOs: 13, 15, 17, 19, 21, 23, 25, 27, 29, 31, 33, 35, 37, 39, 41, 43, 45, 47, 49, 51, 53, or 55). Alternatively the composition contains a polypeptide or nucleic acid which has at least 95% sequence identity to a NOVX polypeptide or nucleic acid. The invention also features methods of preventing or alleviating a symptom of cell migration/angiogenesis related disorder in a subject by identifying a subject suffering from or at risk of developing cell migration/angiogenesis related disorder and administering to the subject a NOVX polypeptide or nucleic acid.

These cell or tissue is contacted in vivo, in vitro, or ex vivo. The cell or tissue is a normal or cancerous. The cell is an endothelial cell, a epithelial cell,a neuronal cells or a mesenchymal cell. For example the cell is a neuroblastoma cell, a renal carcinoma cell, a fibrosarcoma cell,a rhabdosarcoma cell, or a pancreatic cancer cell. The tissue is, for example, endothelial tissue, epithelial tissue neuronal tissue or mesenchymal tissue. Endothelial tissue includes, for example, a vein, an artery, and a microvasculature. Epithelial tissue includes, for example, a kidney tissue, a pancreatic tissue and a renal tissue. Neuronal tissue includes, for example, a glial tissue.

The subject is a mammal such as human, a primate, mouse, rat, dog, cat, cow, horse, pig. The subject is suffering from or at risk of developing cell migration/angiogenesis related disorder. Cell migration/angiogenesis related disorder include for example, cancer such neuroblastoma, renal carcinoma, fibrosarcoma, rhabdosarcoma and pancreatic cancer, wound healing, or tissue regeneration. A subject suffering from or at risk of developing a cell migration/angiogenesis related disorder is identified by methods known in the art, e.g., gross examination of tissue or detection of a tumor.

The invention further provides chimeric proteins. The chimeric proteins include a first and a second polypeptide. The first polypeptide includes a NOVX polypeptide, including SEQ ID NOs: 14, 16, 18, 20, 22, 24, 26, 28, 30, 32, 34, 36, 38, 40, 42, 44, 46, 48, 50, 52, 54, or 56. The second polypeptide is a portion of an immunoglobulin molecule. The portion of the immunoglobulin molecule includes for example the eFc region of the immunoglobulin molecule. For example, the chimeric protein includes SEQ ID NOs:50 or 54.

### BRIEF DESCRIPTION OF DRAWINGS

FIG. 1 is a schematic showing the Pathcalling interaction of proteins with the extracellular domain of CG51896-01 (Semaphorin 6A).

FIG. 2 is a schematic showing the Pathcalling interaction of proteins with the cytoplasmic domain of CG51896-10.

FIG. 3 is a graph showing the effect of CG51896-02 on the migration of human umbilical vein endothelial cells (HUVEC) and human microvascular endothelial cells (HMCEC-d).

FIG. 4 is a bar graph showing the effect of CG51896-02 on the migration of 786-0 cells (renal carcinoma).

FIG. 5 is a bar graph showing the effect of CG51896-02 on the migration of SJCRH30 cells (rhabdosarcoma).

FIG. 6 is a bar graph showing the effect of CG51896-02 on the migration of SK—N—SH cells (neuroblastoma).

FIG. 7. is a bar graph showing the effect of CG51896-02 on the migration of U87-MG cells (neuroblastoma).

FIG. 8. is a bar graph showing the effect of CG51896-02 on the migration of CAKI-2 cells (renal carcinoma).

FIG. 9 is a bar graph showing the effect of CG51896-11 on the migration of SK—N—SH cells (neuroblastoma).

FIG. 10 is a bar graph showing the effect of CG51896-11 on the migration of HT1080 cells (fibrosarcoma).

FIG. 11 is a bar graph showing the effect of CG51896-11 on the migration of U87-MG cells (neuroblastoma).

FIG. 12 is a bar graph showing the effect of CG51896-11 on the migration of human umbilical vein endothelial cells (HUVEC).

FIG. 13 is a bar graph showing the effect of CG51896-11 on the migration of CAKI-2 cells (renal carcinoma).

FIG. 14 is a bar graph showing the effect of CG51896-11 on the migration of Panc-1cells (renal carcinoma).

FIG. 15 is a bar graph showing the effect of CG51896-02 on the invasion of 786-0 cells (renal carcinoma).

FIG. 16 is a series of light micrographs of human umbilical vein endothelial cells (HUVEC) showing the effect of CG51896-02 on actin cytoskeleton. FIG. 16A shows unstimulated cells. FIG. 16BB shows VEGF (10 ng/ml) treated cells. FIG. 16C shows VEGF plus CG51896-02 (Semaphorin 6A extracellular domain) treated cells. FIG. 16D shows VEGF plus Cytochalasin D, an inhibitor of actin filaments.

FIG. 17 is a Western blot showing the effect of CG51896-02 on VEGF-stimulated Src and FAK Phosphorylation.

FIG. 18 is a Western blot showing a coimmunoprecipitation of CG51896-02 and Plexin A1.

FIG. 19 is a bar graph showing the effect of polyclonal antibodies (N-40, I340, C640) on CG51896-02, -11, and -12.

FIG. 20A is a bar graph showing the effect of polyclonal antibodies (S578) on CG51896-02.

FIG. 20B is a bar graph showing the effect of polyclonal antibodies (S578) on CG51896-11 and CG51896-12.

FIG. 21 is a micrograph showing the effect of CG51896-02 on growth cone collapse.

FIG. 22 is a bar graph showing the quantitative analysis of growth cone collapse in the presence of CG51896-02.

FIG. 23 is a photograph showing the effect of CG51896-02 on matrigel plug 786-0-induced angiogenesis in athymic nude mice (gross morphology).

FIG. 24 is a micrograph showing the CD31 staining of matrigel plugs (786-0-induced angiogenesis) following CG51896-02 administration.

FIG. 25 is a bar graph showing the morphometric analysis showing the relative number of vessel lengths following CG51896-02 administration (786-0-induced angiogenesis).

FIG. 26 is a bar graph showing the morphometric analysis showing the relative number of nodes following CG51896-02 administration (786-0-induced angiogenesis).

FIG. 27 is a bar graph showing the morphometric analysis showing the relative number of vessel ends following CG51896-02 administration (786-0-induced angiogenesis).

FIG. 28 is a photograph showing the effect of CG51896-02 on matrigel plug VEGF/bFGF-induced angiogenesis in athymic nude mice (gross morphology).

FIG. 29 is a micrograph showing the CD31 staining of matrigel plugs (VEGF/bFGF-induced angiogenesis) following CG51896-02 administration.

FIG. 30 is a bar graph showing the morphometric analysis showing the relative number of vessel lengths following CG51896-02 administration (VEGF/bFGF-induced angiogenesis).

FIG. 31 is a bar graph showing the morphometric analysis showing the relative number of nodes following CG51896-02 administration (VEGF/bFGF-induced angiogenesis).

FIG. 32 is a bar graph showing the morphometric analysis showing the relative number of vessel ends following CG51896-02 administration (VEGFfbFGF-induced angiogenesis).

FIG. 33 is a scatterplot showing the endpoints for individual mice in every treatment group for an efficacy evaluation of CG51896-02 against U87MG human glioma line grown as a xenograft in nude mice.

FIG. 34 is graphs showing an efficacy evaluation of CG51896-02 against U87MG human glioma line grown as a xenograft in nude mice. The upper panel shows a median tumor growth curve for each treatment group. The lower panel shows a Kaplan-Meier plot for each treatment group.

### DETAILED DESCRIPTION OF THE INVENTION

The present invention provides novel semaphoring nucleotides and polypeptides encoded thereby. Included in the invention are the novel nucleic acid sequences, their encoded polypeptides, antibodies, and other related compounds and their use as anti-angiogenic compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
Sequences and Corresponding SEQ ID Numbers

| | | SEQ | SEQ | |
|---|---|---|---|---|
| | | ID | ID | |
| | | NO | NO | |
| NOVX | Internal | (nucleic | (amino | |
| Assignment | Identification | acid) | acid) | Homology |
| NOV2a | CG51896-04 | 13 | 14 | Human semaphorin |
| | | | | 6A-1 - Homo sapiens |
| NOV2b | 271674560 | 15 | 16 | Human semaphorin |
| | | | | 6A-1 - Homo sapiens |
| NOV2c | 267441133 | 17 | 18 | Human semaphorin |
| | | | | 6A-1 - Homo sapiens |
| NOV2d | 267441137 | 19 | 20 | Human semaphorin |
| | | | | 6A-1 - Homo sapiens |
| NOV2e | 262254987 | 21 | 22 | Human semaphorin |
| | | | | 6A-1 - Homo sapiens |
| NOV2f | 260565761 | 23 | 24 | Human semaphorin |
| | | | | 6A-1 - Homo sapiens |
| NOV2g | 252324008 | 25 | 26 | Human semaphorin |
| | | | | 6A-1 - Homo sapiens |
| NOV2h | 252323542 | 27 | 28 | Human semaphorin |
| | | | | 6A-1 - Homo sapiens |
| NOV2i | 252323483 | 29 | 30 | Human semaphorin |
| | | | | 6A-1 - Homo sapiens |
| NOV2j | CG51896-01 | 31 | 32 | Human semaphorin |
| | | | | 6A-1 - Homo sapiens |
| NOV2k | CG51896-02 | 33 | 34 | Human semaphorin |
| | | | | 6A-1 - Homo sapiens |
| NOV2l | CG51896-03 | 35 | 36 | Human semaphorin |
| | | | | 6A-1 - Homo sapiens |
| NOV2m | CG51896-05 | 37 | 38 | Human semaphorin |
| | | | | 6A-1 - Homo sapiens |
| NOV2n | CG51896-06 | 39 | 40 | Human semaphorin |
| | | | | 6A-1 - Homo sapiens |
| NOV2o | CG51896-07 | 41 | 42 | Human semaphorin |
| | | | | 6A-1 - Homo sapiens |
| NOV2p | CG51896-08 | 43 | 44 | Human semaphorin |
| | | | | 6A-1 - Homo sapiens |
| NOV2q | CG51896-09 | 45 | 46 | Human semaphorin |
| | | | | 6A-1 - Homo sapiens |
| NOV2r | CG51896-10 | 47 | 48 | Human semaphorin |
| | | | | 6A-1 - Homo sapiens |
| NOV2s | CG51896-11 | 49 | 50 | Human semaphorin |
| | | | | 6A-1 - Homo sapiens |
| NOV2t | CG51896-12 | 51 | 52 | Human semaphorin |
| | | | | 6A-1 - Homo sapiens |
| NOV2u | CG51896-13 | 53 | 54 | Human semaphorin |
| | | | | 6A-1 - Homo sapiens |
| NOV2v | CG51896-14 | 55 | 56 | Human semaphorin |
| | | | | 6A-1 - Homo sapiens |

The proteins of the invention are useful in modulating(i.e. inhibiting or promoting) cell migration, actin filament formation and angiogenesis.

Cell migration, actin filament formation or angiogenesis is inhibited by contacting a cell or tissue with a NOVX polypeptide or nucleic acid. Alternatively, the cell or tissue is contacted with a compound that increases the expression or the activity or a NOVX polypeptide or nucleic acid. In contrast, cell migration, actin filament formation is promoted by contacting a cell or tissue with a compound that inhibits the expression or activity of a NOVX polypepeptide or nucleic acid. Compounds that inhibit the expression of a NOVX polypeptide or nucleic acid include for example NOVX specific antibodies or fragments thereof.

The NOVX polypeptide or nucleic acid is full length. Alternatively, the polypeptide or nucleic acid is less than full length (i.e., fragment) but retains the biological activity (e.g., cell migration inhibitio, anti-angiogenic) of the full length polypeptide. Optionally, the NOVX polypeptide or nucleic acid of fragment thereof is linked (e.g. covalently) to a compound that increases the half-life of the NOVX polypeptide of nucleic acid. Compounds that increase the half-life of a polypeptide in vivo are known in the art and include for example the Fc portion of an immunoglobulin molecule.

The cell or tissue is contacted in in vivo, ex vivo or in vitro. Alternatively, the cell or tissue is contacted indirectly (e.g. systemically) The cell or tissue is a normal, i.e. non-malignant. Alternatively, the cell or tissue is a cancerous, i.e., malignant. The cell or tissue is conatcte directly with the compound. The cell is any cell in which it is modulating cell migration or actin filament formation is desired. The cell is an endothelial cell, an epithelial cel, a neuronal cell, or a mesenchymal cell. The endothelial cell is for example a microvascular endothelial cell or a umbilical vein endothelial cell. The epithelial is for example, a renal cell or a pancreatic cell. The neuronal cell is a glial cell, an axonal cell or a dendritic cell. The cancer cell is for example a neuroblastoma cell, a renal carcinoma cell, a fibrosarcoma cell, a rhabdosarcoma cell or a pancreatic cancer cell.

The tissue is any tissue in which modulation of angiogenesis is desired. The tissue is an endothelial tissue (e.g., a vein, an artery or a microvasulature), an epithelial tissue (e.g., kidney, pancreatic or renal tissue), a neuronal tissue (e.g., glial, axonal or dendritic) or a mesenchymal tissue.

Cell migration is measure by methods known in the art. For example cell migration is measured using a chemotactant to attract cells to a lower surface of a membrane from an upper surface. The number of cells that migrate is used to measure the change in the ability of cells to migrate. Cell migration is also measured through assaying growth cone collapse in dorsal root ganglia. Growth cone collapse is measured through inspection of the dorsal root ganglia with a fluorescence microscope.

Angiogenesis is measure by methods known in the art. For example, angiogeneis is measured in vivo, using Matrigel plugs. Matrigel plugs with or without cancer cell lines (e.g. 786-0 cells) are placed in nude mice. After a period of time, the plugs are removed and examined to see if they contain any microvasculature. Alternatively, angiogenesis is measured by implanting nude mice with glioblastomas. The tumors are then monitored for increased vascularization, in the presence or absence of the protein of the invention.

Actin filament formation is measured by methods known in the art, e,g, microscopically.

Methods of Treatment

The invention provides for both prophylactic and therapeutic methods of treating or alleviating a symptom in a subject at risk of (or susceptible to) a disorder related to the modulation of cell migration and/or angiogenesis. NOVX polypeptides or nucleic acids are used to inhibit cell migration or angiogenesis in a subject. Alternatively, inhibitors of the NOVX polypeptides or nucleic acids are used to promote cell migration and angiogenesis.

Cell migration and angiogenesis related disorders are treated by administering to a subject a NOVX polypeptide, a NOVX nucleic acid or an inhibitor thereof. The subject is a mammal such as a human, mouse or rat. Administeration is either local or systemic.

Disorders in which inhibition of cell migration and/or angiogeneis is desired include but are not limited to, e.g., cancer such neuroblastoma, renal carcinoma, fibrosarcoma, rhabdosarcoma and pancreatic cancer. Disorders in which promotion of cell migration and/or angiogenesis include for example wound healing, tissue regeneration, especially nerve tissue regeneration, and promoting immune functions that involve cell mobility including extravasation of certain immune cells including megakaryocytes.

Efficaciousness of treatment is determined in association with any known method for diagnosing or treating the particular cell migration or angiogenesis related disorder. Alleviation of one or more symptoms of the disorder indicates that the compound confers a clinical benefit. Symptoms of cell migration and angiogenesis related disorders include loss of balance, weight loss, slow speech, jaundice, fatigue, pain, blood in urine, anemia, or swollen bones.

The methods described herein lead to a reduction in the severity or the allevialtion of one or more symptoms of cell migration/angiogenesis related disorder such as those described herein. Cell migration/angiogenesis related disorders are diagnosed and or monitored, typically by a physician using standard methodologies.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NOs: 13, 15, 17, 19, 21, 23, 25, 27, 29, 31, 33, 35, 37, 39, 41, 43, 45, 47, 49, 51, 53, and 55, or a complement of this nucleotide sequence, can be isolated using standard molecular biology techniques and the sequence information provided herein. Using all or a portion of the nucleic acid sequence of SEQ ID NOs: 13, 15,17, 19,21,23,25,27,29,31,33,35,37,39,41,43,45,47,49,51,53,and55,as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.).

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOs: 13, 15, 17, 19, 21, 23, 25, 27, 29, 31, 33, 35, 37, 39, 41, 43, 45, 47, 49, 51, 53, and 55, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOs: 13, 15, 17, 19, 21, 23, 25, 27, 29, 31, 33, 35, 37, 39, 41, 43, 45, 47, 49, 51, 53, and 55, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NOs: 13, 15, 17, 19, 21, 23, 25, 27, 29, 31, 33, 35, 37, 39, 41, 43, 45, 47, 49, 51, 53, and 55, is one that is sufficiently complementary to the nucleotide sequence of SEQ ID NOs: 13, 15, 17, 19, 21, 23, 25, 27, 29, 31, 33, 35, 37, 39, 41, 43, 45, 47, 49, 51, 53, and 55, that it can hydrogen bond with few or no mismatches to the nucleotide sequence shown in SEQ I) NOs: 13, 15, 17, 19, 21, 23, 25, 27, 29, 31, 33, 35, 37, 39, 41, 43, 45, 47, 49, 51, 53, and 55, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOs: 13, 15, 17, 19, 21, 23, 25, 27, 29, 31, 33, 35, 37, 39, 41, 43, 45, 47, 49, 51, 53, and 55, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOs: 13, 15, 17, 19, 21, 23, 25, 27, 29, 31, 33, 35, 37, 39, 41, 43, 45, 47, 49, 51, 53, and 55; or an anti-sense strand nucleotide sequence of SEQ ID NOs: 13, 15, 17, 19, 21, 23, 25, 27, 29, 31, 33, 35, 37, 39, 41, 43, 45, 47, 49, 51, 53, and 55; or of a naturally occurring mutant of SEQ ID NOs: 13, 15, 17, 19, 21, 23, 25, 27, 29, 31, 33, 35, 37, 39, 41, 43,45,47, 49, 51, 53, and 55.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NOs: 13, 15, 17, 19, 21, 23, 25, 27, 29, 31, 33, 35, 37, 39, 41, 43, 45, 47, 49, 51, 53, and 55, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NOs: 13, 15, 17, 19, 21, 23, 25, 27, 29, 31, 33, 35, 37, 39, 41, 43, 45, 47, 49, 51, 53, and 55, due to degeneracy of the genetic code and thus encode the same NOVX proteins as that encoded by the nucleotide sequences of SEQ ID NOs: 13, 15, 17, 19, 21, 23, 25, 27, 29, 31, 33, 35, 37, 39, 41, 43, 45, 47,49, 51, 53, and 55. In another embodiment, an isolated nucleic acid molecule of the invention has a nucleotide sequence encoding a protein having an amino acid sequence of SEQ ID NOs: 14, 16, 18, 20, 22, 24, 26, 28, 30, 32, 34, 36, 38, 40, 42, 44, 46, 48, 50, 52, 54, and 56.

In addition to the human NOVX nucleotide sequences of SEQ ID NOs: 13, 15, 17, 19, 21, 23, 25, 27, 29, 31, 33, 35, 37, 39, 41, 43, 45,47, 49, 51, 53, and 55, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NOs: 13, 15, 17, 19, 21, 23, 25, 27, 29, 31, 33, 35, 37, 39, 41, 43, 45, 47, 49, 51, 53, and 55, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOs: 13, 15, 17, 19,21,23,25,27,29, 31,33,35,37,39,41,43, 45,47,49,51,53, and 55. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NOs: 13, 15, 17, 19, 21, 23, 25, 27, 29, 31, 33, 35, 37, 39, 41, 43, 45, 47, 49, 51, 53, and 55, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOs: 13, 15, 17, 19, 21, 23, 25, 27, 29, 31, 33, 35, 37, 39, 41, 43, 45, 47, 49, 51, 53, and 55, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOs: 13, 15, 17, 19, 21, 23, 25, 27, 29, 31, 33, 35, 37, 39, 41, 43, 45, 47, 49, 51, 53, and 55, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NOs: 13, 15, 17, 19, 21, 23, 25, 27, 29, 31, 33, 35, 37, 39, 41, 43, 45, 47, 49, 51, 53, and 55, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NOs: 14, 16, 18, 20, 22, 24, 26, 28, 30, 32, 34, 36, 38, 40, 42, 44, 46, 48, 50, 52, 54, and 56. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOs: 13, 15, 17, 19, 21, 23, 25, 27, 29, 31, 33, 35, 37, 39, 41, 43, 45, 47, 49, 51, 53, and 55, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NOs: 14, 16, 18, 20, 22, 24, 26, 28, 30, 32, 34, 36, 38, 40, 42, 44, 46, 48, 50, 52, 54, and 56. Preferably, the protein encoded by the nucleic acid molecule is at least about 60% homologous to SEQ ID NOs: 14, 16, 18, 20, 22, 24, 26, 28, 30, 32, 34, 36, 38, 40, 42, 44, 46, 48, 50, 52, 54, and 56; more preferably at least about 70% homologous to SEQ ID NOs: 14, 16, 18, 20, 22, 24, 26, 28, 30, 32, 34, 36, 38, 40, 42, 44, 46, 48, 50, 52, 54, and 56; still more preferably at least about 80% homologous to SEQ ID NOs: 14, 16, 18, 20, 22, 24, 26, 28, 30, 32, 34, 36, 38, 40, 42, 44, 46, 48, 50, 52, 54, and 56; even more preferably at least about 90% homologous to SEQ ID NOs: 14, 16, 18, 20, 22, 24, 26, 28, 30, 32, 34, 36, 38, 40, 42, 44, 46, 48, 50, 52, 54, and 56; and most preferably at least about 95% homologous to SEQ ID NOs: 14, 16, 18, 20, 22, 24, 26, 28, 30, 32, 34, 36, 38, 40, 42,44, 46, 48, 50, 52, 54, and 56.

An isolated nucleic acid molecule encoding a NOVX protein homologous to the protein of SEQ ID NOs: 14, 16, 18, 20, 22, 24, 26, 28, 30, 32, 34, 36, 38, 40, 42, 44, 46, 48, 50, 52, 54, and 56, can be created by introducing one or more nucleotide substitutions, additions or deletions into the nucleotide sequence of SEQ ID NOs: 13, 15, 17, 19, 21, 23, 25, 27, 29, 31, 33, 35, 37, 39, 41, 43, 45, 47, 49, 51, 53, and 55, such that one or more amino acid substitutions, additions or deletions are introduced into the encoded protein.

Mutations can be introduced any one of SEQ ID NOs: 13, 15, 17, 19, 21, 23, 25, 27, 29, 31, 33, 35, 37, 39, 41, 43, 45, 47, 49, 51, 53, and 55, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NOs: 13, 15, 17, 19, 21, 23, 25, 27, 29, 31, 33, 35, 37, 39, 41, 43, 45, 47, 49, 51, 53, and 55, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SOND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (NI9)TT, as it is believed that the sequence of the 3′-most nucleotide of, the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX-) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NOs: 13, 15, 17, 19, 21, 23, 25, 27, 29, 31, 33, 35, 37, 39, 41, 43, 45, 47, 49, 51, 53, and 55, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NOs: 14, 16,.18, 20, 22, 24, 26, 28, 30, 32, 34, 36, 38, 40, 42, 44, 46,48, 50, 52, 54, and 56, or antisense nucleic acids complementary to a NOVX nucleic acid sequence of SEQ ID NOs: 13, 15, 17, 19, 21, 23, 25, 27, 29, 31, 33, 35, 37, 39, 41, 43, 45,47, 49, 51, 53, and 55, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NOs: 13, 15, 17, 19, 21, 23, 25, 27, 29, 31, 33, 35, 37, 39, 41, 43, 45, 47, 49, 51, 53, and 55). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NOs: 14, 16, 18, 20, 22, 24, 26, 28, 30, 32, 34, 36, 38, 40, 42, 44, 46, 48, 50, 52, 54, and 56. The invention also includes a mutant or variant protein any of whose residues may be changed from the corresponding residues shown in any one of SEQ ID NOs: 14, 16, 18, 20, 22, 24, 26, 28, 30, 32, 34, 36, 38, 40, 42, 44, 46, 48, 50, 52, 54, and 56, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NOs: 14, 16, 18, 20, 22, 24, 26, 28, 30, 32, 34, 36, 38, 40, 42, 44, 46, 48, 50, 52, 54, and 56) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NOs: 14, 16, 18, 20, 22, 24, 26, 28, 30, 32, 34, 36, 38, 40, 42, 44, 46, 48, 50, 52, 54, and 56. In other embodiments, the NOVX protein is substantially homologous to SEQ ID NOs: 14, 16, 18, 20, 22, 24, 26, 28, 30, 32, 34, 36, 38, 40, 42, 44, 46, 48, 50, 52, 54, and 56, and retains the functional activity of the protein of SEQ ID NOs: 14, 16, 18, 20, 22, 24, 26, 28, 30, 32, 34, 36, 38, 40, 42, 44, 46, 48, 50, 52, 54, and 56, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NOs: 14, 16, 18, 20, 22, 24, 26, 28, 30, 32, 34, 36, 38, 40, 42, 44, 46, 48, 50, 52, 54, and 56, and retains the functional activity of the NOVX proteins of SEQ ID NOs: 14, 16, 18, 20, 22, 24, 26, 28, 30, 32, 34, 36, 38, 40, 42, 44, 46, 48, 50, 52, 54, and 56.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NOs: 13, 15, 17, 19, 21, 23, 25, 27, 29, 31, 33, 35, 37, 39, 41, 43, 45, 47, 49, 51, 53, and 55.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NOs: 14, 16, 18, 20, 22, 24, 26, 28, 30, 32, 34, 36, 38, 40, 42, 44, 46, 48, 50, 52, 54, and 56, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. In one aspect of this embodiment, the immunoglobulin fusion protein is the Fc portion of the immunoglobulin. The Fc portion is fused to the N-terminus or C-terminus of NOVX. In a specific embodiment, the fusion protein is, for example, SEQ ID NOs: 50 and 54. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NOs: 14, 16, 18, 20, 22, 24, 26, 28, 30, 32, 34, 36, 38, 40, 42, 44, 46, 48, 50, 52, 54, and 56, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is <1 μM, preferably <100 nM, more preferably <10 nM, and most preferably <100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp.51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327.(1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison ( Nature 368, 812-13 (1994)); Fishwild et al,( Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS INENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurian and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pV_{L} series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banedji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NOs: 13, 15, 17, 19,21,23,25,27,29,31, 33,35,37,39,41,43,45, 47,49, 51, 53, and 55, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NOs: 13, 15, 17, 19, 21, 23, 25, 27, 29, 31, 33, 35, 37, 39, 41, 43, 45, 47, 49, 51, 53, and 55), but more preferably, is a non-human homologue of a human NOVX gene. For example, a mouse homologue of human NOVX gene of SEQ ID NOs: 13, 15, 17, 19, 21, 23, 25, 27, 29, 31, 33, 35, 37, 39, 41, 43, 45, 47, 49, 51, 53, and 55, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et. al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

The NOV2 clone was analyzed, and the nucleotide and encoded polypeptide sequences are shown in Table 2A.

TABLE 2A
NOV2 Sequence Analysis

| | | |
|---|---|---|
| NOV2a, CG51896-04 | SEQ ID NO: 13 | 4250 bp |
| DNA Sequence | ORF Start: ATG at 250 | ORF Stop: end of sequence |

GAACACATCGCGTTTGCATCCCAGAAAGTAGTCGCCGCGACTATTTCCCCCAAAGAGACAAGCACACA
TGTAGGAATGACAAAGGCTTGCGAAGGAGAGAGCGCAGCCCGCGGCCCGGAGAGATCCCCTCGATAAT
GGATTACTAAATGGGATACACGCTGTACCAGTTCGCTCCGAGCCCCGGCCGCCTGTCCGTCGATGCAC
CGAAAAGGGTGAAGTAGAGAAATAAAGTCTCCCCGCTGAACTACTATGAGGTCAGAAGCCTTGCTGCT
ATATTTCACACTGCTACACTTTGCTGGGGCTGGTTTCCCAGAAGATTCTGAGCCAATCAGTATTTCGC
ATTGCAACTATACAAAACAGTATCCGGTGTTTGTGGGCCACAAGCCAGGACGGAACACCACACAGAGG
CACAGGCTGGACATCCAGATGATTATGATCATGAACGGAACCCTCTACATTGCTGCTAGGGACCATAT
TTATACTGTTGATATAGACACATCACACACGGAAGAAATTTATTGTAGCAAAAAACTGACATGGAAAT
CTAGACAGGCCGATGTAGACACATGCAGAATGAAGGGAAAACATAAGGATGAGTGCCACAACTTTATT
AAAGTTCTTCTAAAGAAAAACGATGATGCATTGTTTGTCTGTGGAACTAATGCCTTCAACCCTTCCTG
CAGAAACTATAAGATGGATACATTGGAACCATTCGGGGATGAATTCAGCGGAATGGCCAGATGCCCAT
ATGATGCCAAACATGCCAACGTTGCACTGTTTGCAGATGGAAAACTATACTCAGCCACAGTGACTGAC
TTCCTTGCCATTGACGCAGTCATTTACCGGAGTCTTGGAGAAAGCCCTACCCTGCGGACCGTCAAGCA
CGATTCAAAATGGTTGAAAGAACCATACTTTGTTCAAGCCGTGGATTACGGAGATTATATCTACTTCT
TCTTCAGGGAAATAGCAGTGGAGTATAACACCATGGGAAAGGTAGTTTTCCCAAGAGTGGCTCAGGTT
TGTAAGAATGATATGGGAGGATCTCAAAGAGTCCTGGAGAAACAGTGGACGTCGTTCCTGAAGGCGCG
CTTGAACTGCTCAGTTCCTGGAGACTCTCATTTTTATTTCAACATTCTCCAGGCAGTTACAGATGTGA
TTCGTATCAACGGGCGTGATGTTGTCCTGGCAACGTTTTCTACACCTTATAACAGCATCCCTGGGTCT
GCAGTCTGTGCCTATGACATGCTTGACATTGCCAGTGTTTTTACTGGGAGATTCAAGGAACAGAAGTC
TCCTGATTCCACCTGGACACCAGTTCCTGATGAACGAGTTCCTAAGCCCAGGCCAGGTTGCTGTGCTG
GCTCATCCTCCTTAGAAAGATATGCAACCTCCAATGAGTTCCCTGATGATACCCTGAACTTCATCAAG
ACGCACCCGCTCATGGATGAGGCAGTGCCCTCCATCTTCAACAGGCCATGGTTCCTGAGAACAATGGT
CAGATACCGCCTTACCAAAATTGCAGTGGACACAGCTGCTGGGCCATATCAGAATCACACTGTGGTTT
TTCTGGGATCAGAGAAGGGAATCATCTTGAAGTTTTTGGCCAGAATAGGAAATAGTGGTTTTCTAAAT
GACAGCCTTTTCCTGGAGGAGATGAGTGTTTACAACTCTGAAAAATGCAGCTATGATGGAGTCGAAGA
CAAAAGGATCATGGGCATGCAGCTGGACAGAGCAAGCAGCTCTCTGTATGTTGCGTTCTCTACCTGTG
TGATAAAGGTTCCCCTTGGCCGGTGTGAACGACATGGGAAGTGTAAAAAAACCTGTATTGCCTCCAGA
GACCCATATTGTGGATGGATAAAGGAAGGTGGTGCCTGCAGCCATTTATCACCCAACAGCAGACTGAC
TTTTGAGCAGGACATAGAGCGTGGCAATACAGATGGTCTGGGGGACTGTCACAATTCCTTTGTGGCAC
TGAATGACATTTCAACTCCTCTACCAGATAATGAAATGTCTTACAACACAGTGTATGGGCATTCCAGT
TCCCTCTTGCCCAGCACAACCACATCAGATTCGACGGCTCAAGAGGGGTATGAGTCTAGGGGAGGAAT
GCTGGACTGGAAGCATCTGCTTGACTCACCTGACAGCACAGACCCTTTGGGGGCAGTGTCTTCCCATA
ATCACCAAGACAAGAAGGGAGTGATTCGGGAAAGTTACCTCAAAGGCCACGACCAGCTGGTTCCCGTC
ACCCTCTTGGCCATTGCAGTCATCCTGGCTTTCGTCATGGGGGCCGTCTTCTCGGGCATCACCGTCTA
CTGCGTCTGTGATCATCGGCGCAAAGACGTGGCTGTGGTGCAGCGCAAGGAGAAGGAGCTCACCCACT
CGCGCCGGGGCTCCATGAGCAGCGTCACCAAGCTCAGCGGCCTCTTTGGGGACACTCAATCCAAAGAC
CCAAAGCCGGAGGCCATCCTCACGCCACTCATGCACAACGGCAAGCTCGCCACTCCCGGCAACACGGC
CAAGATGCTCATTAAAGCAGACCAGCACCACCTGGACCTGACGGCCCTCCCCACCCCAGAGTCAACCC
CAACGCTGCAGCAGAAGCGGAAGCCCAGCCGCGGCAGCCGCGAGTGGGAGAGGAACCAGAACCTCATC
AATGCCTGCACAAAGGACATGCCCCCCATGGGCTCCCCTGTGATTCCCACGGACCTGCCCCTGCGGGC
CTCCCCCAGCCACATCCCCAGCGTGGTGGTCCTGCCCATCACGCAGCAGGGCTACCAGCATGAGTACG
TGGACCAGCCCAAAATGAGCGAGGTGGCCCAGATGGCGCTGGAGGACCAGGCCGCCACACTGGAGTAT
AAGACCATCAAGGAACATCTCAGCAGCAAGAGTCCCAACCATGGGGTGAACCTTGTGGAGAACCTGGA
CAGCCTGCCCCCCAAAGTTCCACAGCGGGAGGCCTCCCTGGGTCCCCCGGGAGCCTCCCTGTCTCAGA
CCGGTCTAAGCAAGCGGCTGGAAATGCACCACTCCTCTTCCTACGGGGTTGACTATAAGAGGAGCTAC
CCCACGAACTCGCTCACGAGAAGCCACCAGGCCACCACTCTCAAAAGAAACAACACTAACTCCTCCAA
TTCCTCTCACCTCTCCAGAAACCAGAGCTTTGGCAGGGGAGACAACCCGCCGCCCGCCCCGCAGAGGG
TGGACTCCATCCAGGTGCACAGCTCCCAGCCATCTGGCCAGGCCGTGACTGTCTCGAGGCAGCCCAGC
CTCAACGCCTACAACTCACTGACAAGGTCGGGGCTGAAGCGTACGCCCTCGCTAAAGCCGGACGTACC
CCCCAAACCATCCTTTGCTCCCCTTTCCACATCCATGAAGCCCAATGATGCGTGTACATAATCCCAGG
GGGAGGGGGTCAGGTGTCGAACCAGCAGGCAAGGCGAGGTGCCCGCTCAGCTCAGCAAGGTTCTCAAC
TGCCTCGAGTACCCACCAGACCAAGAAGGCCTGCGGCAGAGCCGAGGACGCTGGGTCCTCCTCTCTGG
GACACAGGGGTACTCACGAAAACTGGGCCGCGTGGTTTGGTGAAGGTTTGCAACGGCGGGGACTCACC
TTCATTCTCTTCCTTCACTTTCCCCCACACCCTACAACAGGTCGGACCCACAAAAGACTTCAGTTATC
ATCACAAACATGAGCCAAAAGCACATACCTACCCCATCCCCCACCCCCACACACACACACACATGCAC
ACAACACATACACACACACGCACAGAGGTGAACAGAAACTGAAACATTTTGTCCACAACTTCACGGGA
CGTGGCCAGACTGGGTTTGCGTTCCAACCTGCAAAACACAAATACATTTTTTAAAATCAAGAAAATTT
AAAAAGACAAAAAAAAAAGAATTCATTGATAATTCTAACTCAGACTTTAACAATGGCAGAAGTTTACT
ATGCGCAAATACTGTGAAATGCCCGCCAGTGTTACAGCTTTCTGTTGCAGCAGATAAATGCCATGTTG
GGCAACTATGTCATAGATTTCTGCTCCTCCTCTCTTTTAATGAAATAACGTGACCGTTAACGCAAGTA
ACTCTTTATTTATTGTTCACCCTTTTTTTCCTTAAGGAAAGGACTCTTCCAAATATCATCCTATGAAC
AGCTCTTCAGAAAGCCCATTGAAAGTTAAACTATTTAACGTGAAATCCATTAACTGGAATAATTGAGT
TTCTTTATTTTTACAATAAATTCACTGAGTAAAT

| | | | |
|---|---|---|---|
| NOV2a, CG51896-04 | | | |
| Protein Sequence | SEQ ID NO: 14 | 1047 aa | MW at 116354.6 kD |

MRSEALLLYFTLLHFAGAGFPEDSEPISISHCNYTKQYPVFVGHKPGRNTTQRHRLDIQMIMIMNGTL
YIAARDHIYTVDIDTSHTEEIYCSKKLTWKSRQADVDTCRMKGKHKDECHNFIKVLLKKNDDALFVCG
TNAFNPSCRNYKMDTLEPFGDEFSGMARCPYDAKHANVALFADGKLYSATVTDFLAIDAVIYRSLGES
PTLRTVKHDSKWLKEPYFVQAVDYGDYIYFFFREIAVEYNTMGKVVFPRVAQVCKNDMGGSQRVLEKQ
WTSFLKARLNCSVPGDSHFYFNILQAVTDVIRINGRDVVLATFSTPYNSIPGSAVCAYDMLDIASVFT
GRFKEQKSPDSTWTPVPDERVPKPRPGCCAGSSSLERYATSNEFPDDTLNFIKTHPLMDEAVPSIFNR
PWFLRTMVRYRLTKIAVDTAAGPYQNHTVVFLGSEKGIILKFLARIGNSGFLNDSLFLEEMSVYNSEK
CSYDGVEDKRIMGMQLDRASSSLYVAFSTCVIKVPLGRCERHGKCKKTCIASRDPYCGWIKEGGACSH
LSPNSRLTFEQDIERGNTDGLGDCHNSFVALNDISTPLPDNEMSYNTVYGHSSSLLPSTTTSDSTAQE
GYESRGGMLDWKHLLDSPDSTDPLGAVSSHNHQDKKGVIRESYLKGHDQLVPVTLLAIAVILAFVMGA
VFSGITVYCVCDHRRKDVAVVQRKEKELTHSRRGSMSSVTKLSGLFGDTQSKDPKPEAILTPLMHNGK
LATPGNTAKMLIKADQHHLDLTALPTFESTPTLQQKRKPSRGSREWERNQNLINACTKDMPPMGSPVI
PTDLPLRASPSHIPSVVVLPITQQGYQHEYVDQPKMSEVAQMALEDQAATLEYKTIKEHLSSKSPNHG
VNLVENLDSLPPKVPQREASLGPPGASLSQTGLSKRLEMHHSSSYGVDYKRSYPTNSLTRSHQATTLK
RNNTNSSNSSHLSRNQSFGRGDNPPPAPQRVDSIQVHSSQPSGQAVTVSRQPSLNAYNSLTRSGLKRT
PSLKPDVPPKPSFAPLSTSMKPNDACT

| | | |
|---|---|---|
| NOV2b, 271674560 | SEQ ID NO: 15 | 1921 bp |
| DNA Sequence | ORF Start: at 1 | ORF Stop: end of sequence |

GCCGGATCCAGTATTTCGCATTGCAACTATACAAAACAGTATCCGGTGTTTGTGGGCCACAAGCCAGG
ACGGAACACCACACAGAGGCACAGGCTGGACATCCAGATGATTATGATCATGAACGGAACCCTCTACA
TTGCTGCTAGGGACCATATTTATACTGTTGATATAGACACATCACACACGGAAGAAATTTATTGTAGC
AAAAAACTGACATGGAAATCTAGACAGGCCGATGTAGACACATGCAGAATGAAGGGAAAACATAAGGA
TGAGTGCCACAACTTTATTAAAGTTCTTCTAAAGAAAAACGATGATGCATTGTTTGTCTGTGGAACTA
ATGCCTTCAACCCTTCCTGCAGAAACTATAAGATGGATACATTGGAACCATTCGGGGATGAATTCAGC
GGAATGGCCAGATGCCCATATGATGCCAAACATGCCAACGTTGCACTGTTTGCAGATGGAAAACTATA
CTCAGCCACAGTGACTGACTTCCTTGCCATTGACGCAGTCATTTACCGGAGTCTTGGAGAAAGCCCTA
CCCTGCGGACCGTCAAGCACGATTCAAAATGGTTGAAAGAACCATACTTTGTTCAAGCCGTGGATTAC
GGAGATTATATCTACTTCTTCTTCAGGGAAATAGCAGTGGAGTATAACACCATGGGAAAGGTAGTTTT
CCCAAGAGTGGCTCAGGTTTGTAAGAATGATATGGGAGGATCTCAAAGAGTCCTGGAGAAACAGTGGA
CGTCGTTCCTGAAGGCGCGCTTGAACTGCTCAGTTCCTGGAGACTCTCATTTTTATTTCAACATTCTC
CAGGCAGTTACAGATGTGATTCGTATCAACGGGCGTGATGTTGTCCTGGCAACGTTTTCTACACCTTA
TAACAGCATCCCTGGGTCTGCAGTCTGTGCCTATGACATGCTTGACATTGCCAGTGTTTTTACTGGGA
GATTCAAGGAACAGAAGTCTCCTGATTCCACCTGGACACCAGTTCCTGATGAACGAGTTCCTAAGCCC
AGGCCAGGTTGCTGTGCTGGCTCATCCTCCTTAGAAAGATATGCAACCTCCAATGAGTTCCCTGATGA
TACCCTGAACTTCATCAAGACGCACCCGCTCATGGATGAGGCAGTGCCCTCCATCTTCAACAGGCCAT
GGTTCCTGAGAACAATGGTCAGATACCGCCTTACCAAAATTGCAGTGGACACAGCTGCTGGGCCATAT
CAGAATCACACTGTGGTTTTTCTGGGATCAGAGAAGGGAATCATCTTGAAGTTTTTGGCCAGAATAGG
AAATAGTGGTTTTCTAAATGACAGCCTTTTCCTGGAGGAGATGAGTGTTTACAACTCTGAAAAATGCA
GCTATGATGGAGTCGAAGACAAAAGGATCATGGGCATGCAGCTGGACAGAGCAAGCAGCTCTCTGTAT
GTTGCGTTCTCTACCTGTGTGATAAAGGTTCCCCTTGGCCGGTGTGAACGACATGGGAAGTGTAAAAA
AACCTGTATTGCCTCCAGAGACCCGTATTGTGGATGGATAAAGGAAGGTGGTGCCTGCAGCCATTTAT
CACCCAACAGCAGACTGACTTTTGAGCAGGACATAGAGCGTGGCAATACAGATGGTCTGGGGGACTGT
CACAATTCCTTTGTGGCACTGAATGACATTTCAACTCCTCTACCAGATAATGAAATGTCTTACAACAC
AGTGTATGGGCATTCCAGTTCCCTCTTGCCCAGCACAACCACATCAGATTCGACGGCTCAAGAGGGGT
ATGAGTCTAGGGGAGGAATGCTGGACTGGAAGCATCTGCTTGACTCACCTGACAGCACAGACCCTTTG
GGGGCAGTGTCTTCCCATAATCACCAAGACAAGAAGGGAGTGATTCGGGAAAGTTACCTCAAAGGCCA
CGACCAGGTCGACGGTG

| | | | |
|---|---|---|---|
| NOV2b, 271674560 | | | |
| Protein Sequence | SEQ ID NO: 16 | 1640 aa | MW at 71799.4 kD |

AGSSISHCNYTKQYPVFVGHKPGRNTTQRHRLDIQMIMIMNGTLYIAARDHIYTVDIDTSHTEEIYCS
KKLTWKSRQADVDTCRMKGKHKDECHNFIKVLLKKNDDALFVCGTNAFNPSCRNYKMDTLEPFGDEFS
GMARCPYDAKHANVALFADGKLYSATVTDFLAIDAVIYRSLGESPTLRTVKHDSKWLKEPYFVQAVDY
GDYIYFFFREIAVEYNTMGKVVFPRVAQVCKNDMGGSQRVLEKQWTSFLKARLNCSVPGDSHFYFNIL
QAVTDVIRINGRDVVLATFSTPYNSIPGSAVCAYDMLDIASVFTGRFKEQKSPDSTWTPVPDERVPKP
RPGCCAGSSSLERYATSNEFPDDTLNFIKTHPLMDEAVPSIFNRPWFLRTMVRYRLTKIAVDTAAGPY
QNHTVVFLGSEKGIILKFLARIGNSGFLNDSLFLEEMSVYNSEKCSYDGVEDKRIMGMQLDRASSSLY
VAFSTCVIKVPLGRCERHGKCKKTCIASRDPYCGWIKEGGACSHLSPNSRLTFEQDIERGNTDGLGDC
HNSFVALNDISTPLPDNEMSYNTVYGHSSSLLPSTTTSDSTAQEGYESRGGMLDWKHLLDSPDSTDPL
GAVSSHNHQDKKGVIRESYLKGHDQVDG

| | | |
|---|---|---|
| NOV2c, 267441133 | SEQ ID NO:17 | 3106 bp |
| DNA Sequence | ORF Start: at 2 | ORF Stop: end of sequence |

CACCGGATCCGGTTTCCCAGAAGATTCTGAGCCAATCAGTATTTCGCATGGCAACTATACAAAACAGT
ATCCGGTGTTTGTGGGCCACAAGCCAGGACGGAACACCACACAGAGGCACAGGCTGGACATCCAGATG
ATTATGATCATGAACGGAACCCTCTACATTGCTGCTAGGGACCATATTTATACTGTTGATATAGACAC
ATCACACACGGAAGAAATTTATTGTAGCAAAAAACTGACATGGAAATCTAGACAGGCCGATGTAGACA
CATGCAGAATGAAGGGAAAACATAAGGATGAGTGCCACAACTTTATTAAAGTTCTTCTAAAGAAAAAC
GATGATGCATTGTTTGTCTGTGGAACTAATGCCTTCAACCCTTCCTGCAGAAACTATAAGATGGATAC
ATTGGAACCATTCGGGGATGAATTCAGCGGAATGGCCAGATGCCCATATGATGCCAAACATGCCAACG
TTGCACTGTTTGCAGATGGAAAACTATACTCAGCCACAGTGACTGACTTCCTTGCCATTGACGCAGTC
ATTTACCGGAGTCTTGGAGAAAGCCCTACCCTGCGGACCGTCAAGCACGATTCAAAATGGTTGAAAGA
ACCATACTTTGTTCAAGCCGTGGATTACGGAGATTATATCTACTTCTTCTTCAGGGAAATAGCAGTGG
AGTATAACACCATGGGAAAGGTAGTTTTCCCAAGAGTGGCTCAGGTTTGTAAGAATGATATGGGAGGA
TCTCAAAGAGTCCTGGAGAAACAGTGGACGTCGTTCCTGAAGGCGCGCTTGAACTGCTCAGTTCCTGG
AGACTCTCATTTTTATTTCAACATTCTCCAGGCAGTTACAGATGTGATTCGTATCAACGGGCGTGATG
TTGTCCTGGCAACGTTTTCTACACCTTATAACAGCATCCCTGGGTCTGCAGTCTGTGCCTATGACATG
CTTGACATTGCCAGTGTTTTTACTGGGAGATTCAAGGAACAGAAGTCTCCTGATTCCACCTGGACACC
AGTTCCTGATGAACGAGTTCCTAAGCCCAGGCCAGGTTGCTGTGCTGGCTCATCCTCCTTAGAAAGAT
ATGCAACCTCCAATGAGTTCCCTGATGATACCCTGAACTTCATCAAGACGCACCCGCTCATGGATGAG
GCAGTGCCCTCCATCTTCAACAGGCCATGGTTCCTGAGAACAATGGTCAGATACCGCCTTACCAAAAT
TGCAGTGGACACAGCTGCTGGGCCATATCAGAATCACACTGTGGTTTTTCTGGGATCAGAGAAGGGAA
TCATCTTGAAGTTTTTGGCCAGAATAGGAAATAGTGGTTTTCTAAATGACAGCCTTTTCCTGGAGGAG
ATGAGTGTTTACAACTCTGAAAAATGCAGCTATGATGGAGTCGAAGACAAAAGGATCATGGGCATGCA
GCTGGACAGAGCAAGCAGCTCTCTGTATGTTGCGTTCTCTACCTGTGTGATAAAGGTTCCCCTTGGCC
GGTGTGAACGACATGGGAAGTGTAAAAAAACCTGTATTGCCTCCAGAGACCCGTATTGTGGATGGATA
AAGGAAGGTGGTGCCTGCAGCCATTTATCACCCAACAGCAGACTGACTTTTGAGCAGGACATAGAGCG
TGGCAATACAGATGGTCTGGGGGACTGTCACAATTCCTTTGTGGCACTGAATGACATTTCAACTCCTC
TACCAGATAATGAAATGTCTTACAACACAGTGTATGGGCATTCCAGTTCCCTCTTGCCCAGCACAACC
ACATCAGATTCGACGGCTCAAGAGGGGTATGAGTCTAGGGGAGGAATGCTGGACTGGAAGCATCTGCT
TGACTCACCTGACAGCACAGACCCTTTGGGGGCAGTGTCTTCCCATAATCACCAAGACAAGAAGGGAG
TGATTCGGGAAAGTTACCTCAAAGGCCACGACCAGCTGGTTCCCGTCACCCTCTTGGCCATTGCAGTC
ATCCTGGCTTTCGTCATGGGGGCCGTCTTCTCGGGCATCACCGTCTACTGCGTCTGTGATCATCGGCG
CAAAGACGTGGCTGTGGTGCAGCGCAAGGAGAAGGAGCTCACCCACTCGCGCCGGGGCTCCATGAGCA
GCGTCACCAAGCTCAGCGGCCTCTTTGGGGACACTCAATCCAAAGACCCAAAGCCGGAGGCCATCCTC
ACGCCACTCATGCACAACGGCAAGCTCGCCACTCCCGGCAACACGGCCAAGATGCTCATTAAAGCAGA
CCAGCACCACCTGGACCTGACGGCCCTCCCCACCCCAGAGTCAACCCCAACGCTGCAGCAGAAGCGGA
AGCCCAGCCGCGGCAGCCGCGAGTGGGAGAGGAACCAGAACCTCATCAATGCCTGCACAAAGGACATG
CCCCCCATGGGCTCCCCTGTGATTCCCACGGACCTGCCCCTGCGGGCCTCCCCCAGCCACATCCCCAG
CGTGGTGGTCCTGCCCATCACGCAGCAGGGCTACCAGCATGAGTACGTGGACCAGCCCAAAATGAGCG
AGGTGGCCCAGATGGCGCTGGAGGACCAGGCCGCCACACTGGAGTATAAGACCATCAAGGAACATCTC
AGCAGCAAGAGTCCCAACCATGGGGTGAACCTTGTGGAGAACCTGGACAGCCTGCCCCCCAAAGTTCC
ACAGCGGGAGGCCTCCCTGGGTCCCCCGGGAGCCTCCCTGTCTCAGACCGGTCTAAGCAAGCGGCTGG
AAATGCACCACTCCTCTTCCTACGGGGTTGACTATAAGAGGAGCTACCCCACGAACTCGCTCACGAGA
AGCCACCAGGCCACCACTCTCAAAAGAAACAACACTAACTCCTCCAATTCCTCTCACCTCTCCAGAAA
CCAGAGCTTTGGCAGGGGAGACAACCCGCCGCCCGCCCCGCAGAGGGTGGACTCCATCCAGGTGCACA
GCTCCCAGCCATCTGGCCAGGCCGTGACTGTCTCGAGGCAGCCCAGCCTCAACGCCTACAACTCACTG
ACAAGGTCGGGGCTGAAGCGTACGCCCTCGCTAAAGCCGGACGTACCCCCCAAACCATCCTTTGCTCC
CCTTTCCACATCCATGAAGCCCAATGATGCGTGTACAGTCGACGGC

| | | | |
|---|---|---|---|
| NOV2c, 267441133 | | | |
| Protein Sequence | SEQ ID NO: 18 | 1035 aa | MW at 114789.6 kD |

TGSGFPEDSEPISISHGNYTKQYPVFVGHKPGRNTTQRHRLDIQMIMIMNGTLYIAARDHIYTVDIDT
SHTEEIYCSKKLTWKSRQADVDTCRMKGKHKDECHNFIKVLLKKNDDALFVCGTNAFNPSCRNYKMDT
LEPFGDEFSGMARCPYDAKHANVALFADGKLYSATVTDFLAIDAVIYRSLGESPTLRTVKHDSKWLKE
PYFVQAVDYGDYIYFFFREIAVEYNTMGKVVFPRVAQVCKNDMGGSQRVLEKQWTSFLKARLNCSVPG
DSHFYFNILQAVTDVIRINGRDVVLATFSTPYNSIPGSAVCAYDMLDIASVFTGRFKEQKSPDSTWTP
VPDERVPKPRPGCCAGSSSLERYATSNEFPDDTLNFIKTHPLMDEAVPSIFNRPWFLRTMVRYRLTKI
AVDTAAGPYQNHTVVFLGSEKGIILKFLARIGNSGFLNDSLFLEEMSVYNSEKCSYDGVEDKRIMGMQ
LDRASSSLYVAFSTCVIKVPLGRCERHGKCKKTCIASRDPYCGWIKEGGACSHLSPNSRLTFEQDIER
GNTDGLGDCHNSFVALNDISTPLPDNEMSYNTVYGHSSSLLPSTTTSDSTAQEGYESRGGMLDWKHLL
DSPDSTDPLGAVSSHNHQDKKGVIRESYLKGHDQLVPVTLLAIAVILAFVMGAVFSGITVYCVCDHRR
KDVAVVQRKEKELTHSRRGSMSSVTKLSGLFGDTQSKDPKPEAILTPLMHNGKLATPGNTAKMLIKAD
QHHLDLTALPTPESTPTLQQKRKPSRGSREWERNQNLINACTKDMPPMGSPVIPTDLPLRASPSHIPS
VVVLPITQQGYQHEYVDQPKMSEVAQMALEDQAATLEYKTIKEHLSSKSPNHGVNLVENLDSLPPKVP
QREASLGPPGASLSQTGLSKRLEMHHSSSYGVDYKRSYPTNSLTRSHQATTLKRNNTNSSNSSHLSRN
QSFGRGDNPPPAPQRVDSIQVHSSQPSGQAVTVSRQPSLNAYNSLTRSGLKRTPSLKPDVPPKPSFAP
LSTSMKPNDACTVDG

| | | |
|---|---|---|
| NOV2d, 267441137 | SEQ ID NO: 19 | 2995 bp |
| DNA Sequence | ORF Start: at 2 | ORF Stop: end of sequence |

CACCGGATCCCTGGACATCCAGATGATTATGATCATGAACGGAACCCTCTACATTGCTGCTAGGGACC
ATATTTATACTGTTGATATAGACACATCACACACGGAAGAAATTTATTGTAGCAAAAAACTGACATGG
AAATCTAGACAGGCCGATGTAGACACATGCAGAATGAAGGGAAAACATAAGGATGAGTGCCACAACTT
TATTAAAGTTCTTCTAAAGAAAAACGATGATGCATTGTTTGTCTGTGGAACTAATGCCTTCAACCCTT
CCTGCAGAAACTATAAGATGGATACATTGGAACCATTCGGGGATGAATTCAGCGGAATGGCCAGATGC
CCATATGATGCCAAACATGCCAACGTTGCACTGTTTGCAGATGGAAAACTATACTCAGCCACAGTGAC
TGACTTCCTTGCCATTGACGCAGTCATTTACCGGAGTCTTGGAGAAAGCCCTACCCTGCGGACCGTCA
AGCACGATTCAAAATGGTTGAAAGAACCATACTTTGTTCAAGCCGTGGATTACGGAGATTATATCTAC
TTCTTCTTCAGGGAAATAGCAGTGGAGTATAACACCATGGGAAAGGTAGTTTTCCCAAGAGTGGCTCA
GGTTTGTAAGAATGATATGGGAGGATCTCAAAGAGTCCTGGAGAAACAGTGGACGTCGTTCCTGAAGG
CGCGCTTGAACTGCTCAGTTCCTGGAGACTCTCATTTTTATTTCAACATTCTCCAGGCAGTTACAGAT
GTGATTCGTATCAACGGGCGTGATGTTGTCCTGGCAACGTTTTCTACACCTTATAACAGCATCCCTGG
GTCTGCAGTCTGTGCCTATGACATGCTTGACATTGCCAGTGTTTTTACTGGGAGATTCAAGGAACAGA
AGTCTCCTGATTCCACCTGGACACCAGTTCCTGATGAACGAGTTCCTAAGCCCAGGCCAGGTTGCTGT
GCTGGCTCATCCTCCTTAGAAAGATATGCAACCTCCAATGAGTTCCCTGATGATACCCTGAACTTCAT
CAAGACGCACCCGCTCATGGATGAGGCAGTGCCCTCCATCTTCAACAGGCCATGGTTCCTGAGAACAA
TGGTCAGATACCGCCTTACCAAAATTGCAGTGGACACAGCTGCTGGGCCATATCAGAATCACACTGTG
GTTTTTCTGGGATCAGAGAAGGGAATCATCTTGAAGTTTTTGGCCAGAATAGGAAATAGTGGTTTTCT
AAATGACAGCCTTTTCCTGGAGGAGATGAGTGTTTACAACTCTGAAAAATGCAGCTATGATGGAGTCG
AAGACAAAAGGATCATGGGCATGCAGCTGGACAGAGCAAGCAGCTCTCTGTATGTTGCGTTCTCTACC
TGTGTGATAAAGGTTCCCCTTGGCCGGTGTGAACGACATGGGAAGTGTAAAAAAACCTGTATTGCCTC
CAGAGACCCGTATTGTGGATGGATAAAGGAAGGTGGTGCCTGCAGCCATTTATCACCCAACAGCAGAC
TGACTTTTGAGCAGGACATAGAGCGTGGCAATACAGATGGTCTGGGGGACTGTCACAATTCCTTTGTG
GCACTGAATGACATTTCAACTCCTCTACCAGATAATGAAATGTCTTATAACACAGTGTATGGGCATTC
CAGTTCCCTCTTGCCCAGCACAACCACATCAGATTCGACGGCTCAAGAGGGGTATGAGTCTAGGGGAG
GAATGCTGGACTGGAAGCATCTGCTTGACTCACCTGACAGCACAGACCCTTTGGGGGCAGTGTCTTCC
CACAATCACCAAGACAAGAAGGGAGTGATTCGGGAAAGTTACCTCAAAGGCCACGACCAGCTGGTTCC
CGTCACCCTCTTGGCCATTGCAGTCATCCTGGCTTTCGTCATGGGGGCCGTCTTCTCGGGCATCACCG
TCTACTGCGTCTGTGATCATCGGCGCAAAGACGTGGCTGTGGTGCAGCGCAAGGAGAAGGAGCTCACC
CACTCGCGCCGGGGCTCCATGAGCAGCGTCACCAAGCTCAGCGGCCTCTTTGGGGACACTCAATCCAA
AGACCCAAAGCCGGAGGCCATCCTCACGCCACTCATGCACAACGGCAAGCTCGCCACTCCCGGCAACA
CGGCCAAGATGCTCATTAAAGCAGACCAGCACCACCTGGACCTGACGGCCCTCCCCACCCCAGAGTCA
ACCCCAACGCTGCAGCAGAAGCGGAAGCCCAGCCGCGGCAGCCGCGAGTGGGAGAGGAACCAGAACCT
CATCAATGCCTGCACAAAGGACATGCCCCCCATGGGCTCCCCTGTGATTCCCACGGACCTGCCCCTGC
GGGCCTCCCCCAGCCACATCCCCAGCGTGGTGGTCCTGCCCATCACGCAGCAGGGCTACCAGCATGAG
TACGTGGACCAGCCCAAAATGAGCGAGGTGGCCCAGATGGCGCTGGAGGACCAGGCCGCCACACTGGA
GTATAAGACCATCAAGGAACATCTCAGCAGCAAGAGTCCCAACCATGGGGTGAACCTTGTGGAGAACC
TGGACAGCCTGCCCCCCAAAGTTCCACAGCGGGAGGCCTCCCTGGGTCCCCCGGGAGCCTCCCTGTCT
CAGACCGGTCTAAGCAAGCGGCTGGAAATGCACCACTCCTCTTCCTACGGGGTTGACTATAAGAGGAG
CTACCCCACGAACTCGCTCACGAGAAGCCACCAGGCCACCACTCTCAAAAGAAACAACACTAACTCCT
CCAATTCCTCTCACCTCTCCAGAAACCAGAGCTTTGGCAGGGGAGACAACCCGCCGCCCGCCCCGCAG
AGGGTGGACTCCATCCAGGTGCACAGCTCCCAGCCATCTGGCCAGGCCGTGACTGTCTCGAGGCAGCC
CAGCCTCAACGCCTACAACTCACTGACAAGGTCGGGGCTGAAGCGTACGCCCTCGCTAAAGCCGGACG
TACCCCCCAAACCATCCTTTGCTCCCCTTTCCACATCCATGAAGCCCAATGATGCGTGTACAGTCGAC
GGC

| | | | |
|---|---|---|---|
| NOV2d,267441137 | | | |
| Protein Sequence | SEQ ID NO: 20 | 998 aa | MW at 110569.0 kD |

TGSLDIQMIMIMNGTLYIAARDHIYTVDIDTSHTEEIYCSKKLTWKSRQADVDTCRMKGKHKDECHNF
IKVLLKKNDDALFVCGTNAFNPSCRNYKMDTLEPFGDEFSGMARCPYDAKHANVALFADGKLYSATVT
DFLAIDAVIYRSLGESPTLRTVKHDSKWLKEPYFVQAVDYGDYIYFFFREIAVEYNTMGKVVFPRVAQ
VCKNDMGGSQRVLEKQWTSFLKARLNCSVPGDSHFYFNILQAVTDVIRINGRDVVLATFSTPYNSIPG
SAVCAYDMLDIASVFTGRFKEQKSPDSTWTPVPDERVPKPRPGCCAGSSSLERYATSNEFPDDTLNFI
KTHPLMDEAVPSIFNRPWFLRTMVRYRLTKIAVDTAAGPYQNHTVVFLGSEKGIILKFLARIGNSGFL
NDSLFLEEMSVYNSEKCSYDGVEDKRIMGMQLDRASSSLYVAFSTCVIKVPLGRCERHGKCKKTCIAS
RDPYCGWIKEGGACSHLSPNSRLTFEQDIERGNTDGLGDCHNSFVALNDISTPLPDNEMSYNTVYGHS
SSLLPSTTTSDSTAQEGYESRGGMLDWKHLLDSPDSTDPLGAVSSHNHQDKKGVIRESYLKGHDQLVP
VTLLAIAVILAFVMGAVFSGITVYCVCDHRRKDVAVVQRKEKELTHSRRGSMSSVTKLSGLFGDTQSK
DPKPEAILTPLMHNGKLATPGNTAKMLIKADQHHLDLTALPTPESTPTLQQKRKPSRGSREWERNQNL
ENACTKDMPPMGSPVIPTDLPLRASPSHIPSVVVLPITQQGYQHEYVDQPKMSEVAQMALEDQAATLE
YKTIKEHLSSKSPNHGVNLVENLDSLPPKVPQREASLGPPGASLSQTGLSKRLEMHHSSSYGVDYKRS
YPTNSLTRSHQATTLKRNNTNSSNSSHLSRNQSFGRGDNPPPAPQRVDSIQVHSSQPSGQAVTVSRQP
SLNAYNSLTRSGLKRTPSLKPDVPPKPSFAPLSTSMKPNDACTVDG

| | | |
|---|---|---|
| NOV2e, 262254987 | SEQ ID NO: 21 | 1327 bp |
| DNA Sequence | ORF Start: at 2 | ORF Stop: end of sequence |

CACCGGATCCCTGGACATCCAGATGATTATGATCATGAACGGAACCCTCTACATTGCTGCTAGGGACC
ATATTTATACTGTTGATATAGACACATCACACACGGAAGAAATTTATTGTAGCAAAAAACTGACATGG
AAATCTAGACAGGCCGATGTAGACACATGCAGAATGAAGGGAAAACATAAGGATGAGTGCCACAACTT
TATTAAAGTTCTTCTAAAGAAAAACGATGATGCATTGTTTGTCTGTGGAACTAATGCCTTCAACCCTT
CCTGCAGAAACTATAAGATGGATACATTGGAACCATTCGGGGATGAATTCAGCGGAATGGCCAGATGC
CCATATGATGCCAAACATGCCAACGTTGCACTGTTTGCAGATGGAAAACTATACTCAGCCACAGTGAC
TGACTTCCTTGCCATTGACGCAGTCATTTACCGGAGTCTTGGAGAAAGCCCTACCCTGCGGACCGTCA
AGCACGATTCAAAATGGTTGAAAGAACCATACTTTGTTCAAGCCGTGGATTACGGAGATTATATCTAC
TTCTTCTTCAGGGAAATAGCAGTGGAGTATAACACCATGGGAAAGGTAGTTTTCCCAAGAGTGGCTCA
GGTTTGTAAGAATGATATGGGAGGATCTCAAAGAGTCCTGGAGAAACAGTGGACGTCGTTCCTGAAGG
CGCGCTTGAACTGCTCAGTTCCTGGAGACTCTCATTTTTATTTCAACATTCTCCAGGCAGTTACAGAT
GTGATTCGTATCAACGGGCGTGATGTTGTCCTGGCAACGTTTTCTACACCTTATAACAGCATCCCTGG
GTCTGCAGTCTGTGCCTATGACATGCTTGACATTGCCAGTGTTTTTACTGGGAGATTCAAGGAACAGA
AGTCTCCTGATTCCACCTGGACACCAGTTCCTGATGAACGAGTTCCTAAGCCCAGGCCAGGTTGCTGT
GCTGGCTCATCCTCCTTAGAAAGATATGCAACCTCCAATGAGTTCCCTGATGATACCCTGAACTTCAT
CAAGACGCACCCGCTCATGGATGAGGCAGTGCCCTCCATCTTCAACAGGCCATGGTTCCTGAGAACAA
TGGTCAGATACCGCCTTACCAAAATTGCAGTGGACACAGCTGCTGGGCCATATCAGAATCACACTGTG
GTTTTTCTGGGATCAGAGAAGGGAATCATCTTGAAGTTTTTGGCCAGAATAGGAAATAGTGGTTTTCT
AAATGACAGCCTTTTCCTGGAGGAGATGAGTGTTTACAACTCTGAAAAATGCAGCTATGATGGAGTCG
AAGACAAAAGGATCATGGGCATGCAGGTCGACGGC

| | | | |
|---|---|---|---|
| NOV2e, 262254987 | | | |
| Protein Sequence | SEQ ID NO: 22 | 442 aa | MW at 49986.5 kD |

TGSLDIQMIMIMNGTLYIAARDHIYTVDIDTSHTEEIYCSKKLTWKSRQADVDTCRMKGKNKDECHNF
IKVLLKKNDDALFVCGTNAFNPSCRNYKMDTLEPFGDEFSGMARCPYDAKHANVALFADGKLYSATVT
DFLAIDAVIYRSLGESPTLRTVKNDSKWLKEPYFVQAVDYGDYIYFFFREIAVEYNTMGKVVFPRVAQ
VCKNDMGGSQRVLEKQWTSFLKARLNCSVPGDSNFYFNILQAVTDVIRINGRDVVLATFSTPYNSIPG
SAVCAYDMLDIASVFTGRFKEQKSPDSTWTPVPDERVPKPRPGCCAGSSSLERYATSNEFPDDTLNFI
KTHPLMDEAVPSIFNRPWFLRTMVRYRLTKIAVDTAAGPYQNHTVVFLGSEKGIILKFLARIGNSGFL
NDSLFLEEMSVYNSEKCSYDGVEDKRIMGMQVDG

| | | |
|---|---|---|
| NOV2f, 260565761 | SEQ ID NO: 23 | 1492 bp |
| DNA Sequence | ORF Start: at 2 | ORF Stop: end of sequence |

CACCGGATCCATGAGGTCAGAAGCCTTGCTGCTATATTTCACACTGCTACACTTTGCTGGGGCTGGTT
TCCCAGAAGATTCTGAGCCAATCAGTATTTCGCATGGCAACTATACAAAACAGTATCCGGTGTTTGTG
GGCCACAAGCCAGGACGGAACACCACACAGAGGCACAGGCTGGACATCCAGATGATTATGATCATGAA
CGGAACCCTCTACATTGCTGCTAGGGACCATATTTATACTGTTGATATAGACACATCACACACGGAAG
AAATTTATTGTAGCAAAAAACTGACATGGAAATCTAGACAGGCCGATGTAGACACATGCAGAATGAAG
GGAAAACATAAGGATGAGTGCCACAACTTTATTAAAGTTCTTCTAAAGAAAAACGATGATGCATTGTT
TGTCTGTGGAACTAATGCCTTCAACCCTTCCTGCAGAAACTATAAGATGGATACATTGGAACCATTCG
GGGATGAATTCAGCGGAATGGCCAGATGCCCATATGATGCCAAACATGCCAACGTTGCACTGTTTGCA
GATGGAAAACTATACTCAGCCACAGTGACTGACTTCCTTGCCATTGACGCAGTCATTTACCGGAGTCT
TGGAGAAAGCCCTACCCTGCGGACCGTCAAGCACGATTCAAAATGGTTGAAAGAACCATACTTTGTTC
AAGCCGTGGATTACGGAGATTATATCTACTTCTTCTTCAGGGAAATAGCAGTGGAGTATAACACCATG
GGAAAGGTAGTTTTCCCAAGAGTGGCTCAGGTTTGTAAGAATGATATGGGAGGATCTCAAAGAGTCCT
GGAGAAACAGTGGACGTCGTTCCTGAAGGCGCGCTTGAACTGCTCAGTTCCTGGAGACTCTCATTTTT
ATTTCAACATTCTCCAGGCAGTTACAGATGTGATTCGTATCAACGGGCGTGATGTTGTCCTGGCAACG
TTTTCTACACCTTATAACAGCATCCCTGGGTCTGCAGTCTGTGCCTATGACATGCTTGACATTGCCAG
TGTTTTTACTGGGAGATTCAAGGAACAGAAGTCTCCTGATTCCACCTGGACACCAGTTCCTGATGAAC
GAGTTCCTAAGCCCAGGCCAGGTTGCTGTGCTGGCTCATCCTCCTTAGAAAGATATGCAACCTCCAAT
GAGTTCCCTGATGATACCCTGAACTTCATCAAGACGCACCCGCTCATGGATGAGGCAGTGCCCTCCAT
CTTCAACAGGCCATGGTTCCTGAGAACAATGGTCAGATACCGCCTTACCAAAATTGCAGTGGACACAG
CTGCTGGGCCATATCAGAATCACACTGTGGTTTTTCTGGGATCAGAGAAGGGAATCATCTTGAAGTTT
TTGGCCAGAATAGGAAATAGTGGTTTTCTAAATGACAGCCTTTTCCTGGAGGAGATGAGTGTTTACAA
CTCTGAAAAATGCAGCTATGATGGAGTCGAAGACAAAAGGATCATGGGCATGCAGGTCGACGGC

| | | | |
|---|---|---|---|
| NOV2f, 260565761 | | | |
| Protein Sequence | SEQ ID NO: 24 | 497 aa | MW at 56242.5 kD |

TGSMRSEALLLYFTLLHFAGAGFPEDSEPISISHGNYTKQYPVFVGHKPGRNTTQRHRLDIQMIMIMN
GTLYIAARDHIYTVDIDTSHTEEIYCSKKLTWKSRQADVDTCRMKGKHKDECHNFIKVLLKKNDDALF
VCGTNAFNPSCRNYKMDTLEPFGDEFSGMARCPYDAKHANVALFADGKLYSATVTDFLAIDAVIYRSL
GESPTLRTVKHDSKWLKEPYFVQAVDYGDYIYFFFREIAVEYNTMGKVVFPRVAQVCKNDMGGSQRVL
EKQWTSFLKARLNCSVPGDSHFYFMILQAVTDVIRINGRDVVLATFSTPYNSIPGSAVCAYDMLDIAS
VFTGRFKEQKSPDSTWTPVPDERVPKPRPGCCAGSSSLERYATSNEFPDDTLNFIKTHPLMDEAVPSI
FNRPWFLRTMVRYRLTKIAVDTAAGPYQNHTVVFLGSEKGIILKFLARIGNSGFLNDSLFLEEMSVYN
SEKCSYDGVEDKRIMGMQVDG

| | | |
|---|---|---|
| NOV2g, 252324008 | SEQ ID NO: 25 | 1438 bp |
| DNA Sequence ORF Start: at 2 | ORF Stop: end of sequence | |

CACCGGATCCGGTTTCCCAGAAGATTCTGAGCCAATCAGTATTTCGCATGGCAACTATACAAAACAGT
ATCCGGTGTTTGTGGGCCACAAGCCAGGACGGAACACCACACAGAGGCACAGGCTGGACATCCAGATG
ATTATGATCATGAACGGAACCCTCTACATTGCTGCTAGGGACCATATTTATACTGTTGATATAGACAC
ATCACACACGGAAGAAATTTATTGTAGCAAAAAACTGACATGGAAATCTAGACAGGCCGATGTAGACA
CATGCAGAATGAAGGGAAAACATAAGGATGAGTGCCACAACTTTATTAAAGTTCTTCTAAAGAAAAAC
GATGATGCATTGTTTGTCTGTGGAACTAATGCCTTCAACCCTTCCTGCAGAAACTATAAGATGGATAC
ATTGGAACCATTCGGGGATGAATTCAGCGGAATGGCCAGATGCCCATATGATGCCAAACATGCCAACG
TTGCACTGTTTGCAGATGGAAAACTATACTCAGCCACAGTGACTGACTTCCTTGCCATTGACGCAGTC
ATTTACCGGAGTCTTGGAGAAAGCCCTACCCTGCGGACCGTCAAGCACGATTCAAAATGGTTGAAAGA
ACCATACTTTGTTCAAGCCGTGGATTACGGAGATTATATCTACTTCTTCTTCAGGGAAATAGCAGTGG
AGTATAACACCATGGGAAAGGTAGTTTTCCCAAGAGTGGCTCAGGTTTGTAAGAATGATATGGGAGGA
TCTCAAAGAGTCCTGGAGAAACAGTGGACGTCGTTCCTGAAGGCGCGCTTGAATTGCTCAGTTCCTGG
AGACTCTCATTTTTATTTCAACATTCTCCAGGCAGTTACAGATGTGATTCGTATCAACGGGCGTGATG
TTGTCCTGGCAACGTTTTCTACACCTTATAACAGCATCCCTGGGTCTGCAGTCTGTGCCTATGACATG
CTTGACATTGCCAGTGTTTTTACTGGGAGATTCAAGGAACAGAAGTCTCCTGATTCCACCTGGACACC
AGTTCCTGATGAACGAGTTCCTAAGCCCAGGCCAGGTTGCTGTGCTGGCTCATCCTCCTTAGAAAGAT
ATGCAACCTCCAATGAGTTCCCTGATGATACCCTGAACTTCATCAAGACGCACCCGCTCATGGATGAG
GCAGTGCCCTCCATCTTCAACAGGCCATGGTTCCTGAGAACAATGGTCAGATACCGCCTTACCAAAAT
TGCAGTGGACACAGCTGCTGGGCCATATCAGAATCACACTGTGGTTTTTCTGGGATCAGAGAAGGGAA
TCATCTTGAAGTTTTTGGCCAGAATAGGAAATAGTGGTTTTCTAAATGACAGCCTTTTCCTGGAGGAG
ATGAGTGTTTACAACTCTGAAAAATGCAGCTATGATGGAGTCGAAGACAAAAGGATCATGGGCATGCA
GGTCGACGGC

| | | | |
|---|---|---|---|
| NOV2g, 252324008 | | | |
| Protein Sequence | SEQ ID NO: 26 | 479 aa | MW at 54207.1 kD |

TGSGFPEDSEPISISHGNYTKQYPVFVGHKPGRNTTQRHRLDIQMIMIMNGTLYIAARDHIYTVDIDT
SHTEEIYCSKKLTWKSRQADVDTCRMKGKHKDECHNFIKVLLKKNDDALFVCGTNAFNPSCRNYKMDT
LEPFGDEFSGMARCPYDAKHANVALFADGKLYSATVTDFLAIDAVIYRSLGESPTLRTVKHDSKWLKE
PYFVQAVDYGDYIYFFFREIAVEYNTMGKVVFPRVAQVCKNDMGGSQRVLEKQWTSFLKARLNCSVPG
DSHFYFNILQAVTDVIRINGRDVVLATFSTPYNSIPGSAVCAYDMLDIASVFTGRFKEQKSPDSTWTP
VPDERVPKPRPGCCAGSSSLERYATSNEFPDDTLNFIKTHPLMDEAVPSIFNRPWFLRTMVRYRLTKI
AVDTAAGPYQNHTVVFLGSEKGIILKFLARIGNSGFLNDSLFLEEMSVYNSEKCSYDGVEDKRIMGMQ
VDG

| | | |
|---|---|---|
| NOV2h, 252323542 | SEQ ID NO: 27 | 3055 bp |
| DNA Sequence | ORF Start: at 2 | ORF Stop: end of sequence |

CACCGGATCCGGTTTCCCAGAAGATTCTGAGCCAATCAGTATTTCGCATGGCAACTATACAAAACAGT
ATCCGGTGTTTGTGGGCCACAAGCCAGGACGGAACACCACACAGAGGCACAGGCTGGACATCCAGATG
ATTATGATCATGAACGGAACCCTCTACATTGCTGCTAGGGACCATATTTATACTGTTGATATAGACAC
ATCACACACGGAAGAAATTTATTGTAGCAAAAAACTGACATGGAAATCTAGACAGGCCGATGTAGACA
CATGCAGAATGAAGGGAAAACATAAGGATGAGTGCCACAACTTTATTAAAGTTCTTCTAAAGAAAAAC
GATGATGCATTGTTTGTCTGTGGAACTAATGCCTTCAACCCTTCCTGCAGAAACTATAAGATGGATAC
ATTGGAACCATTCGGGGATGAATTCAGCGGAATGGCCAGATGCCCATATGATGCCAAACATGCCAACG
TTGCACTGTTTGCAGATGGAAAACTATACTCAGCCACAGTGACTGACTTCCTTGCCATTGACGCAGTC
ATTTACCGGAGTCTTGGAGAAAGCCCTACCCTGCGGACCGTCAAGCACGATTCAAAATGGTTGAAAGA
ACCATACTTTGTTCAAGCCGTGGATTACGGAGATTATATCTACTTCTTCTTCAGGGAAATAGCAGTGG
AGTATAACACCATGGGAAAGGTAGTTTTCCCAAGAGTGGCTCAGGTTTGTAAGAATGATATGGGAGGA
TCTCAAAGAGTCCTGGAGAAACAGTGGACGTCGTTCCTGAAGGCGCGCTTGAACTGCTCAGTTCCTGG
AGACTCTCATTTTTATTTCAACATTCTCCAGGCAGTTACAGATGTGATTCGTATCAACGGGCGTGATG
TTGTCCTGGCAACGTTTTCTACACCTTATAACAGCATCCCTGGGTCTGCAGTCTGTGCCTATGACATG
CTTGACATTGCCAGTGTTTTTACTGGGAGATTCAAGGAACAGAAGTCTCCTGATTCCACCTGGACACC
AGTTCCTGATGAACGAGTTCCTAAGCCCAGGCCAGGTTGCTGTGCTGGCTCATCCTCCTTAGAAAGAT
ATGCAACCTCCAATGAGTTCCCTGATGATACCCTGAACTTCATCAAGACGCACCCGCTCATGGATGAG
GCAGTGCCCTCCATCTTCAACAGGCCATGGTTCCTGAGAACAATGGTCAGATACCGCCTTACCAAAAT
TGCAGTGGACACAGCTGCTGGGCCATATCAGAATCACACTGTGGTTTTTCTGGGATCAGAGAAGGGAA
TCATCTTGAAGTTTTTGGCCAGAATAGGAAATAGTGGTTTTCTAAATGACAGCCTTTTCCTGGAGGAG
ATGAGTGTTTACAACTCTGAAAAATGCAGCTATGATGGAGTCGAAGACAAAAGGATCATGGGCATGCA
GCTGGACAGAGCAAGCAGCTCTCTGTATGTTGCGTTCTCTACCTGTGTGATAAAGGTTCCCCTTGGCC
GGTGTGAACGACATGGGAAGTGTAAAAAAACCTGTATTGCCTCCAGAGACCCATATTGTGGATGGATA
AAGGAAGGTGGTGCCTGCAGCCATTTATCACCCAACAGCAGACTGACTTTTGAGCAGGACATAGAGCG
TGGCAATACAGATGGTCTGGGGGACTGTCACAATTCCTTTGTGGCACTGAATGGGCATTCCAGTTCCC
TCTTGCCCAGCACAACCACATCAGATTCGACGGCTCAAGAGGGGTATGAGTCTAGGGGAGGAATGCTG
GACTGGAAGCATCTGCTTGACTCACCTGACAGCACAGACCCTTTGGGGGCAGTGTCTTCCCATAATCA
CCAAGACAAGAAGGGAGTGATTCGGGAAAGTTACCTCAAAGGCCACGACCAGCTGGTTCCCGTCACCC
TCTTGGCCATTGCAGTCATCCTGGCTTTCGTCATGGGGGCCGTCTTCTCGGGCATCACCGTCTACTGC
GTCTGTGATCATCGGCGCAAAGACGTGGCTGTGGTGCAGCGCAAGGAGAAGGAGCTCACCCACTCGCG
CCGGGGCTCCATGAGCAGCGTCACCAAGCTCAGCGGCCTCTTTGGGGACACTCAATCCAAAGACCCAA
AGCCGGAGGCCATCCTCACGCCACTCATGCACAACGGCAAGCTCGCCACTCCCGGCAACACGGCCAAG
ATGCTCATTAAAGCAGACCAGCACCACCTGGACCTGACGGCCCTCCCCACCCCAGAGTCAACCCCAAC
GCTGCAGCAGAAGCGGAAGCCCAGCCGCGGCAGCCGCGAGTGGGAGAGGAACCAGAACCTCATCAATG
CCTGCACAAAGGACATGCCCCCCATGGGCTCCCCTGTGATTCCCACGGACCTGCCCCTGCGGGCCTCC
CCCAGCCACATCCCCAGCGTGGTGGTCCTGCCCATCACGCAGCAGGGCTACCAGCATGAGTACGTGGA
CCAGCCCAAAATGAGCGAGGTGGCCCAGATGGCGCTGGAGGACCAGGCCGCCACACTGGAGTATAAGA
CCATCAAGGAACATCTCAGCAGCAAGAGTCCCAACCATGGGGTGAACCTTGTGGAGAACCTGGACAGC
CTGCCCCCCAAAGTTCCACAGCGGGAGGCCTCCCTGGGTCCCCCGGGAGCCTCCCTGTCTCAGACCGG
TCTAAGCAAGCGGCTGGAAATGCACCACTCCTCTTCCTACGGGGTTGACTATAAGAGGAGCTACCCCA
CGAACTCGCTCACGAGAAGCCACCAGGCCACCACTCTCAAAAGAAACAACACTAACTCCTCCAATTCC
TCTCACCTCTCCAGAAACCAGAGCTTTGGCAGGGGAGACAACCCGCCGCCCGCCCCGCAGAGGGTGGA
CTCCATCCAGGTGCACAGCTCCCAGCCATCTGGCCAGGCCGTGACTGTCTCGAGGCAGCCCAGCCTCA
ACGCCTACAACTCACTGACAAGGTCGGGGCTGAAGCGTACGCCCTCGCTAAAGCCGGACGTACCCCCC
AAACCATCCTTTGCTCCCCTTTCCACATCCATGAAGCCCAATGATGCGTGTACAGTCGACGGC

| | | | |
|---|---|---|---|
| NOV2h, 252323542 | | | |
| Protein Sequence | SEQ ID NO: 28 | 1018 aa | MW at 112848.6 kD |

TGSGFPEDSEPISISHGNYTKQYPVFVGHKPGRNTTQRHRLDIQMIMIMNGTLYIAARDHIYTVDIDT
SHTEEIYCSKKLTWKSRQADVDTCRMDGKHKDECHNFIKVLLKKNDDALFVCGTNAFNPSCRNYKMDT
LEPFGDEFSGMARCPYDAKHAIVALFADGKLYSATVTDFLAIDAVIYRSLGESPTLRTVKHDSKWLKE
PYFVQAVDYGDYIYFFFREIAVEYNTMGKVVFPRVAQVCKNDMGGSQRVLEKQWTSFLKARLNCSVPG
DSHFYFNILQAVTDVIRINGRDVVLATFSTPYNSIPGSAVCAYDMLDIASVFTGRFKEQKSPDSTWTP
VPDERVPKPRPGCCAGSSSLERYATSNEFPDDTLNFIKTHPLMDEAVPSIFNRPWFLRTMVRYRLTKI
AVDTAAGPYQNHTVVFLGSEKGIILKFLARIGNSGFLNDSLFLEEMSVYMSEKCSYDGVEDKRIMGMQ
LDRASSSLYVAFSTCVIKVPLGRCERHGKCKKTCIASRDPYCGWIKEGGACSHLSPNSRLTFEQDIER
GNTDGLGDCHNSFVALNGHSSSLLPSTTTSDSTAQEGYESRGGMLDWKHLLDSPDSTDPLGAVSSHNH
QDKKGVIRESYLKGHDQLVPVTLLAIAVILAFVMGAVFSGITVYCVCDHRRKDVAVVQRKEKELTHSR
RGSMSSVTKLSGLFGDTQSKDPKPEAILTPLMHNGKLATPGNTAKMLIKADQHHLDLTALPTPESTPT
LQQKRKPSRGSREWERNQNLINACTKDMPPMGSPVIPTDLPLRASPSHIPSVVVLPITQQGYQHEYVD
QPKMSEVAQMALEDQAATLEYKTIKEHLSSKSPNHGVMLVENLDSLPPKVPQREASLGPPGASLSQTG
LSKRLEMHHSSSYGVDYKRSYPTNSLTRSHQATTLKRNNTNSSNSSHLSRNQSFGRGDNPPPAPQRVD
SIQVHSSQPSGQAVTVSRQPSLNAYNSLTRSGLKRTPSLKPDVPPKPSFAPLSTSMKPNDACTVDG

| | | |
|---|---|---|
| NOV2i, 252323483 | SEQ ID NO: 29 | 2944 bp |
| DNA Sequence | ORF Start: at 2 | ORF Stop: end of sequence |

CACCGGATCCATGAGGTCAGAAGCCTTGCTGCTATATTTCACACTGCTACACTTTGCTGGGGCTGGTT
TCCCAGAAGATTCTGAGCCAATCAGTATTTCGCATGGCAACTATACAAAACAGTATCCGGTGTTTGTG
GGCCACAAGCCAGGACGGAACACCACACAGAGGCACAGGCTGGACATCCAGATGATTATGATCATGAA
CGGAACCCTCTACATTGCTGCTAGGGACCATATTTATACTGTTGATATAGACACATCACACACGGAAG
AAATTTATTGTAGCAAAAAACTGACATGGAAATCTAGACAGGCCGATGTAGACACATGCAGAATGAAG
GGAAAACATAAGGATGAGTGCCACAACTTTATTAAAGTTCTTCTAAAGAAAAACGATGATGCATTGTT
TGTCTGTGGAACTAATGCCTTCAACCCTTCCTGCAGAAACTATAAGATGGATACATTGGAACCATTCG
GGGATGAATTCAGCGGAATGGCCAGATGCCCATATGATGCCAAACATGCCAACGTTGCACTGTTTGCA
GATGGAAAACTATACTCAGCCACAGTGACTGACTTCCTTGCCATTGACGCAGTCATTTACCGGAGTCT
TGGAGAAAGCCCTACCCTGCGGACCGTCAAGCACGATTCAAAATGGTTGAAAGAACCATACTTTGTTC
AAGCCGTGGATTACGGAGATTATATCTACTTCTTCTTCAGGGAAATAGCAGTGGAGTATAACACCATG
GGAAAGGTAGTTTTCCCAAGAGTGGCTCAGGTTTGTAAGAATGATATGGGAGGATCTCAAAGAGTCCT
GGAGAAACAGTGGACGTCGTTCCTGAAGGCGCGCTTGAACTGCTCAGTTCCTGGAGACTCTCATTTTT
ATTTCAACATTCTCCAGGCAGTTACAGATGTGATTCGTATCAAGGGGCGTGATGTTGTCCTGGCAACG
TTTTCTACACCTTATAACAGCATCCCTGGGTCTGCAGTCTGTGCCTATGACATGCTTGACATTGCCAG
TGTTTTTACTGGGAGATTCAAGGAACAGAAGTCTCCTGATTCCACCTGGACACCAGTTCCTGATGAAC
GAGTTCCTAAGCCCAGGCCAGGTTGCTGTGCTGGCTCATCCTCCTTAGAAAGATATGCAACCTCCAAT
GAGTTCCCTGACGATACCCTGAACTTCATCAAGACGCACCCGCTCATGGATGAGGCAGTGCCCTCCAT
CTTCAACAGGCCATGGTTCCTGAGAACAATGGTCAGATACCGCCTTACCAAAATTGCAGTGGACACAG
CTGCTGGGCCATATCAGAATCACACTGTGGTTTTTCTGGGATCAGAGAAGGGAATCATCTTGAAGTTT
TTGGCCAGAATAGGAAATAGTGGTTTTCTAAATGACAGCCTTTTCCTGGAGGAGATGAGTGTTTACAA
CTCTGAAAAATGCAGCTATGATGGAGTCGAAGACAAAAGGATCATGGGCATGCAGCTGGACAGAGCAA
GCAGCTCTCTGTATGTTGCGTTCTCTACCTGTGTGATAAAGGTTCCCCTTGGCCGGTGTGAACGACAT
GGGAAGTGTAAAAAAACCTGTATTGCCTCCAGAGACCCATATTGTGGATGGATAAAGGAAGGTGGTGC
CTGCAGCCATTTATCACCCAACAGCAGACTGACTTTTGAGCAGGACATAGAGCGTGGCAATACAGATG
GTCTGGGGGACTGTCACAATTCCTTTGTGGCACTGAATGGAGTGATTCGGGAAAGTTACCTCAAAGGC
CACGACCAGCTGGTTCCCGTCACCCTCTTGGCCATTGCAGTCATCCTGGCTTTCGTCATGGGGGCCGT
CTTCTCGGGCATCACCGTCTACTGCGTCTGTGATCATCGGCGCAAAGACGTGGCTGTGGTGCAGCGCA
AGGAGAAGGAGCTCACCCACTCGCGCCGGGGCTCCATGAGCAGCGTCACCAAGCTCAGCGGCCTCTTT
GGGGACACTCAATCCAAAGACCCAAAGCCGGAGGCCATCCTCACGCCACTCATGCACAACGGCAAGCT
CGCCACTCCCGGCAACACGGCCAAGATGCTCATTAAAGCAGACCAGCACCACCTGGACCTGACGGCCC
TCCCCACCCCAGAGTCAACCCCAACGCTGCAGCAGAAGCGGAAGCCCAGCCGCGGCAGCCGCGAGTGG
GAGAGGAACCAGAACCTCATCAATGCCTGCACAAAGGACATGCCCCCCATGGGCTCCCCTGTGATTCC
CACGGACCTGCCCCTGCGGGCCTCCCCCAGCCACATCCCCAGCGTGGTGGTCCTGCCCATCACGCAGC
AGGGCTACCAGCATGAGTACGTGGACCAGCCCAAAATGAGCGAGGTGGCCCAGATGGCGCTGGAGGAC
CAGGCCGCCACACTGGAGTATAAGACCATCAAGGAACATCTCAGCAGCAAGAGTCCCAACCATGGGGT
GAACCTTGTGGAGAACCTGGACAGCCTGCCCCCCAAAGTTCCACAGCGGGAGGCCTCCCTGGGTCCCC
CGGGAGCCTCCCTGTCTCAGACCGGTCTAAGCAAGCGGCTGGAAATGCACCACTCCTCTTCCTACGGG
GTTGACTATAAGAGGAGCTACCCCACGAACTCGCTCACGAGAAGCCACCAGGCCACCACTCTCAAAAG
AAACAACACTAACTCCTCCAATTCCTCTCACCTCTCCAGAAACCAGAGCTTTGGCAGGGGAGACAACC
CGCCGCCCGCCCCGCAGAGGGTGGACTCCATCCAGGTGCACAGCTCCCAGCCATCTGGCCAGGCCGTG
ACTGTCTCGAGGCAGCCCAGCCTCAACGCCTACAACTCACTGACAAGGTCGGGGCTGAAGCGTACGCC
CTCGCTAAAGCCGGACGTACCCCCCAAACCATCCTTTGCTCCCCTTTCCACATCCATGAAGCCCAATG
ATGCGTGTACAGTCGACGGC

| | | | |
|---|---|---|---|
| NOV2i, 252323483 | | | |
| Protein Sequence | SEQ ID NO: 30 | 981 aa | MW at 109048.9 kD |

TGSMRSEALLLYFTLLHFAGAGFPEDSEPISISHGNYTKQYPVFVGHKPGRNTTQRHRLDIQMIMIMN
GTLYIAARDHIYTVDIDTSHTEEIYCSKKLTWKSRQADVDTCRMKGKHKDECHNFIKVLLKKNDDALF
VCGTNAFNPSCRNYKMDTLEPFGDEFSGMARCPYDAKHANVALFADGKLYSATVTDFLAIDAVIYRSL
GESPTLRTVKHDSKWLKEPYFVQAVDYGDYIYFFFREIAVEYNTMGKVVFPRVAQVCKNDMGGSQRVL
EKQWTSFLKARLNCSVPGDSHFYFNILQAVTDVIRIKGRDVVLATFSTPYNSIPGSAVCAYDMLDIAS
VFTGRFKEQKSPDSTWTPVPDERVPKPRPGCCAGSSSLERYATSNEFPDDTLNFIKTHPLMDEAVPSI
FNRPWFLRTMVRYRLTKIAVDTAAGPYONHTVVFLGSEKGIILKFLARIGNSGFLNDSLFLEEMSVYN
SEKCSYDGVEDKRIMGMQLDRASSSLYVAFSTCVIKVPLGRCERHGKCKKTCIASRDPYCGWIKEGGA
CSHLSPNSRLTFEQDIERGNTDGLGDCHNSFVALNGVIRESYLKGHDQLVPVTLLAIAVILAFVMGAV
FSGITVYCVCDHRRKDVAVVQRKEKELTHSRRGSMSSVTKLSGLFGDTQSKDPKPEAILTPLMHNGKL
ATPGNTAKMLIKADOHHLDLTALPTPESTPTLQQKRKPSRGSREWERNQNLINACTKDMPPMGSPVIP
TDLPLRASPSHIPSVVVLPITQQGYQHEYVDQPKMSEVAQMALEDQAATLEYKTIKEHLSSKPSNHGV
NLVENLDSLPPKVPQREASLGPPGASLSQTGLSKRLEMHHSSSYGVDYKRSYPTNSLTRSHQATTLKR
NNTNSSNSSHLSRNQSFGRGDNPPPAPQRVDSIQVHSSQPSGQAVTVSRQPSLNAYNSLTRSGLKRTP
SLKPDVPPKPSFAPLSTSMDPNDACTVDG

| | | |
|---|---|---|
| NOV2j, CG51896-01 | SEQ ID NO: 3 | 3498 bp |
| DNA Sequence | ORF Start: ATG at 214 | ORF Stop: end of sequence |

GCGACTATTTCCCCCAAAGAGACAAGCACACATGTAGGAATGACAAAGGCTTGCGAAGGAGAGAGCGC
AGCCCGCGGCCCGGAGAGATCCCCTCGATAATGGATTACTAAATGGGATACACGCTGTACCAGTTCGC
TCCGAGCCCCGGCCGCCTGTCCGTCGATGCACCGAAAAGGGTGAAGTAGAGAAATAAAGTCTCCCCGC
TGAACTACTATGAGGTCAGAAGCCTTGCTGCTATATTTCACACTGCTACACTTTGCTGGGGCTGGTTT
CCCAGAAGATTCTGAGCCAATCAGTATTTCGCATGGCAACTATACAAAACAGTATCCGGTGTTTGTGG
GCCACAAGCCAGGACGGAACACCACACAGAGGCACAGGCTGGACATCCAGATGATTATGATCATGAAC
GGAACCCTCTACATTGCTGCTAGGGACCATATTTATACTGTTGATATAGACACATCACACACGGAAGA
AATTTATTGTAGCAAAAAACTGACATGGAAATCTAGACAGGCCGATGTAGACACATGCAGAATGAAGG
GAAAACATAAGGATGAGTGCCACAACTTTATTAAAGTTCTTCTAAAGAAAAACGATGATGCATTGTTT
GTCTGTGGAACTAATGCCTTCAACCCTTCCTGCAGAAACTATAAGATGGATACATTGGAACCATTCGG
GGATGAATTCAGCGGAATGGCCAGATGCCCATATGATGCCAAACATGCCAACGTTGCACTGTTTGCAG
ATGGAAAACTATACTCAGCCACAGTGACTGACTTCCTTGCCATTGACGCAGTCATTTACCGGAGTCTT
GGAGAAAGCCCTACCCTGCGGACCGTCAAGCACGATTCAAAATGGTTGAAAGAACCATACTTTGTTCA
AGCCGTGGATTACGGAGATTATATCTACTTCTTCTTCAGGGAAATAGCAGTGGAGTATAACACCATGG
GAAAGGTAGTTTTCCCAAGAGTGGCTCAGGTTTGTAAGAATGATATGGGAGGATCTCAAAGAGTCCTG
GAGAAACAGTGGACGTCGTTCCTGAAGGCGCGCTTGAACTGCTCAGTTCCTGGAGACTCTCATTTTTA
TTTCAACATTCTCCAGGCAGTTACAGATGTGATTCGTATCAACGGGCGTGATGTTGTCCTGGCAACGT
TTTCTACACCTTATAACAGCATCCCTGGGTCTGCAGTCTGTGCCTATGACATGCTTGACATTGCCAGT
GTTTTTACTGGGAGATTCAAGGAACAGAAGTCTCCTGATTCCACCTGGACACCAGTTCCTGATGAACG
AGTTCCTAAGCCCAGGCCAGGTTGCTGTGCTGGCTCATCCTCCTTAGAAAGATATGCAACCTCCAATG
AGTTCCCTGATGATACCCTGAACTTCATCAAGACGCACCCGCTCATGGATGAGGCAGTGCCCTCCATC
TTCAACAGGCCATGGTTCCTGAGAACAATGGTCAGATACCGCCTTACCAAAATTGCAGTGGACACAGC
TGCTGGGCCATATCAGAATCACACTGTGGTTTTTCTGGGATCAGAGAAGGGAATCATCTTGAAGTTTT
TGGCCAGAATAGGAAATAGTGGTTTTCTAAATGACAGCCTTTTCCTGGAGGAGATGAGTGTTTACAAC
TCTGAAAAATGCAGCTATGATGGAGTCGAAGACAAAAGGATCATGGGCATGCAGCTGGACAGAGCAAG
CAGCTCTCTGTATGTTGCGTTCTCTACCTGTGTGATAAAGGTTCCCCTTGGCCGGTGTGAACGACATG
GGAAGTGTAAAAAAACCTGTATTGCCTCCAGAGACCCATATTGTGGATGGATAAAGGAAGGTGGTGCC
TGCAGCCATTTATCACCCAACAGCAGACTGACTTTTGAGCAGGACATAGAGCGTGGCAATACAGATGG
TCTGGGGGACTGTCACAATTCCTTTGTGGCACTGAATGGGCATTCCAGTTCCCTCTTGCCCAGCACAA
CCACATCAGATTCGACGGCTCAAGAGGGGTATGAGTCTAGGGGAGGAATGCTGGACTGGAAGCATCTG
CTTGACTCACCTGACAGCACAGACCCTTTGGGGGCAGTGTCTTCCCATAATCACCAAGACAAGAAGGG
AGTGATTCGGGAAAGTTACCTCAAAGGCCACGACCAGCTGGTTCCCGTCACCCTCTTGGCCATTGCAG
TCATCCTGGCTTTCGTCATGGGGGCCGTCTTCTCGGGCATCACCGTCTACTGCGTCTGTGATCATCGG
CGCAAAGACGTGGCTGTGGTGCAGCGCAAGGAGAAGGAGCTCACCCACTCGCGCCGGGGCTCCATGAG
CAGCGTCACCAAGCTCAGCGGCCTCTTTGGGGACACTCAATCCAAAGACCCAAAGCCGGAGGCCATCC
TCACGCCACTCATGCACAACGGCAAGCTCGCCACTCCCGGCAACACGGCCAAGATGCTCATTAAAGCA
GACCAGCACCACCTGGACCTGACGGCCCTCCCCACCCCAGAGTCAACCCCAACGCTGCAGCAGAAGCG
GGAACCCAGCCGCGGCACCCGCGAGTGGGAGAGGAACCAGAACCTCATCAATGCCTGCACAAAGGACA
TGCCCCCCATGGGCTCCCCTGTGATTCCCACGGACCTGCCCCTGCGGGCCTCCCCCAGCCACATCCCC
AGCGTGGTGGTCCTGCCCATCACGCAGCAGGGCTACCAGCATGAGTACGTGGACCAGCCCAAAATGAG
CGAGGTGGCCCAGATGGCGCTGGAGGACCAGGCCGCCACACTGGAGTATAAGACCATCAAGGAACATC
TCAGCAGCAAGAGTCCCAACCATGGGGTGAACCTTGTGGAGAACCTGGACAGCCTGCCCCCCAAAGTT
CCACAGCGGGAGGCCTCCCTGGGTCCCCCGGGAGCCTCCCTGTCTCAGACCGGTCTAAGCAAGCGGCT
GGAAATGCACCACTCCTCTTCCTACGGGGTTGACTATAAGAGGAGCTACCCCACGAACTCGCTCACGA
GAAGCCACCTGACCACCTACTCTCATCAGAAGCAACACTAACCCCGACAATTCANCTCTGACTTCAAA
GGGACCAGAGCTTTGGCAGGGGAGACAACCCGCCGCCCGCCCCGCAGAGGGTGGACTCCATCCAGGTG
CACAGCTCCCAGCCATCTGGCCAGGCCGTGACTGTCTCGAGGCAGCCCAGCCTCAACGCCTACAACTC
ACTGACAAGGTCGGGGCTGAAGCGTACGCCCTCGCTAAAGCCGGACGTACCCCCCAAACCATCCTTTG
CTCCCCTTTCCACATCCATGAAGCCCAATGATGCGTGTACATAATCCCAGGGGGAGGGGGTCAGGTGT
CGAACCAGCAGGCAAGGCGAGGTGTCCGCTCAGCTCAGCAAGGTTCTCAACTGCCTCGAGTACCCACC
AAACCAAAAAGGCCTGCGGCAGAACCGAGGGACGCTGGGTCCTCCTCTCTGGGACACAGGGGTACTCA
CGAAAACTGGGCCGCGTGGTTTGGTGAAAG

| | | | |
|---|---|---|---|
| NOV2j, CG51896-01 | | | |
| Protein Sequence | SEQ ID NO: 32 | 939 aa | MW at 104828.0 kD |

MRSEALLLYFTLLHFAGAGFPEDSEPISISHGNYTKQYPVFVGHKPGRNTTQRHRLDIQMIMIMNGTL
YIAARDHIYTVDIDTSHTEEIYCSKKLTWKSRQADVDTCRMKGKHKDECHNFIKVLLKKNDDALFVCG
TNAFNPSCRNYKMDTLEPFGDEFSGMARCPYDAKHANVALFADGKLYSATVTDFLAIDAVIYRSLGES
PTLRTVKHDSKWLKEPYFVQAVDYGDYIYFFFREIAVEYNTMGKVVFPRVAQVCKNDMGGSQRVLEKQ
WTSFLKARLNCSVPGDSHFYFNILQAVTDVIRINGRDVVLATFSTPYNSIPGSAVCAYDMLDIASVFT
GRFKEQKSPDSTWTPVPDERVPKPRPGCCAGSSSLERYATSNEFPDDTLNFIKTHPLMDEAVPSIFNR
PWFLRTMVRYRLTKIAVDTAAGPYQNHTVVFLGSEKGIILKFLARIGNSGFLNDSLFLEEMSVYNSEK
CSYDGVEDKRIMGMQLDRASSSLYVAFSTCVIKVPLGRCERHGKCKKTCIASRDPYCGWIKEGGACSH
LSPNSRLTFEQDIERGNTDGLGDCHNSFVALNGHSSSLLPSTTTSDSTAQEGYESRGGMLDWKHLLDS
PDSTDPLGAVSSHNHQDKKGVIRESYLKGHDQLVPVTLLAIAVILAFVMGAVFSGITVYCVCDHRRKD
VAVVQRKEKELTHSRRGSMSSVTKLSGLFGDTQSKDPKPEAILTPLMHNGKLATPGNTAKMLIKADQH
HLDLTALPTPESTPTLQQKREPSRGTREWERNQNLINACTKDMPPMGSPVIPTDLPLRASPSHIPSVV
VLPITQQGYQHEYVDQPKMSEVAQMALEDQAATLEYKTIKEHLSSKSPNHGVNLVENLDSLPPKVPQR
EASLGPPGASLSQTGLSKRLEMHHSSSYGVDYKRSYPTNSLTRSHLTTYSHQKQH

| | | |
|---|---|---|
| NOV2k, CG51896-02 | SEQ ID NO: 33 | 1878 bp |
| DNA Sequence | ORF Start: at 1 | ORF Stop: end of sequence |

GGTTTCCCAGAAGATTCTGAGCCAATCAGTATTTCGCATGGCAACTATACAAAACAGTATCCGGTGTT
TGTGGGCCACAAGCCAGGACGGAACACCACACAGAGGCACAGGCTGGACATCCAGATGATTATGATCA
TGAACGGAACCCTCTACATTGCTGCTAGGGACCATATTTATACTGTTGATATAGACACATCACACACG
GAAGAAATTTATTGTAGCAAAAAACTGACATGGAAATCTAGACAGGCCGATGTAGACACATGCAGAAT
GAAGGGAAAACATAAGGATGAGTGCCACAACTTTATTAAAGTTCTTCTAAAGAAAAACGATGATGCAT
TGTTTGTCTGTGGAACTAATGCCTTCAACCCTTCCTGCAGAAACTATAAGATGGATACATTGGAACCA
TTCGGGGATGAATTCAGCGGAATGGCCAGATGCCCATATGATGCCAAACATGCCAACGTTGCACTGTT
TGCAGATGGAAAACTATACTCAGCCACAGTGACTGACTTCCTTGCCATTGACGCAGTCATTTACCGGA
GTCTTGGAGAAAGCCCTACCCTGCGGACCGTCAAGCACGATTCAAAATGGTTGAAAGAACCATACTTT
GTTCAAGCCGTGGATTACGGAGATTATATCTACTTCTTCTTCAGGGAAATAGCAGTGGAGTATAACAC
CATGGGAAAGGTAGTTTTCCCAAGAGTGGCTCAGGTTTGTAAGAATGATATGGGAGGATCTCAAAGAG
TCCTGGAGAAACAGTGGACGTCGTTCCTGAAGGCGCGCTTGAACTGCTCAGTTCCTGGAGACTCTCAT
TTTTATTTCAACATTCTCCAGGCAGTTACAGATGTGATTCGTATCAACGGGCGTGATGTTGTCCTGGC
AACGTTTTCTACACCTTATAACAGCATCCCTGGGTCTGCAGTCTGTGCCTATGACATGCTTGACATTG
CCAGTGTTTTTACTGGGAGATTCAAGGAACAGAAGTCTCCTGATTCCACCTGGACACCAGTTCCTGAT
GAACGAGTTCCTAAGCCCAGGCCAGGTTGCTGTGCTGGCTCATCCTCCTTAGAAAGATATGCAACCTC
CAATGAGTTCCCTGATGATACCCTGAACTTCATCAAGACGCACCCGCTCATGGATGAGGCAGTGCCCT
CCATCTTCAACAGGCCATGGTTCCTGAGAACAATGGTCAGATACCGCCTTACCAAAATTGCAGTGGAC
ACAGCTGCTGGGCCATATCAGAATCACACTGTGGTTTTTCTGGGATCAGAGAAGGGAATCATCTTGAA
GTTTTTGGCCAGAATAGGAAATAGTGGTTTTCTAAATGACAGCCTTTTCCTGGAGGAGATGAGTGTTT
ACAACTCTGAAAAATGCAGCTATGATGGAGTCGAAGACAAAAGGATCATGGGCATGCAGCTGGACAGA
GCAAGCAGCTCTCTGTATGTTGCGTTCTCTACCTGTGTGATAAAGGTTCCCCTTGGCCGGTGTGAACG
ACATGGGAAGTGTAAAAAAACCTGTATTGCCTCCAGAGACCCATATTGTGGATGGATAAAGGAAGGTG
GTGCCTGCAGCCATTTATCACCCAACAGCAGACTGACTTTTGAGCAGGACATAGAGCGTGGCAATACA
GATGGTCTGGGGGACTGTCACAATTCCTTTGTGGCACTGAATGGGCATTCCAGTTCCCTCTTGCCCAG
CACAACCACATCAGATTCGACGGCTCAAGAGGGGTATGAGTCTAGGGGAGGAATGCTGGACTGGAAGC
ATCTGCTTGACTCACCTGACAGCACAGACCCTTTGGGGGCAGTGTCTTCCCATAATCACCAAGACAAG
AAGGGAGTGATTCGGGAAAGTTACCTCAAAGGCCACGACCAG

| | | | |
|---|---|---|---|
| NOV2k, CG51896-02 | | | |
| Protein Sequence | SEQ ID NO: 34 | 1626 aa | MW at 70297.8 kD |

GFPEDSEPISISHGNYTKQYPVFVGHKPGRNTTQRHRLDIQMIMIMNGTLYIAARDHIYTVDIDTSHT
EEIYCSKKLTWKSRQADVDTCRMKGKHKDECHNFIKVLLKKNDDALFVCGTNAFNPSCRNYKMDTLEP
FGDEFSGMARCPYDAKHANVALFADGKLYSATVTDFLAIDAVIYRSLGESPTLRTVKHDSKWLKEPYF
VQAVDYGDYIYFFFREIAVEYNTMGKVVFPRVAQVCKNDMGGSQRVLEKQWTSFLKARLNCSVPGDSH
FYFNILQAVTDVIRINGRDVVLATFSTPYNSIPGSAVCAYDMLDIASVFTGRFKEQKSPDSTWTPVPD
ERVPKPRPGCCAGSSSLERYATSNEFPDDTLNFIKTHPLMDEAVPSIFNRPWFLRTMVRYRLTKIAVD
TAAGPYQNHTVVFLGSEKGIILKFLARIGNSGFLNDSLFLEEMSVYNSEKCSYDGVEDKRIMGMQLDR
ASSSLYVAFSTCVIKVPLGRCERHGKCKKTCIASRDPYCGWIKEGGACSHLSPNSRLTFEQDIERGNT
DGLGDCHNSFVALNGHSSSLLPSTTTSDSTAQEGYESRGGMLDWKHLLDSPDSTDPLGAVSSHNHQDK
KGVIRESYLKGHDQ

| | | |
|---|---|---|
| NOV21l CG51896-03 | SEQ ID NO:35 | 1908 bp |
| DNA Sequence | ORF Start: at 1 | ORF Stop: end of sequence |

GGTTTCCCAGAAGATTCTGAGCCAATCAGTATTTCGCATGGCAACTATACAAAACAGTATCCGGTGTT
TGTGGGCCACAAGCCAGGACGGAACACCACACAGAGGCACAGGCTGGACATCCAGATGATTATGATCA
TGAACGGAACCCTCTACATTGCTGCTAGGGACCATATTTATACTGTTGATATAGACACATCACACACG
GAAGAAATTTATTGTAGCAAAAAACTGACATGGAAATCTAGACAGGCCGATGTAGACACATGCAGAAT
GAAGGGAAAACATAAGGATGAGTGCCACAACTTTATTAAAGTTCTTCTAAAGAAAAACGATGATGCAT
TGTTTGTCTGTGGAACTAATGCCTTCAACCCTTCCTGCAGAAACTATAAGATGGATACATTGGAACCA
TTCGGGGATGAATTCAGCGGAATGGCCAGATGCCCATATGATGCCAAACATGCCAACGTTGCACTGTT
TGCAGATGGAAAACTATACTCAGCCACAGTGACTGACTTCCTTGCCATTGACGCAGTCATTTACCGGA
GTCTTGGAGAAAGCCCTACCCTGCGGACCGTCAAGCACGATTCAAAATGGTTGAAAGAACCATACTTT
GTTCAAGCCGTGGATTACGGAGATTATATCTACTTCTTCTTCAGGGAAATAGCAGTGGAGTATACCAC
CATGGGAAAGGTAGTTTTCCCAAGAGTGGCTCAGGTTTGTAAGAATGATATGGGAGGATCTCAAAGAG
TCCTGGAGAAACAGTGGACGTCGTTCCTGAAGGCGCGCTTGAACTGCTCAGTTCCTGGAGACTCTCAT
TTTTATTTCAACATTCTCCAGGCAGTTACAGATGTGATTCGTATCAACGGGCGTGATGTTGTCCTGGC
AACGTTTTCTACACCTTATAACAGCATCCCTGGGTCTGCAGTCTGTGCCTATGACATGCTTGACATTG
CCAGTGTTTTTACTGGGAGATTCAAGGAACAGAAGTCTCCTGATTCCACCTGGACACCAGTTCCTGAT
GAACGAGTTCCTAAGCCCAGGCCAGGTTGCTGTGCTGGCTCATCCTCCTTAGAAAGATATGCAACCTC
CAATGAGTTCCCTGATGATACCCTGAACTTCATCAAGACGCACCCGCTCATGGATGAGGCAGTGCCCT
CCATCTTCAACAGGCCATGGTTCCTGAGAACAATGGTCAGATGCAGCTATGATGGAGTCGAAGACAAA
AGGATCATGGGCATGCAGCTGGACAGAGCAAGCAGCTCTCTGTATGTTGCGTTCTCTACCTGTGTGAT
AAAGGTCCCCCTTGGCCGGTGTGAACGACATGGGAAGTGTAAAAAAACCTGTATTGCCTCCAGAGACC
CATATTGTGGATGGATAAAGGAAGGTGGTGCCTGCAGCCATTTATCACCCAACAGCAGACTGACTTTT
GAGCAGGACATAGAGCGTGGCAATACAGATGGTCTGGGGGACTGTCACAATTCCTTTGTGGCACTGAA
TGGGCATTCCAGTTCCCTCTTGCCCAGCACAACCACATCAGATTCGACGGCTCAAGAGGGGTATGAGT
CTAGGGGAGGAATGCTGGACTGGAAGCATCTGCTTGACTCACCTGACAGCACAGACCCTTTGGGGGCA
GTGTCTTCCCATAATCACCAAGACAAGAAGGGAGTGATTCGGGAAAGTTACCTCAAAGGCCACGACCA
GCTGGTTCCCGTCACCCTCTTGGCCATTGCAGTCATCCTGGCTTTCGTCATGGGGGCCGTCTTCTCGG
GCATCACCGTCTACTGCGTCTGTGATCATCGGCGCAAAGACGTGGCTGTGGTGCAGCGCAAGGAGAAG
GAGCTCACCCACTCGCGCCGGGGCTCCATGAGCAGCGTCACCAAGCTCAGCGGCCTCTTTGGGGACAC
TCAA

| | | | |
|---|---|---|---|
| NOV2l, CG51896-03 | | | |
| Protein Sequence | SEQ ID NO: 36 | 636 aa | MW at 71237.1 kD |

GFPEDSEPISISHGNYTKQYPVFVGHKPGRNTTQRHRLDIQMIMIMNGTLYIAARDHIYTVDIDTSHT
EEIYCSKKLTWKSRQADVDTCRMKGKHKDECH&FIKVLLKKNDDALFVCGTNAFNPSCRNYKMDTLEP
FGDEFSGMARCPYDAKHANVALFADGKLYSATVTDFLAIDAVIYRSLGESPTLRTVKHDSKWLKEPYF
VQAVDYGDYIYFFFREIAVEYNTMGKVVFPRVAQVCKNDMGGSQRVLEKQWTSFLKARLNCSVPGDSH
FYFNILQAVTDVIRINGRDVVLATFSTPYNSIPGSAVCAYDMLDIASVFTGRFKEQKSPDSTWTPVPD
ERVPKPRPGCCAGSSSLERYATSNEFPDDTLNFIKTHPLMDEAVPSIFNRPWFLRTMVRCSYDGVEDK
RIMGMQLDRASSSLYVAFSTCVIKVPLGRCERHGKCKKTCIASRDPYCGWIKEGGACSHLSPNSRLTF
EQDIERGNTDGLGDCHNSFVALNGHSSSLLPSTTTSDSTAQEGYESRGGMLDWKHLLDSPDSTDPLGA
VSSHNHQDKKGVIRESYLKGHDQLVPVTLLAIAVILAFVMGAVFSGITVYCVCDHRRKDVAVVQRKEK
ELTHSRRGSMSSVTKLSGLFGDTQ

| | | |
|---|---|---|
| NOV2m, CG51896-05 | SEQ ID NO: 37 | 54 bp |
| DNA Sequence | ORF Start: at 1 | ORF Stop: at 55 |

GGAGAAAGCCCTACCCTGCGGACCGTCAAGCACGATTCAAAATGGTTGAAAGAA

| | | | |
|---|---|---|---|
| NOV2m, CG51896-05 | | | |
| Protein Sequence | SEQ ID NO: 38 | 18 aa | MW at 2111.4 kD |

GESPTLRTVKHDSKWLKE

| | | |
|---|---|---|
| NOV2n, CG51896-06 | SEQ ID NO: 39 | 54 bp |
| DNA Sequence | ORE Start: at 1 | ORF Stop: end of sequence |

GGAGAAAGCCCTACCCTGCGGACCGTCAAGCACGATTCAAAATGGTTGAAAGAA

| | | | |
|---|---|---|---|
| NOV2n, CG51896-06 | | | |
| Protein Sequence | SEQ ID NO: 40 | 18 aa | MW at 2111.4 kD |

GESPTLRTVKHDSKWLKE

| | | |
|---|---|---|
| NOV2o, CG51896-07 | SEQ ID NO: 41 | 51 bp |
| DNA Sequence | ORF Start: at 1 | ORF Stop: end of sequence |

TCATCCTCCTTAGAAAGATATGCAACCTCCAATGAGTTCCCTGATGATACC

| | | | |
|---|---|---|---|
| NOV2o, CG51896-07 | | | |
| Protein Sequence | SEQ ID NO: 42 | 117 aa | MW at 1918.9 kD |

SSSLERYATSNEFPDDT

| | | |
|---|---|---|
| NOV2p, CG51896-08 | SEQ ID NO: 43 | 60 bp |
| DNA Sequence | ORF Start: at 1 | ORF Stop: end of sequence |

GAGGAGATGAGTGTTTACAACTCTGAAAAATGCAGCTATGATGGAGTCGAAGACAAAAGG

| | | | |
|---|---|---|---|
| NOV2p, CG51896-08 | | | |
| Protein Sequence | SEQ ID NO: 44 | 20 aa | MW at 2368.5 kD |

EEMSVYNSEKCSYDGVEDKR

| | | |
|---|---|---|
| NOV2q, CG51896-09 | SEQ ID NO: 45 | 3983 bp |
| DNA Sequence | ORF Start: ATG at 214 | ORF Stop: end of sequence |

GCGACTATTTCCCCCAAAGAGACAAGCACACATGTAGGAATGACAAAGGCTTGCGAAGGAGAGAGCGC
AGCCCGCGGCCCGGAGAGATCCCCTCGATAATGGATTACTAAATGGGATACACGCTGTACCAGTTCGC
TCCGAGCCCCGGCCGCCTGTCCGTCGATGCACCGAAAAGGGTGAAGTAGAGAAATAAAGTCTCCCCGC
TGAACTACTATGAGGTCAGAAGCCTTGCTGCTATATTTCACACTGCTACACTTTGCTGGGGCTGGTTT
CCCAGAAGATTCTGAGCCAATCAGTATTTCGCATGGCAACTATACAAAACAGTATCCGGTGTTTGTGG
GCCACAAGCCAGGACGGAACACCACACAGAGGCACAGGCTGGACATCCAGATGATTATGATCATGAAC
GGAACCCTCTACATTGCTGCTAGGGACCATATTTATACTGTTGATATAGACACATCACACACGGAAGA
AATTTATTGTAGCAAAAAACTGACATGGAAATCTAGACAGGCCGATGTAGACACATGCAGAATGAAGG
GAAAACATAAGGATGAGTGCCACAACTTTATTAAAGTTCTTCTAAAGAAAAACGATGATGCATTGTTT
GTCTGTGGAACTAATGCCTTCAACCCTTCCTGCAGAAACTATAAGATGGATACATTGGAACCATTCGG
GGATGAATTCAGCGGAATGGCCAGATGCCCATATGATGCCAAACATGCCAACGTTGCACTGTTTGCAG
ATGGAAAACTATACTCAGCCACAGTGACTGACTTCCTTGCCATTGACGCAGTCATTTACCGGAGTCTT
GGAGAAAGCCCTACCCTGCGGACCGTCAAGCACGATTCAAAATGGTTGAAAGAACCATACTTTGTTCA
AGCCGTGGATTACGGAGATTATATCTACTTCTTCTTCAGGGAAATAGCAGTGGAGTATAACACCATGG
GAAAGGTAGTTTTCCCAAGAGTGGCTCAGGTTTGTAAGAATGATATGGGAGGATCTCAAAGAGTCCTG
GAGAAACGGTGGACGTCGTTCCTGAAGGCGCGCTTGAACTGCTCAGTTCCTGGAGACTCTCATTTTTA
TTTCAACATTCTCCAGGCAGTTACAGATGTGATTCGTATCAACGGGCGTGATGTTGTCCTGGCAACGT
TTTCTACACCTTATAACAGCATCCCTGGGTCTGCAGTCTGTGCCTATGACATGCTTGACATTGCCAGT
GTTTTTACTGGGAGATTCAAGGAACAGAAGTCTCCTGATTCCACCTGGACACCAGTTCCTGATGAACG
AGTTCCTAAGCCCAGGCCAGGTTGCTGTGCTGGCTCATCCTCCTTAGAAAGATATGCAACCTCCAATG
AGTTCCCTGATGATACCCTGAACTTCATCAAGACGCACCCGCTCATGGATGAGGCAGTGCCCTCCATC
TTCAACAGGCCATGGTTCCTGAGAACAATGGTCAGATGCAGCTATGATGGAGTCGAAGACAAAAGGAT
CATGGGCATGCAGCTGGACAGAGCAAGCAGCTCTCTGTATGTTGCGTTCTCTACCTGTGTGATAAAGG
TTCCCCTTGGCCGGTGTGAACGACATGGGAAGTGTAAAAAAACCTGTATTGCCTCCAGAGACCCATAT
TGTGGATGGATAAAGGAAGGTGGTGCCTGCAGCCATTTATCACCCAACAGCAGACTGACTTTTGAGCA
GGACATAGAGCGTGGCAATACAGATGGTCTGGGGGACTGTCACAATTCCTTTGTGGCACTGAATGGGC
ATTCCAGTTCCCTCTTGCCCAGCACAACCACATCAGATTCGACGGCTCAAGAGGGGTATGAGTCTAGG
GGAGGAATGCTGGACTGGAAGCATCTGCTTGACTCACCTGACAGCACAGACCCTTTGGGGGCAGTGTC
TTCCCATAATCACCAAGACAAGAAGGGAGTGATTCGGGAAAGTTACCTCAAAGGCCACGACCAGCTGG
TTCCCGTCACCCTCTTGGCCATTGCAGTCATCCTGGCTTTCGTCATGGGGGCCGTCTTCTCGGGCATC
ACCGTCTACTGCGTCTGTGATCATCGGCGCAAAGACGTGGCTGTGGTGCAGCGCAAGGAGAAGGAGCT
CACCCACTCGCGCCGGGGCTCCATGAGCAGCGTCACCAAGCTCAGCGGCCTCTTTGGGGACACTCAAT
CCAAAGACCCAAAGCCGGAGGCCATCCTCACGCCACTCATGCACAACGGCAAGCTCGCCACTCCCGGC
AACACGGCCAAGATGCTCATTAAAGCAGACCAGCACCACCTGGACCTGACGGCCCTCCCCACCCCAGA
GTCAACCCCAACGCTGCAGCAGAAGCGGAAGCCCAGCCGCGGCAGCCGCGAGTGGGAGAGGAACCAGA
ACCTCATCAATGCCTGCACAAAGGACATGCCCCCCATGGGCTCCCCTGTGATTCCCACGGACCTGCCC
CTGCGGGCCTCCCCCAGCCACATCCCCAGCGTGGTGGTCCTGCCCATCACGCAGCAGGGCTACCAGCA
TGAGTACGTGGACCAGCCCAAAATGAGCGAGGTGGCCCAGATGGCGCTGGAGGACCAGGCCGCCACAC
TGGAGTATAAGACCATCAAGGAACATCTCAGCAGCAAGAGTCCCAACCATGGGGTGAACCTTGTGGAG
AACCTGGACAGCCTGCCCCCCAAAGTTCCACAGCGGGAGGCCTCCCTGGGTCCCCCGGGAGCCTCCCT
GTCTCAGACCGGTCTAAGCAAGCGGCTGGAAATGCACCACTCCTCTTCCTACGGGGTTGACTATAAGA
GGAGCTACCCCACGAACTCGCTCACGAGAAGCCACCAGGCCACCACTCTCAAAAGAAACAACACTAAC
TCCTCCAATTCCTCTCACCTCTCCAGAAACCAGAGCTTTGGCAGGGGAGACAACCCGCCGCCCGCCCC
GCAGAGGGTGGACTCCATCCAGGTGCACAGCTCCCAGCCATCTGGCCAGGCCGTGACTGTCTCGAGGC
AGCCCAGCCTCAACGCCTACAACTCACTGACAAGGTCGGGGCTGAAGCGTACGCCCTCGCTAAAGCCG
GACGTACCCCCCAAACCATCCTTTGCTCCCCTTTCCACATCCATGAAGCCCAATGATGCGTGTACATA
ATCCCAGGGGGAGGGGGTCAGGTGTCGAACCAGCAGGCAAGGCGAGGTGCCCGCTCAGCTCAGCAAGG
TTCTCAACTGCCTCGAGTACCCACCAGACCAAGAAGGCCTGCGGCAGAGCCGAGGACGCTGGGTCCTC
CTCTCTGGGACACAGGGGTACTCACGAAAACTGGGCCGCGTGGTTTGGTGAAGGTTTGCAACGGCGGG
GACTCACCTTCATTCTCTTCCTTCACTTTCCCCCACACCCTACAACAGGTCGGACCCACAAAAGACTT
CAGTTATCATCACAAACATGAGCCAAAAGCACATACCTACCCCATCCCCCACCCCCACACACACACAC
ATGCACACAACACATACACACACACGCACAGAGGTGAACAGAAACTGAAACATTTTGTCCACAACTTC
ACGGGACGTGGCCAGACTGGGTTTGCGTTCCAACCTGCAAAACACAAATACATTTTTTAAAATCAAGA
AAATTTAAAAAGACAAAAAAAAGAATTCATTGATAATTCTAACTCAGACTTTAACAATGGCAGAAGTT
TACTATGCGCAAATACTGTGAAATGCCCGCCAGTGTTACAGCTTTCTGTTGCAGCAGATAAATGCCAT
GTTGGGCAGCTATGTCATAGATTTCTGCTCCTCCTCTCTTGTCATAGATTTCTGCTCCTCCTCTCTTG
TCATAGATTTCTGCTCCTCCTCTCTTGTCATAGATTTCTGCTCCTCCTCTCTTGTCATAGATTTCTGC
TCCTCCTCTCTTGTCATAGATTTCTGCTCCTCCTCTCTTGTCATAGATTTCTGCTCCTCCTCTCTTGT
CATAGATTTCTGCTCCTCCTCTCTTGTCATAGATTTCTG

| | | | |
|---|---|---|---|
| NOV2q, CG51896-09 | | | |
| Protein Sequence | SEQ ID NO: 46 | 971 aa | MW at 107846.1 kD |

MRSEALLLYFTLLHFAGAGFPEDSEPISISHGNYTKQYPVFVGHKPGRNTTQRHRLDIQMIMIHNGTL
YIAARDHIYTVDIDTSHTEEIYCSKKLTWKSRQADVDTCRMKGKHKDECHNFIKVLLKKNDDALFVCG
TNAFNPSCRNYKMDTLEPFGDEFSGMARCPYDAKHANVALFADGKLYSATVTDFLAIDAVIYRSLGES
PTLRTVKHDSKWLKEPYFVQAVDYGDYIYFFFREIAVEYNTMGKVVFPRVAQVCKNDMGGSQRVLEKR
WTSFLKARLNCSVPGDSHFYFNILQAVTDVIRINGRDVVLATFSTPYNSIPGSAVCAYDMLDIASVFT
GRFKEQKSPDSTWTPVPDERVPKPRPGCCAGSSSLERYATSNEFPDDTLNFIKTHPLMDEAVPSIFNR
PWFLRTMVRCSYDGVEDKRIMGMQLDRASSSLYVAFSTCVIKVPLGRCERHGKCKKTCIASRDPYCGW
IKEGGACSHLSPNSRLTFEQDIERGNTDGLGDCHNSFVALNGHSSSLLPSTTTSDSTAQEGYESRGGM
LDWKHLLDSPDSTDPLGAVSSHNHQDKKGVIRESYLKGHDQLVPVTLLAIAVILAFVMGAVFSGITVY
CVCDHRRKDVAVVQRKEKELTHSRRGSMSSVTKLSGLFGDTQSKDPKPEAILTPLMHNGKLATPGNTA
KMLIKADQHHLDLTALPTPESTPTLQQKRKPSRGSREWERNQNLINACTKDMPPMGSPVIPTDLPLRA
SPSHIPSVVVLPITQQGYQHEYVDQPKMSEVAQMALEDQAATLEYKTIKEHLSSKSPNHGVNLVENLD
SLPPKVPQREASLGPPGASLSQTGLSKRLEMHHSSSYGVDYKRSYPTNSLTRSHQATTLKRNNTNSSN
SSHLSRNQSFGRGDNPPPAPQRVDSIQVHSSQPSGQAVTVSRQPSLNAYNSLTRSGLKRTPSLKPDVP
PKPSFAPLSTSMKPNDACT

| | | |
|---|---|---|
| NOV2r, CG51896-10 | SEQ ID NO: 47 | 3165 bp |
| DNA Sequence | ORF Start: ATG at 13 | ORF Stop: end of sequence |

CAGCGCGGATCCATGAGGTCAGAAGCCTTGCTGCTGTATTTCACACTGCTACACTTTGCTGGGGCTGG
TTTCCCAGAAGATTCTGAGCCAATCAGTATTTCGCATGGCAACTATACAAAACAGTATCCGGTGTTTG
TGGGCCACAAGCCAGGACGGAACACCACACAGAGGCACAGGCTGGACATCCAGATGATTATGATCATG
AACGGAACCCTCTACATTGCTGCTAGGGACCATATTTATACTGTTGATATAGACACATCACACACGGA
AGAAATTTATTGTAGCAAAAAACTGACATGGAAATCTAGACAGGCCGATGTAGACACATGCAGAATGA
AGGGAAAACATAAGGATGAGTGCCACAACTTTATTAAAGTTCTTCTAAAGAAAAACGATGATGCATTG
TTTGTCTGTGGAACTAATGCCTTCAACCCTTCCTGCAGAAACTATAAGATGGATACATTGGAACCATT
CGGGGATGAATTCAGCGGAATGGCCAGATGCCCATATGATGCCAAACATGCCAACGTTGCACTGTTTG
CAGATGGAAAACTATACTCAGCCACAGTGACTGACTTCCTTGCCATTGACGCAGTCATTTACCGGAGT
CTTGGAGAAAGCCCTACCCTGCGGACCGTCAAGCACGATTCAAAATGGTTGAAAGAACCATACTTTGT
TCAAGCCGTGGATTACGGAGATTATATCTACTTCTTCTTCAGGGAAATAGCAGTGGAGTATAACACCA
TGGGAAAGGTAGTTTTCCCAAGAGTGGCTCAGGTTTGTAAGAATGATATGGGAGGATCTCAAAGAGTC
CTGGAGAAACAGTGGACGTCGTTCCTGAAGGCGCGCTTGAACTGCTCAGTTCCTGGAGACTCTCATTT
TTATTTCAACATTCTCCAGGCAGTTACAGATGTGATTCGTATCAACGGGCGTGATGTTGTCCTGGCAA
CGTTTTCTACACCTTATAACAGCATCCCTGGGTCTGCAGTCTGTGCCTATGACATGCTTGACATTGCC
AGTGTTTTTACTGGGAGATTCAAGGAACAGAAGTCTCCTGATTCCACCTGGACACCAGTTCCTGATGA
ACGAGTTCCTAAGCCCAGGCCAGGTTGCTGTGCTGGCTCATCCTCCTTAGAAAGATATGCAACCTCCA
ATGAGTTCCCTGATGATACCCTGAACTTCATCAAGACGCACCCGCTCATGGATGAGGCAGTGCCCTCC
ATCTTCAACAGGCCATGGTTCCTGAGAACAATGGTCAGATACCGCCTTACCAAAATTGCAGTGGACAC
AGCTGCTGGGCCATATCAGAATCACACTGTGGTTTTTCTGGGATCAGAGAAGGGAATCATCTTGAAGT
TTTTGGCCAGAATAGGAAATAGTGGTTTTCTAAATGACAGCCTTTTCCTGGAGGAGATGAGTGTTTAC
AACTCTGAAAAATGCAGCTATGATGGAGTCGAAGACAAAAGGATCATGGGCATGCAGCTGGACAGAGC
AAGCAGCTCTCTGTATGTTGCGTTCTCTACCTGTGTGATAAAGGTTCCCCTTGGCCGGTGTGAACGAC
ATGGGAAGTGTAAAAAAACCTGTATTGCCTCCAGAGACCCGTATTGTGGATGGATAAAGGAAGGTGGT
GCCTGCAGCCATTTATCACCCAACAGCAGACTGACTTTTGAGCAGGACATAGAGCGTGGCAATACAGA
TGGTCTGGGGGACTGTCACAATTCCTTTGTGGCACTGAATGACATTTCAACTCCTCTACCAGATAATG
AAATGTCTTACAACACAGTGTATGGGCATTCCAGTTCCCTCTTGCCCAGCACAACCACATCAGATTCG
ACGGCTCAAGAGGGGTATGAGTCTAGGGGAGGAATGCTGGACTGGAAGCATCTGCTTGACTCACCTGA
CAGCACAGACCCTTTGGGGGCAGTGTCTTCCCATAATCACCAAGACAAGAAGGGAGTGATTCGGGAAA
GTTACCTCAAAGGCCACGACCAGCTGGTTCCCGTCACCCTCTTGGCCATTGCAGTCATCCTGGCTTTC
GTCATGGGGGCCGTCTTCTCGGGCATCACCGTCTACTGCGTCTGTGATCATCGGCGCAAAGACGTGGC
TGTGGTGCAGCGCAAGGAGAAGGAGCTCACCCACTCGCGCCGGGGCTCCATGAGCAGCGTCACCAAGC
TCAGCGGCCTCTTTGGGGACACTCAATCCAAAGACCCAAAGCCGGAGGCCATCCTCACGCCACTCATG
CACAACGGCAAGCTCGCCACTCCCGGCAACACGGCCAAGATGCTCATTAAAGCAGACCAGCACCACCT
GGACCTGACGGCCCTCCCCACCCCAGAGTCAACCCCAACGCTGCAGCAGAAGCGGAAGCCCAGCCGCG
GCAGCCGCGAGTGGGAGAGGAACCAGAACCTCATCAATGCCTGCACAAAGGACATGCCCCCCATGGGC
TCCCCTGTGATTCCCACGGACCTGCCCCTGCGGGCCTCCCCCAGCCACATCCCCAGCGTGGTGGTCCT
GCCCATCACGCAGCAGGGCTACCAGCATGAGTACGTGGACCAGCCCAAAATGAGCGAGGTGGCCCAGA
TGGCGCTGGAGGACCAGGCCGCCACACTGGAGTATAAGACCATCAAGGAACATCTCAGCAGCAAGAGT
CCCAACCATGGGGTGAACCTTGTGGAGAACCTGGACAGCCTGCCCCCCAAAGTTCCACAGCGGGAGGC
CTCCCTGGGTCCCCCGGGAGCCTCCCTGTCTCAGACCGGTCTAAGCAAGCGGCTGGAAATGCACCACT
CCTCTTCCTACGGGGTTGACTATAAGAGGAGCTACCCCACGAACTCGCTCACGAGAAGCCACCAGGCC
ACCACTCTCAAAAGAAACAACACTAACTCCTCCAATTCCTCTCACCTCTCCAGAAACCAGAGCTTTGG
CAGGGGAGACAACCCGCCGCCCGCCCCGCAGAGGGTGGACTCCATCCAGGTGCACAGCTCCCAGCCAT
CTGGCCAGGCCGTGACTGTCTCGAGGCAGCCCAGCCTCAACGCCTACAACTCACTGACAAGGTCGGGG
CTGAAGCGTACGCCCTCGCTAAAGCCGGACGTACCCCCCAAACCATCCTTTGCTCCCCTTTCCACATC
CATGAAGCCCAATGATGCGTGTACAGTCGACGCGCTG

| | | | |
|---|---|---|---|
| NOV2r, CG51896-10 | | | |
| Protein Sequence | SEQ ID NO: 48 | 1047 aa | MW at 116308.5 kD |

MRSEALLLYFTLLHFAGAGFPEDSEPISISHGNYTKQYPVFVGHKPGRNTTQRHRLDIQMIMIMNGTL
YIAARDHIYTVDIDTSHTEEIYCSKKLTWKSRQADVDTCRMKGKHKDECHNFIKVLLKKNDDALFVCG
TNAFNPSCRNYKMDTLEPFGDEFSGMARCPYDAKHANVALFADGKLYSATVTDFLAIDAVIYRSLGES
PTLRTVKHDSKWLKEPYFVQAVDYGDYIYFFFREIAVEYNTMGKVVFPRVAQVCKNDMGGSQRVLEKQ
WTSFLKARLNCSVPGDSHFYFNILQAVTDVIRINGRDVVLATFSTPYNSIPGSAVCAYDMLDIASVFT
GRFKEQKSPDSTWTPVPDERVPKPRPGCCAGSSSLERYATSNEFPDDTLNFIKTHPLMDEAVPSIFNR
PWFLRTMVRYRLTKIAVDTAAGPYQNHTVVFLGSEKGIILKFLARIGNSGFLNDSLFLEEMSVYNSEK
CSYDGVEDKRIMGMQLDRASSSLYVAFSTCVIKVPLGRCERHGKCKKTCIASRDPYCGWIKEGGACSH
LSPNSRLTFEQDIERGMTDGLGDCHNSFVALNDISTPLPDNEMSYNTVYGHSSSLLPSTTTSDSTAQE
GYESRGGMLDWKHLLDSPDSTDPLGAVSSHNHQDKKGVIRESYLKGHDQLVPVTLLAIAVILAFVMGA
VFSGITVYCVCDHRRKDVAVVQRKEKELTHSRRGSMSSVTKLSGLFGDTQSKDPKPEAILTPLMHNGK
LATPGNTAKMLIKADQHHLDLTALPTPESTPTLQQKRKPSRGSREWERNQNLINACTKDMPPMGSPVI
PTDLPLRASPSHIPSVVVLPITQQGYQHEYVDQPKMSEVAQMALEDQAATLEYKTIKEHLSSKSPNHG
VNLVENLDSLPPKVPQREASLGPPGASLSQTGLSKRLEMHHSSSYGVDYKRSYPTNSLTRSHQATTLK
RNNTNSSNSSHLSRNQSFGRGDNPPPAPQRVDSIQVHSSQPSGQAVTVSRQPSLNAYNSLTRSGLKRT
PSLKPDVPPKPSFAPLSTSMKPNDACT

| | | |
|---|---|---|
| NOV2s, CG51896-11 | SEQ ID NO: 49 | 1948 bp |
| DNA Sequence | ORF Start: at 2 | ORF Stop: end of sequence |

CACCGGATCCGGTTTCCCAGAAGATTCTGAGCCAATCAGTATTTCGCATGGCAACTATACAAAACAGT
ATCCGGTGTTTGTGGGCCACAAGCCAGGACGGAACACCACACAGAGGCACAGGCTGGACATCCAGATG
ATTATGATCATGAACGGAACCCTCTACATTGCTGCTAGGGACCATATTTATACTGTTGATATAGACAC
ATCACACACGGAAGAAATTTATTGTAGCAAAAAACTGACATGGAAATCTAGACAGGCCGATGTAGACA
CATGCAGAATGAAGGGAAAACATAAGGATGAGTGCCACAACTTTATTAAAGTTCTTCTAAAGAAAAAC
GATGATGCATTGTTTGTCTGTGGAACTAATGCCTTCAACCCTTCCTGCAGAAACTATAAGATGGATAC
ATTGGAACCATTCGGGGATGAATTCAGCGGAATGGCCAGATGCCCATATGATGCCAAACATGCCAACG
TTGCACTGTTTGCAGATGGAAAACTATACTCAGCCACAGTGACTGACTTCCTTGCCATTGACGCAGTC
ATTTACCGGAGTCTTGGAGAAAGCCCTACCCTGCGGACCGTCAAGCACGATTCAAAATGGTTGAAAGA
ACCATACTTTGTTCAAGCCGTGGATTACGGAGATTATATCTACTTCTTCTTCAGGGAAATAGCAGTGG
AGTATAACACCATGGGAAAGGTAGTTTTCCCAAGAGTGGCTCAGGTTTGTAAGAATGATATGGGAGGA
TCTCAAAGAGTCCTGGAGAAACAGTGGACGTCGTTCCTGAAGGCGCGCTTGAACTGCTCAGTTCCTGG
AGACTCTCATTTTTATTTCAACATTCTCCAGGCAGTTACAGATGTGATTCGTATCAACGGGCGTGATG
TTGTCCTGGCAACGTTTTCTACACCTTATAACAGCATCCCTGGGTCTGCAGTCTGTGCCTATGACATG
CTTGACATTGCCAGTGTTTTTACTGGGAGATTCAAGGAACAGAAGTCTCCTGATTCCACCTGGACACC
AGTTCCTGATGAACGAGTTCCTAAGCCCAGGCCAGGTTGCTGTGCTGGCTCATCCTCCTTAGAAAGAT
ATGCAACCTCCAATGAGTTCCCTGATGATACCCTGAACTTCATCAAGACGCACCCGCTCATGGATGAG
GCAGTGCCCTCCATCTTCAACAGGCCATGGTTCCTGAGAACAATGGTCAGATACCGCCTTACCAAAAT
TGCAGTGGACACAGCTGCTGGGCCATATCAGAATCACACTGTGGTTTTTCTGGGATCAGAGAAGGGAA
TCATCTTGAAGTTTTTGGCCAGAATAGGAAATAGTGGTTTTCTAAATGACAGCCTTTTCCTGGAGGAG
ATGAGTGTTTACAACTCTGAAAAATGCAGCTATGATGGAGTCGAAGACAAAAGGATCATGGGCATGCA
GCTGGACAGAGCAAGCAGCTCTCTGTATGTTGCGTTCTCTACCTGTGTGATAAAGGTTCCCCTTGGCC
GGTGTGAACGACATGGGAAGTGTAAAAAAACCTGTATTGCCTCCAGAGACCCGTATTGTGGATGGATA
AAGGAAGGTGGTGCCTGCAGCCATTTATCACCCAACAGCAGACTGACTTTTGAGCAGGACATAGAGCG
TGGCAATACAGATGGTCTGGGGGACTGTCACAATTCCTTTGTGGCACTGAATGACATTTCAACTCCTC
TACCAGATAATGAAATGTCTTATAACACAGTGTATGGGCATTCCAGTTCCCTCTTGCCCAGCACAACC
ACATCAGATTCGACGGCTCAAGAGGGGTATGAGTCTAGGGGAGGAATGCTGGACTGGAAGCATCTGCT
TGACTCACCTGACAGCACAGACCCTTTGGGGGCAGTGTCTTCCCACAATCACCAAGACAAGAAGGGAG
TGATTCGGGAAAGTTACCTCAAAGGCCACGACCAGGTCGACGGC

| | | | |
|---|---|---|---|
| NOV2s, CG51896-11 | | | |
| Protein Sequence | SEQ ID NO: 50 | 649 aa | MW at 72755.3 kD |

TGSGFPEDSEPISISHGNYTKQYPVFVGHKPGRNTTQRHRLDIQMIMINNGTLYIAARDHIYTVDIDT
SHTEEIYCSKKLTWKSRQADVDTCRMKGKHKDECHNFIKVLLKKNDDALFVCGTNAFNPSCRNYKMDT
LEPFGDEFSGMARCPYDAKHANVALFADGKLYSATVTDFLAIDAVIYRSLGESPTLRTVKHDSKWLKE
PYFVQAVDYGDYIYFFFREIAVEYNTMGKVVFPRVAQVCKNDMGGSQRVLEKQWTSFLKARLNCSVPG
DSHFYFNILQAVTDVIRINGRDVVLATFSTPYNSIPGSAVCAYDMLDIASVFTGRFKEQKSPDSTWTP
VPDERVPKPRPGCCAGSSSLERYATSNEFPDDTLNFIKTHPLMDEAVPSIFNRPWFLRTMVRYRLTKI
AVDTAAGPYQNHTVVFLGSEKGIILKFLARIGNSGFLWDSLFLEEMSVYNSEKCSYDGVEDKRIMGMQ
LDRASSSLYVAFSTCVIKVPLGRCERHGKCKKTCIASRDPYCGWIKEGGACSHLSPNSRLTFEQDIER
GNTDGLGDCHNSFVALNDISTPLPDNEMSYNTVYGHSSSLLPSTTTSDSTAQEGYESRGGMLDWKHLL
DSPDSTDPLGAVSSHNHQDKKGVIRESYLKGHDQVDG

| | | |
|---|---|---|
| NOV2t, CG51896-12 | SEQ ID NO: 51 | 2583 bp |
| DNA Sequence | ORF Start: at 1 | ORF Stop: end of sequence |

GACAAAACTCACACATGCCCACCGTGCCCAGCACCTGAACTCCTGGGGGGACCGTCAGTCTTCCTCTT
CCCCCCAAAACCCAAGGACACCCTCATGATCTCCCGGACCCCTGAGGTCACATGCGTGGTGGTGGACG
TGAGCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCGGGAT
GAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTAA
AGGCGGCGGCGGCGGCGGCGGCGGCGGTTTCCCAGAAGATTCTGAGCCAATCAGTATTTCGCATGGCA
ACTATACAAAACAGTATCCGGTGTTTGTGGGCCACAAGCCAGGACGGAACACCACACAGAGGCACAGG
CTGGACATCCAGATGATTATGATCATGAACGGAACCCTCTACATTGCTGCTAGGGACCATATTTATAC
TGTTGATATAGACACATCACACACGGAAGAAATTTATTGTAGCAAAAAACTGACATGGAAATCTAGAC
AGGCCGATGTAGACACATGCAGAATGAAGGGAAAACATAAGGATGAGTGCCACAACTTTATTAAAGTT
CTTCTAAAGAAAAACGATGATGCATTGTTTGTCTGTGGAACTAATGCCTTCAACCCTTCCTGCAGAAA
CTATAAGATGGATACATTGGAACCATTCGGGGATGAATTCAGCGGAATGGCCAGATGCCCATATGATG
CCAAACATGCCAACGTTGCACTGTTTGCAGATGGAAAACTATACTCAGCCACAGTGACTGACTTCCTT
GCCATTGACGCAGTCATTTACCGGAGTCTTGGAGAAAGCCCTACCCTGCGGACCGTCAAGCACGATTC
AAAATGGTTGAAAGAACCATACTTTGTTCAAGCCGTGGATTACGGAGATTATATCTACTTCTTCTTCA
GGGAAATAGCAGTGGAGTATAACACCATGGGAAAGGTAGTTTTCCCAAGAGTGGCTCAGGTTTGTAAG
AATGATATGGGAGGATCTCAAAGAGTCCTGGAGAAACAGTGGACGTCGTTCCTGAAGGCGCGCTTGAA
CTGCTCAGTTCCTGGAGACTCTCATTTTTATTTCAACATTCTCCAGGCAGTTACAGATGTGATTCGTA
TCAACGGGCGTGATGTTGTCCTGGCAACGTTTTCTACACCTTATAACAGCATCCCTGGGTCTGCAGTC
TGTGCCTATGACATGCTTGACATTGCCAGTGTTTTTACTGGGAGATTCAAGGAACAGAAGTCTCCTGA
TTCCACCTGGACACCAGTTCCTGATGAACGAGTTCCTAAGCCCAGGCCAGGTTGCTGTGCTGGCTCAT
CCTCCTTAGAAAGATATGCAACCTCCAATGAGTTCCCTGATGATACCCTGAACTTCATCAAGACGCAC
CCGCTCATGGATGAGGCAGTGCCCTCCATCTTCAACAGGCCATGGTTCCTGAGAACAATGGTCAGATA
CCGCCTTACCAAAATTGCAGTGGACACAGCTGCTGGGCCATATCAGAATCACACTGTGGTTTTTCTGG
GATCAGAGAAGGGAATCATCTTGAAGTTTTTGGCCAGAATAGGAAATAGTGGTTTTCTAAATGACAGC
CTTTTCCTGGAGGAGATGAGTGTTTACAACTCTGAAAAATGCAGCTATGATGGAGTCGAAGACAAAAG
GATCATGGGCATGCAGCTGGACAGAGCAAGCAGCTCTCTGTATGTTGCGTTCTCTACCTGTGTGATAA
AGGTTCCCCTTGGCCGGTGTGAACGACATGGGAAGTGTAAAAAAACCTGTATTGCCTCCAGAGACCCA
TATTGTGGATGGATAAAGGAAGGTGGTGCCTGCAGCCATTTATCACCCAACAGCAGACTGACTTTTGA
GCAGGACATAGAGCGTGGCAATACAGATGGTCTGGGGGACTGTCACAATTCCTTTGTGGCACTGAATG
GGCATTCCAGTTCCCTCTTGCCCAGCACAACCACATCAGATTCGACGGCTCAAGAGGGGTATGAGTCT
AGGGGAGGAATGCTGGACTGGAAGCATCTGCTTGACTCACCTGACAGCACAGACCCTTTGGGGGCAGT
GTCTTCCCATAATCACCAAGACAAGAAGGGAGTGATTCGGGAAAGTTACCTCAAAGGCCACGACCAG

| | | | |
|---|---|---|---|
| NOV2t, CG51896-12 | | | |
| Protein Sequence | SEQ ID NO: 52 | 861 aa | MW at 96283.9 kD |

DKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVFS
CSVMHEALHNHYTQKSLSLSPGKGGGGGGGGGFPEDSEPISISHGNYTKQYPVFVGHKPGRNTTQRHR
LDIQMIMIMNGTLYIAARDHIYTVDIDTSHTEEIYCSKKLTWKSRQADVDTCRMKGKHKDECHNFIKV
LLKKNDDALFVCGTNAFNPSCRNYKMDTLEPFGDEFSGMARCPYDAKHAVVALFADGKLYSATVTDFL
AIDAVIYRSLGESPTLRTVKHDSKWLKEPYFVQAVDYGDYIYFFFREIAVEYNTMGKVVFPRVAQVCK
NDMGGSQRVLEKQWTSFLKARLNCSVPGDSHFYFNILQAVTDVIRINGRDVVLATFSTPYNSIPGSAV
CAYDMLDIASVFTGRFKEQKSPDSTWTPVPDERVPKPRPGCCAGSSSLERYATSNEFPDDTLNFIKTH
PLMDEAVPSIFNRPWFLRTMVRYRLTKIAVDTAAGPYQNHTVVFLGSEKGIILKFLARIGNSGFLNDS
LFLEEMSVYNSEKCSYDGVEDKRIMGMQLDRASSSLYVAFSTCVIKVPLGRCERHGKCKKTCIASRDP
YCGWIKEGGACSHLSPNSRLTFEQDIERGNTDGLGDCHNSFVALNGHSSSLLPSTTTSDSTAQEGYES
RGGMLDWKHLLDSPDSTDPLGAVSSHNHQDKKGVIRESYLKGHDQ

| | | |
|---|---|---|
| NOV2u, CG51896-13 | SEQ ID NO: 53 | 2634 bp |
| DNA Sequence | ORF Start: at 1 | ORF Stop: end of sequence |

GACAAAACTCACACATGCCCACCGTGCCCAGCACCTGAACTCCTGGGGGGACCGTCAGTCTTCCTCTT
CCCCCCAAAACCCAAGGACACCCTCATGATCTCCCGGACCCCTGAGGTCACATGCGTGGTGGTGGACG
TGAGCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCGGGAT
GAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTAA
AGGCGGCGGCGGCGGCGGCGGCGGCGGTTTCCCAGAAGATTCTGAGCCAATCAGTATTTCGCATGGCA
ACTATACAAAACAGTATCCGGTGTTTGTGGGCCACAAGCCAGGACGGAACACCACACAGAGGCACAGG
CTGGACATCCAGATGATTATGATCATGAACGGAACCCTCTACATTGCTGCTAGGGACCATATTTATAC
TGTTGATATAGACACATCACACACGGAAGAAATTTATTGTAGCAAAAAACTGACATGGAAATCTAGAC
AGGCCGATGTAGACACATGCAGAATGAAGGGAAAACATAAGGATGAGTGCCACAACTTTATTAAAGTT
CTTCTAAAGAAAAACGATGATGCATTGTTTGTCTGTGGAACTAATGCCTTCAACCCTTCCTGCAGAAA
CTATAAGATGGATACATTGGAACCATTCGGGGATGAATTCAGCGGAATGGCCAGATGCCCATATGATG
CCAAACATGCCAACGTTGCACTGTTTGCAGATGGAAAACTATACTCAGCCACAGTGACTGACTTCCTT
GCCATTGACGCAGTCATTTACCGGAGTCTTGGAGAAAGCCCTACCCTGCGGACCGTCAAGCACGATTC
AAAATGGTTGAAAGAACCATACTTTGTTCAAGCCGTGGATTACGGAGATTATATCTACTTCTTCTTCA
GGGAAATAGCAGTGGAGTATAACACCATGGGAAAGGTAGTTTTCCCAAGAGTGGCTCAGGTTTGTAAG
AATGATATGGGAGGATCTCAAAGAGTCCTGGAGAAACAGTGGACGTCGTTCCTGAAGGCGCGCTTGAA
CTGCTCAGTTCCTGGAGACTCTCATTTTTATTTCAACATTCTCCAGGCAGTTACAGATGTGATTCGTA
TCAACGGGCGTGATGTTGTCCTGGCAACGTTTTCTACACCTTATAACAGCATCCCTGGGTCTGCAGTC
TGTGCCTATGACATGCTTGACATTGCCAGTGTTTTTACTGGGAGATTCAAGGAACAGAAGTCTCCTGA
TTCCACCTGGACACCAGTTCCTGATGAACGAGTTCCTAAGCCCAGGCCAGGTTGCTGTGCTGGCTCAT
CCTCCTTAGAAAGATATGCAACCTCCAATGAGTTCCCTGATGATACCCTGAACTTCATCAAGACGCAC
CCGCTCATGGATGAGGCAGTGCCCTCCATCTTCAACAGGCCATGGTTCCTGAGAACAATGGTCAGATA
CCGCCTTACCAAAATTGCAGTGGACACAGCTGCTGGGCCATATCAGAATCACACTGTGGTTTTTCTGG
GATCAGAGAAGGGAATCATCTTGAAGTTTTTGGCCAGAATAGGAAATAGTGGTTTTCTAAATGACAGC
CTTTTCCTGGAGGAGATGAGTGTTTACAACTCTGAAAAATGCAGCTATGATGGAGTCGAAGACAAAAG
GATCATGGGCATGCAGCTGGACAGAGCAAGCAGCTCTCTGTATGTTGCGTTCTCTACCTGTGTGATAA
AGGTTCCCCTTGGCCGGTGTGAACGACATGGGAAGTGTAAAAAAACCTGTATTGCCTCCAGAGACCCG
TATTGTGGATGGATAAAGGAAGGTGGTGCCTGCAGCCATTTATCACCCAACAGCAGACTGACTTTTGA
GCAGGACATAGAGCGTGGCAATACAGATGGTCTGGGGGACTGTCACAATTCCTTTGTGGCACTGAATG
ACATTTCAACTCCTCTACCAGATAATGAAATGTCTTATAACACAGTGTATGGGCATTCCAGTTCCCTC
TTGCCCAGCACAACCACATCAGATTCGACGGCTCAAGAGGGGTATGAGTCTAGGGGAGGAATGCTGGA
CTGGAAGCATCTGCTTGACTCACCTGACAGCACAGACCCTTTGGGGGCAGTGTCTTCCCACAATCACC
AAGACAAGAAGGGAGTGATTCGGGAAAGTTACCTCAAAGGCCACGACCAG

| | | | |
|---|---|---|---|
| NOV2u, CG51896-13 | | | |
| Protein Sequence | SEQ ID NO: 54 | 878 aa | MW at 98225.0 kD |

DKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVFS
CSVMHEALNNHYTQKSLSLSPGKGGGGGGGGGFPEDSEPISISHGNYTKQYPVFVGHKPGRNTTQRHR
LDIQMIMIMNGTLYIAARDHIYTVDIDTSHTEEIYCSKKLTWKSRQADVDTCRMKGKHKDECHNFIKV
LLKKNDDALFVCGTNAFNPSCRNYKMDTLEPFGDEFSGMARCPYDAKHANVALFADGKLYSATVTDFL
AIDAVIYRSLGESPTLRTVKHDSKWLKEPYFVQAVDYGDYIYFFFREIAVEYNTMGKVVFPRVAQVCK
NDMGGSQRVLEKQWTSFLKARLNCSVPGDSHFYFNILQAVTDVIRINGRDVVLATFSTPYNSIPGSAV
CAYDMLDIASVFTGRFKEQKSPDSTWTPVPDERVPKPRPGCCAGSSSLERYATSNEFPDDTLNFIKTH
PLMDEAVPSIFNRPWFLRTMVRYRLTKIAVDTAAGPYQNHTVVFLGSEKGIILKFLARIGNSGFLNDS
LFLEEMSVYNSEKCSYDGVEDKRIMGMQLDRASSSLYVAFSTCVIKVPLGRCERHGKCKKTCIASRDP
YCGWIKEGGACSHLSPNSRLTFEQDIERGNTDGLGDCHNSFVALNDISTPLPDNEMSYNTVYGHSSSL
LPSTTTSDSTAQEGYESRGGMLDWKHLLDSPDSTDPLGAVSSHNHQDKKGVIRESYLKGHDQ

| | | |
|---|---|---|
| NOV2v, CG51896-14 | SEQ ID NO: 55 | 2113 bp |
| DNA Sequence | ORF Start: at 1 | ORF Stop: end of sequence |

GCCACCATGGAGACAGACACACTCCTGCTATGGGTACTGCTGCTCTGGGTTCCAGGTTCCACTGGTGA
CGGTTTCCCAGAAGATTCTGAGCCAATCAGTATTTCGCATGGCAACTATACAAAACAGTATCCGGTGT
TTGTGGGCCACAAGCCAGGACGGAACACCACACAGAGGCACAGGCTGGACATCCAGATGATTATGATC
ATGAACGGAACCCTCTACATTGCTGCTAGGGACCATATTTATACTGTTGATATAGACACATCACACAC
GGAGGAAATTTATTGTAGCAAAAAACTGACATGGAAATCTAGACAGGCCGATGTAGACACATGCAGAA
TGAAGGGAAAACATAAGGATGAGTGCCACAACTTTATTAAAGTTCTTCTAAAGAAAAACGATGATGCA
TTGTTTGTCTGTGGAACTAATGCCTTCAACCCTTCCTGCAGAAACTATAAGATGGATACATTGGAACC
ATTCGGGGATGAATTCAGCGGAATGGCCAGATGCCCATATGATGCCAAACATGCCAACGTTGCACTGT
TTGCAGATGGAAAACTATACTCAGCCACAGTGACTGACTTCCTTGCCATTGACGCAGTCATTTACCGG
AGTCTTGGAGAAAGCCCTACCCTGCGGACCGTCAAGCACGATTCALAATGGTTGAAAGAACCATACTT
TGTTCAAGCCGTGGATTACGGAGATTATATCTACTTCTTCTTCAGGGAAATTGCAGTGGAGTATAACA
CCATGGGAAAGGTAGTTTTCCCAAGAGTGGCTCAGGTTTGTAAGAATGATATGGGAGGATCTCAAAGA
GTCCTGGAGAAACAGTGGACGTCGTTCCTGAAGGCGCGCTTGAACTGCTCAGTTCCTGGAGACTCTCA
TTTTTATTTCAACATTCTCCAGGCAGTTACAGATGTGATTCGTATCAACGGGCGTGATGTTGTCCTGG
CAACGTTTTCTACACCTTATAACAGCATCCCTGGGTCTGCAGTCTGTGCCTATGACATGCTTGACATT
GCCAGTGTTTTTACTGGGAGATTCAAGGAACAGAAGTCTCCTGATTCCACCTGGACACCAGTTCCTGA
TGAACGAGTTCCTAAGCCCAGGCCAGGTTGCTGTGCTGGCTCATCCTCCTTAGAAAGATATGCAACCT
CCAATGAGTTCCCTGATGATACCCTGAACTTCATCAAGACGCACCCGCTCATGGATGAGGCAGTGCCC
TCCATCTTCAACAGGCCATGGTTCCTGAGAACAATGGTCAGATACCGCCTTACCAAAATTGCAGTGGA
CACAGCTGCTGGGCCATATCAGAATCACACTGTGGTTTTTCTGGGATCAGAGAAGGGAATCATCTTGA
AGTTTTTGGCCAGAATAGGAAATAGTGGTTTTCTAAATGACAGCCTTTTCCTGGAGGAGATGAGTGTT
TACAACTCTGAAAAATGCAGCTATGATGGAGTCGAAGACAAAAGGATCATGGGCATGCAGCTGGACAG
AGCAAGCAGCTCTCTGTATGTTGCGTTCTCTACCTGTGTGATAAAGGTTCCCCTTGGCCGGTGTGAAC
GACATGGGAAGTGTAAAAAAACCTGTATTGCCTCCAGAGACCCGTATTGTGGATGGATAAAGGAAGGT
GGTGCCTGCAGCCATTTATCACCCAACAGCAGACTGACTTTTGAGCAGGACATAGAGCGTGGCAATAC
AGATGGTCTGGGGGACTGTCACAATTCCTTTGTGGCACTGAATGACATTTCAACTCCTCTACCAGATA
ATGAAATGTCTTACAACACAGTGTATGGGCATTCCAGTTCCCTCTTGCCCAGCACAACCACATCAGAT
TCGACGGCTCAAGAGGGGTATGAGTCTAGGGGAGGAATGCTGGACTGGAAGCATCTGCTTGACTCACC
TGACAGCACAGACCCTTTGGGGGCAGTGTCTTCCCATAATCACCAAGACAAGAAGGGAGTGATTCGGG
AAAGTTACCTCAAAGGCCACGACCAGTGACTCGAGGACTACAAGGATGACGATGACAAGGATTACAAA
GACGACGATGATAAGGACTATAAGGATGATGACGACAAATAATAGCAATTCCTCGACGCTGCATAGGG
TTACA

| | | | |
|---|---|---|---|
| NOV2v, CG51896-14 | | | |
| Protein Sequence | SEQ ID NO: 56 | 1666 aa | MW at 74752.7 kD |

ATMETDTLLLWVLLLWVPGSTGDGFPEDSEPISISHGNYTKQYPVFVGHKPGRNTTQRHRLDIQMIMI
MNGTLYIAARDHIYTVDIDTSHTEEIYCSKKLTWKSRQADVDTCRMKGKHKDECHNFIKVLLKKNDDA
LFVCGTNAFNPSCRNYKMDTLEPFGDEFSGMARCPYDAKHANVALFADGKLYSATVTDFLAIDAVIYR
SLGESPTLRTVKHDSKWLKEPYFVQAVDYGDYIYFFFREIAVEYNTMGKVVFPRVAQVCKNDMGGSQR
VLEKQWTSFLKARLNCSVPGDSHFYFNILQAVTDVIRINGRDVVLATFSTPYMSIPGSAVCAYDMLDI
ASVFTGRFKEQKSPDSTWTPVPDERVPKPRPGCCAGSSSLERYATSNEFPDDTLNFIKTHPLMDEAVP
SIFNRPWFLRTMVRYRLTKIAVDTAAGPYQNHTWFLGSEKGIILKFLARIGNSGFLNDSLFLEEMSV
YNSEKCSYDGVEDKRIMGMQLDRASSSLYVAFSTCVIKVPLGRCERHGKCKKTCIASRDPYCGWIKEG
GACSHLSPNSRLTFEQDIERGNTDGLGDCHNSFVALNDISTPLPDNEMSYNTVYGHSSSLLPSTTTSD
STAQEGYESRGGMLDWKHLLDSPDSTDPLGAVSSHNHQDKKGVIRESYLKGHDQ

Description of CG51896-11 (SEQ ID NO: 50) and CG51896-13 (SEQ ID NO: 54)

CG51896-11 polypeptide was tagged to Fc regions either at the 5′ end or the 3′ end. The resulting variants were cloned into appropriate expression vectors. Similarly, CG51896-13 nucleic acid and protein were tagged to Fc on the 5′ end (that runs from 1-705 in the nucleic acid sequence and 1 to 235 in the protein sequence) and the Fc regions in nucleic acid and the polypeptide are shown (highlighted) below:

Nucleic acid sequence of CG51896-13 tagged with Fc:

Nucleic acid sequence of CG51896-13 tagged with Fc:
(SEQ ID NO: 53)
GACAAAACTCACACATGCCCACCGTGCCCAGCACCTGAACTCCTGGGGGGACCGTCAGTCTTCCTCTT
CCCCCCAAA&CCCAAGGACACCCTCATGATCTCCCGGACCCCTGAGGTCACATGCGTGGTGGTGGACG
TGAGCCACGAAQkCCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCGGGAT
GAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACG
GCTCCTTCTTCCTCTACAGCAAGCTCACCGTGG&CAkGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTAA
AGGCGGCGGCGGCGGCGGCGGCGGCGGTTTCCCAGAAGATTCTGAGCCAATCAGTATTTCGCATGGCA
ACTATACAAAACAGTATCCGGTGTTTGTGGGCCACAAGCCAGGACGGAACACCACACAGAGGCACAGG
CTGGACATCCAGATGATTATGATCATGAACGGAACCCTCTACATTGCTGCTAGGGACCATATTTATAC
TGTTGATATAGACACATCACACACGGAAGAAATTTATTGTAGCAAAAAACTGACATGGAAATCTAGAC
AGGCCGATGTAGACACATGCAGAATGAAGGGAAAACATAAGGATGAGTGCCACAACTTTATTAAAGTT
CTTCTAAAGAAAAACGATGATGCATTGTTTGTCTGTGGAACTAATGCCTTCAACCCTTCCTGCAGAAA
CTATAAGATGGATACATTGGAACCATTCGGGGATGAATTCAGCGGAATGGCCAGATGCCCATATGATG
CCAAACATGCCAACGTTGCACTGTTTGCAGATGGAAAACTATACTCAGCCACAGTGACTGACTTCCTT
GCCATTGACGCAGTCATTTACCGGAGTCTTGGAGAAAGCCCTACCCTGCGGACCGTCAAGCACGATTC
AAAATGGTTGAAAGAACCATACTTTGTTCAAGCCGTGGATTACGGAGATTATATCTACTTCTTCTTCA
GGGAAATAGCAGTGGAGTATAACACCATGGGAAAGGTAGTTTTCCCAAGAGTGGCTCAGGTTTGTAAG
AATGATATGGGAGGATCTCAAAGAGTCCTGGAGAAACAGTGGACGTCGTTCCTGAAGGCGCGCTTGAA
CTGCTCAGTTCCTGGAGACTCTCATTTTTATTTCAACATTCTCCAGGCAGTTACAGATGTGATTCGTA
TCAACGGGCGTGATGTTGTCCTGGCAACGTTTTCTACACCTTATAACAGCATCCCTGGGTCTGCAGTC
TGTGCCTATGACATGCTTGACATTGCCAGTGTTTTTACTGGGAGATTCAAGGAACAGAAGTCTCCTGA
TTCCACCTGGACACCAGTTCCTGATGAACGAGTTCCTAAGCCCAGGCCAGGTTGCTGTGCTGGCTCAT
CCTCCTTAGAAAGATATGCAACCTCCAATGAGTTCCCTGATGATACCCTGAACTTCATCAAGACGCAC
CCGCTCATGGATGAGGCAGTGCCCTCCATCTTCAACAGGCCATGGTTCCTGAGAACAATGGTCAGATA
CCGCCTTACCAAAATTGCAGTGGACACAGCTGCTGGGCCATATCAGAATCACACTGTGGTTTTTCTGG
GATCAGAGAAGGGAATCATCTTGAAGTTTTTGGCCAGAATAGGAAATAGTGGTTTTCTAAATGACAGC
CTTTTCCTGGAGGAGATGAGTGTTTACAACTCTGAAAAATGCAGCTATGATGGAGTCGAAGACAAAAG
GATCATGGGCATGCAGCTGGACAGAGCAAGCAGCTCTCTGTATGTTGCGTTCTCTACCTGTGTGATAA
AGGTTCCCCTTGGCCGGTGTGAACGACATGGGAAGTGTAAAAAAACCTGTATTGCCTCCAGAGACCCG
TATTGTGGATGGATAAAGGAAGGTGGTGCCTGCAGCCATTTATCACCCAACAGCAGACTGACTTTTGA
GCAGGACATAGAGCGTGGCAATACAGATGGTCTGGGGGACTGTCACAATTCCTTTGTGGCACTGAATG
ACATTTCAACTCCTCTACCAGATAATGAAATGTCTTATAACACAGTGTATGGGCATTCCAGTTCCCTC
TTGCCCAGCACAACCACATCAGATTCGACGGCTCAAGAGGGGTATGAGTCTAGGGGAGGAATGCTGGA
CTGGAAGCATCTGCTTGACTCACCTGACAGCACAGACCCTTTGGGGGCAGTGTCTTCCCACAATCACC
AAGACAAGAAGGGAGTGATTCGGGAAAGTTACCTCAAAGGCCACGACCAG
Protein sequence of CG51896-13 tagged with Fc:
(SEQ ID NO: 54)
DKTETCPPCPAPELLGGPSVFLFPPKPKDTLMISRTPEVTCVVVDVBHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVFS
CSVMHEALHNHYTQRSLSLSPGKGGGGGGGGGFPEDSEPISISHGNYTKQYPVFVGHKPGRNTTQRHR
LDIQMIMIMNGTLYIAARDHIYTVDIDTSHTEEIYCSKKLTWKSRQADVDTCRMKGKHKDECHNFIKV
LLKKNDDALFVCGTNAFNPSCRNYKMDTLEPFGDEFSGMARCPYDAKHANVALFADGKLYSATVTDFL
AIDAVIYRSLGESPTLRTVKHDSKWLKEPYFVQAVDYGDYIYFFFREIAVEYNTMGKVVFPRVAQVCK
NDMGGSQRVLEKQWTSFLKARLNCSVPGDSHFYFNILQAVTDVIRINGRDVVLATFSTPYNSIPGSAV
CAYDMLDIASVFTGRFKEQKSPDSTWTPVPDERVPKPRPGCCAGSSSLERYATSNEFPDDTLNFIKTH
PLMDEAVPSIFNRPWFLRTMVRYRLTKIAVDTAAGPYQNHTVVFLGSEKGIILKFLARIGNSGFLNDS
LFLEEMSVYNSEKCSYDGVEDKRIMGMQLDRASSSLYVAFSTCVIKVPLGRCERHGKCKKTCIASRDP
YCGWIKEGGACSHLSPNSRLTFEQDIERGNTDGLGDCHNSFVALNDISTPLPDNEMSYNTVYGHSSSL
LPSTTTSDSTAQEGYESRGGMLDWKHLLDSPDSTDPLGAVSSHNHQDKKGVIRESYLKGHDQ

A ClustalW comparison of the above protein sequences yields the following sequence alignment shown in Table 2B.

TABLE 2B
Comparison of the NOV2 Protein sequences.hz,1/42

NOV2a ------------------------------------------------------------
NOV2b ------------------------------------------------------------
NOV2c ------------------------------------------------------------
NOV2d ------------------------------------------------------------
NOV2e ------------------------------------------------------------
NOV2f ------------------------------------------------------------
NOV2g ------------------------------------------------------------
NOV2h ------------------------------------------------------------
NOV2i ------------------------------------------------------------
NOV2j ------------------------------------------------------------
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q ------------------------------------------------------------
NOV2r ------------------------------------------------------------
NOV2s ------------------------------------------------------------
NOV2t DKTHTCPPCPAPELLGGPSVFLFPPKPKDTLMISRTPEVTCVVVDVSHEDPEVKFNWYVD
NOV2u DKTHTCPPCPAPELLGGPSVFLFPPKPKDTLMISRTPEVTCVVVDVSHEDPEVKFNWYVD
NOV2v ------------------------------------------------------------
NOV2a ------------------------------------------------------------
NOV2b ------------------------------------------------------------
NOV2c ------------------------------------------------------------
NOV2d ------------------------------------------------------------
NOV2e ------------------------------------------------------------
NOV2f ------------------------------------------------------------
NOV2g ------------------------------------------------------------
NOV2h ------------------------------------------------------------
NOV2i ------------------------------------------------------------
NOV2j ------------------------------------------------------------
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q ------------------------------------------------------------
NOV2r ------------------------------------------------------------
NOV2s ------------------------------------------------------------
NOV2t GVEVHNAKTKPREEQYNSTYRVVSVLTVLHQDWLNGKEYKCKVSNKALPAPIEKTISKAK
NOV2u GVEVHNAKTKPREEQYNSTYRVVSVLTVLHQDWLNGKEYKCKVSNKALPAPIEKTISKAK
NOV2v ------------------------------------------------------------
NOV2a ------------------------------------------------------------
NOV2b ------------------------------------------------------------
NOV2c ------------------------------------------------------------
NOV2d ------------------------------------------------------------
NOV2e ------------------------------------------------------------
NOV2f ------------------------------------------------------------
NOV2g ------------------------------------------------------------
NOV2h ------------------------------------------------------------
NOV2i ------------------------------------------------------------
NOV2j ------------------------------------------------------------
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q ------------------------------------------------------------
NOV2r ------------------------------------------------------------
NOV2s ------------------------------------------------------------
NOV2t GQPREPQVYTLPPSRDELTKNQVSLTCLVKGFYPSDIAVEWESNGQPENNYKTTPPVLDS
NOV2u GQPREPQVYTLPPSRDELTKNQVSLTCLVKGFYPSDIAVEWESNGQPENNYKTTPPVLDS
NOV2v ------------------------------------------------------------
NOV2a ----------------------------------MRSEALLLYFTLLHFAG---AGFPED
NOV2b ------------------------------------------------------------
NOV2c -------------------------------------------------TG---SGFPED
NOV2d ------------------------------------------------------------
NOV2e ------------------------------------------------------------
NOV2f -------------------------------TGSMRSEALLLYFTLLHFAG---AGFPED
NOV2g -------------------------------------------------TG---SGFPED
NOV2h -------------------------------------------------TG---SGFPED
NOV2i -------------------------------TGSMRSEALLLYFTLLHFAG---AGFPED
NOV2j ----------------------------------MRSEALLLYFTLLHFAG---AGFPED
NOV2k -------------------------------------------------------GFPED
NOV2l -------------------------------------------------------GFPED
NOV2m ------------------------------------------------------------
NOV2n ------------------------------------------------------------
NOV2o ------------------------------------------------------------
NOV2p ------------------------------------------------------------
NOV2q ----------------------------------MRSEALLLYFTLLHFAG---AGFPED
NOV2r ----------------------------------MRSEALLLYFTLLHFAG---AGFPED
NOV2s -------------------------------------------------TG---SGFPED
NOV2t DGSFFLYSKLTVDKSRWQQGNVFSCSVMHEALHNHYTQKSLSLSPGKGGGGGGGGGFPED
NOV2u DGSFFLYSKLTVDKSRWQQGNVFSCSVMHEALHNHYTQKSLSLSPGKGGGGGGGGGFPED
NOV2v --------------------------------ATMETDTLLLWVLLLWVPGSTGDGFPED
NOV2a SEPISISHCNYTKQYPVFVGHKPGRNTTQRHRLDIQMIMIMNGTLYIAARDHIYTVDIDT
NOV2b -AGSSISHCNYTKQYPVFVGHKPGRNTTQRHRLDIQMIMIMNGTLYIAARDHIYTVDIDT
NOV2c SEPISISHGNYTKQYPVFVGHKPGRNTTQRHRLDIQMIMIMNGTLYIAARDHIYTVDIDT
NOV2d -----------------------------TGSLDIQMIMIMNGTLYIAARDHIYTVDIDT
NOV2e -----------------------------TGSLDIQMIMIMNGTLYIAARDHIYTVDIDT
NOV2f SEPISISHGNYTKQYPVFVGHKPGRNTTQRHRLDIQMIMIMNGTLYIAARDHIYTVDIDT
NOV2g SEPISISHGNYTKQYPVFVGHKPGRNTTQRHRLDIQMIMIMNGTLYIAARDHIYTVDIDT
NOV2h SEPISISHGNYTKQYPVFVGHKPGRNTTQRHRLDIQMIMIMNGTLYIAARDHIYTVDIDT
NOV2i SEPISISHGNYTKQYPVFVGHKPGRNTTQRHRLDIQMIMIMNGTLYIAARDHIYTVDIDT
NOV2j SEPISISHGNYTKQYPVFVGHKPGRNTTQRHRLDIQMIMIMNGTLYIAARDHIYTVDIDT
NOV2k SEPISISHGNYTKQYPVFVGHKPGRNTTQRHRLDIQMIMIMNGTLYIAARDHIYTVDIDT
NOV21 SEPISISHGNYTKQYPVFVGHKPGRNTTQRHRLDIQMIMINNGTLYIAARDHIYTVDIDT
NOV2m ------------------------------------------------------------
NOV2n ------------------------------------------------------------
NOV2o ------------------------------------------------------------
NOV2p ------------------------------------------------------------
NOV2q SEPISISHGNYTKQYPVFVGHKPGRNTTQRHRLDIQMIMINNGTLYIAARDHIYTVDIDT
NOV2r SEPISISHGNYTKQYPVFVGHKPGRNTTQRHRLDIQMIMIMNGTLYIAARDHIYTVDIDT
NOV2s SEPISISHGNYTKQYPVFVGHKPGRNTTQRHRLDIQMIMIMNGTLYIAARDHIYTVDIDT
NOV2t SEPISISHGNYTKQYPVFVGHKPGRNTTQRHRLDIQMIMIMNGTLYIAARDHIYTVDIDT
NOV2u SEPISISHGNYTKQYPVFVGHKPGRNTTQRHRLDIQMIMINNGTLYIAARDHIYTVDIDT
NOV2v SEPISISHGNYTKQYPVFVGHKPGRNTTQRHRLDIQMIMIMNGTLYIAARDHIYTVDIDT
NOV2a SHTEEIYCSKKLTWKSRQADVDTCRMKGKHKDECHNFIKVLLKKNDDALFVCGTNAFNPS
NOV2b SHTEEIYCSKKLTWKSRQADVDTCRMKGKHKDECHNFIKVLLKKNDDALFVCGTNAFNPS
NOV2c SHTEEIYCSKKLTWKSRQADVDTCRNKGKHKDECHNFIKVLLKKNDDALFVCGTNAFNPS
NOV2d SHTEEIYCSKKLTKSRQADVDTCRMKGKHKDECHNFIKVLLKKIIDDALFVCGTNAFNPS
NOV2e SHTEEIYCSKKLTWKSRQADVDTCRMKGKHKDECHNFIKVLLKKNDDALFVCGTNAFNPS
NOV2f SHTEEIYCSKKLTWKSRQADVDTCRMKGKHKDECHNFIKVLLKKNDDALFVCGTNAFNPS
NOV2g SHTEEIYCSKKLTWKSRQADVDTCRMKGKHKDECHNFIKVLLKKNDDALFVCGTNAFNPS
NOV2h SHTEEIYCSKKLTWKSRQADVDTCRMKGKHKDECHNFIKVLLKKNDDALFVCGTNAFNPS
NOV2i SHTEEIYCSKKLTWKSRQADVDTCRMKGKHKDECHNFIKVLLKKNDDALFVCGTNAFNPS
NOV2j SHTEEIYCSKKLTWKSRQADVDTCRMKGKNKDECHNFIKVLLKINDDALFVCGTNAFNPS
NOV2k SHTEEIYCSKKLTWKSRQADVDTCRMKGKHKDECHNFIKVLLKKNDDALFVCGTNAFNPS
NOV2l SHTEEIYCSKKLTWKSRQADVDTCRMKGKHKDECHNFIKVLLKKNDDALFVCGTNAFNPS
NOV2m ------------------------------------------------------------
NOV2n ------------------------------------------------------------
NOV2o ------------------------------------------------------------
NOV2p ------------------------------------------------------------
NOV2q SHTEEIYCSKKLTWKSRQADVDTCRMKGKHKDECHNFIKVLLKKNDDALFVCGTNAFNPS
NOV2r SHTEEIYCSKKLTWKSRQADVDTCRMKGKNKDECHNFIKVLLKKNDDALFVCGTNAFNPS
NOV2s SHTEEIYCSKKLTWKSRQADVDTCRMKGKHKDECHNFIKVLLKKNDDALFVCGTNAFNPS
NOV2t SHTEEIYCSKKLTWKSRQADVDTCRMKGKHKDECHNFIKVLLKKNDDALFVCGTNAFNPS
NOV2u SHTEEIYCSKKLTWKSRQADVDTCRMKGKHKDECHNFEKVLLKKNDDALFVCGTNAFNPS
NOV2v SHTEEIYCSKKLTWKSRQADVDTCRNKGKHKDECHNFIKVLLKKNDDALFVCGTNAFNPS
NOV2a CRNYKMDTLEPFGDEFSGMARCPYDAKHANVALFADGKLYSATVTDFLAIDAVIYRSLGE
NOV2b CRNYKMDTLEPFGDEFSGMARCPYDAKHANVALFADGKLYSATVTDFLAIDAVIYRSLGE
NOV2c CRNYKMDTLEPFGDEFSGMARCPYDAKHANVALFADGKLYSATVTDFLAIDAVIYRSLGE
NOV2d CRNYKMDTLEPFGDEFSGMARCPYDAKHANVALFADGKLYSATVTDFLAIDAVIYRSLGE
NOV2e CRNYKMDTLEPFGDEFSGMARCPYDAKHANVALFADGKLYSATVTDFLAIDAVIYRSLGE
NOV2f CRNYKMDTLEPFGDEFSGMARCPYDAKHANVALFADGKLYSATVTDFLAIDAVIYRSLGE
NOV2g CRNYKMDTLEPFGDEFSGMARCPYDAKHANVALFADGKLYSATVTDFLAIDAVIYRSLGE
NOV2h CRNYKMDTLEPFGDEFSGMARCPYDAKHANVALFADGKLYSATVTDFLAIDAVIYRSLGE
NOV2i CRNYKMDTLEPFGDEFSGMARCPYDAKHANVALFADGKLYSATVTDFLAIDAVIYRSLGE
NOV2j CRNYKMDTLEPFGDEFSGMARCPYDAKHANVALFADGKLYSATVTDFLAIDAVIYRSLGE
NOV2k CRNYKMDTLEPFGDEFSGMARCPYDAKHANVALFADGKLYSATVTDFLAIDAVIYRSLGE
NOV2l CRNYKMDTLEPFGDEFSGMARCPYDAKHANVALFADGKLYSATVTDFLAIDAVIYRSLGE
NOV2m ----------------------------------------------------------GE
NOV2n ----------------------------------------------------------GE
NOV2o ------------------------------------------------------------
NOV2p ------------------------------------------------------------
NOV2q CRNYKMDTLEPFGDEFSGMARCPYDAKHANVALFADGKLYSATVTDFLAIDAVIYRSLGE
NOV2r CRNYKNDTLEPFGDEFSGMARCPYDAKHANVALFADGKLYSATVTDFLAIDAVIYRSLGE
NOV2s CRNYKMDTLEPFGDEFSGMARCPYDAKHANVALFADGKLYSATVTDFLAIDAVIYRSLGE
NOV2t CRNYKMDTLEPFGDEFSGMARCPYDAKHANVALFADGKLYSATVTDFLAIDAVIYRSLGE
NOV2u CRNYKMDTLEPFGDEFSGMARCPYDAKHANVALFADGKLYSATVTDFLAIDAVIYRSLGE
NOV2v CRNYKMDTLEPFGDEFSGMARCPYDAKHANVALFADGKLYSATVTDFLAIDAVIYRSLGE
NOV2a SPTLRTVKHDSKWLKEPYFVQAVDYGDYIYFFFREIAVEYNTMGKVVFPRVAQVCKNDMG
NOV2b SPTLRTVKHDSKWLKEPYFVQAVDYGDYIYFFFREIAVEYNTMGKVVFPRVAQVCKNDMG
NOV2c SPTLRTVKHDSKWLKEPYFVQAVDYGDYIYFFFREIAVEYNTMGKVVFPRVAQVCKNDMG
NOV2d SPTLRTVKHDSKWLKEPYFVQAVDYGDYIYFFFREIAVEYNTMGKVVFPRVAQVCKNDMG
NOV2e SPTLRTVKHDSKWLKEPYFVQAVDYGDYIYFFFREIAVEYNTMGKVVFPRVAQVCKNDMG
NOV2f SPTLRTVKHDSKWLKEPYFVQAVDYGDYIYFFFREIAVEYNTMGKVVFPRVAQVCKNDMG
NOV2g SPTLRTVKHDSKWLKEPYFVQAVDYGDYIYFFFREIAVEYNTMGKVVFPRVAQVCKNDMG
NOV2h SPTLRTVKHDSKWLKEPYFVQAVDYGDYIYFFFREIAVEYNTMGKVVFPRVAQVCKNDMG
NOV2i SPTLRTVKHDSKWLKEPYFVQAVDYGDYIYFFFREIAVEYNTMGKVVFPRVAQVCKNDMG
NOV2j SPTLRTVKHDSKWLKEPYFVQAVDYGDYIYFFFREIAVEYNTMGKVVFPRVAQVCKNDMG
NOV2k SPTLRTVKHDSKWLKEPYFVQAVDYGDYIYFFFREIAVEYNTMGKVVFPRVAQVCKNDMG
NOV2l SPTLRTVKHDSKWLKEPYFVQAVDYGDYIYFFFREIAVEYNTMGKVVFPRVAQVCKNDMG
NOV2m SPTLRTVKHDSKWLKE--------------------------------------------
NOV2n SPTLRTVKHDSKWLKE--------------------------------------------
NOV2o ------------------------------------------------------------
NOV2p ------------------------------------------------------------
NOV2q SPTLRTVKHDSKWLKEPYFVQAVDYGDYIYFFFREIAVEYNTMGKVVFPRVAQVCKNDMG
NOV2r SPTLRTVKHDSKWLKEPYFVQAVDYGDYIYFFFREIAVEYNTMGKVVFPRVAQVCKNDMG
NOV2s SPTLRTVKHDSKWLKEPYFVQAVDYGDYIYFFFREIAVEYNTMGKVVFPRVAQVCKNDMG
NOV2t SPTLRTVKHDSKWLKEPYFVQAVDYGDYIYFFFREIAVEYNTMGKVVFPRVAQVCKNDMG
NOV2u SPTLRTVKHDSKWLKEPYFVQAVDYGDYIYFFFREIAVBYNTMGKVVFPRVAQVCKNDMG
NOV2v SPTLRTVKHDSKWLKEPYFVQAVDYGDYIYFFFREIAVEYNTMGKVVFPRVAQVCKNDMG
NOV2a GSQRVLEKQWTSFLKARLNCSVPGDSHFYFNILQAVTDVIRINGRDVVLATFSTPYNSIP
NOV2b GSQRVLEKQWTSFLKARLNCSVPGDSHFYFNILQAVTDVIRINGRDVVLATFSTPYNSIP
NOV2c GSQRVLEKQWTSFLKARLNCSVPGDSHFYFNILQAVTDVIRINGRDVVLATFSTPYNSIP
NOV2d GSQRVLEKQWTSFLKARLNCSVPGDSHFYFNILQAVTDVIRINGRDVVLATFSTPYNSIP
NOV2e GSQRVLEKQWTSFLKARLNCSVPGDSHFYFNILQAVTDVIRINGRDVVLATFSTPYNSIP
NOV2f GSQRVLEKQWTSFLKARLNCSVPGDSHFYFNILQAVTDVIRINGRDVVLATFSTPYNSIP
NOV2g GSQRVLEKQWTSFLKARLNCSVPGDSHFYFNILQAVTDVIRINGRDVVLATFSTPYNSIP
NOV2h GSQRVLEKQWTSFLKARLNCSVPGDSHFYFNILQAVTDVIRINGRDVVLATFSTPYNSIP
NOV2i GSQRVLEKQWTSFLKARLNCSVPGDSHFYFNILQAVTDVIRIKGRDVVLATFSTPYNSIP
NOV2j GSQRVLEKQWTSFLKARLNCSVPGDSHFYFNILQAVTDVIRINGRDVVLATFSTPYNSIP
NOV2k GSQRVLEKQWTSFLKARLNCSVPGDSHFYFNILQAVTDVIRINGRDVVLATFSTPYNSIP
NOV2l GSQRVLEKQWTSFLKARLNCSVPGDSHFYFNILQAVTDVIRINGRDVVLATFSTPYNSIP
NOV2m ------------------------------------------------------------
NOV2n ------------------------------------------------------------
NOV2o ------------------------------------------------------------
NOV2p ------------------------------------------------------------
NOV2q GSQRVLEKRWTSFLKARLNCSVPGDSHFYFNILQAVTDVIRINGRDVVLATFSTPYNSIP
NOV2r GSQRVLEKQWTSFLKARLNCSVPGDSHFYFNILQAVTDVIRINGRDVVLATFSTPYNSIP
NOV2s GSQRVLEKQWTSFLKARLNCSVPGDSHFYFNILQAVTDVIRINGRDVVLATFSTPYNSIP
NOV2t GSQRVLEKQWTSFLKARLNCSVPGDSHFYFNILQAVTDVIRINGRDVVLATFSTPYNSIP
NOV2u GSQRVLEKQWTSFLKARLNCSVPGDSHFYFNILQAVTDVIRINGRDVVLATFSTPYNSIP
NOV2v GSQRVLEKQWTSFLKARLNCSVPGDSHFYFNILQAVTDVIRINGRDVVLATFSTPYNSIP
NOV2a GSAVCAYDMLDIASVFTGRFKEQKSPDSTWTPVPDERVPKPRPGCCAGSSSLERYATSNE
NOV2b GSAVCAYDMLDIASVFTGRFKEQKSPDSTWTPVPDERVPKPRPGCCAGSSSLERYATSNE
NOV2c GSAVCAYDMLDIASVFTGRFKEQKSPDSTWTPVPDERVPKPRPGCCAGSSSLERYATSNE
NOV2d GSAVCAYDMLDIASVFTGRFKEQKSPDSTWTPVPDERVPKPRPGCCAGSSSLERYATSNE
NOV2e GSAVCAYDMLDIASVFTGRFKEQKSPDSTWTPVPDERVPKPRPGCCAGSSSLERYATSNE
NOV2f GSAVCAYDMLDIASVFTGRFKEQKSPDSTWTPVPDERVPKPRPGCCAGSSSLERYATSNE
NOV2g GSAVCAYDMLDIASVFTGRFKEQKSPDSTWTPVPDERVPKPRPGCCAGSSSLERYATSNE
NOV2h GSAVCAYDMLDIASVFTGRFKEQKSPDSTWTPVPDERVPKPRPGCCAGSSSLERYATSNE
NOV2i GSAVCAYDMLDIASVFTGRFKEQKSPDSTWTPVPDERVPKPRPGCCAGSSSLERYATSNE
NOV2j GSAVCAYDMLDIASVFTGRFKEQKSPDSTWTPVPDERVPKPRPGCCAGSSSLERYATSNE
NOV2k GSAVCAYDMLDIASVFTGRFKEQKSPDSTWTPVPDERVPKPRPGCCAGSSSLERYATSNE
NOV2l GSAVCAYDMLDIASVFTGRFKEQKSPDSTWTPVPDERVPKPRPGCCAGSSSLERYATSNE
NOV2m ------------------------------------------------------------
NOV2n ------------------------------------------------------------
NOV2o ------------------------------------------------SSSLERYATSNE
NOV2p ------------------------------------------------------------
NOV2q GSAVCAYDMLDIASVFTGRFKEQKSPDSTWTPVPDERVPKPRPGCCAGSSSLERYATSNE
NOV2r GSAVCAYDMLDIASVFTGRFKEQKSPDSTWTPVPDERVPKPRPGCCAGSSSLERYATSNE
NOV2s GSAVCAYDMLDIASVFTGRFKEQKSPDSTWTPVPDERVPKPRPGCCAGSSSLERYATSNE
NOV2t GSAVCAYDMLDIASVFTGRFKEQKSPDSTWTPVPDERVPKPRPGCCAGSSSLERYATSNE
NOV2u GSAVCAYDMLDIASVFTGRFKEQKSPDSTWTPVPDERVPKPRPGCCAGSSSLERYATSNE
NOV2v GSAVCAYDMLDIASVFTGRFKEQKSPDSTWTPVPDERVPKPRPGCCAGSSSLERYATSNE
NOV2a FPDDTLNFIKTHPLMDEAVPSIFNRPWFLRTMVRYRLTKIAVDTAAGPYQNHTVVFLGSE
NOV2b FPDDTLNFIKTHPLMDEAVPSIFNRPWFLRTMVRYRLTKIAVDTAAGPYQNHTVVFLGSE
NOV2c FPDDTLNFIKTHPLMDEAVPSIFNRPWFLRTMVRYRLTKIAVDTAAGPYQNHTVVFLGSE
NOV2d FPDDTLNFIKTHPLMDEAVPSIFNRPWFLRTMVRYRLTKIAVDTAAGPYQNHTVVFLGSE
NOV2e FPDDTLNFIKTHPLMDEAVPSIFNRPWFLRTMVRYRLTKIAVDTAAGPYQNHTVVFLGSE
NOV2f FPDDTLNFIKTHPLMDEAVPSIFNRPWFLRTMVRYRLTKIAVDTAAGPYQNHTVVFLGSE
NOV2g FPDDTLNFIKTHPLMDEAVPSIFNRPWFLRTMVRYRLTKIAVDTAAGPYQNHTVVFLGSE
NOV2h FPDDTLNFIKTHPLMDEAVPSIFNRPWFLRTMVRYRLTKIAVDTAAGPYQNHTVVFLGSE
NOV2i FPDDTLNFIKTHPLMDEAVPSIFNRPWFLRTMVRYRLTKIAVDTAAGPYQNHTVVFLGSE
NOV2j FPDDTLNFIKTHPLMDEAVPSIFNRPWFLRTMVRYRLTKIAVDTAAGPYQNHTVVFLGSE
NOV2k FPDDTLNFIKTHPLHDEAVPSIFNRPWFLRTMVRYRLTKIAVDTAAGPYQNHTVVFLGSE
NOV2l FPDDTLNFIKTHPLMDEAVPSIFNRPWFLRTMVR -------------------------
NOV2m ------------------------------------------------------------
NOV2n ------------------------------------------------------------
NOV2o FPDDT-------------------------------------------------------
NOV2p ------------------------------------------------------------
NOV2q FPDDTLNFIKTHPLMDEAVPSIFNRPWFLRTMVR--------------------------
NOV2r FPDDTLNFIKTHPLMDEAVPSIFNRPWFLRTMVRYRLTKIAVDTAAGPYQNHTVVFLGSE
NOV2s FPDDTLNFIKTHPLMDEAVPSIFNRPWFLRTMVRYRLTKIAVDTAAGPYQNHTVVFLGSE
NOV2t FPDDTLNFIKTHPLMDEAVPSIFNRPWFLRTMVRYRLTKIAVDTAAGPYQNHTVVFLGSE
NOV2u FPDDTLNFIKTHPLMDEAVPSIFNRPWFLRTMVRYRLTKIAVDTAAGPYQNHTVVFLGSE
NOV2v FPDDTLNFIKTHPLMDEAVPSIFNRPWFLRTMVRYRLTKIAVDTAAGPYQNHTVVFLGSE
NOV2a KGIILKFLARIGNSGFLNDSLFLEEMSVYNSEKCSYDGVEDKRIMGMQLDRASSSLYVAF
NOV2b KGIILKFLARIGNSGFLNDSLFLEEMSVYNSEKCSYDGVEDKRIMGMQLDRASSSLYVAF
NOV2c KGIILKFLARIGNSGFLNDSLFLEEMSVYNSEKCSYDGVEDKRIMGMQLDRASSSLYVAF
NOV2d KGIILKFLARIGNSGFLNDSLFLEEMSVYNSEKCSYDGVEDKRIMGMQLDRASSSLYVAF
NOV2e KGIILKFLARIGNSGFLNDSLFLEEMSVYNSEKCSYDGVEDKRIMGMQVDG---------
NOV2f KGIILKFLARIGNSGFLNDSLFLEEMSVYNSEKCSYDGVEDKRIMGMQVDG---------
NOV2g KGIILKFLARIGNSGFLNDSLFLEEMSVYNSEKCSYDGVEDKRIMGMQVDG---------
NOV2h KGIILKFLARIGNSGFLNDSLFLEEMSVYNSEKCSYDGVEDKRIMGMQLDRASSSLYVAF
NOV2i KGIILKFLARIGNSGFLNDSLFLEEMSVYNSEKCSYDGVEDKRIMGMQLDRASSSLYVAF
NOV2j KGIILKFLARIGNSGFLNDSLFLEEMSVYNSEKCSYDGVEDKRIMGMQLDRASSSLYVAF
NOV2k KGIILKFLARIGNSGFLNDSLFLEEMSVYNSEKCSYDGVEDKRIMGMQLDRASSSLYVAF
NOV2l ---------------------------------CSYDGVEDKRIMGMQLDHASSSLYVAF
NOV2m ------------------------------------------------------------
NOV2n ------------------------------------------------------------
NOV2o ------------------------------------------------------------
NOV2p -----------------------EEMSVYNSEKCSYDGVEDKR-----------------
NOV2q ---------------------------------CSYDGVEDKRIMGMQLDRASSSLYVAF
NOV2r KGIILKFLARIGNSGFLNDSLFLEEMSVYNSEKCSYDGVEDKRIMGMQLDRASSSLYVAF
NOV2s KGIILKFLARIGNSGFLNDSLFLEEMSVYNSEKCSYDGVEDKRIMGMQLDRASSSLYVAF
NOV2t KGIILKFLARIGNSGFLNDSLFLEEMSVYNSEKCSYDGVEDKRIMGMQLDRASSSLYVAF
NOV2u KGIILKFLARIGNSGFLNDSLFLEEMSVYNSEKCSYDGVEDKRIMGMQLDRASSSLYVAF
NOV2v KGIILKFLARIGNSGFLNDSLFLEEMSVYNSEKCSYDGVEDKRIMGMQLDRASSSLYVAF
NOV2a STCVIKVPLGRCERHGKCKKTCIASRDPYCGWIKEGGACSHLSPNSRLTFEQDIERGNTD
NOV2b STCVIKVPLGRCERHGKCKKTCIASRDPYCGWIKEGGACSHLSPNSRLTFEQDIERGNTD
NOV2c STCVIKVPLGRCERHGKCKKTCIASRDPYCGWIKEGGACSHLSPNSRLTFEQDIERGNTD
NOV2d STCVIKVPLGRCERHGKCKKTCIASRDPYCGWIKEGGACSHLSPNSRLTFEQDIERGNTD
NOV2e ------------------------------------------------------------
NOV2f ------------------------------------------------------------
NOV2g ------------------------------------------------------------
NOV2h STCVIKVPLGRCERHGKCKKTCIASRDPYCGWIKEGGACSHLSPNSRLTFEQDIERGNTD
NOV2i STCVIKVPLGRCERHGKCKKTCIASRDPYCGWIKEGGACSHLSPNSRLTFEQDIERGNTD
NOV2j STCVIKVPLGRCERHGKCKKTCIASRDPYCGWIKEGGACSHLSPNSRLTFEQDIERGNTD
NOV2k STCVIKVPLGRCERHGKCKKTCIASRDPYCGWIKEGGACSHLSPNSRLTFEQDIERGNTD
NOV2l STCVIKVPLGRCERHGKCKKTCIASRDPYCGWIKEGGACSHLSPNSRLTFEQDIERGNTD
NOV2m ------------------------------------------------------------
NOV2n ------------------------------------------------------------
NOV2o ------------------------------------------------------------
NOV2p ------------------------------------------------------------
NOV2q STCVIKVPLGRCERHGKCKKTCIASRDPYCGWIKEGGACSHLSPNSRLTFEQDIERGNTD
NOV2r STCVIKVPLGRCERHGKCKKTCIASRDPYCGWIKEGGACSHLSPNSRLTFEQDIERGNTD
NOV2s STCVIKVPLGRCERHGKCKKTCIASRDPYCGWIKEGGACSHLSPNSRLTFEQDIERGNTD
NOV2t STCVIKVPLGRCERHGKCKKTCIASRDPYCGWIKEGGACSHLSPNSRLTFEQDIERGNTD
NOV2u STCVIKVPLGRCERHGKCKKTCIASRDPYCGWIKEGGACSHLSPNSRLTFEQDIERGNTD
NOV2v STCVIKVPLGRCERHGKCKKTCIASRDPYCGWIKEGGACSHLSPNSRLTFEQDIERGNTD
NOV2a GLGDCHNSFVALNDISTPLPDNEMSYNTVYGHSSSLLPSTTTSDSTAQEGYESRGGMLDW
NOV2b GLGDCHNSFVALNDISTPLPDNEMSYNTVYGHSSSLLPSTTTSDSTAQEGYESRGGMLDW
NOV2c GLGDCHNSFVALNDISTPLPDNEMSYNTVYGHSSSLLPSTTTSDSTAQEGYESRGGMLDW
NOV2d GLGDCHNSFVALNDISTPLPDNEMSYNTVYGHSSSLLPSTTTSDSTAQEGYESRGGMLDW
NOV2e ------------------------------------------------------------
NOV2f ------------------------------------------------------------
NOV2g ------------------------------------------------------------
NOV2h GLGDCHNSFVALN-----------------GHSSSLLPSTTTSDSTAQEGYESRGGMLDW
NOV2i GLGDCHNSFVALN-----------------------------------------------
NOV2j GLGDCHNSFVALNG-----------------HSSSLLPSTTTSDSTAQEGYESRGGMLDW
NOV2k GLGDCHNSFVALN-----------------GHSSSLLPSTTTSDSTAQEGYESRGGMLDW
NOV2l GLGDCHNSFVALN-----------------GHSSSLLPSTTTSDSTAQEGYESRGGMLDW
NOV2m ------------------------------------------------------------
NOV2n ------------------------------------------------------------
NOV2o ------------------------------------------------------------
NOV2p ------------------------------------------------------------
NOV2q GLGDCHNSFVALN-----------------GHSSSLLPSTTTSDSTAQEGYESRGGMLDW
NOV2r GLGDCHNSFVALNDISTPLPDNEMSYNTVYGHSSSLLPSTTTSDSTAQEGYESRGGMLDW
NOV2s GLGDCHNSFVALNDISTPLPDNEMSYNTVYGHSSSLLPSTTTSDSTAQEGYESRGGMLDW
NOV2t GLGDCHNSFVALN-----------------GHSSSLLPSTTTSDSTAQEGYESRGGMLDW
NOV2u GLGDCHNSFVALNDISTPLPDNEMSYNTVYGHSSSLLPSTTTSDSTAQEGYESRGGMLDW
NOV2v GLGDCHNSFVALNDISTPLPDNEMSYNTVYGHSSSLLPSTTTSDSTAQEGYESRGGMLDW
NOV2a KHLLDSPDSTDPLGAVSSHNHQDKKGVIRESYLKGHDQLVPVTLLAIAVILAFVMGAVFS
NOV2b KNLLDSPDSTDPLGAVSSHNHQDKKGVIRESYLKGHDQVDG-------------------
NOV2c KHLLDSPDSTDPLGAVSSHNHQDKKGVIRESYLKGHDQLVPVTLLAIAVILAFVMGAVFS
NOV2d KHLLDSPDSTDPLGAVSSHNHQDKKGVIRESYLKGHDQLVPVTLLAIAVILAFVMGAVFS
NOV2e ------------------------------------------------------------
NOV2f ------------------------------------------------------------
NOV2g ------------------------------------------------------------
NOV2h KHLLDSPDSTDPLGAVSSHNHQDKKGVIRESYLKGHDQLVPVTLLAIAVILAFVMGAVFS
NOV2i -------------------------GVIRESYLKGHDQLVPVTLLAIAVILAFVMGAVFS
NOV2j KHLLDSPDSTDPLGAVSSHNHQDKKGVIRESYLKGHDQLVPVTLLAIAVILAFVMGAVFS
NOV2k KHLLDSPDSTDPLGAVSSHNHQDKKGVIRESYLKGHDQ----------------------
NOV2l KHLLDSPDSTDPLGAVSSHNHQDKKGVIRESYLKGHDQLVPVTLLAIAVILAFVMGAVFS
NOV2m ------------------------------------------------------------
NOV2n ------------------------------------------------------------
NOV2o ------------------------------------------------------------
NOV2p ------------------------------------------------------------
NOV2q KHLLDSPDSTDPLGAVSSHNHQDKKGVIRESYLKGHDQLVPVTLLAIAVILAFVMGAVFS
NOV2r KHLLDSPDSTDPLGAVSSHNHQDKKGVIRESYLKGHDQLVPVTLLAIAVILAFVMGAVFS
NOV2s KHLLDSPDSTDPLGAVSSHNHQDKKGVIRESYLKGHDQVDG-------------------
NOV2t KHLLDSPDSTDPLGAVSSHNHQDKKGVIRESYLKGHDQ----------------------
NOV2u KHLLDSPDSTDPLGAVSSHNHQDKKGVIRESYLKGHDQ----------------------
NOV2v KHLLDSPDSTDPLGAVSSHNHQDKKGVIRESYLKGHDQ----------------------
NOV2a GITVYCVCDHRRKDVAVVQRKEKELTHSRRGSMSSVTKLSGLFGDTQSKDPKPEAILTPL
NOV2b ------------------------------------------------------------
NOV2c GITVYCVCDHRRKDVAVVQRKEKELTHSRRGSMSSVTKLSGLFGDTQSKDPKPEAILTPL
NOV2d GITVYCVCDHRRKDVAVVQRKEKELTHSRRGSMSSVTKLSGLFGDTQSKDPKPEAILTPL
NOV2e ------------------------------------------------------------
NOV2f ------------------------------------------------------------
NOV2g ------------------------------------------------------------
NOV2h GITVYCVCDHRRKDVAVVQRKEKELTHSRRGSMSSVTKLSGLFGDTQSKDPKPEAILTPL
NOV2i GITVYCVCDHRRKDVAVVQRKEKELTHSRRGSMSSVTKLSGLFGDTQSKDPKPEAILTPL
NOV2j GITVYCVCDHRRKDVAVVQRKEKELTHSRRGSMSSVTKLSGLFGDTQSKDPKPEAILTPL
NOV2k ------------------------------------------------------------
NOV2l GITVYCVCDHRRKDVAVVQRKEKELTHSRRGSMSSVTKLSGLFGDTQ-------------
NOV2m ------------------------------------------------------------
NOV2n ------------------------------------------------------------
NOV2o ------------------------------------------------------------
NOV2p ------------------------------------------------------------
NOV2q GITVYCVCDHRRKDVAVVQRKEKELTHSRRGSMSSVTKLSGLFGDTQSKDPKPEAILTPL
NOV2r GITVYCVCDHRRKDVAVVQRKEKELTHSRRGSMSSVTKLSGLFGDTQSKDPKPEAILTPL
NOV2s ------------------------------------------------------------
NOV2t ------------------------------------------------------------
NOV2u ------------------------------------------------------------
NOV2v ------------------------------------------------------------
NOV2a MHNGKLATPGNTAKMLIKADQHHLDLTALPTPESTPTLQQKRKPSRGSREWERNQNLINA
NOV2b ------------------------------------------------------------
NOV2c MHNGKLATPGNTAKMLIKADQHHLDLTALPTPESTPTLQQKRKPSRGSREWERNQNLINA
NOV2d MHNGKLATPGNTAKMLIKADQHHLDLTALPTPESTPTLQQKRKPSRGSREWERNQNLINA
NOV2e ------------------------------------------------------------
NOV2f ------------------------------------------------------------
NOV2g ------------------------------------------------------------
NOV2h MHNGKLATPGNTAKMLIKADQHHLDLTALPTPESTPTLQQKRKPSRGSREWERNQWLINA
NOV2i MHNGKLATPGNTAKMLIKADQHHLDLTALPTPESTPTLQQKRKPSRGSREWERNQNLINA
NOV2j MHNGKLATPGNTAKMLIKADQHHLDLTALPTPESTPTLQQKREPSRGTREWERNQNLINA
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q MHNGKLATPGNTAKMLIKADQHHLDLTALPTPESTPTLOQKRKPSRGSREWERNQNLINA
NOV2r MHNGKLATPGNTAKMLIKADQHHLDLTALPTPESTPTLQQKRKPSRGSREWERNQNLINA
NOV2s ------------------------------------------------------------
NOV2t ------------------------------------------------------------
NOV2u ------------------------------------------------------------
NOV2v ------------------------------------------------------------
NOV2a CTKDMPPMGSPVIPTDLPLRASPSHIPSVVVLPITQQGYQHEYVDOPKMSEVAQMALEDQ
NOV2b ------------------------------------------------------------
NOV2c CTKDMPPMGSPVIPTDLPLRASPSHIPSVVVLPITQQGYQHEYVDQPKMSEVAQMALEDQ
NOV2d CTKDMPPMGSPVIPTDLPLRASPSHIPSVVVLPITQQGYQHEYVDQPKMSEVAQMALEDQ
NOV2e ------------------------------------------------------------
NOV2f ------------------------------------------------------------
NOV2g ------------------------------------------------------------
NOV2h CTKDMPPMGSPVIPTDLPLRASPSHIPSVVVLPITQQGYQHEYVDQPKMSEVAQMALEDQ
NOV2i CTKDMPPMGSPVIPTDLPLRASPSHIPSVVVLPITQQGYQHEYVDQPKMSEVAQMALEDQ
NOV2j CTKDMPPMGSPVIPTDLPLRASPSHIPSVVVLPITQQGYQHEYVDQPKMSEVAQMALEDQ
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q CTKDMPPMGSPVIPTDLPLRASPSHIPSVVVLPITQQGYQHEYVDQPKMSEVAQMALEDQ
NOV2r CTKDMPPMGSPVIPTDLPLRASPSHIPSVVVLPITQQGYQHEYVDQPKMSEVAQMALEDQ
NOV2s ------------------------------------------------------------
NOV2t ------------------------------------------------------------
NOV2u ------------------------------------------------------------
NOV2v ------------------------------------------------------------
NOV2a AATLEYKTIKEHLSSKSPNHGVNLVENLDSLPPKVPQREASLGPPGASLSQTGLSKRLEN
NOV2b ------------------------------------------------------------
NOV2c AATLEYKTIKEHLSSKSPNHGVNLVENLDSLPPKVPQREASLGPPGASLSQTGLSKRLEM
NOV2d AATLEYKTIKEHLSSKSPNHGVNLVENLDSLPPKVPQREASLGPPGASLSQTGLSKRLEM
NOV2e ------------------------------------------------------------
NOV2f ------------------------------------------------------------
NOV2g ------------------------------------------------------------
NOV2h AATLEYKTIKEHLSSKSPNHGVNLVENLDSLPPKVPQREASLGPPGASLSQTGLSKRLEM
NOV2i AATLEYKTIKEHLSSKSPNHGVNLVENLDSLPPKVPQREASLGPPGASLSQTGLSKRLEM
NOV2j AATLEYKTIKEHLSSKSPNHGVNLVENLDSLPPKVPQREASLGPPGASLSQTGLSKRLEM
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q AATLEYKTIKEHLSSKSPNHGVNLVENLDSLPPKVPQREASLGPPGASLSQTGLSKRLEM
NOV2r AATLEYKTIKEHLSSKSPNHGVNLVENLDSLPPKVPQREASLGPPGASLSQTGLSKRLEM
NOV2s ------------------------------------------------------------
NOV2t ------------------------------------------------------------
NOV2u ------------------------------------------------------------
NOV2v ------------------------------------------------------------
NOV2a HHSSSYGVDYKRSYPTNSLTRSHQATTLKRNNTNSSNSSHLSRNQSFGRGDNPPPAPQRV
NOV2b ------------------------------------------------------------
NOV2c HHSSSYGVDYKRSYPTNSLTRSHQATTLKRNNTNSSNSSHLSRNQSFGRGDNPPPAPQRV
NOV2d HHSSSYGVDYKRSYPTNSLTRSHQATTLKRNNTNSSNSSHLSRNQSFGRGDNPPPAPQRV
NOV2e ------------------------------------------------------------
NOV2f ------------------------------------------------------------
NOV2g ------------------------------------------------------------
NOV2h HHSSSYGVDYKRSYPTNSLTRSHQATTLKRNNTNSSNSSHLSRNQSFGRGDNPPPAPQRV
NOV2i HHSSSYGVDYKRSYPTNSLTRSHQATTLKRNNTNSSNSSHLSRNQSFGRGDNPPPAPQRV
NOV2j HHSSSYGVDYKRSYPTNSLTRSHLTTYSHQKQH---------------------------
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q HHSSSYGVDYKRSYPTNSLTRSHQATTLKRNNTNSSNSSHLSRNQSFGRGDNPPPAPQRV
NOV2r HHSSSYGVDYKRSYPTNSLTRSHQATTLKRNNTNSSNSSHLSRNQSFGRGDNPPPAPQRV
NOV2s ------------------------------------------------------------
NOV2t ------------------------------------------------------------
NOV2u ------------------------------------------------------------
NOV2v ------------------------------------------------------------
NOV2a DSIQVHSSQPSGQAVTVSRQPSLNAYNSLTRSGLKRTPSLKPDVPPKPSFAPLSTSMKPN
NOV2b ------------------------------------------------------------
NOV2c DSIQVHSSQPSGQAVTVSRQPSLNAYNSLTRSGLKRTPSLKPDVPPKPSFAPLSTSMKPN
NOV2d DSIQVHSSQPSGQAVTVSRQPSLNAYNSLTRSGLKRTPSLKPDVPPKPSFAPLSTSMKPN
NOV2e ------------------------------------------------------------
NOV2f ------------------------------------------------------------
NOV2g ------------------------------------------------------------
NOV2h DSIQVHSSQPSGQAVTVSRQPSLNAYNSLTRSGLKRTPSLKPDVPPKPSFAPLSTSMKPN
NOV2i DSIQVHSSQPSGQAVTVSRQPSLNAYNSLTRSGLKRTPSLKPDVPPKPSFAPLSTSMKPN
NOV2j ------------------------------------------------------------
NOV2k ------------------------------------------------------------
NOV2l ------------------------------------------------------------
NOV2m ------------------------------------------------------------
NOV2n ------------------------------------------------------------
NOV2o ------------------------------------------------------------
NOV2p ------------------------------------------------------------
NOV2q DSIQVHSSQPSGQAVTVSRQPSLNAYNSLTRSGLKRTPSLKPDVPPKPSFAPLSTSMKPN
NOV2r DSIQVHSSQPSGQAVTVSRQPSLNAYNSLTRSGLKRTPSLKPDVPPKPSFAPLSTSMKPN
NOV2s ------------------------------------------------------------
NOV2t ------------------------------------------------------------
NOV2u ------------------------------------------------------------
NOV2v ------------------------------------------------------------
NOV2a DACT---
NOV2b -------
NOV2c DACTVDG
NOV2d DACTVDG
NOV2e -------
NOV2f -------
NOV2g -------
NOV2h DACTVDG
NOV2i DACTVDG
NOV2j -------
NOV2k -------
NOV2l -------
NOV2m -------
NOV2n -------
NOV2o -------
NOV2p -------
NOV2q DACT---
NOV2r DACT---
NOV2s -------
NOV2t -------
NOV2u -------
NOV2v -------
NOV2a (SEQ ID NO: 14)
NOV2b (SEQ ID NO: 16)
NOV2c (SEQ ID NO: 18)
NOV2d (SEQ ID NO: 20)
NOV2e (SEQ ID NO: 22)
NOV2f (SEQ ID NO: 24)
NOV2g (SEQ ID NO: 26)
NOV2h (SEQ ID NO: 28)
NOV2i (SEQ ID NO: 30)
NOV2j (SEQ ID NO: 32)
NOV2k (SEQ ID NO: 34)
NOV2l (SEQ ID NO: 36)
NOV2m (SEQ ID NO: 38)
NOV2n (SEQ ID NO: 40)
NOV2o (SEQ ID NO: 42)
NOV2p (SEQ ID NO: 44)
NOV2q (SEQ ID NO: 46)
NOV2r (SEQ ID NO: 48)
NOV2s (SEQ ID NO: 50)
NOV2t (SEQ ID NO: 52)
NOV2u (SEQ ID NO: 54)
NOV2v (SEQ ID NO: 56)

Further analysis of the NOV2a protein yielded the following properties shown in Table 2C.

TABLE 2C
Protein Sequence Properties NOV2a

| | |
|---|---|
| SignalP analysis: | Cleavage site between residues 19 and 20 |
| PSORT II analysis: | |

| | |
|---|---|
| PSG: | a new signal peptide prediction method |
| | N-region: length 4; pos. chg 1; neg. chg 1 |
| | H-region: length 17; peak value 9.51 |
| | PSG score: 5.11 |
| GvH: | von Heijne's method for signal seq. recognition |
| | GvH score (threshold: −2.1): 1.58 |
| | possible cleavage site: between 18 and 19 |

>>> Seems to have a cleavable signal peptide (1 to 18)

| | |
|---|---|
| ALOM: | Klein et al's method for TM region allocation |
| | Init position for calculation: 19 |
| | Tentative number of TMS(s) for the threshold 0.5: 1 |
| | Number of TMS(s) for threshold 0.5: 1 |
| | INTEGRAL Likelihood = −11.62 Transmembrane 662-678 |
| | PERIPHERAL Likelihood = 2.28 (at 436) |
| | ALOM score: −11.62 (number of TMSs: 1) |
| MTOP: | Prediction of membrane topology (Hartmann et al.) |
| | Center position for calculation: 9 |
| | Charge difference: −3.5 C(−2.5) - N(1.0) |
| | N >= C: N-terminal side will be inside |

>>> membrane topology: type 1a (cytoplasmic tail 679 to 1047)

| | |
|---|---|
| MITDISC: | discrimination of mitochondrial targeting seq |

| | |
|---|---|
| R content: 1 | Hyd Moment(75): 3.63 |
| Hyd Moment(95): 2.72 | G content: 2 |
| D/E content: 2 | S/T content: 2 |
| Score: −7.22 | |

| | |
|---|---|
| Gavel: | prediction of cleavage sites for mitochondrial preseq |
| | R-2 motif at 12 MRS\|EA |
| NUCDISC: | discrimination of nuclear localization signals |
| | pat4: HRRK (3) at 693 |
| | pat4: KRKP (4) at 784 |
| | pat7: none |
| | bipartite: none |
| | content of basic residues: 11.4% |
| | NLS Score: −0.03 |
| KDEL: | ER retention motif in the C-terminus: none |

ER Membrane Retention Signals:
XXRR-like motif in the N-terminus: RSEA none

| | |
|---|---|
| SKL: | peroxisomal targeting signal in the C-terminus: none |
| PTS2: | 2nd peroxisomal targeting signal: none |
| VAC: | possible vacuolar targeting motif: none |

RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| | total: 0 residues |

Final Results (k = 9/23):

44.4%: extracellular, including cell wall
22.2%: Golgi
22.2%: endoplasmic reticulum
11.1%: plasma membrane

>> prediction for CG51896-04 is exc (k = 9)

A search of the NOV2a protein against the Geneseq database, a proprietary database that contains sequences published in patents and patent publication, yielded several homologous proteins shown in Table 2D.

TABLE 2D
Geneseq Results for NOV2a

| | | | Identities/ | |
|---|---|---|---|---|
| | | | Similari- | |
| | | NOV2a | ties | |
| | Protein/ | Residues/ | for the | |
| Geneseq | Organism/Length | Match | Matched | Expect |
| Identifier | [Patent #, Date] | Residues | Region | Value |
| AAY71460 | Human semaphorin | 1 . . . 1047 | 1029/1047 | 0.0 |
| | 6A-1 - Homo | 1 . . . 1030 | (98%) | |
| | sapiens, 1030 aa. | | 1029/1047 | |
| | [WO200031252-A1, | | (98%) | |
| | 02 JUN. 2000] | | | |
| AAB23030 | Human semaphorin | 1 . . . 949 | 927/949 | 0.0 |
| | protein-like | 1 . . . 932 | (97%) | |
| | splice variant, | | 929/949 | |
| | SECX 2864933-1 - | | (97%) | |
| | Homo sapiens, | | | |
| | 939 aa. | | | |
| | [WO200053742-A2, | | | |
| | 14 SEP. 2000] | | | |
| AAB95139 | Human protein | 332 . . . 1047 | 699/716 | 0.0 |
| | sequence SEQ | 1 . . . 699 | (97%) | |
| | ID NO: 17154 - | | 699/716 | |
| | Homo sapiens, | | (97%) | |
| | 699 aa. | | | |
| | [EP1074617-A2, | | | |
| | 07 FEB. 2001] | | | |
| AAB23043 | Human semaphorin | 17 . . . 662 | 627/646 | 0.0 |
| | protein-like | 1 . . . 629 | (97%) | |
| | splice variant, | | 628/646 | |
| | SECX pCR2.1- | | (97%) | |
| | 2864933 - | | | |
| | Homo sapiens, | | | |
| | 630 aa. | | | |
| | [WO200053742-A2, | | | |
| | 14 SEP. 2000] | | | |
| AAB90731 | Human CJ145_1 | 1 . . . 578 | 575/578 | 0.0 |
| | protein sequence | 1 . . . 578 | (99%) | |
| | SEQ ID 161 - | | 576/578 | |
| | Homo sapiens, | | (99%) | |
| | 975 aa. | | | |
| | [WO200119988-A1, | | | |
| | 22 MAR. 2001] | | | |

In a BLAST search of public sequence datbases, the NOV2a protein was found to have homology to the proteins shown in the BLASTP data in Table 2E.

TABLE 2E
Public BLASTP Results for NOV2a

| | | | Identities/ | |
|---|---|---|---|---|
| | | | Similari- | |
| | | NOV2a | ties | |
| Protein | | Residues/ | for the | |
| Accession | Protein/ | Match | Matched | Expect |
| Number | Organism/Length | Residues | Portion | Value |
| Q9P2H9 | Hypothetical protein | 1 . . . 1047 | 1046/1047 | 0.0 |
| | KIAA1368 - | 3 . . . 1049 | (99%) | |
| | Homo sapiens | | 1046/1047 | |
| | (Human), 1049 aa | | (99%) | |
| | (fragment). | | | |
| Q9H2E6 | Semaphorin | 1 . . . 1047 | 1029/1047 | 0.0 |
| | SEMA6A1 - | 1 . . . 1030 | (98%) | |
| | Homo sapiens | | 1029/1047 | |
| | (Human), 1030 aa. | | (98%) | |
| Q9EQ71 | Axon guidance | 1 . . . 1047 | 947/1048 | 0.0 |
| | signal SEMA6A1 - | 1 . . . 1005 | (90%) | |
| | Mus musculus | | 973/1048 | |
| | (Mouse), 1005 aa. | | (92%) | |
| O35464 | Semaphorin 6A | 1 . . . 880 | 815/881 | 0.0 |
| | precursor (Semaphorin | 1 . . . 864 | (92%) | |
| | VIA) (Sema VIA) | | 839/881 | |
| | (Semaphorin Q) | | (94%) | |
| | (Sema Q) - Mus | | | |
| | musculus (Mouse), | | | |
| | 888 aa. | | | |
| Q96SW4 | Hypothetical | 332 . . . 1047 | 699/716 | 0.0 |
| | protein FLJ14595 - | 1 . . . 699 | (97%) | |
| | Homo sapiens | | 699/716 | |
| | (Human), 699 aa. | | (97%) | |

PFam analysis predicts that the NOV2a protein contains the domains shown in the Table 2F.

TABLE 2F
Domain Analysis of NOV2a

| | | Identities/ | |
|---|---|---|---|
| Pfam | NOV2a | Similarities | |
| Domain | Match Region | for the Matched Region | Expect Value |
| Sema | 56 . . . 491 | 203/497 (41%) | 4.5e−212 |
| | | 390/497 (78%) | |
| PSI | 514 . . . 557 | 14/68 (21%) | 0.42 |
| | | 29/68 (43%) | |

### Example 2

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and 10 referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel SD/SI (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. Expression with CT values below 28 is considered as high expression, CT values between 28 and 32 is considered moderate and CT value between 32 to 35 is considered as low expression. All the relative expression with CT values above 35 is not considered as significant expression.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4, v1.5, v1.6 and 1.7

The plates for Panels 1.4, 1.5, 1.6 and 1.7 include 2 control wells (genomic DNA control and chemistry control) and 88 to 94 wells containing cDNA from various samples. The samples in Panels 1.4, 1.5, 1.6 and 1.7 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, 1.6 and 1.7 are widely available through the American Type Culture Collection (ATCC), a repository for cultured cell lines, and were cultured using the conditions recommended by the ATCC. The normal tissues found on Panels 1.4, 1.5, 1.6 and 1.7 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

Panel 3D, 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4. ID) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, WFN gamma at approximately 20-50 ng/ml, IL-4 at approximately 5-10 ng/ml, EL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and [IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-EFN gamma (1 μg/ml) were used to direct to Th2 and IIL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁻⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80° C.

Expression of gene CG51896-04 was assessed using the primer-probe sets Ag2772, Ag88 and Ag6309, described in Tables 3A, 3B and 3C. Results of the RTQ-PCR runs are shown in Tables 3D, 3E, 3F, 3G, 3H, 31, 3J, 3K and L.

TABLE 3A
Probe Name Ag2772

| | | | Start | SEQ ID |
|---|---|---|---|---|
| Primers | Sequences | Length | Position | No |
| Forward | 5′-actggaagcatctgcttgact-3′ | 21 | 2117 | 1 |
| Probe | TET-5′-cacctgacagcacagaccctttgg-3′-TAMRA | 24 | 2093 | 2 |
| Reverse | 5′-atcactcccttcttgtcttggt-3′ | 22 | 2050 | 3 |

TABLE 3B

Probe Name Ag88

| | | | Start | SEQ ID |
|---|---|---|---|---|
| Primers | Sequences | Length | Position | No |
| Forward | 5′-catcttcaacaggccatggtt-3′ | 21 | 2770 | 4 |
| Probe | TET-5′-tgagaacaatggtcagataccgccttaccaa-3′-TAMRA | 31 | 2737 5 | |
| Reverse | 5′-agcagctgtgtccactgcaa-3′ | 20 | 2715 | 6 |

TABLE 3C
Probe Name Ag6309

| | | | | |
|---|---|---|---|---|
| | | | Start | SEQ ID |
| Primers | Sequences | Length | Position | No |

| | | | | |
|---|---|---|---|---|
| Forward | 5′-atacactgtgttgtaagacatttcattatc-3′ | 30 | 2223 | 17 |
| Probe | TET-5′-tggcactgaatgacatttcaactcctc-3′-TAMRA | 27 | 2258 | 18 |
| Reverse | 5′-gggactgtcacaattcctttg-3′ | 21 | 2285 | 158 |

TABLE 3D
CNS_neurodegeneration_v1.0
Column A - Rel. Exp.(%) Ag2772, Run 208699007
Column B - Rel. Exp.(%) Ag6309, Run 259476984

| Tissue Name | A | B |
|---|---|---|
| AD 1 Hippo | 3.1 | 8.0 |
| AD 2 Hippo | 41.8 | 31.6 |
| AD 3 Hippo | 12.8 | 2.3 |
| AD 4 Hippo | 12.3 | 7.9 |
| AD 5 Hippo | 60.7 | 19.6 |
| AD 6 Hippo | 76.8 | 72.2 |
| Control 2 Hippo | 67.8 | 52.9 |
| Control 4 Hippo | 21.9 | 26.6 |
| Control (Path) 3 Hippo | 0.9 | 9.7 |
| AD 1 Temporal Ctx | 48.3 | 11.1 |
| AD 2 Temporal Ctx | 32.1 | 32.1 |
| AD 3 Temporal Ctx | 8.5 | 3.4 |
| AD 4 Temporal Ctx | 17.0 | 35.1 |
| AD 5 Inf Temporal Ctx | 22.5 | 48.0 |
| AD 5 Sup Temporal Ctx | 48.6 | 41.5 |
| AD 6 Inf Temporal Ctx | 66.4 | 45.4 |
| AD 6 Sup Temporal Ctx | 61.1 | 18.4 |
| Control 1 Temporal Ctx | 10.3 | 13.2 |
| Control 2 Temporal Ctx | 39.0 | 44.4 |
| Control 3 Temporal Ctx | 33.0 | 27.4 |
| Control 3 Temporal Ctx | 3.4 | 12.9 |
| Control (Path) 1 Temporal Ctx | 47.0 | 38.2 |
| Control (Path) 2 Temporal Ctx | 28.9 | 31.0 |
| Control (Path) 3 Temporal Ctx | 6.0 | 5.3 |
| Control (Path) 4 Temporal Ctx | 4.7 | 19.6 |
| AD 1 Occipital Ctx | 37.6 | 7.5 |
| AD 2 Occipital Ctx (Missing) | 0.0 | 0.0 |
| AD 3 Occipital Ctx | 5.6 | 4.7 |
| AD 4 Occipital Ctx | 43.8 | 37.6 |
| AD 5 Occipital Ctx | 38.2 | 33.4 |
| AD 6 Occipital Ctx | 0.0 | 14.7 |
| Control 1 Occipital Ctx | 4.5 | 4.1 |
| Control 2 Occipital Ctx | 77.4 | 61.6 |
| Control 3 Occipital Ctx | 29.1 | 24.0 |
| Control 4 Occipital Ctx | 22.8 | 17.3 |
| Control (Path) 1 Occipital Ctx | 100.0 | 100.0 |
| Control (Path) 2 Occipital Ctx | 4.1 | 16.3 |
| Control (Path) 3 Occipital Ctx | 10.4 | 4.2 |
| Control (Path) 4 Occipital Ctx | 13.4 | 111.0 |
| Control 1 Parietal Ctx | 22.4 | 13.1 |
| Control 2 Parietal Ctx | 52.9 | 33.4 |
| Control 3 Parietal Ctx | 3.1 | 19.5 |
| Control (Path) 1 Parietal Ctx | 41.2 | 38.7 |
| Control (Path) 2 Parietal Ctx | 10.2 | 28.5 |
| Control (Path) 3 Parietal Ctx | 6.8 | 5.1 |
| Control (Path) 4 Parietal Ctx | 42.0 | 29.3 |

TABLE 3E
General_screening_panel_v1.5
Column A - Rel. Exp. (%) Ag6309, Run 259428262

| Tissue Name | A | Tissue Name | A |
|---|---|---|---|
| Adipose | 2.4 | Renal ca. TK-10 | 1.1 |
| Melanoma* Hs688(A).T | 0.0 | Bladder | 0.5 |
| Melanoma* Hs688(B).T | 0.0 | Gastric ca. (liver met.) | 0.0 |
| | | NCI-N87 | |
| Melanoma* M14 | 1.0 | Gastric ca. KATO III | 0.0 |
| Melanoma* LOXIMVI | 0.0 | Colon ca. SW-948 | 0.0 |
| Melanoma* SK-MEL-5 | 7.1 | Colon ca. SW480 | 0.0 |
| Squamous cell carcinoma | 0.0 | Colon ca.* (SW480 met) | 0.1 |
| SCC-4 | | SW620 | |
| Testis Pool | 0.8 | Colon ca. HT29 | 0.0 |
| Prostate ca.* (bone met) | 0.0 | Colon ca. HCT-116 | 0.0 |
| PC-3 | | | |
| Prostate Pool | 0.6 | Colon ca. CaCo-2 | 1.4 |
| Placenta | 1.8 | Colon cancer tissue | 1.0 |
| Uterus Pool | 0.5 | Colon ca. SW1116 | 0.0 |
| Ovarian ca. OVCAR-3 | 0.0 | Colon ca. Colo-205 | 0.3 |
| Ovarian ca. SK-OV-3 | 0.2 | Colon ca. SW-48 | 0.0 |
| Ovarian ca. OVCAR-4 | 0.0 | Colon Pool | 0.3 |
| Ovarian ca. OVCAR-5 | 0.7 | Small Intestine Pool | 0.8 |
| Ovarian ca. IGROV-1 | 5.8 | Stomach Pool | 0.6 |
| Ovarian ca. OVCAR-8 | 0.0 | Bone Marrow Pool | 0.3 |
| Ovary | 0.0 | Fetal Heart | 0.5 |
| Breast ca. MCF-7 | 0.0 | Heart Pool | 0.4 |
| Breast ca. MDA-MB-231 | 0.0 | Lymph Node Pool | 0.0 |
| Breast ca. BT 549 | 0.0 | Fetal Skeletal Muscle | 0.6 |
| Breast ca. T47D | 0.8 | Skeletal Muscle Pool | 0.6 |
| Breast ca. MDA-N | 0.0 | Spleen Pool | 0.2 |
| Breast Pool | 0.0 | Thymus Pool | 0.0 |
| Trachea | 0.2 | CNS cancer (glio/astro) | 0.0 |
| | | U87-MG | |
| Lung | 0.4 | CNS cancer (glio/astro) | 0.0 |
| | | U-118-MG | |
| Fetal Lung | 10.4 | CNS cancer (neuro;met) | 0.4 |
| | | SK-N-AS | |
| Lung ca. NCI-N417 | 0.6 | CNS cancer (astro) SF-539 | 0.2 |
| Lung ca. LX-1 | 0.0 | CNS cancer (astro) SNB-75 | 0.0 |
| Lung ca. NCI-H 146 | 0.0 | CNS cancer (glio) SNB-19 | 4.1 |
| Lung ca. SHP-77 | 0.0 | CNS cancer (glio) SF-295 | 0.0 |
| Lung ca. A549 | 0.0 | Brain (Amygdala) Pool | 51.1 |
| Lung ca. NCI-H526 | 0.0 | Brain (cerebellum) | 86.5 |
| Lung ca. NCI-H23 | 0.7 | Brain (fetal) | 80.7 |
| Lung ca. NCI-H460 | 0.4 | Brain (Hippocampus) Pool | 53.6 |
| Lung ca. HOP-62 | 0.0 | Cerebral Cortex Pool | 54.3 |
| Lung ca. NCI-H522 | 0.0 | Brain (Substantia nigra) Pool | 32.5 |
| Liver | 0.0 | Brain (Thalamus) Pool | 72.2 |
| Fetal Liver | 0.0 | Brain (whole) | 61.1 |
| Liver ca. HepG2 | 0.0 | Spinal Cord Pool | 100.0 |
| Kidney Pool | 0.5 | Adrenal Gland | 2.4 |
| Fetal Kidney | 2.1 | Pituitary gland Pool | 0.4 |
| Renal ca. 786-0 | 4.2 | Salivary Gland | 0.5 |
| Renal ca. A498 | 0.0 | Thyroid (female) | 0.0 |
| Renal ca. ACHN | 0.2 | Pancreatic ca. CAPAN2 | 0.0 |
| Renal ca. UO-31 | 0.0 | Pancreas Pool | 0.1 |

TABLE 3F
HASS Panel v1.0
Column A - Rel. Exp.(%) Ag2772, Run 264977485

| Tissue Name | A | Tissue Name | A |
|---|---|---|---|
| MCF-7 C1 | 3.1 | U87-MG F1 (B) | 0.1 |
| MCF-7 C2 | 6.0 | U87-MG F2 | 0.0 |
| MCF-7 C3 | 3.0 | U87-MG F3 | 0.3 |
| MCF-7 C4 | 6.2 | U87-MG F4 | 0.1 |
| MCF-7 C5 | 3.8 | U87-MG F5 | 0.2 |
| MCF-7 C6 | 2.5 | U87-MG F6 | 1.1 |
| MCF-7 C7 | 5.5 | U87-MG F7 | 0.2 |
| MCF-7 C9 | 3.2 | U87-MG F8 | 0.6 |
| MCF-7 C10 | 6.6 | U87-MG F9 | 0.1 |
| MCF-7 C11 | 1.1 | U87-MG F10 | 0.5 |
| MCF-7 C12 | 1.7 | U87-MG F11 | 0.7 |
| MCF-7 C13 | 3.6 | U87-MG F12 | 0.2 |
| MCF-7 C15 | 2.1 | U87-MG F13 | 0.2 |
| MCF-7 C16 | 2.7 | U87-MG F14 | 0.7 |
| MCF-7 C17 | 2.3 | U87-MG F15 | 0.2 |
| T24 D1 | 0.7 | U87-MG F16 | 0.2 |
| T24 D2 | 0.0 | U87-MG F17 | 0.3 |
| T24 D3 | 0.1 | LnCAP A1 | 29.9 |
| T24 D4 | 0.1 | LnCAP A2 | 26.1 |
| T24 D5 | 0.0 | LnCAP A3 | 46.7 |
| T24 D6 | 0.0 | LnCAP A4 | 26.6 |
| T24 D7 | 0.0 | LnCAP A5 | 39.8 |
| T24 D9 | 0.0 | LnCAP A6 | 32.8 |
| T24 D10 | 0.0 | LnCAP A7 | 16.4 |
| T24 D11 | 0.0 | LnCAP A8 | 42.9 |
| T24 D12 | 0.0 | LnCAP A9 | 18.9 |
| T24 D13 | 0.0 | LnCAP A10 | 20.0 |
| T24 D15 | 0.1 | LnCAP A11 | 45.1 |
| T24 D16 | 0.0 | LnCAP A12 | 7.9 |
| T24 D17 | 0.0 | LnCAP A13 | 3.5 |
| CAPaN B1 | 1.6 | LnCAP A14 | 2.9 |
| CAPaN B2 | 0.5 | LnCAP A15 | 5.3 |
| CAPaN B3 | 0.5 | LnCAP A16 | 54.7 |
| CAPaN B4 | 0.7 | LnCAP A17 | 48.0 |
| CAPaN B5 | 0.7 | Primary Astrocytes | 2.3 |
| CAPaN B6 | 0.8 | Primary Renal Proximal Tubule Epithelial | 13.1 |
| | | cell A2 | |
| CAPaN B7 | 0.4 | Primary melanocytes A5 | 31.0 |
| CAPaN B8 | 0.1 | 126443 - 341 medullo | 1.5 |
| CAPaN B9 | 0.4 | 126444 - 487 medullo | 100.0 |
| CAPaN B10 | 0.8 | 126445 - 425 medullo | 1.3 |
| CAPaN B11 | 1.4 | 126446 - 690 medullo | 46.7 |
| CAPaN B12 | 1.0 | 126447 - 54 adult glioma | 0.1 |
| CAPaN B13 | 1.0 | 126448 - 245 adult glioma | 15.1 |
| CAPaN B14 | 0.1 | 126449 - 317 adult glioma | 20.6 |
| CAPaN B15 | 0.2 | 126450 - 212 glioma | 48.6 |
| CAPaN B16 | 1.1 | 126451 - 456 glioma | 84.7 |
| CAPaN B17 | 1.2 | | |

TABLE 3G
Panel 1
Column A - Rel. Exp.(%) Ag88, Run 87586103

| Tissue Name | A | Tissue Name | A |
|---|---|---|---|
| Endothelial cells | 0.4 | Renal ca. 786-0 | 66.9 |
| Endothelial cells (treated) | 1.6 | Renal ca. A498 | 0.8 |
| Pancreas | 18.2 | Renal ca. RXF 393 | 19.8 |
| Pancreatic ca. CAPAN 2 | 0.2 | Renal ca. ACHN | 8.1 |
| Adrenal gland | 51.8 | Renal ca. UO-31 | 0.2 |
| Thyroid | 4.6 | Renal ca. TK-10 | 18.3 |
| Salivary gland | 8.5 | Liver | 3.7 |
| Pituitary gland | 1.8 | Liver (fetal) | 2.1 |
| Brain (fetal) | 8.5 | Liver ca. (hepatoblast) HepG2 | 3.3 |
| Brain (whole) | 46.3 | Lung | 5.7 |
| Brain (amygdala) | 9.4 | Lung (fetal) | 8.0 |
| Brain (cerebellum) | 100.0 | Lung ca. (small cell) LX-1 | 0.0 |
| Brain (hippocampus) | 34.4 | Lung ca. (small cell) | 15.7 |
| | | NCI-H69 | |
| Brain (substantia nigra) | 50.7 | Lung ca. (s. cell var.) SHP-77 | 0.0 |
| Brain (thalamus) | 15.4 | Lung ca. (large cell) | 0.0 |
| | | NCI-H460 | |
| Brain (hypothalamus) | 2.9 | Lung ca. (non-sm. cell) A549 | 0.0 |
| Spinal cord | 22.5 | Lung ca. (non-s. cell) | 0.5 |
| | | NCI-H23 | |
| glio/astro U87-MG | 0.1 | Lung ca. (non-s. cell) HOP-62 | 0.4 |
| glio/astro U-118-MG | 0.0 | Lung ca. (non-s. cl) | 0.1 |
| | | NCI-H522 | |
| astrocytoma SW1783 | 0.0 | Lung ca. (squam.) SW 900 | 0.7 |
| neuro*; met SK-N-AS | 4.6 | Lung ca. (squam.) NCI-H596 | 30.8 |
| astrocytoma SF-539 | 0.2 | Mammary gland | 18.8 |
| astrocytoma SNB-75 | 1.1 | Breast ca.* (pl. ef) MCF-7 | 1.5 |
| glioma SNB-19 | 7.1 | Breast ca.* (pl. ef) | 0.0 |
| | | MDA-MB-231 | |
| glioma U251 | 0.6 | Breast ca.* (pl. ef) T47D | 30.6 |
| glioma SF-295 | 0.1 | Breast ca. BT-549 | 0.0 |
| Heart | 3.3 | Breast ca. MDA-N | 0.0 |
| Skeletal muscle | 1.1 | Ovary | 7.0 |
| Bone marrow | 0.9 | Ovarian ca. OVCAR-3 | 1.2 |
| Thymus | 20.6 | Ovarian ca. OVCAR-4 | 0.0 |
| Spleen | 2.5 | Ovarian ca. OVCAR-5 | 11.8 |
| Lymph node | 3.2 | Ovarian ca. OVCAR-8 | 0.4 |
| Colon (ascending) | 11.5 | Ovarian ca. IGROV-1 | 12.0 |
| Stomach | 11.4 | Ovarian ca. (ascites) | 0.9 |
| | | SK-OV-3 | |
| Small intestine | 5.5 | Uterus | 6.4 |
| Colon ca. SW480 | 0.1 | Placenta | 43.8 |
| Colon ca.* SW620 (SW480 | 0.4 | Prostate | 3.1 |
| met) | | | |
| Colon ca. HT29 | 0.0 | Prostate ca.* (bone met) PC-3 | 0.0 |
| Colon ca. HCT-116 | 0.0 | Testis | 35.6 |
| Colon ca. CaCo-2 | 19.6 | Melanoma Hs688(A).T | 0.0 |
| Colon ca. HCT-15 | 0.0 | Melanoma* (met) Hs688(B).T | 0.0 |
| Colon ca. HCC-2998 | 1.1 | Melanoma UACC-62 | 1.4 |
| Gastric ca.* (liver met) | 0.3 | Melanoma M14 | 11.4 |
| NCI-N87 | | | |
| Bladder | 1.6 | Melanoma LOX IMVI | 0.8 |
| Trachea | 5.0 | Melanoma* (met) SK-MEL-5 | 18.9 |
| Kidney | 4.7 | Melanoma SK-MEL-28 | 30.6 |
| Kidney (fetal) | 13.7 | | |

TABLE 3H
Panel 1.3D
Column A - Rel. Exp.(%) Ag2772, Run 164024167

| Tissue Name | A | Tissue Name | A |
|---|---|---|---|
| Liver adenocarcinoma | 2.4 | Kidney (fetal) | 13.6 |
| Pancreas | 5.3 | Renal ca. 786-0 | 36.6 |
| Pancreatic ca. | 0.2 | Renal ca. A498 | 2.1 |
| CAPAN 2 | | | |
| Adrenal gland | 22.2 | Renal ca. RXF 393 | 27.0 |
| Thyroid | 4.4 | Renal ca. ACHN | 10.0 |
| Salivary gland | 5.1 | Renal ca. UO-31 | 0.2 |
| Pituitary gland | 2.4 | Renal ca. TK-10 | 6.9 |
| Brain (fetal) | 3.3 | Liver | 1.8 |
| Brain (whole) | 15.9 | Liver (fetal) | 3.1 |
| Brain (amygdala) | 14.9 | Liver ca. (hepatoblast) HepG2 | |
| Brain (cerebellum) | 8.8 | Lung | 14.9 |
| Brain (hippocampus) | 27.0 | Lung (fetal) | 10.7 |
| Brain | 11.1 | Lung ca. (small cell) LX- 1 | 0.4 |
| (substantia nigra) | | | |
| Brain (thalamus) | 18.2 | Lung ca. (small cell) NCI-H69 | 9.3 |
| Cerebral Cortex | 52.1 | Lung ca. (s. cell var.) SHP-77 | 0.4 |
| Spinal cord | 66.0 | Lung ca. (large cell)NCI-H460 | 3.0 |
| glio/astro U87-MG | 0.5 | Lung ca. (non-sm. cell) A549 | 0.2 |
| glio/astro U-118-MG | 0.3 | Lung ca. (non-s. cell) NCI-H23 | 1.6 |
| astrocytoma SW1783 | 0.0 | Lung ca. (non-s. cell) HOP-62 | 0.8 |
| neuro*; met SK-N-AS | 3.2 | Lung ca. (non-s. cl) NCI-H522 | 0.0 |
| astrocytoma SF-539 | 0.4 | Lung ca. (squam.) SW 900 | 0.8 |
| astrocytoma SNB-75 | 1.0 | Lung ca. (squam.) NCI-H596 | 19.3 |
| glioma SNB-19 | 15.1 | Mammary gland | 6.5 |
| glioma U251 | 1.0 | Breast ca.* (pl. ef) MCF-7 | 2.5 |
| glioma SF-295 | 0.2 | Breast ca.* (pl. ef) MDA-MB-231 | 0.1 |
| Heart (fetal) | 5.8 | Breast ca.* (pl. ef) T47D | 16.4 |
| Heart | 4.5 | Breast ca. BT-549 | 0.4 |
| Skeletal muscle (fetal) | 100.0 | Breast ca. MDA-N | 0.0 |
| Skeletal muscle | 5.5 | Ovary | 48.0 |
| Bone marrow | 0.7 | Ovarian ca. OVCAR-3 | 1.8 |
| Thymus | 14.2 | Ovarian ca. OVCAR-4 | 0.0 |
| Spleen | 5.9 | Ovarian ca. OVCAR-5 | 7.0 |
| Lymph node | 1.2 | Ovarian ca. OVCAR-8 | 0.7 |
| Colorectal | 31.9 | Ovarian ca. IGROV-1 | 11.7 |
| Stomach | 1.3 | Ovarian ca.* (ascites) SK-OV-3 | 1.9 |
| Small intestine | 9.3 | Uterus | 5.2 |
| Colon ca. SW480 | 0.4 | Placenta | 30.6 |
| Colon ca. *SW620 | 0.7 | Prostate | |
| (SW480 met) | | | |
| Colon ca. HT29 | 0.2 | Prostate ca.* (bone met) PC-3 | 0.1 |
| Colon ca. HCT-116 | 1.5 | Testis | 5.6 |
| Colon ca. CaCo-2 | 32.1 | Melanoma Hs688(A).T | 0.2 |
| Colon ca. tissue | 5.7 | Melanoma* (met) Hs688(B).T | 0.1 |
| (ODO3866) | | | |
| Colon ca. HCC-2998 | 1.8 | Melanoma UACC-62 | 1.7 |
| Gastric ca.* | 0.1 | Melanoma M14 | 3.4 |
| (liver met) NCI-N87 | | | |
| Bladder | 14.1 | Melanoma LOX IMVI | 0.2 |
| Trachea | 5.7 | Melanoma* (met) SK-MEL-5 | 15.4 |
| Kidney | 6.1 | Adipose | 6.6 |

TABLE 3I
Panel 2D
Column A - Rel. Exp.(%) Ag2772, Run 162440317
Column B - Rel. Exp.(%) Ag88, Run 144771649

| Tissue Name | A | B |
|---|---|---|
| Normal Colon | 46.7 | 28.9 |
| CC Well to Mod Diff (ODO3866) | 2.2 | 0.8 |
| CC Margin (ODO3866) | 9.7 | 9.7 |
| CC Gr.2 rectosigmoid (ODO3868) | 2.2 | 3.0 |
| CC Margin (ODO3868) | 2.7 | 2.1 |
| CC Mod Diff (ODO3920) | 2.2 | 1.3 |
| CC Margin (ODO3920) | 13.1 | 11.5 |
| CC Gr.2 ascend colon (ODO3921) | 16.5 | 7.8 |
| CC Margin (ODO3921) | 10.2 | 5.8 |
| CC from Partial Hepatectomy | 6.7 | 23.7 |
| (ODO4309) Mets | | |
| Liver Margin (ODO4309) | 9.5 | 2.7 |
| Colon mets to lung (OD04451-01) | 4.5 | 2.6 |
| Lung Margin (OD04451-02) | 4.8 | 4.7 |
| Normal Prostate 6546-1 | 27.5 | 6.8 |
| Prostate Cancer (OD04410) | 17.4 | 14.2 |
| Prostate Margin (OD04410) | 10.2 | 6.7 |
| Prostate Cancer (OD04720-01) | 6.7 | 2.8 |
| Prostate Margin (OD04720-02) | 12.8 | 8.4 |
| Normal Lung 061010 | 22.8 | 15.7 |
| Lung Met to Muscle (ODO4286) | 0.6 | 0.8 |
| Muscle Margin (ODO4286) | 1.5 | 1.6 |
| Lung Malignant Cancer (OD03126) | 3.5 | 4.8 |
| Lung Margin (OD03126) | 36.3 | 17.7 |
| Lung Cancer (OD04404) | 3.3 | 3.1 |
| Lung Margin (OD04404) | 6.8 | 8.7 |
| Lung Cancer (OD04565) | 1.2 | 1.6 |
| Lung Margin (OD04565) | 6.8 | 6.3 |
| Lung Cancer (OD04237-01) | 4.2 | 4.2 |
| Lung Margin (OD04237-02) | 10.8 | 9.9 |
| Ocular Mel Met to Liver | 100.0 | 100.0 |
| (ODO4310) | | |
| Liver Margin (ODO4310) | 5.4 | 5.8 |
| Melanoma Mets to Lung | 65.5 | 55.5 |
| (ODO4321) | | |
| Lung Margin (OD04321) | 28.9 | 26.6 |
| Normal Kidney | 18.4 | 18.4 |
| Kidney Ca, Nuclear grade 2 | 27.7 | 17.3 |
| (OD04338) | | |
| Kidney Margin (OD04338) | 5.9 | 6.1 |
| Kidney Ca Nuclear grade 1/2 | 12.4 | 9.3 |
| (OD04339) | | |
| Kidney Margin (OD04339) | 10.7 | 12.6 |
| Kidney Ca, Clear cell type | 58.2 | 44.4 |
| (OD04340) | | |
| Kidney Margin (OD04340) | 10.7 | 15.4 |
| Kidney Ca, Nuclear grade 3 | 1.5 | 1.6 |
| (OD04348) | | |
| Kidney Margin (OD04348) | 6.7 | 9.5 |
| Kidney Cancer (OD04622-01) | 12.5 | 21.3 |
| Kidney Margin (OD04622-03) | 2.0 | 1.9 |
| Kidney Cancer (OD04450-01) | 34.2 | 35.4 |
| Kidney Margin (OD04450-03) | 5.8 | 7.2 |
| Kidney Cancer 8120607 | 6.8 | 5.1 |
| Kidney Margin 8120608 | 2.1 | 3.1 |
| Kidney Cancer 8120613 | 0.8 | 2.0 |
| Kidney Margin 8120614 | 2.8 | 1.3 |
| Kidney Cancer 9010320 | 8.9 | 10.3 |
| Kidney Margin 9010321 | 11.0 | 10.0 |
| Normal Uterus | 1.7 | 2.0 |
| Uterus Cancer 064011 | 5.9 | 5.2 |
| Normal Thyroid | 3.4 | 6.2 |
| Thyroid Cancer 064010 | 1.1 | 2.3 |
| Thyroid Cancer A302152 | 1.1 | 1.3 |
| Thyroid Margin A302153 | 5.0 | 4.4 |
| Normal Breast | 12.7 | 19.2 |
| Breast Cancer (OD04566) | 1.3 | 0.8 |
| Breast Cancer (OD04590-01) | 3.8 | 2.3 |
| Breast Cancer Mets | 5.1 | 4.8 |
| (OD04590-03) | | |
| Breast Cancer Metastasis | 22.8 | 24.1 |
| (OD04655-05) | | |
| Breast Cancer 064006 | 3.0 | 2.3 |
| Breast Cancer 1024 | 8.2 | 4.4 |
| Breast Cancer 9100266 | 6.1 | 5.5 |
| Breast Margin 9100265 | 6.9 | 5.4 |
| Breast Cancer A209073 | 9.0 | 3.9 |
| Breast Margin A209073 | 9.7 | 11.4 |
| Normal Liver | 3.3 | 2.6 |
| Liver Cancer 064003 | 1.4 | 0.6 |
| Liver Cancer 1025 | 3.2 | 3.4 |
| Liver Cancer 1026 | 1.7 | 1.7 |
| Liver Cancer 6004-T | 3.9 | 5.0 |
| Liver Tissue 6004-N | 1.5 | 0.7 |
| Liver Cancer 6005-T | 1.6 | 1.7 |
| Liver Tissue 6005-N | 1.6 | 1.3 |
| Normal Bladder | 8.2 | 6.6 |
| Bladder Cancer 1023 | 0.5 | 0.6 |
| Bladder Cancer A302173 | 6.7 | 4.4 |
| Bladder Cancer (OD04718- | 0.5 | 0.2 |
| 01) | | |
| Bladder Normal Adjacent | 5.0 | 3.7 |
| (OD04718-03) | | |
| Normal Ovary | 5.6 | 3.4 |
| Ovarian Cancer 064008 | 9.3 | 6.7 |
| Ovarian Cancer (OD04768- | 1.7 | 0.6 |
| 07) | | |
| Ovary Margin (OD04768-08) | 2.8 | 3.4 |
| Normal Stomach | 12.9 | 7.0 |
| Gastric Cancer 9060358 | 1.7 | 2.2 |
| Stomach Margin 9060359 | 2.7 | 1.2 |
| Gastric Cancer 9060395 | 3.0 | 2.8 |
| Stomach Margin 9060394 | 4.3 | 2.3 |
| Gastric Cancer 9060397 | 27.9 | 11.0 |
| Stomach Margin 9060396 | 1.3 | 0.4 |
| Gastric Cancer 064005 | 6.7 | 2.0 |

TABLE 3J
Panel 3D
Column A - Rel. Exp.(%) Ag88, Run 153109696

| Tissue Name | A | Tissue Name | A |
|---|---|---|---|
| Daoy- Medulloblastoma | 1.0 | Ca Ski- Cervical epidermoid | 0.0 |
| | | carcinoma (metastasis) | |
| TE671- Medulloblastoma | 37.4 | ES-2- Ovarian clear cell | 0.0 |
| | | carcinoma | |
| D283 Med- | 1.3 | Ramos- Stimulated with | 0.0 |
| Medulloblastoma | | PMA/ionomycin 6h | |
| PFSK-1- Primitive | 17.8 | Ramos- Stimulated with | 0.0 |
| Neuroectodermal | | PMA/ionomycin 14h | |
| XF-498- CNS | 4.0 | MEG-01- Chronic | 4.9 |
| | | myelogenous leukemia | |
| | | (megokaryoblast) | |
| SNB-78- Glioma | 0.0 | Raji- Burkitt's lymphoma | 0.0 |
| SF-268- Glioblastoma | 0.0 | Daudi- Burkitt's lymphoma | 0.3 |
| T98G- Glioblastoma | 1.0 | U266- B-cell plasmacytoma | 0.0 |
| SK-N-SH- Neuroblastoma | 7.2 | CA46- Burkitt's lymphoma | 0.0 |
| (metastasis) | | | |
| SF-295- Glioblastoma | 1.0 | RL- non-Hodgkin's B-cell | 0.0 |
| | | lymphoma | |
| Cerebellum | 58.2 | JM1- pre-B-cell lymphoma | 0.0 |
| Cerebellum | 44.8 | Jurkat- T cell leukemia | 0.0 |
| NCI-H292- | 0.0 | TF-1- Erythroleukemia | 2.2 |
| Mucoepidermoid | | | |
| lung carcinoma | | | |
| DMS-114- Small cell lung | 0.3 | HUT 78- T-cell lymphoma | 0.1 |
| cancer | | | |
| DMS-79- Small cell lung | 49.0 | U937- Histiocytic lymphoma | 0.7 |
| cancer | | | |
| NCI-H146- Small cell lung | 18.0 | KU-812- Myelogenous | 0.6 |
| cancer | | leukemia | |
| NCI-H526- Small cell lung | 0.2 | 769-P- Clear cell renal | 100.0 |
| cancer | | carcinoma | |
| NCI-N417- Small cell lung | 50.0 | Caki-2- Clear cell renal | 5.9 |
| cancer | | carcinoma | |
| NCI-H82- Small cell lung | 9.3 | SW 839- Clear cell renal | 79.6 |
| cancer | | carcinoma | |
| NCI-H157- Squamous cell | 0.0 | Rhabdoid kidney tumor | 0.0 |
| lung cancer (metastasis) | | | |
| NCI-H1155- Large cell | 0.4 | Hs766T- Pancreatic | 0.0 |
| lung cancer | | carcinoma (LN metastasis) | |
| NCI-H1299- Large cell | 5.7 | CAPAN-1- Pancreatic | 0.0 |
| lung cancer | | adenocarcinoma | |
| | | (liver metastasis) | |
| NCI-H727- Lung | 7.4 | SU86.86- Pancreatic | 1.3 |
| carcinoid | | carcinoma (liver metastasis) | |
| NCI-UMC-11- Lung | 28.1 | BxPC-3- Pancreatic | 0.0 |
| carcinoid | | adenocarcinoma | |
| LX-1- Small cell lung | 0.3 | HPAC- Pancreatic | 0.1 |
| cancer | | adenocarcinoma | |
| Colo-205- Colon cancer | 6.4 | MIA PaCa-2- Pancreatic | 0.0 |
| | | carcinoma | |
| KM12- Colon cancer | 2.9 | CFPAC-1- Pancreatic ductal | 0.7 |
| | | adenocarcinoma | |
| KM20L2- Colon cancer | 0.0 | PANC-1- Pancreatic | 0.2 |
| | | epithelioid ductal carcinoma | |
| NCI-H716- Colon cancer | 33.9 | T24- Bladder carcinma | 0.0 |
| | | (transitional cell) | |
| SW-48- Colon | 2.8 | 5637- Bladder carcinoma | 1.3 |
| adenocarcinoma | | | |
| SW1116- Colon | 0.0 | HT-1197- Bladder carcinoma | 3.0 |
| adenocarcinoma | | | |
| LS 174T- Colon | 0.6 | UM-UC-3- Bladder carcinma | 0.0 |
| adenocarcinoma | | (transitional cell) | |
| SW-948- Colon | 0.3 | A204- Rhabdomyosarcoma | 0.0 |
| adenocarcinoma | | | |
| SW-480- Colon | 0.0 | HT-1080- Fibrosarcoma | 0.7 |
| adenocarcinoma | | | |
| NCI-SNU-5- Gastric | 0.3 | MG-63- Osteosarcoma | 0.0 |
| carcinoma | | | |
| KATO III- Gastric | 0.0 | SK-LMS-1- Leiomyosarcoma | 0.0 |
| carcinoma | | (vulva) | |
| NCI-SNU-16- Gastric | 1.0 | SJRH30- Rhabdomyosarcoma | 50.7 |
| carcinoma | | (met to bone marrow) | |
| NCI-SNU-1- Gastric | 14.7 | A431 - Epidermoid carcinoma | 0.0 |
| carcinoma | | | |
| RF-1- Gastric | 2.8 | WM266-4- Melanoma | 2.5 |
| adenocarcinoma | | | |
| RF-48- Gastric | 2.4 | DU 145- Prostate carcinoma | 0.0 |
| adenocarcinoma | | (brain metastasis) | |
| MKN-45- Gastric | 0.3 | MDA-MB-468- Breast | 0.0 |
| carcinoma | | adenocarcinoma | |
| NCI-N87- Gastric | 0.0 | SCC-4- Squamous cell | 0.0 |
| carcinoma | | carcinoma of tongue | |
| OVCAR-5- Ovarian | 0.0 | SCC-9- Squamous cell | 0.0 |
| carcinoma | | carcinoma of tongue | |
| RL95-2- Uterine carcinoma | 0.0 | SCC-15- Squamous cell | 0.0 |
| | | carcinoma of tongue | |
| HelaS3- Cervical | 0.0 | CAL 27- Squamous cell | 0.0 |
| adenocarcinoma | | carcinoma of tongue | |

TABLE 3K
Panel 4D
Column A - Rel. Exp.(%) Ag2772, Run 161924079
Column B - Rel. Exp.(%) Ag88, Run 139410561

| Tissue Name | A | B |
|---|---|---|
| Secondary Th1 act | 0.8 | 0.0 |
| Secondary Th2 act | 1.5 | 0.4 |
| Secondary Tr1 act | 2.8 | 0.0 |
| Secondary Th1 rest | 0.7 | 0.0 |
| Secondary Th2 rest | 1.7 | 0.0 |
| Secondary Tr1 rest | 1.0 | 0.0 |
| Primary Th1 act | 3.2 | 0.0 |
| Primary Th2 act | 2.6 | 0.0 |
| Primary Tr1 act | 5.0 | 0.0 |
| Primary Th1 rest | 7.1 | 0.0 |
| Primary Th2 rest | 2.8 | 0.0 |
| Primary Tr1 rest | 2.5 | 0.0 |
| CD45RA CD4 lymphocyte | 1.4 | 0.6 |
| act | | |
| CD45RO CD4 lymphocyte | 1.6 | 0.3 |
| act | | |
| CD8 lymphocyte act | 2.0 | 0.0 |
| Secondary CD8 lymphocyte | 2.7 | 0.0 |
| rest | | |
| Secondary CD8 lymphocyte | 0.3 | 0.2 |
| act | | |
| CD4 lymphocyte none | 0.7 | 0.0 |
| 2ry Th1/Th2/Tr1_anti-CD95 | 1.5 | 0.0 |
| CH11 | | |
| LAK cells rest | 2.6 | 0.4 |
| LAK cells IL-2 | 2.6 | 0.0 |
| LAK cells IL-2 + IL-12 | 2.5 | 1.3 |
| LAK cells IL-2 + IFN gamma | 5.6 | 0.8 |
| LAK cells IL-2 + IL-18 | 4.2 | 0.9 |
| LAK cells PMA/ionomycin | 1.8 | 0.0 |
| NK Cells IL-2 rest | 2.4 | 0.0 |
| Two Way MLR 3 day | 2.7 | 0.0 |
| Two Way MLR 5 day | 1.7 | 0.0 |
| Two Way MLR 7 day | 0.7 | 0.4 |
| PBMC rest | 1.0 | 0.0 |
| PBMC PWM | 12.9 | 10.3 |
| PBMC PHA-L | 2.1 | 1.4 |
| Ramos (B cell) none | 1.6 | 0.0 |
| Ramos (B cell) ionomycin | 12.7 | 0.0 |
| B lymphocytes PWM | 5.8 | 3.8 |
| B lymphocytes CD40L and | 7.2 | 0.4 |
| IL-4 | | |
| EOL-1 dbcAMP | 1.3 | 0.0 |
| EOL-1 dbcAMP | 1.9 | 0.0 |
| PMA/ionomycin | | |
| Dendritic cells none | 0.9 | 0.4 |
| Dendritic cells LPS | 1.5 | 1.4 |
| Dendritic cells anti-CD40 | 2.3 | 0.3 |
| Monocytes rest | 1.4 | 0.0 |
| Monocytes LPS | 1.7 | 0.7 |
| Macrophages rest | 2.9 | 0.5 |
| Macrophages LPS | 0.4 | 0.0 |
| HUVEC none | 2.0 | 1.0 |
| HUVEC starved | 4.8 | 0.8 |
| HUVEC IL-1beta | 0.5 | 1.1 |
| HUVEC IFN gamma | 2.5 | 4.1 |
| HUVEC TNF alpha + IFN gamma | 1.1 | 0.0 |
| HUVEC TNF alpha + IL4 | 2.0 | 2.4 |
| HUVEC IL-11 | 3.0 | 3.0 |
| Lung Microvascular EC none | 1.8 | 0.9 |
| Lung Microvascular EC TNFalpha + | 1.5 | 0.3 |
| IL-1beta | | |
| Microvascular Dermal EC none | 3.9 | 3.3 |
| Microsvasular Dermal EC TNFalpha | 2.5 | 2.4 |
| + IL-1beta | | |
| Bronchial epithelium TNFalpha + | 18.3 | 22.5 |
| IL1beta | | |
| Small airway epithelium none | 1.2 | 5.1 |
| Small airway epithelium TNFalpha + | 24.0 | 23.3 |
| IL-1beta | | |
| Coronery artery SMC rest | 1.1 | 1.8 |
| Coronery artery SMC TNFalpha + | 0.6 | 0.9 |
| IL-1beta | | |
| Astrocytes rest | 19.8 | 14.1 |
| Astrocytes TNFalpha + IL-1beta | 3.4 | 4.4 |
| KU-812 (Basophil) rest | 2.4 | 1.2 |
| KU-812 (Basophil) PMA/ionomycin | 11.3 | 11.3 |
| CCD1106 (Keratinocytes) none | 0.1 | 0.2 |
| CCD1106 (Keratinocytes) TNFalpha | 0.0 | 1.3 |
| + IL-1beta | | |
| Liver cirrhosis | 11.9 | 15.0 |
| Lupus kidney | 7.3 | 23.8 |
| NCI-H292 none | 1.0 | 0.0 |
| NCI-H292 IL-4 | 2.0 | 0.0 |
| NCI-H292 IL-9 | 1.9 | 0.0 |
| NCI-H292 IL-13 | 1.1 | 0.2 |
| NCI-H292 IFN gamma | 1.1 | 0.0 |
| HPAEC none | 1.2 | 1.7 |
| HPAEC TNF alpha + IL-1 beta | 2.3 | 1.3 |
| Lung fibroblast none | 0.4 | 0.4 |
| Lung fibroblast TNF alpha + IL-1 | 1.9 | 1.9 |
| beta | | |
| Lung fibroblast IL-4 | 2.4 | 0.4 |
| Lung fibroblast IL-9 | 0.9 | 1.9 |
| Lung fibroblast IL-13 | 2.0 | 1.3 |
| Lung fibroblast IFN gamma | 0.9 | 1.7 |
| Dermal fibroblast CCD1070 rest | 3.8 | 2.6 |
| Dermal fibroblast CCD1070 TNF | 3.1 | 0.5 |
| alpha | | |
| Dermal fibroblast CCD1070 IL-1 | 0.2 | 0.3 |
| beta | | |
| Dermal fibroblast IFN gamma | 2.3 | 0.9 |
| Dermal fibroblast IL-4 | 1.6 | 1.0 |
| IBD Colitis 2 | 1.0 | 4.1 |
| IBD Crohn's | 4.9 | 6.9 |
| Colon | 100.0 | 87.1 |
| Lung | 18.4 | 24.3 |
| Thymus | 34.4 | 100.0 |
| Kidney | 20.9 | 34.4 |

TABLE 3L
general oncology screening panel_v_2.4
Column A - Rel. Exp.(%) Ag63O9, Run 259804334
Column B - Rel. Exp.(%) Ag88, Run 262228151

| Tissue Name | A | B |
|---|---|---|
| Colon cancer 1 | 5.0 | 14.4 |
| Colon NAT 1 | 7.0 | 9.2 |
| Colon cancer 2 | 0.0 | 1.2 |
| Colon NAT 2 | 18.3 | 12.3 |
| Colon cancer 3 | 15.9 | 11.3 |
| Colon NAT 3 | 33.9 | 25.0 |
| Colon malignant cancer 4 | 0.0 | 7.7 |
| Colon NAT 4 | 13.9 | 9.3 |
| Lung cancer 1 | 8.0 | 0.9 |
| Lung NAT 1 | 12.5 | 1.2 |
| Lung cancer 2 | 100.0 | 100.0 |
| Lung NAT 2 | 31.2 | 4.3 |
| Squamous cell carcinoma 3 | 20.0 | 2.5 |
| Lung NAT 3 | 5.0 | 0.2 |
| Metastatic melanoma | 10.0 | 14.9 |
| Melanoma 2 | 0.0 | 1.1 |
| Melanoma 3 | 0.0 | 1.9 |
| Metastatic melanoma 4 | 0.0 | 18.8 |
| Metastatic melanoma 5 | 7.5 | 32.1 |
| Bladder cancer 1 | 0.0 | 0.6 |
| Bladder NAT 1 | 0.0 | 0.0 |
| Bladder cancer 2 | 0.0 | 1.0 |
| Bladder NAT 2 | 0.0 | 0.3 |
| Bladder NAT 3 | 0.0 | 0.1 |
| Bladder NAT 4 | 6.5 | 1.6 |
| Prostate adenocarcinoma 1 | 0.0 | 7.7 |
| Prostate adenocarcinoma 2 | 0.0 | 1.0 |
| Prostate adenocarcinoma 3 | 0.0 | 4.6 |
| Prostate adenocarcinoma 4 | 0.0 | 2.1 |
| Prostate NAT 5 | 7.5 | 0.5 |
| Prostate adenocarcinoma 6 | 0.0 | 2.7 |
| Prostate adenocarcinoma 7 | 0.0 | 2.8 |
| Prostate adenocarcinoma 8 | 0.0 | 0.6 |
| Prostate adenocarcinoma 9 | 0.0 | 6.4 |
| Prostate NAT 10 | 0.0 | 0.3 |
| Kidney cancer 1 | 33.4 | 12.9 |
| Kidney NAT 1 | 0.0 | 5.8 |
| Kidney cancer 2 | 40.6 | 80.7 |
| Kidney NAT 2 | 0.0 | 4.6 |
| Kidney cancer 3 | 6.3 | 49.3 |
| Kidney NAT 3 | 0.0 | 2.4 |
| Kidney cancer 4 | 6.8 | 19.1 |
| Kidney NAT 4 | 0.0 | 4.3 |

CNS_neurodegeneration_v1.0 Summary: Ag2772/Ag6309 This panel confirms the expression of this gene at low levels in the brains of an independent group of individuals.

General_screening_panel_v1.5 Summary: Ag6309 Highest expression of this gene is detected in spinal cord (CT=29.4). Moderate expression of this gene is mainly seen in all the region of central nervous system examined, including amygdala, hippocampus, substantia nigra, thalamus, cerebellum, cerebral cortex, and spinal cord. This gene codes for semaphorin 6A protein (Sema6A). Sema6A is shown to be expressed in thalamocortical neurons and required for their axons to project properly (Leighton Pa., Mitchell K J, Goodrich L V, Lu X, Pinson K, Scherz P, Skarnes W C, Tessier-Lavigne M. 2001, Nature 410(6825):174-9). Therefore, therapeutic modulation of this gene product may be useful in the treatment of central nervous system disorders such as Alzheimer's disease, Parkinson's disease, epilepsy, multiple sclerosis, schizophrenia and depression.

Low expression of this gene is also seen in number of cancer cell lines derived from brain, ovarian, melanoma and a renal cancer. Therefore, therapeutic modulation of the expression of this gene or Sema6A protien encoded by this gene through the use of small molecules or antibodies may be useful in the treatment of these cancers, especially in inhibiting migration of these cancer cell lines.

HASS Panel v1.0 Summary: Ag2772 Highest expression of this gene is seen in a brain cancer (487 medullo) sample (CT=27.3). High to moderate expression of this gene is seen in medulloblastoma and glioma brain cancer samples and prostate cancer (LnCAP) cell line. Expression of this gene is downregulated in LnCAP cells under acidic plus hypoxic environment. In addition, low expression of this gene is also seen in MCF7 cells. Therefore, therapeutic modulation of this gene or its protein product may be useful in the treatment of brain, prostate and breast cancers.

Panel 1 Summary: Ag88 Highest expression of this gene is seen in cerebellum (CT=24.5). High expression of this gene is mainly seen in all the regions of central nervous system examined. Please see panel 1.5 for further discussion of this gene.

High to moderate expression of this gene is also seen in tissues with metabolic/endocrine functions including, pancreas, thyroid, adrenal gland, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

High to moderate expression of this gene is also seen in number of cancer cell lines derived from melanoma,ovarian, renal, colon, liver and brain cancers. Therefore therapeutic modulation of this gene or its protein product may be useful in the treatment of these cancers.

Panel 1.3D Summary: Ag2772 Highest expression of this gene is seen in fetal skeletal muscle (CT=27.4). Interestingly, this gene is expressed at much higher levels in fetal (CT=27.4) when compared to adult skeletal muscle (CT=31.5). This observation suggests that expression of this gene can be used to distinguish fetal from adult skeletal muscle. In addition, the relative overexpression of this gene in fetal tissue suggests that the protein product may enhance muscle growth or development in the fetus and thus may also act in a regenerative capacity in the adult. Therefore, therapeutic modulation of the protein encoded by this gene could be useful in treatment of muscle related diseases.

Some expression pattern correlates with (ex: cancer cell lines) that seen in panel 1.

Panel 2D Summary: Ag2772/Ag88 Two experiments with different probe primer sets are in excellent agreement, with highest expression of this gene seen in a liver cancer (OD04310) sample (CTs=25-28). This gene shows a widespread expression in this panel, with high to moderate expression in normal and cancer samples from stomach, ovary, bladder, colon, liver, lung, metastatic melanoma, kidney, uterus, thyroid and breast. Interestingly, expression of this gene is upregulated in metastatic melanoma, gastric, liver and kidney cancers. Therefore, expression of this gene may be used as marker to detect the presence of metastatic melanoma, gastric, liver and kidney cancers, furthermore, therapeutic modulation of this gene or its protein product may be useful in the treatment of these cancers.

Panel 3D Summary: Ag88 Highest expression of this gene is detected in a renal cancer cell line (CT=30). Moderate expression of this gene is also seen number of cancer cell lines derived from brain, lung, colon, gastric, renal and bone cancers. Therefore, therapeutic modulation of this gene or its protein product may be useful in the treatment of these cancers.

Panel 4D Summary: Ag2772/Ag88 Two experiments with different probe-primer sets are in good agreement with highest expression of this gene seen in colon and thymus (CTs 27-30). This gene shows moderate to low expression in most of samples in this panel. Expression of this gene is upregulated in activated bronchial and small airway epithelium, basophils, liver cirrhosis and lupus kidney. Therefore therapeutic modulation of this gene or its protein product may be useful in the treatment of asthma, allergies, chronic obstructive pulmonary disease, Crohn's disease, ulcerative colitis, liver cirrhosis and lupus erythematosus.

General oncology screening panel_v_{—}2.4 Summary: Ag6309/Ag88 Highest expression of this gene is seen in lung cancer sample (CTs=27-34.7). Moderate to low expression of this gene is seen in normal and cancer samples from lung, colon, metastatic melanoms, prostate, and kidney. Expression of this gene is upregulated in kidney, metastatic melanoma and lung cancers, which is in agreement with expression seen in panel 2D. Please see panel 2D for further discussion of this gene.

### Example 3

Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000). Variants are reported individually but any combination of all or a select subset of variants are also included as contemplated NOVX embodiments of the invention.

Nine polymorphic variants of CG51896-04 have been identified and are shown in Table 4.

TABLE 4
SNP Variants for CG51896-04.

| Seq ID | | | | | | | |
|---|---|---|---|---|---|---|---|
| Number | | Nucleotides | | | Amino Acids | | |
| (DNA/Protein) | Variant | Position | Initial | Modified | Position | Initial | Modified |
| 131/132 | 13379621 | 272 | T | C | 8 | Leu | Pro |
| 133/134 | 13376060 | 410 | A | G | 54 | His | Arg |
| 135/136 | 13376059 | 416 | T | C | 56 | Leu | Pro |
| 137/138 | 13374940 | 523 | A | G | 92 | Ser | Gly |
| 139/140 | 13375101 | 869 | T | C | 207 | Leu | Pro |
| 141/142 | 13379747 | 967 | G | C | 240 | Ala | Pro |
| 143/144 | 13381632 | 2366 | A | G | 706 | Lys | Arg |
| 145/146 | 13381633 | 2921 | T | C | 891 | Leu | Pro |
| 147/148 | 13381634 | 3018 | G | A | 923 | Met | Ile |

### Example 4

Molecular Cloning of CG51896-02, CG51896-11 and CG51896-13

The open reading frame of CG51896-02 codes for the 626 amino acid long extracellular domain of semaphorin 6A. Oligonucleotide primers were designed to PCR amplify a DNA segment, representing an ORF, coding for CG51896-02. The forward primer includes, a BamHI restriction site while the reverse primer contains an, in frame, XhoI restriction site for further subcloning purposes.

The open reading frame of CG51896-11 codes for the 649 amino acid long extracellular domain of semaphorin 6A. Oligonucleotide primers were designed to PCR amplify a DNA segment, representing an ORF, coding for CG51896-1 1. The forward primer includes, a SalI restriction site while the reverse primer contains an, in frame, BamHI restriction site for further subcloning purposes.

The open reading frame of CG51896-13 codes for the 878 amino acid long extracellular domain of semaphorin 6A. Oligonucleotide primers were designed to PCR amplify a DNA segment, representing an ORF, coding for CG51896-13. The forward primer includes, a BamHI restriction site while the reverse primer contains an, in frame, XhoI restriction site for further subcloning purposes.

PCR reactions using the specific primers for each of CG51896-02, CG51896-11, CG51896-13 were set up using a total of 5 ng cDNA template containing equal parts of cDNA samples derived from human testis, human mammary, human skeletal muscle, and fetal brain; 1 μM of each of the Sem6A FORW and Sem6A FL-REV primers, 5 micromoles dNT? (Clontech Laboratories, Palo Alto Calif.) and 1 l of 50×Advantage-HF 2 polymerase (Clontech Laboratories, Palo Alto Calif.) in 50 μl volume. An approximately 1 kbp large amplified product was isolated from agarose gel and ligated to pCR2.1 vector (Invitrogen, Carlsbad, Calif.). The cloned insert was sequenced, using vector specific, M13 Forward (−40) and M13 Reverse primers and verified as an open reading frame coding for CG51896-02, CG51896-11 or CG51896-13.

### Example 5

Expression of CG51896-02

Expression of CG51896-02 in Escherichia coli strain E281

A 1.8 kb BamHI-XhoI fragment containing the CG51896-02 sequence was subcloned into BamHI-XhoI digested pET32a (Invitrogen) to generate plasmid 1954. The resulting plasmid 1954 was transformed into E. coli using the standard transformation protocol. The cell pellet and supernatant were harvested 2 h post induction with IPTG and examined for CG51896-02 expression by Western blot (reducing conditions) using an anti-HIS antibody.

Expression of CG51896-02 in Human Embryonic Kidney 293 Cells

A 1.8 kb BamHI-Xhol fragment containing the CG51896-02 sequence was subcloned into BamHI-XhoI digested pCEP4/Sec to generate plasmid 169. The resulting plasmid 169 was transfected into 293 cells using the LipofectaminePlus reagent following the manufacturer's instructions (Gibco/BRL). The cell pellet and supernatant were harvested 72h post transfection and examined for CG51896-02 expression by Western blot (reducing conditions) using an anti-V5 antibody. CG51896-02 is expressed as an approximately 95 kDa protein secreted by 293 cells.

Expression of CG51896-02 in Stable CHO-K1 Cells

A 1.8 kb BamHI-XhoI fragment containing the CG51896-02 sequence was subcloned into BamHI-XhoI digested pEE14.4Sec to generate plasmid 1610. The resulting plasmid 1610 was transfected into CHO-K1 cells using the LipofectaminePlus reagent following the manufacturer's instructions (Invitrogen/Gibco). Stable clones were selected based on resistance against methionine sulfoximine. The expression and secretion levels of the selected clones were assessed by Western blot analysis using HRP conjugated V5 antibody. (The V5 epitope is fused to the gene of interest at the Cter, in the pEE14.4Sec vector.) CG51896-02 is expressed as an approximately 98 kDa protein secreted by CHO cells.

### Example 6

Expression of CG51896-11

Expression of CG51896-11 in Stable CHO-K1 Cells

A 1.9 kb SalI-BamHI fragment containing the CG51896-11 sequence was subcloned into BamHI-XhoI digested pEE14.4Sec to generate plasmid 2797. The resulting plasmid 2797 was transfected into CHO-K1 cells using the LipofectaminePlus reagent following the manufacturer's instructions (Invitrogen/Gibco). Stable clones were selected based on resistance against methionine sulfoximine. The expression and secretion levels of the selected clones were assessed by Western blot analysis using HRP conjugated V5 antibody. (The V5 epitope is fused to the gene of interest at the Cter, in the pEE14.4Sec vector.) CG51896-11 is expressed as a 116 kDa protein secreted by CHO cells.

Expression of CG51896-11 in Human Embryonic Kidney 293 Cells

A 1.9 kb SalI-BamHI fragment containing the CG51896-11 sequence was subcloned into BamHI-XhoI digested pCEP4/Sec to generate plasmid 2282. The resulting plasmid 2282 was transfected into 293 cells using the LipofectaminePlus reagent following the manufacturer's instructions (Gibco/BRL). The cell pellet and supernatant were harvested 72 h post transfection and examined for CG5 1896-11 expression by Western blot (reducing conditions) using an anti-V5 antibody. CG51896-11 is expressed as a 100 kDa protein secreted by 293 cells.

### Example 7

Expression of CG51896-13 in Human Embryonic Kidney 293 Cells.

A 2.6 kb BamHI-XhoI fragment containing the CG51896-13 sequence was subcloned into BamHI-Xhol digested pCEP4/Sec to generate plasmid 3128. The resulting plasmid 3128 was transfected into 293 cells using the LipofectaminePlus reagent following the manufacturer's instructions (Gibco/BRL). The cell pellet and supernatant were harvested 72 h post transfection and examined for CG51896-13 expression by Western blot (reducing conditions) using an anti-V5 antibody. CG51896-13 is expressed as a 130 kDa protein secreted by 293 cells.

### Example 8

Relevant Pathways

PathCalling™ Technology: The sequence of Acc. No CG51896-02 was derived by laboratory screening of cDNA library by the two-hybrid approach. cDNA fragments covering either the full length of the DNA sequence, or part of the sequence, or both, were sequenced. In silico prediction was based on sequences available in CuraGen Corporation's proprietary sequence databases or in the public human sequence databases, and provided either the full-length DNA sequence, or some portion thereof.

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries (as well as commercially available cDNA libraries from Clontech (Palo Alto, Calif.)) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

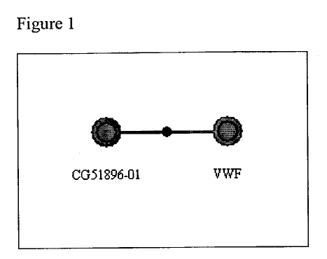

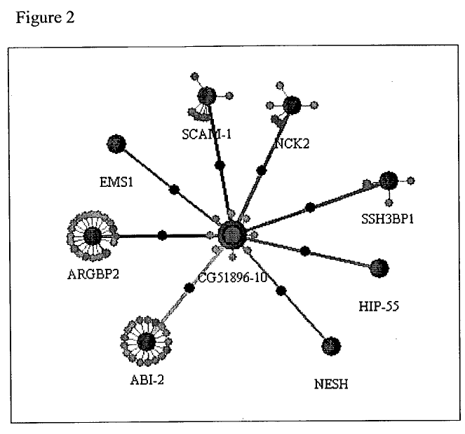

Interacting protein pairs are added to CuraGen's PathCallinge Protein Interaction Database. This database allows for the discovery of novel pharmaceutical drug targets by virtue of their interactions and/or presence in pathologically related signaling pathways. Protein interactions are subsequently analyzed using bioinformatic tools within GeneScape™, which provides a means of visualization of binary protein interactions, protein complex formation, as well as complete cellular signaling pathways. Specifically, as shown in FIG. 1 and FIG. 2, the sequences, which encode proteins CG51896-01 (Semaphorin 6A), VWF (Von Willebrand Factor), NCK2, HIP-55 and ARGBP2a proteins were found to interact and can result in the formation of a protein complex, or may constitute a series of complexes, which form in order to propagate a cellular signal, which is physiologically relevant to a disease pathology. The specific interactions, which constitute the specific complexes, may also be useful for therapeutic intervention through the use of recombinant protein or antibody therapies, small molecule drugs, or gene therapy approaches.

Protein interactions, which are identified through the mining of the PathCalling™ database, can be screened in vitro and in vivo to provide expression, functional, biochemical, and phenotypic information. Assays may be used alone or in conjunction and include, but are not limited to the following technologies; RTQ-PCR, transfection of recombinant proteins, co-immunoprecipitation and mass spectrometry, FRET, affinity chromatography, immunohistochemisty or immunocytochemistry, gene CHIP hybridizations, antisense (i.e. knock-down, knock-up), GeneCalling experiments, and/or biochemical assays (phosphorylation, dephosphorylation, protease, etc.).

Matrix Mating Haploid cells for the PathCalling matrix mating assay are grown up individually in 384 well plates using selective liquid media. After 1-2 days, the mating is done entirely in a nutrient rich media. The resulting diploid cells are then selected using a selective liquid media. The optical density (O.D.) of the diploid cells is measured using a spectrophotometer to measure and the cells are then transferred to a fresh plate for a Beta-gal assay. This assay is performed to determine if there is an interaction between the two proteins being tested. The Beta-gal assay is performed as follows:

1. 30 microliters (μl) of diploid cells are transferred to a new plate (384 well flat bottom plate) using the GenMate 96 well pipetter.

2. The β-gal buffer is made using the following (per 384 well plate): 7.1 ml Sigma water, 7.5 ml 4X Z-buffer, 0.3 ml 20% IGEPAL, 30 mg CPRG, 75 μl Lyticase (10,000 U/ml) and 3.30 μl of β-gal buffer is added to each well of the plate made in step 1 using a multi-drop 384.

3. The plates are placed in a 30° C. incubator for 24 hours.

4. After the 24 hour incubation period, each plate is read on a Bio-Tek plate reader at wavelengths 660 nm and 580 nm.

5. The delta OD (660-580) is used along with visual inspection to determine positive interactions (color change from yellow to red).

As shown in FIG. 1, PathCalling data shows that the extracellular domain of CG51896-01 interacts with Von Willebrand Factor (VWF), a glycoprotein that functions both as an anti-hemophilic factor carrier and a platelet-vessel wall mediator in the blood coagulation system. Table 5 summarizes the amino acid sequences of the bait and prey used in seven independent experiments to detect this novel interaction. Lian et. al has shown that the glycoprotein Ibα mediates endothelial cell migration on von Willebrand factor-containing substrata and that this migratory activity is much higher in TNFα-treated endothelial cells (Lian et. Al, Exp Cell Res 1999, 252(1): 114-22). Since CG51896-01 is upregulated upon TNFα-treatment, it may mediate the increase in migratory activity.

As shown in FIG. 2, PathCalling data shows that the cytoplasmic domain of CG51896-01 interacts with HIP-55, an SH3 actin-binding protein, and two SH3 containing proteins that are in the c-Abl pathway, NCK2 and ARGBP2a. Table 6 summarizes the domains used to detect the intracellular interactions in the screening and matrix 1×1 assays. The number of positive interactions detected and their detection in both orientations with respect to yeast two-hybrid fusion proteins confirms the discovery of a novel interaction between CG51896-01 and the two SH3 containing proteins. NCK2 is an SH2/SH3 adaptor protein that associates with receptor tyrosine kinases, interacts with focal adhesion kinase and regulates cell motility. It also activates c-Abl and modulates Abl transforming activity. ARGBP2a is Arg/Abl-interacting protein and belongs to a family of adaptor proteins that regulate cell adhesion, cytoskeletal organization, and growth factor signaling by linking Abl family kinases to cytoskeleton. A second bait of CG51896-01 was also shown to interact with two c-Abl-interacting proteins, ABI-1 and ABI-2. These two proteins are SH3-containing proteins that regulate actin organization and cell motility, and modulate c-Abl transforming activity. These interactions demonstrate that the CG51896-01 intracellular signaling pathway may involve the c-Abl pathway to regulate cell migration.

TABLE 5
Yeast Two-hybrid Extracellular Interaction Information

| | | | Number |
|---|---|---|---|
| | CG51896-01 | VWF | of Yeast |
| Interaction | Interation | Interaction | Colonies |
| Frame | Domain (aa) | Domain (aa) | Observed |
| 3 (+) | Bait: 475-626 | Prey: 2231-2764 | 1 |
| 1 (+) | Bait: 475-626 | Prey: 2699-2813 | 2 |
| 1 (+) | Bait: 475-626 | Prey: 2659-2813 | 1 |
| 1 (+) | Bait: 475-626 | Prey: 2687-2813 | 1 |
| 3 (+) | Bait: 475-626 | Prey: 1896-2813 | 1 |
| 2 (+) | Bait: 475-626 | Prey: 1896-2813 | 1 |
| 3 (+) | Bait: 475-626 | Prey: 2243-2813 | 1 |

TABLE 6
Summary of Intracellular Screen and Matrix Assay Results

| | Amino | | Amino | | |
|---|---|---|---|---|---|
| | Acids | | Acids | | Positive |
| | in the | | in the | Positive | in |
| | BD | | AD | in | Matrix |
| Binding Domain (BD) | Fusion | Activation Domain | Fusion | Screen | Assay |
| Fusion Protein | Protein | (AD) Fusion Protein | Protein | (Y/N) | (Y/N) |
| CG51896-10, intracellular domain | 690-1047 | NCK2 | 1-380 | | + |
| CG51896-10, intracellular domain | 805-1047 | NCK2 | 1-380 | + | + |
| CG51896-10, intracellular domain | 950-1047 | NCK2 | 1-380 | | + |
| CG51896-10, intracellular domain | 690-808 | NCK2 | 1-380 | | − |
| NCK2 | 1-380 | CG51896-10 | 690-1047 | | − |
| NCK2 | 1-380 | CG51896-10 | 805-1047 | | + |
| NCK2 | 1-380 | CG51896-10 | 950-1047 | | − |
| NCK2 | 1-380 | CG51896-10 | 690-808 | | − |
| CG51896-10 | 805-1047 | ARGBP2, Arg/Abl binding | 319-1100 | + | |
| | | protein | | | |
| ARGBP2 | 802-1100 | CG51896-10 | 690-1047 | | + |
| ARGBP2 | 802-1100 | CG51896-10 | 950-1047 | | + |
| ARGBP2 | 802-1100 | CG51896-10 | 805-1047 | | − |
| ARGBP2 | 802-1100 | CG51896-10 | 690-808 | | − |
| CG51896 | NA | ABl-2, Abl-interactor 2 | 136-513 | + | |
| CG51896 | NA | ABl-1, Abl-interactor 1 | −32-451 | + | |
| | | (gbh_af260262) | | | |
| DAB2, disabled homolog 2 | 564-770 | ArgBP2a | | + | |
| DAB2, disabled homolog 2 | 564-770 | ABl-1, Abl-interactor 1 | −32-451 | + | |
| | | (gbh_af260262) | | | |
| NCK2 | 1-380 | DAB2, disabled homolog 2 | 528-769 | | + |
| ARGBP2 | 802-1100 | DAB2, disabled homolog 2 | 528-769 | | + |
| ARGBP2 | 802-1100 | ABI-, Abl-interactor 1 | −4-451 | | + |
| | | (gbh_af26O262) | | | |
| ARGBP2 | 802-1100 | NCK2 | 1-380 | | + |
| ARGBP2 | 802-1100 | ARGBP2 | | | + |

### Example 9

Migration and Invasion

CG51896-02 was expressed in a number of tissues, with the highest level of expression found in vascularized tissues and normal brain. The mRNA expression profile of CG51896-02 (Example 2) was striking in that it was elevated in renal and lung tumor tissues as well as in HUVEC and in a majority of renal clear cell carcinoma (RCC) cell lines. CG51896-02 is also elevated in a number of melanoma cell lines. These observations suggested that CG51896-02 plays a role in endothelial cell processes and potentially tumor neovascularization. Migration of endothelial cells is one of the important process in angiogenic cascade. Thus role of CG51896-02 polypeptide on the migration processes was tested as described below.

Migration Assay

To determine if Semaphorin proteins CG51896-02 and CG51896-11 influence cell migration, cell lines were screened for cell motility in response to various treatments. Cell lines tested include: HWVEC (human umbilical vein endothelial cells), HMVEC-d (human microvascular endothelial cells), U87MG (neuroblastoma), 786-0 (renal carcinoma, epithelial), HT1080 (fibrosarcoma), SJCRH30 (rhabdosarcoma), SK—N—SH (neuroblastoma), and CAKI-2 (renal carcinoma). 24-well transwell (BD Biosciences, Bedford, Mass.) migration chambers (8 μm pore size) were used. Briefly, 4×10⁴ cells in serum free medium (Medium 200 for HUVEC, Medium 131 for HMVEC-d, and DMEM high glucose/1% Penicillin/Streptomycin/10% FBS for the cancer cell lines) containing 0.1% BSA were added to wells in the upper chamber (300 μl). The chambers were pre-coated with Type I Collagen at 10 μg/ml for 1 h at 37° C. The lower chamber was filled with chemotactant (1% FBS supplemented with 10 ng/ml of VEGF). CG51896-02 or CG519896-11 in various concentrations ranging from 1 ng/ml to 100 ng/ml was added to the upper chamber and the cells allowed to migrate at 37° C. Following incubation, cells on the upper surface of the membrane (non-migrated cells) were scraped with a cotton swab. Cells on the lower side of the membrane (migrated cells) were stained with 0.2% Crystal Violet dye (Fisher Scientific, Springfield, N.J.) in 70% ethanol for 30 min. The cells were then de-stained in PBS, pH 7.4 and the membrane was left to air dry at room temperature. Migrated cells were counted using a Zeiss Axiovert 100 inverted microscope. Three independent areas per filter were counted and the mean number of migrated cells was calculated. An RGD control peptide (Invitrogen; Cat. No. 12135-018) with the amino acid sequence “GRGDSP” was used as a positive control for the endothelial cell lines, and fetal bovine serum (FBS) ranging from 0.5% to 2% (with or without VEGF, depending on the cell line) was used as a positive control for the cancer cell lines. Serum free media (SFM) was used as a negative control.

Results and Conclusion

Migration of endothelial cells is one of the important processes in angiogenic cascade and thus inhibition of migration indicates that CG51896-02 polypeptide would inhibit the growth of new blood vessels and thus will be an ideal candidate for anti-angiogenic therapy. From the results detailed below, use of CG51896-02 polypeptide as a therapeutic for glioblastoma and renal cancer is proposed.

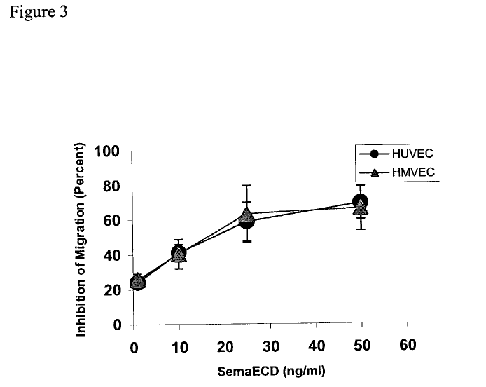

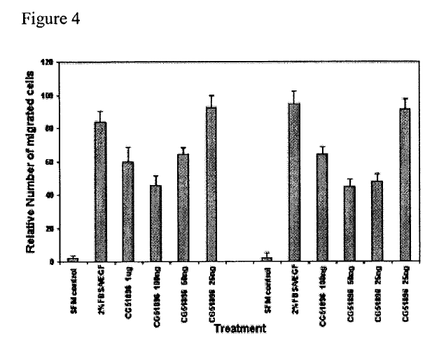

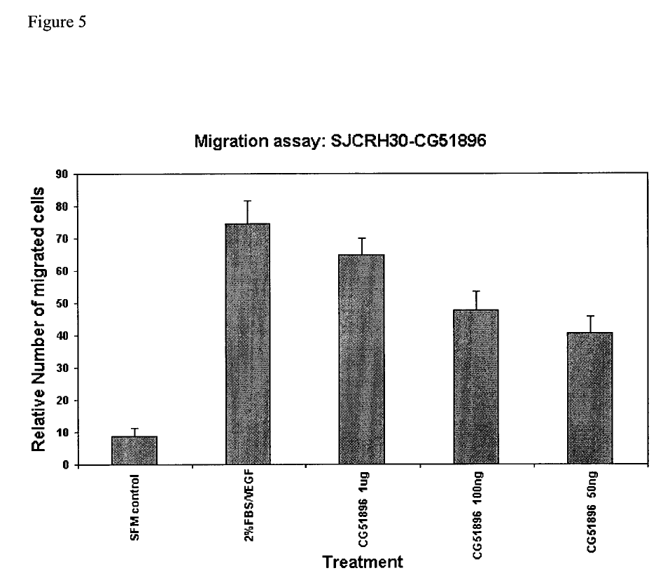

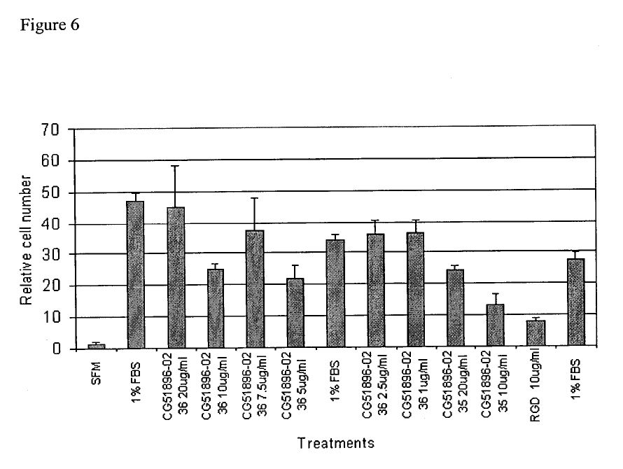

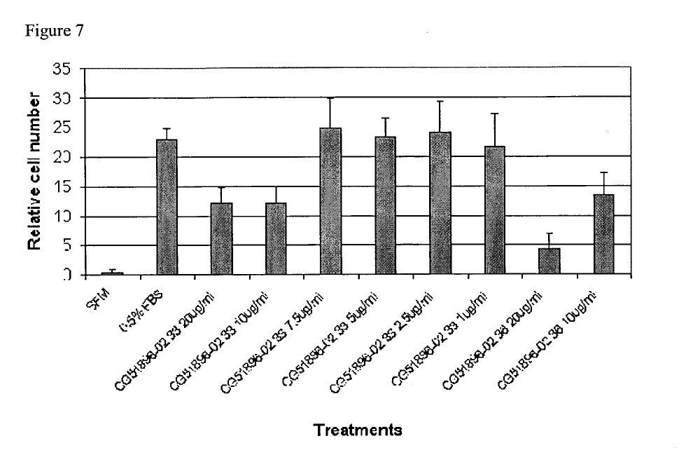

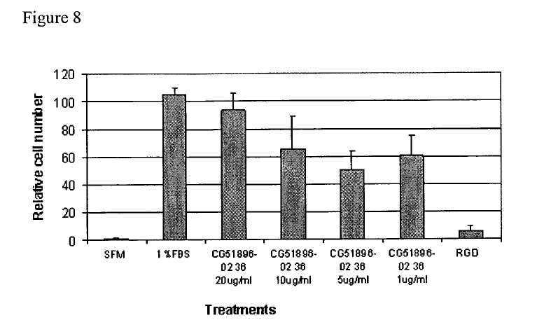

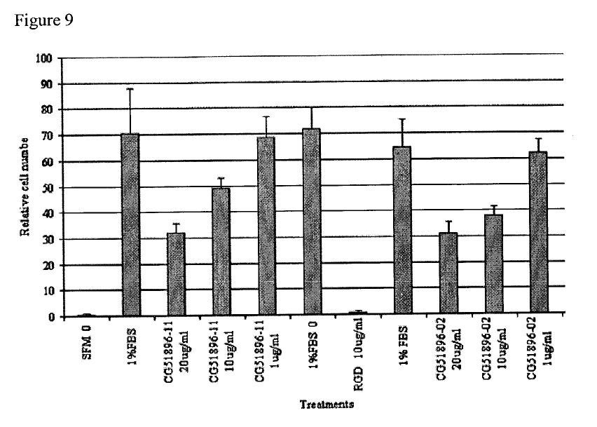

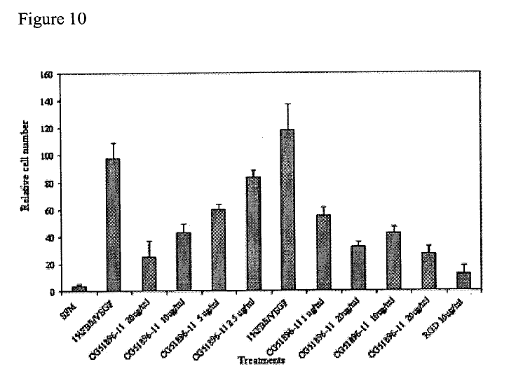

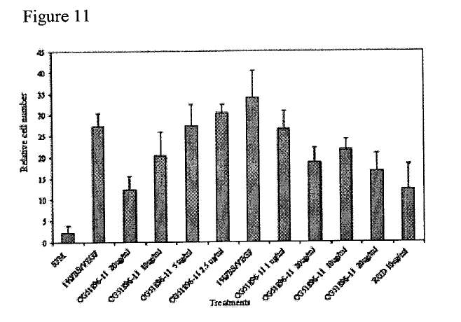

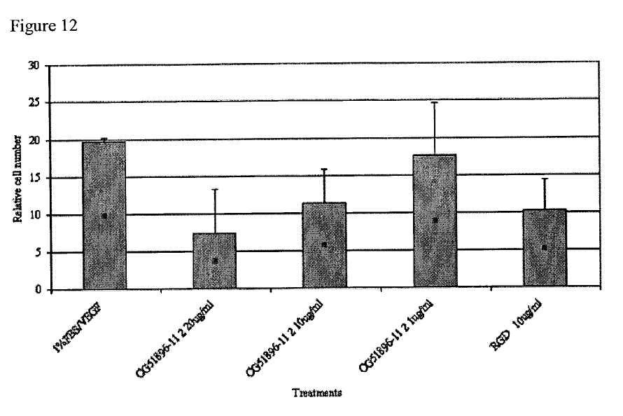

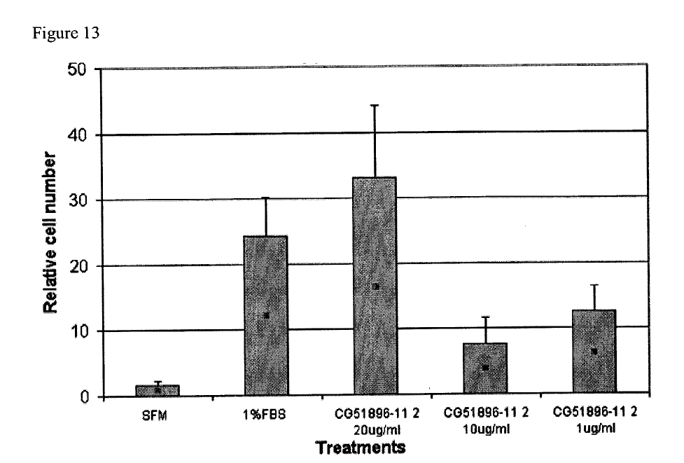

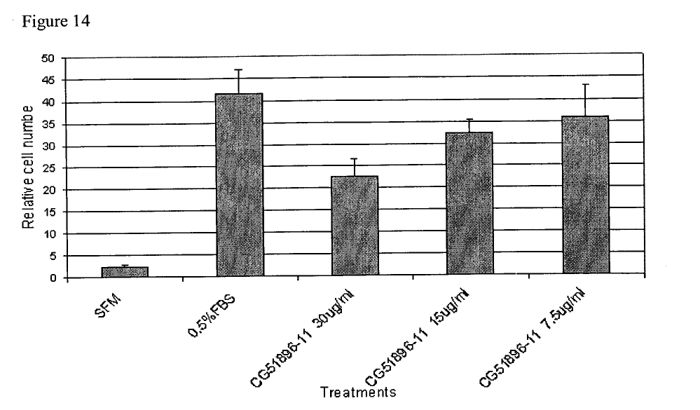

Soluble semaphorin significantly inhibited the VEGF-induced migration of endothelial cells in a dose-dependent manner. The inhibition of migration was seen in human umbilical vein (HUVEC) as well as microvascular endothelial cells (HMVEC-d). A concentration of 50 ng/ml of semaphorin resulted in approximately 60% inhibition of migration of HUVEC and HMVEC-d cells (FIG. 3). These results demonstrate that the extra-cellular domain of semaphorin specifically inhibits the VEGF-mediated migration of endothelial cells. In addition, CG51896-02 also inhibited the migration of human renal carcinoma (786-0), rhabdosarcoma (SJCRH30), and neuroblastoma (SK—N—SH, U87MG) and Caki-2 cell lines (FIG. 4 through FIG. 8). The activity of the novel splice variant CG51896-11 was benchmarked against the CG51896-02 variant using the SK—N—SH neuroblastoma cell line. FIG. 9 demonstrates that the CG51896-11 novel splice variant inhibited the migration of tumor cell in a dose dependent manner with activity that was comparable to the CG51896-02 variant. FIG. 10 through FIG. 13 further demonstrate that CG51896-11 inhibited migration in a fibrosarcoma, renal carcinoma, endothelial and a neuroblastoma cell line. In addition, G51896-11 inhibited migration of Panc-1 cell line suggesting the anti-angiogenic role of the protein in pancreatic cancer (FIG. 14).

Table 7 provides a summary of the effect of CG51896-02 or CG51896-11 on various cell lines as regards to inhibition of migration.

TABLE 7
Inhibition of Migration by CG51896-02 and CG51896-11

| | | | CG51896-02 | CG51896-11 |
|---|---|---|---|---|
| Cell line | Tumor/Cell type | Species | Activity | Activity |
| M14 | Melanoma | Human | − | NA |
| SKMe128 | Melanoma | Human | − | NA |
| B16F10 | Melanoma | Mouse | − | NA |
| UACC-62 | Melanoma | Human | − | NA |
| 786-0 | Renal carcinoma | Human | + | + |
| Caki-2 | Renal carcinoma | Human | + | + |
| SJCRH30 | Rhabdosarcoma | Human | + | NA |
| SK-N-SH | Neuroblastoma | Human | + | + |
| U78MG | Neuroblastoma | Human | + | + |
| HUVEC | Endothelial | Human | + | + |
| HMVEC-d | Endothelial | Human | + | NA |
| C-PAE | Endothelial | Bovine | + | NA |
| BAE | Endothelial | Bovine | + | NA |
| HASMC | Smooth muscle | Human | − | NA |
| HT1080 | Fibrosarcoma | Human | NA | + |

− inhibition of migration not observed
+ inhibition of migration observed
N/A experiment was not done

Invasion Assay

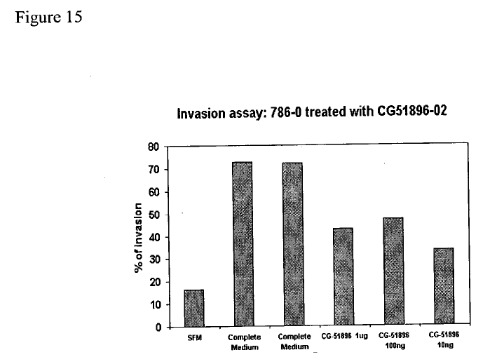

Matrigel coated invasion inserts (Becton, Dickinson) were rehydrated with 400 μl PBS buffer and incubated at room temperature for one hour. Cells were suspended in 10 ml basal media (DMEM basal medium +2.5% FBS. GIBCO-BRL) containing 0.1% BSA (diluent) and centrifuged for 5 minutes at 1000 RPM. The cells were re-suspended in diluent, counted, and diluted to 6×10⁴ cells/ml or 1×10⁵ cells/ml with diluent. 0.02 ml of conditioned media containing a 10× stock of CG51896-02 was added into a microtube with 0.18 ml of cells at the appropriate density. Samples were analyzed in quadruplicate. 0.2 ml of cell suspension were placed into each insert along with the purified proteins (40,000 cells in 180 μl of assay medium +20 μl of 10× concentrated purified protein) incubated for 20 hrs. VEGF (10 ng/ml) (R&D Systems) acted as a positive control motility factor for endothelial cells. To determine non-specific invasion, basal medium containing 0.1% BSA was added to the lower chamber. Complete medium containing all the necessary growth factors was used as positive control. After the 20 h incubation period, the cells were removed from the upper side of the insert using a cotton swab. The cells adhering to the underside of the filter were stained with 0.2% crystal violet in 70% ethanol for 30 min at room temperature and washed with distilled water. The adherent (invading) cells were counted under the microscope. Three random different fields were chosen and the number of cells that migrated in that region were counted. CG51896-02 affected the invasion of the 786-0 cells in a dose dependent manner (FIG. 15).

### Example 10

Semaphorin Inhibits Cytoskeletal Reorganization

From the results obtained in migration assays (Example 9), it is clear that CG51896-02 polypeptide affected the migration of both endothelial and 786-0 RCC tumor cell lines. From the literature, it is known that the migrating cells reorganize their cytoskeleton during migration. Thus the effect of CG51896-02 on actin cytoskeleton reorganization was examined to indicate the biochemical mechanism for inhibition of migration.

Human umbilical vein endothelial cells (HUVEC) were fixed to examinine F actin organization using the procedure described in Miao et al. (Miao et al., J Cell Biol. 146:233-42, 1999). Briefly, 4×10⁴ cells were seeded in 8-chamber Nunc glass slides (Fisher Scientific, Springfield, N.J.) pre-coated with fibronectin at 10 μg/ml and serum starved overnight at 37° C. The cells were treated with concentrations of CG51896-02, ranging from 0.1 ug/ml to 10 μg/ml, in the presence or absence of VEGF₁₆₅ for 30 min. VEGF+Cytochalasin D, a fungal metabolite that acts as a potent inhibitor of actin filament and contractile microfilaments, was used as a negative control. Cells were washed with prewarmed serum free medium, fixed in 3.7% paraformaldehyde and permeabilized with 0.1% Triton-X100. After washing three times with PBS, pH 7.4, the cells were then blocked with heat inactivated BSA (1%) for 30 min at room temperature. The cell actin cytoskeleton was stained with Rhodamine phalloidin (Molecular Probe, Eugene, Oreg.) and counterstained with Sytox green nuclei stain (Molecular Probe, Eugene, Oreg.). After staining, the cells were washed with PBS, pH 7.4, and mounted using a fluoromount (Fisher Scientific, Springfield, N.J.). The samples were examined in a Zeiss Axiovert 100 microscope with the Kodak camera. Digital images were captured and analyzed using the Photoshop 5.5 program.

Results and Conclusion

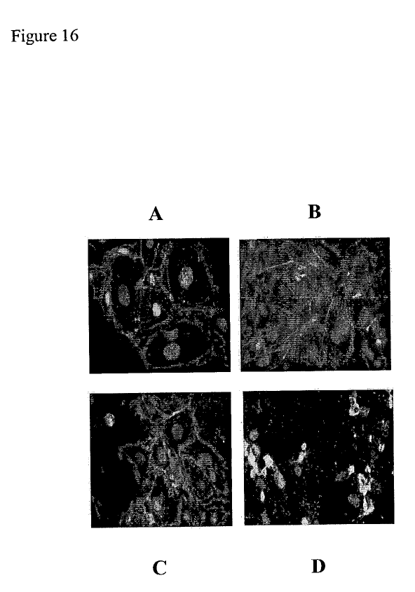

FIG. 16 shows that there was less filamentous actin in the unstimulated control endothelial cells (A) compared to the VEGF stimulated cells (B). The actin stress fiber formation in CG51896-02 treated cells without stimulation was comparable to unstimulated cells (Data not shown). VEGF at 10 ng/ml stimulated an increase in actin filament formation and in particular increased the number of transverse filament bundles that crossed the cells (Photo B), whereas VEGF+Cytochalasin D effectively inhibited this process (D). However, in the presence of CG51896-02 (10 μg/ml) (Photo C), there was a marked retraction in the actin filament.

The results indicate that CG51896-02 inhibits actin filament formation and have a role in the cytoskeletal reorganization. It is also important to note that the proteins ABI-1, ABI-2, NCK2, DAB2 and ArgBP1, that are shown to interact with CG51896-02 (Example 8, pathcalling data) are involved in the actin cytoskeletal organization and migration.

### Example 11

Semaphorin Inhibits Src Tyrosine Kinase (Src) and Focal Adhesion Kinase (FAK) Phosphorylation

To understand the role of CG51896-02 protein in signaling pathway, receptor activation was studied by measuring the incorporation of phosphotyrosine. Confluent endothelial cells were starved overnight in 0.1% FBS and pretreated with 1 μg/ml or 10 μg/ml of CG51896-02 for 30 min before stimulating cells with recombinant VEGF₁₆₅ at 10 ng/ml. Confluent endothelial cells were then trypsinized and plated onto a 10 cm² petri dish coated with fibronectin at 10 μg/ml. One million cells in serum free medium were seeded onto pre-coated plates along with each of the concentrations of soluble CG51896-02 for 30 min in the presence of VEGF at 10 ng/ml. The cells were stimulated with VEGF₁₆₅ 10 min before the harvest. As a control, one million cells were kept in suspension in serum free medium stimulated with VEGF₁₆₅ 10 minutes before harvest. The non-adherent cells were removed and the attached cells were solublized on the plate with lysis buffer (10 mM Tris, pH 7.4, 150 mM NaCl, 1% Triton X-100, 0.5% NP-40 supplemented with the cocktail of protease inhibitors (Roche Molecular Biochemicals, Indianapolis, Ind.) along with 1 mM sodium orthovandate and 1 mM NaF). The cells were lysed for 30 min at 4° C. The lysates were centrifuged at 12,000xg for 20 min at 4° C. The supernatant corresponding to the same number of cells was subjected to immunoprecipitation with one of the following antibodies: p-Src (specific for Src kinase, Calbiochem, San Diego, Calif.), p-Src-TYR416 (Calbiochem), p-FAK (specific for focal adhesion kinase, Santa Cruz Biotechnology, Calif.), p-FAK-Tyr-397 (specific for phosphorylated tyrosine 347, Santa Cruz Biotechnology), p-FAK-Tyr-861 (specific for phosphorylated tyrosine861, Santa Cruz Biotechnology). Immunoprecipitation was performed by adding precleared lysate to protein A Sepharose beads (Amersham Pharmacia, Piscataway, N.J.) to which the appropriate antibodies (see above) had been added. After incubation for 2 h at 4° C. with continuous mixing, the Sepharose bound immune complexes were washed 4x with lysis buffer and then boiled in reducing sample buffer and analyzed by SDS-PAGE and immunoblotting.

Total cellular extracts or immunoprecipitated proteins (VEGF receptor) were separated by SDS-PAGE (4-20%) gel, transferred into nitrocellulose membranes, blocked with 5% non fat dry milk in PBS, pH 7.4 containing 0.1% Tween-20. The membrane was then incubated with the appropriate primary antibodies (lh at room temperature or overnight at 4° C.). Immunoreactive bands were visualized by peroxidase conjugated secondary antibodies and the ECL western blot detection system (Amersham).

Results and Conclusion:

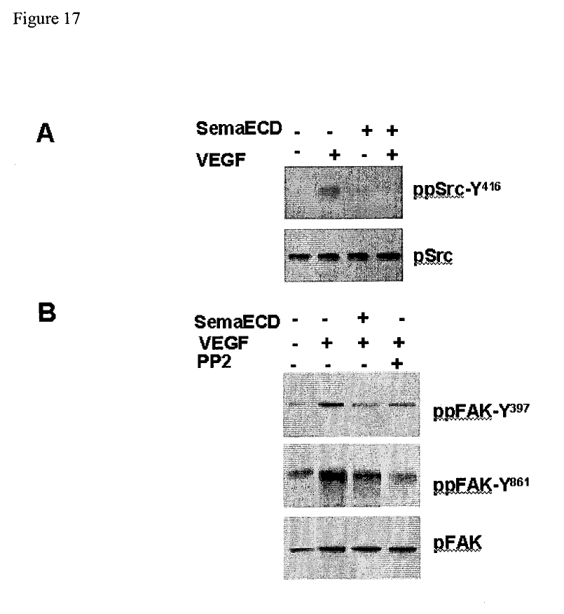

FIG. 17A is a Western blot of the cell lysates prepared from the cells that adhered to fibronectin (as described above), with lanes containing from left to right: untreated lysate, VEGF treated lysate, lysate with CG51896-02, and lysate with both VEGF and CG51896-02. These results demonstrate that VEGF stimulates activation of Src, as assayed by tyrosine phosphorylation of SrcY⁴¹⁶, whereas treatinent of cells with CG51896-02 (100 ng/ml) produces a significant reduction in SrcY⁴¹⁶ phosphorylation (A, Top Panel). The total concentration of Src protein, as measured by a panSrc antibody, remained unchanged under these conditions (A, Bottom Panel). These results suggest that CG51896-02 blocks VEGF-mediated Src activation.

In addition, a second experiment (FIG. 17B) showed that VEGF caused a marked increase in phosphorylation of pFAK³⁹⁷ and pFAK⁸⁶¹ (B, Top and Middle Panels). In contrast, treatment with SemaECD (100 ng/ml) caused a marked reduction in FAK phosphorylation. This inhibitory effect on FAK phosphorylation is a consequence of the inhibition of Src phosphorylation seen above. These effects were comparable to PP2 (2 μM), which inhibits Src mediated FAK phosphorylation. Furthermore, the total amount of FAK protein remained unchanged under these conditions (B, Bottom Panel). The above data indicates that, CG51896-02 inhibits VEGF-mediated phosphorylation of both Src and FAK.

### Example 12

Co-immunoprecipitation of CG51896-02 and Plexin A1

Receptor dimerization or complex formation is a measure of receptor activation and often indicative of a bipartite interaction. CG51896-02-responsive cells were serum starved and stimulated with CG51896-02 for 10 min. Cells were washed once with PBS, 100 μM sodium orthovanadate. Whole cell lysates were prepared by solubilization in RIPA buffer [50 mM Tris pH 7.4, 50 mM NaCl, 1.0% Triton X-100, 5 mM EDTA, 10 mM sodium pyrophosphate, 50 mM sodium fluoride, 1 mM sodium orthovanadate, 1 mM phenylmethylsulfonylfluoride, leupeptin (10 μg/mL), pepstatin (10 μg/mL), and aprotinin (1 μg/mL)], sonication, and incubation on ice for 30 min. Lysates were cleared by centrifugation at 14,000 rpm for 10 min. Lysates containing equivalent amounts of total protein were incubated with anti-receptor antibody for 2 h. Next, 100 μL of a 1:1 slurry of protein G Sepharose was added for 2 h. Immunocomplexes were washed 3 times with RIPA buffer. Non-denaturing polyacrylamide gel electrophoresis (PAGE) sample buffer was added, and the samples were fractionated on 4-15% polyacrylamide gels without boiling.

After electrophoretic transfer to Immobilon P membranes, filters were blocked in TTBS (20 mM Tris pH 7.4, 150 mM NaCl, 0.05% Tween 20), 3% nonfat milk. Membranes were then incubated with anti-receptor serum (1:1000) or anti-phosphotyrosine (1:1000) for 1-2 h in TTBS, 1% BSA, and washed four times with TTBS. Bound antibody was detected by incubation with anti-rabbit (1: 10,000) or anti-mouse antibody (1:10,000) conjugated to horseradish peroxidase (Amersham, Arlington Heights, Ill.) for 30 min and subsequently washing four times with TTBS. Enhanced chemiluminescence (Amersham) was performed according to the manufacturer's protocol.

Results and conclusion

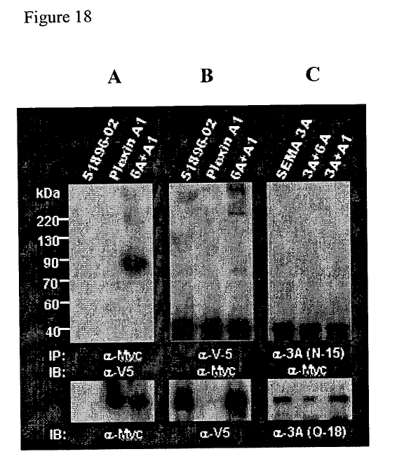

Co-immunoprecipitation experiment demonstrates that CG51896-02 and Plexin A1 interact with each other. CG51896-02 was synthesized with a V5-His epitope and Plexin A1 has a c-myc epitope. In FIG. 18, panel A, co-transfection of both CG51896-02 and Plexin A1 results in detection of CG51896-02 when immunoprecipitated with c-myc antibody and immunoblotted with a V5-his antibody. Visualization of the complex demonstrates the interaction between CG51896-02 and Plexin A1. FIG. 18, panel B demonstrates the results when the immunoprecipitation is done with the anti-V5-his antibody and immunobloted with c-myc antibody. FIG. 18, panel C shows an irrelevant antibody and is the negative control. The results from two different immunoprecipitations clearly demonstrate that CG51896-02 physically interacts with Plexin A1.

### Example 13

Quantification of Membrane Bound CGG51896-02 Protein by Flow Cytometry

FACS analysis was performed to quantify binding of exogenous CG51896-02 to cell lines previously identified as responders to semaphorin (Table 7, Example 9). The analysis was performed on two cell lines, U87-MG (neuroblastoma) and 786-0 (renal cell carcinoma). The cells were lifted from the culture dish using Versene or cell scraper. The cells were washed with PBS buffer and blocked in FACS binding buffer containing 10% goat serum on ice for one hour. After blocking, the cells were centrifuged and resuspended in FACS binding buffer. For each binding reaction, a minimum of 100,000 cells was used. The cells were incubated with CG51896-02 or CG51896-11 (Fc tagged at the 3′end), at various concentrations ranging from 0.1 μg/mL -60 μg/mL, (Table 8) for one hour on ice. Following the incubation period, the cells were washed with FACS binding buffer and incubated with antibody (control: V5His mAb or human Fc specific antibody, Jackson Immunochemical) for one hour at 4° C. Following the wash step, the cells were incubated with a secondary antibody conjugated to Phycoerthrein flurophore for one hour. At the end of incubation, cells were washed in FACS binding buffer and fixed in 1% paraformaldehyde and analyzed using FACS Calibur. Table 8 summarizes the results and shows that both the 786-0 and the U87-MG cell lines bind CG51896-02 or CG51896-16 in a concentration dependent manner.

TABLE 8
Summary of Cell Surface Binding of two isoforms of CG51896

| | | | Geo Mean for | Geo Mean for |
|---|---|---|---|---|
| Gene Name | Concentration | | U87-MG cell line | 786-0 cell line |
| Control | | | 5.43 | 7.22 |
| CG51896-02 | 60 | ug/ml | 111.8 | 74.21 |
| CG51896-02 | 30 | ug/ml | 75.99 | 69.64 |
| CG51896-02 | 15 | ug/ml | 70.61 | 56.73 |
| CG51896-02 | 1 | ug/ml | 33.36 | 15.2 |
| CG51896-02 | 0.1 | ug/ml | 8.24 | 6.9 |
| Control | | | 5.8 | 6.49 |
| CG51896-11 | 60 | ug/ml | 38.3 | 42.9 |
| CG51896-11 | 30 | ug/ml | 30.875.8 | 23.99 |
| CG51896-11 | 15 | ug/ml | 28 | 21.78 |
| CG51896-11 | 1 | ug/ml | 10.25 | 7.01 |
| CG51896-11 | 0.1 | ug/ml | 6.2 | 6.31 |

CG51896-11 used here was tagged to Fc at the 3′ end

### Example 14

Anti-CG51896-02 and -11 Polyclonal Antibody Production

Peptide-based antibodies directed against human CG51896-02 or CG51896-11 was generated by using a 15-18 amino acid peptide after conjugation to a carrier KLH molecule. The conjugated peptide was immunized using standard protocol. Terminal bleed from rabbits were carried after administering two booster injection with the conjugated peptides. The polyclonal antibodies generated were purified from the serum using Protein A affinity column. The purified polyclonal antibody was used in in vitro screening, FACS staining and immunoblots.

1)N27-N40: VGHK PGRNTTQRHRC (SEQ ID NO: 9)

2)I327-I340: CRFKE QKSPDSTWTP (SEQ ID NO: 10)

3) S562-S578: CNDISTPLPDN EMSYNTVYG (SEQ ID NO: 11)

4) C624-C640: CSHNHQ DKKGVIRESY (SEQ ID NO: 12)

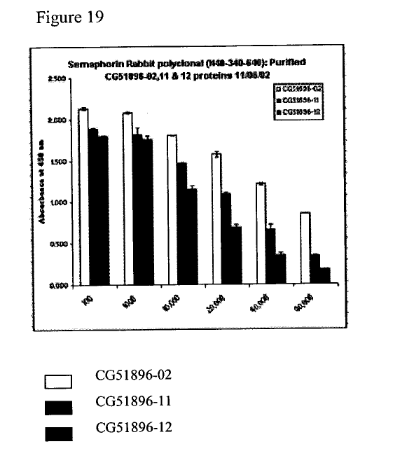

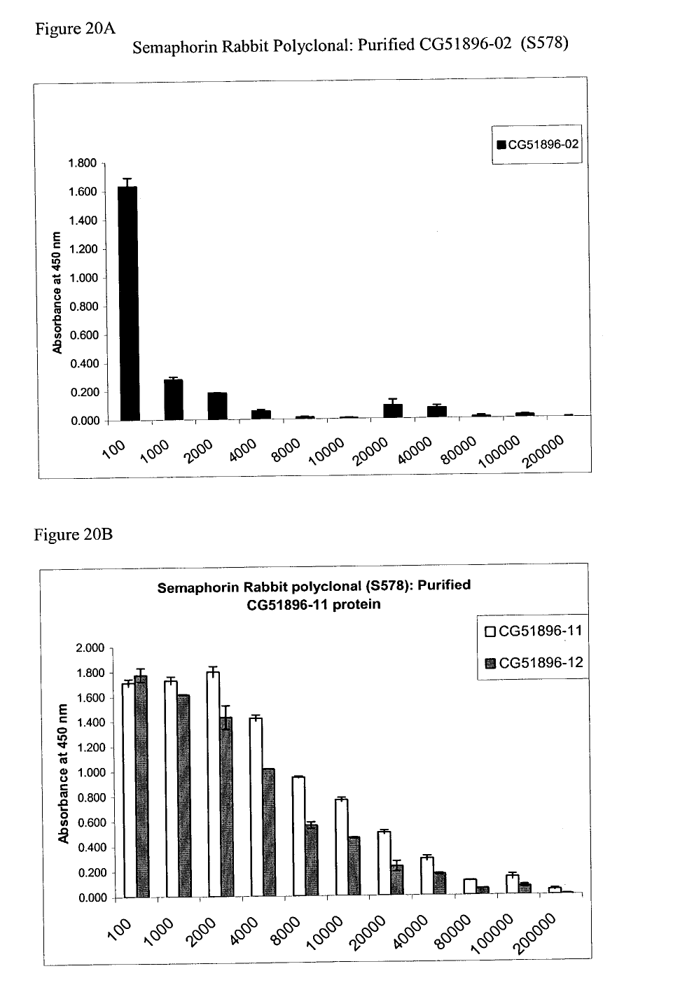

FIG. 19 shows that the mixture of N40, 1340 and C640 sera blocked CG51896-02, 11 and 12 coated at a concentration of 10 μg/ml (read by ELISA). Polyclonal S578 was specific to the splice variant CG51896-11 (and CG51896-12) as shown by FIG. 20A and FIG. 20B.

### Example: 15

Growth Cone Collapse

CG51896-02 protein was assayed for growth cone collapsing activity on explanted chick embryonic day 7 (E7) for dorsal root ganglia. Briefly, explants were dissected from chick embryos and incubated in culture medium supplemented with nerve growth factor (NGF) on eight-well chamber slides precoated with Laminin. The following day, purified CG51896-02 proteins were added to the explanted culture. After 1 h incubation, the explants were fixed in 4% paraformaldehyde at room temperature for 30 min. Explants were then washed in PBS and stained with 3 U/ml of Rhodamine Phalloidin (Molecular Probes, Eugene, Oreg.) in PBS at room temperature for 1 h. Growth cones were visualized under fluorescence microscope and scored as being either normal or collapsed. The percentage of collapsed growth cones were plotted against the concentration of purified protein added to the cultured explant.

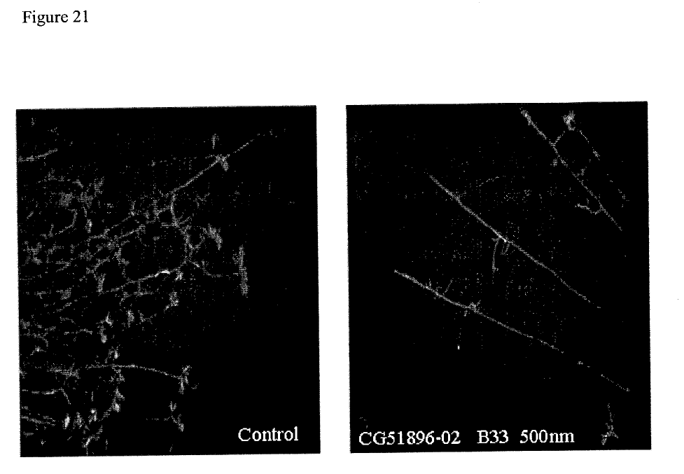

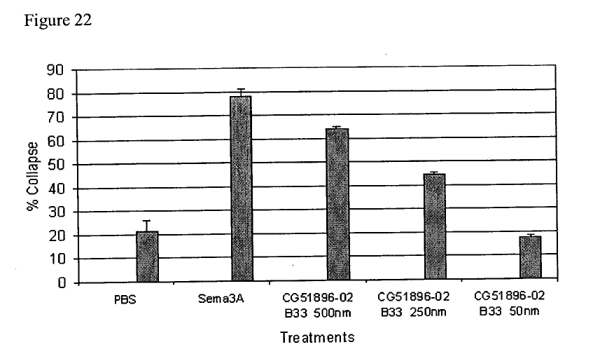

Control E7 explants show the presence of growth cones. However, in the presence of CG51896-02 there is a significant reduction in the number of growth cones (FIG. 21). (FIG. 22) indicates that CG51896-02 is able to induce growth cone collapse with an IC50 value of around 50 nM.

### Example 16

Deorphanization of receptor for CG51896-02 (Prophetic Example)

To determine the mechanism by which the extracellular domain of semaphorin 6A inhibits tumor cell migration and angiogenesis, responder and non-responder cell lines were identified by in vitro analysis followed by binding and FACS analysis. The mechanism by which CG51896-02 inhibits cell migration, blocks angiogenesis and exerts an anti-tumorigenic effect may be due either to the binding of Semaphorin 6A to a specific cell-surface receptor and subsequent inhibition of the receptor function, possibly in cell migration. Alternatively, the extracellular domain of Semaphorin 6A may bind and sequester ligands that normally signal cells to migrate. Expression analysis is performed to determine whether the Sema6A-ECD is exerting its effect by antagonizing endogenous semaphorin signaling, or binding to an as-yet unidentified cell surface receptor.

Analysis of Expression Data

If Sema6A-ECD is exerting a dominant negative effect on endogenous semaphorin signaling, cells that respond to the inhibitory effect of the ECD are likely to express cell-surface proteins involved in semaphorin signaling. In contrast, cells that do not respond to the ECD will have critical molecules missing. Expression data is analyzed to identify the missing molecules. Both microarray and RTQ-PCR data are used in this analysis. A complete list of the signaling proteins in this pathway is shown in Table 9. In conjunction with the focused mining of signaling proteins, differential expression analysis of all cell surface proteins shall be carried out in search of identifying putative novel binding partners.

TABLE 9

| Receptors | Soluble semaphorins | TM semaphorins |
|---|---|---|
| MET receptor | Sema2 (LOC56920) | Sema4A |
| MET receptor like | Sema3A | Sema4B |
| Plexin A1 | Sema3B | Sema4C |
| Plexin A2 | Sema3C | Sema4D |
| Plexin A3 | Sema3D | Sema4E |
| Plexin A4 | Sema3E | Sema4F |
| Plexin 3 | Sema3F | Sema4G |
| Plexin B1 | Sema5A | |
| Plexin B2 | Sema5B | |
| Plexin B3 | Sema6A | |
| Plexin C1 | | Sema6B-isoform1 |
| Plexin D1 | | Sema6B-isoform2 |
| CD72 | | Sema6B-isoform3 |
| Tirn-2 | | Sema6C |
| L1 neural cell adhesion | | Sema6D-isoform1 |
| molecule | | |
| | | Sema6D-isoform2 |
| | | Sema6D-isoform3 |
| | | Sema6D-isoform4 |
| | | Sema6D-isoform5 |
| | | Sema6D-isoform6 |
| | | Sema7A (GPI anchored) |

Selection of Cell Lines

Based on the results of cellular assays (inhibition of migration, Example 9), a list of cell lines responding positively and negatively to semaphorin6A ECD has been compiled (Table 10) Total RNA is prepared from these cell lines for expression analysis.

TABLE 10

| Responder cells | Non responder cells |
|---|---|
| HUVEC (endothelial) | M14 (melanoma) |
| 786-O (kidney epithel | MDA-MB231 |
| U-87 (glimoa) | MDA-MB268 |

Yeast Two-Hybrid Analysis

Yeast two hybrid assays are designed between the semaphorin 6A ECD and known surface receptors involved in semaphorin signaling. Based on the expression pattern of receptors and ligands of the semaphorin pathway in responder cells, the extracellular domains of signaling proteins are cloned from cDNA libraries and used in the experiment. In addition, screening of customized libraries prepared from responding cells may identify novel binding partners. Finally, the interactors from the screens are put into matrix assays to confirm the interactions, determine their specificity and extend pathways. Matrix assays are designed to include other members of ligand/receptor families identified by genomic approaches to address the question of specificity and function.

Homology Mining of Receptors

Mining for receptors shall be undertaken based on the general observation that ligand-receptor pairs are organized into distinct families, such that ligands belonging to one family interact with receptors that are members of another family. Such organization suggests that the three-dimensional conformation of the receptor-ligand binding surface is conserved, although the genes themselves may have diverged during evolution.

The putative receptors identified by expression analysis above are analyzed to determine whether they belong to any known family of receptors. Information from the literature and expression data is superimposed on members of each family of receptors to identify potential families that satisfy the disease rationale associated with the this protein. Application of such stringent restrictions greatly reduces the search-space, and permits detailed analysis and characterization of a subset of receptor proteins by two-hybrid and knockdown experiments.

Cross-Linking and Immunoprecipitation

After identification of responder and non-responder cell lines, the cell surface of target cells or proteins is labeled with biotin or flurophore for subsequent binding studies. Initial binding studies are followed by cleavable or non-cross linker and the cross-linked complexes are pulled out using target specific antibody. Specific complexes that are pulled down only in the responder cell lines are further confirmed as receptors specific to CG51896-02 by competition with unlabeled CG51896-02. If competitive displacement is observed, binding and specificity are confirmed. The responder cell line is identified through LC/MS system or by traditional N-terminal sequencing.

Expression Cloning

Expression cloning screens for cloned receptors based on their ability to elicit a functional response. The expression cloning technique requires introduction of either mRNA or cDNA into a cell that does not normally express the target receptor. After allowing sufficient time for transcription and translation, the transfected cell is tested for a property or functional characteristic of the receptor. Functional analysis can include ligand binding or biological response induced by the presence of the receptor in a non-responder cell line. After determining that introduction of the RNA or cDNA imparts the desired function, the clone is obtained and the sequence is determined. Initially, high quality poly(A) RNA is isolated from cells known to contain the functional receptor. This material is then subdivided into pools and each pool is tested for a functional response.

Coprecipitation and Mass Spectrometry

An approach based on immunoprecipitation of expressed tagged proteins (entry points) followed by identification of proteins complexed with the entry point by mass spectrometry (IP/MS) is be broadly applicable, measures low affinity and transient interactions, measures complex non-binary interactions, captures interactions within every cellular compartment, and measures interactions in relevant cellular milieu.

The gene of interest (bait) is cloned into a mammalian expression vector fused either N- or C-terminally to a tag sequence, such as FLAG or HIS. After transfection and expression of the tagged protein in a revelevant cell line, the cells are lysed under mild conditions, such as in the presence of non-ionic detergents, to solubilize the cells without disrupting native protein-protein interactions. Subsequently the bait protein is captured through affinity purification using e.g. anti-FLAG or anti-HIS antibody-coupled beads, and the complex is washed to remove potential non-specific interactors. Depending on the nature of the bait protein and strength of the intermolecular interactions being analyzed a set of lysis, coimmunoprecipitation and washing conditions are typically explored at this stage to enrich for genuine physiological interactors. Elution of the immunocomplex from the beads is typically done using an elution reagent that specifically releases the bait protein and its interactors, or alternatively with a more general reagent, such as low pH or detergent, that may increase recovery but normally also increases the presence of non-specifically bound proteins. To evaluate the success of the immunocapture the complex is initially analysed by SDS-PAGE combined with silver staining to reveal the complexity of the immunocomplex and the abundance of each constituent.

Proteins captured in the immunocomplex are identified by mass spectrometry. Two methods are used to reduce the complexity of the immunoprecipitants: SDS-PAGE electrophoresis followed by proteolytic digestion of gel bands or 2-D chromatography of the resultant peptide fractions. For the SDS-PAGE method the immunocomplex is run on a gel, and after staining, the bands are excised and digested using trypsin. The resultant peptide mixture is then analyzed using liquid chromatography-electrospray ionization-ion trap mass spectrometry (LC/ESI/IT/MS). The molecular mass and amino acid sequence information obtained from the peptide mixture are then used to identify the immuno-complex proteins by comparison to an annotated database. The search engine utilized for this purpose is MASCOT (Matrix Technologies). For the 2-D chromatography fractionation approach, the immuno-complex is digested in solution using trypsin, and separated using two tandem chromatographic columns (e.g., strong cation exchange-reverse phase). The output of the tandem columns is directed towards the ESI/IT/MS system; the molecular mass and sequence information is then used to provide protein identification.

### Example: 17

Effect of CG51896-02 in Matrigel Plug 786-0 Renal Carcinoma Induced Angiogenesis in Athymic Nude Mice (N-208)

Effect of CG51896-02 polypeptide in matrigel plug assays in a athymic mouse model was done to optimize a quantifiable measure of growth factor-mediated angiogenic response.

The specific goal of this study was to evaluate the effects of CG51896-02 on 786-0, renal carcinoma cell-induced angiogenesis in matrigel plug assay. Stock matrigel preparation containing 786-0 (2×10⁶/ml ) was made in a 50 ml, sterile culture tube. From the stock solution, 0.5 ml of the suspension was injected per mouse, subcutaneously, under aseptic conditions. Control group received equal volume of Matrigel plus vehicle alone. Female athymic nude mice (nu/nu) 8 weeks old were used in this study. Each group had five mice. The experimental design is shown in Table 11.

TABLE 11
Experimental Design for CG51896-02 on 786-0 Renal
carcinoma induced angiogenesis in Athymic nude mice.

| Group | | Number of | Matrigel |
|---|---|---|---|
| Number | Treatment^{a} | Animals | Volume/Mouse |
| 1 | Matrigel Alone | 5 | 0.5 mL/Mouse |
| 2 | Matrigel plus 786-0 | 5 | 0.5 mL/Mouse |
| | cells + vehicle | | |
| 3 | Matrigel plus 786-0 | 5 | 0 5 mL/Mouse |
| | cells, CG51896-02, | | |
| | 1.0 mg/kg, twice | | |
| | daily, IP. | | |
| 4 | Matrigel plus 786-0 | 5 | 0.5 mL/Mouse |
| | cells, CG51896-02, | | |
| | 5.0 mg/kg, twice | | |
| | daily, IP. | | |
| 5 | Matrigel plus 786-0 | 5 | 0.5 mL/Mouse |
| | cells, CG51896-02, 10 | | |
| | mg/kg, twice daily IP. | | |

At the end of 7 days, mice were anesthetized by Ketamine and Xylazine mixture, and the matrigel plugs were removed carefully using microsurgical instruments. Gels were photographed under transillumination. One part of the gel was then fixed in buffered 10% formaldehyde (Sigma Chemicals) overnight and processed for paraffin embedded sectioning. Sections were cut at three different levels and stained with H/E. Another part of the gel was snap frozen in liquid nitrogen and then 10 Jim sections of were prepared. Frozen sections were used for immunocytochemical staining with rat monoclonal antibody directed against mouse CD31 antigen conjugated with phycoerythrin. DAPI staining was used to identify nucleated cells infiltrating the Matrigel plugs. H+E stained slides were evaluated for the formation of distinct, endothelial lined capillaries. Anti-CD31-PE stained slides were observed under fluorescence microscope using appropriate filters. Images were captured digitally using Metamorph software program. Same areas were photographed under red and UV filters to acquire images from CD-31 PE and DAPI staining. Microvessel density was determined by the method published by Wild et al. (Wild et al., 2000, Microvasc. Res. 59(3):368-376). DAPI images were superimposed with respective CD31-PE images to localize blood vessels.

Results and Conclusion

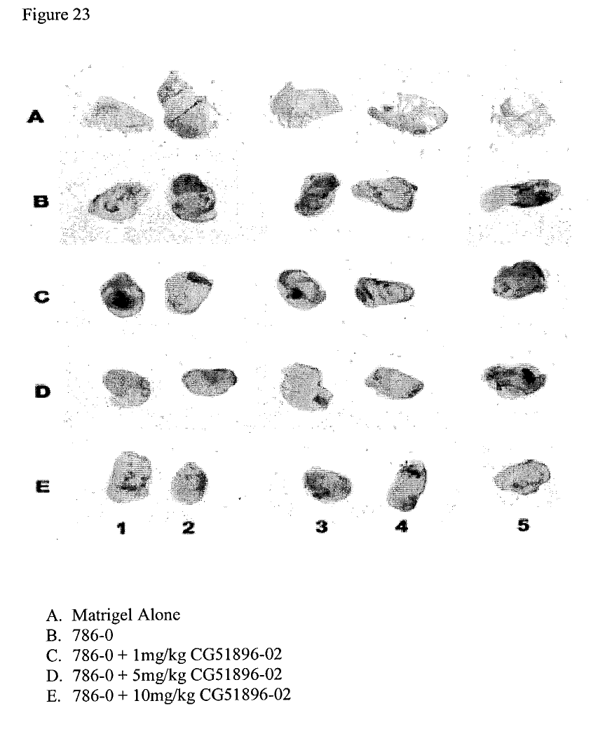

Gross morphology of the matrigel plugs indicates that, there is inhibition of 786-0 renal carcinoma induced angiogenesis in athymic nude mice in the presence of CG51896-02 (FIG. 23). Histology of matrigels from Group E treated with 10 mg/kg of CG51896-02 (FIG. 23, E) shows that most of the area is devoid of any vasculature, indicating that the polypeptide of the present invention is anti-angiogenic and could be used as a therapeutic for renal cancer.

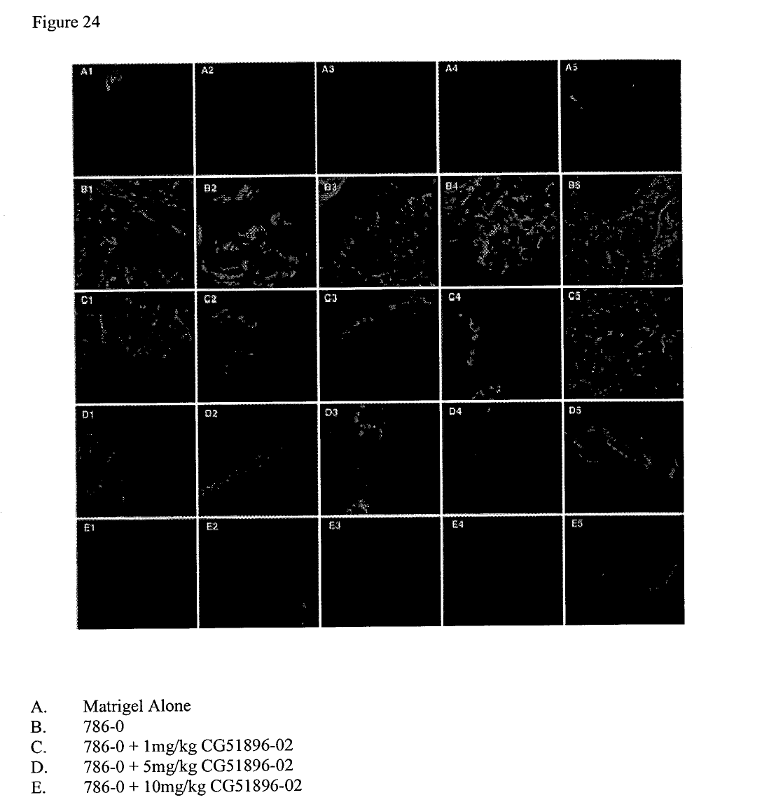

FIG. 24 shows CD31 staining of matrigel plugs demonstrating in vivo inhibition of 786-0 neovacularization, when administered with CG51896-02. DAPI (blue) staining shows infiltrating nucleated cells. Red staining corresponds to CD31-positive endothelial cells. The results further indicated that, at all the three dose levels (1, 5, 10 mg/kg), there was significant reduction in blood vessels. Furthermore, there appears to be a dose response among the treatment groups.

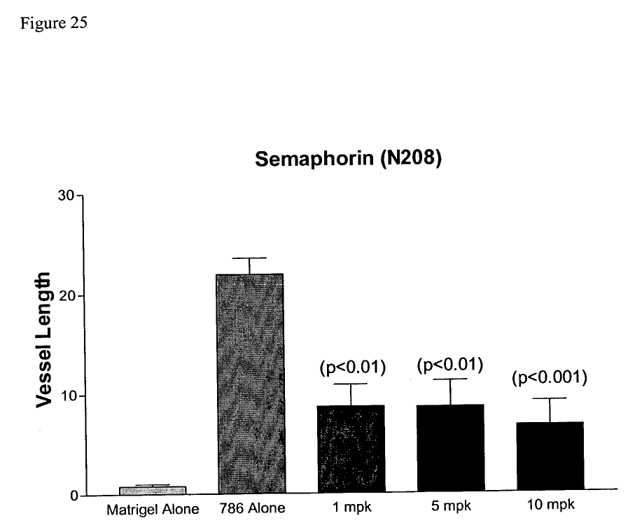

Data from morphometric analysis is summarized in FIGS. 25, 26 and 27. FIG. 25 shows the relative length of blood vessels from each group. Compared to control group, 786-0 cancer cell-containing gels showed a 27-fold increase in total vessel length (0.81 Vs 21.94). Mice treated with CG51896-02 showed marked inhibition in total vessel length. CG51896-02 at 1.0 mg/kg reduced the vessel length by 62.76% when compared to the positive control. Higher doses had further decrease in vessel length. Maximum effect was seen at 10.0 mg/kg dose (71.85% inhibition).

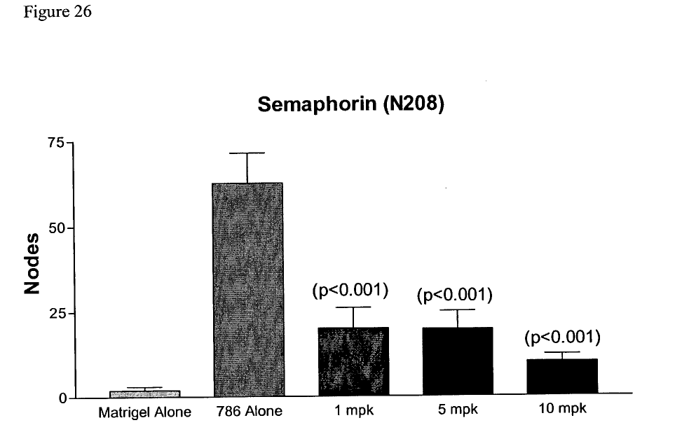

Data in FIG. 26 show comparative angiogenic response (number of nodes) in individual groups. Control group (matrigel alone) showed a mean number of 1.96 nodes per unit area. Inclusion of 786-0 cells in the gels stimulated neovascularization. Number of nodes increased to 62.58 (a 31.92-fold increase). When CG51896-02 was administered to mice cancer cell-induced vascularization was inhibited significantly. At 1.0 mg/kg and 5.0 mg/kg dose, respectively there was a 70.3% and 70.7% reduction in the number of nodes as compared to the positive control. At 10.0 mg/kg dose maximum inhibition was seen (86.63%).

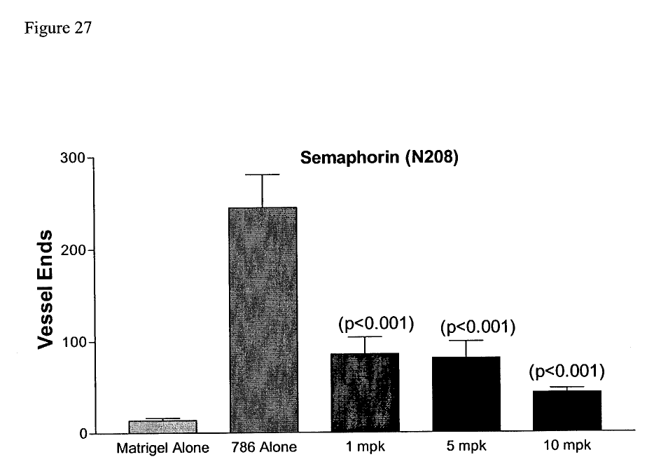

Data in FIG. 27 show the relative number of vessel ends. Control gels (gel alone) had a mean number of 13.86 vessel ends. 786-0 cells increased the number of vessel ends by 17.67-fold (244.92). Treatment with CG51896-02 significantly reduced the number of vessel ends. At 1.0 mg/kg dose, vessel ends were reduced to 85.46 and at 5.0 mg/kg dose, 70.92 vessel ends were seen per field. At the highest concentration tested, 43.14 vessel ends were seen. This corresponds to about 87.33% inhibition of angiogenesis when compared to the positive control group. Inhibition in vessel ends was statistically significant in all the three treatment groups.

Effect of CG51896-11 protein (SEQ ID NO: 50), a novel splice variant, in matrigel plug 786-0 renal carcinoma induced angiogenesis in athymic nude mice revealed comparable results to CG51896-02 in inhibition of in vivo neovascularization, CD3 1 -PE staining and morphometric analysis (data not shown). Matrigel plug 786-0 renal carcinoma induced angiogenesis results thus demonstrate the anti-angiogenic nature of CG51896-02 and -11 polypeptides and their use as a therapeutic for renal cancer.

### Example: 18

Effect of CG51896-02 in Matrigel Plug VEGF/bFGF Induced Angiogenesis in Athymic Nude Mice (N-207)

The protocol of matrigel preparation containing growth factors and administration to athymic mice were as described in Example 17. Table 12 describes the study design that was followed. Immunocytochemical staining with CD31 antibody, DAPI staining and H/E staining were performed as described in Example 17.

TABLE 12
Experimental design for CG51896-02 in Matrigel plug VEGF/bFGF
induced angiogenesis in atymic nude mice.

| Group | | Number of Animals | | Matrigel^{b} |
|---|---|---|---|---|
| Number | Treatmemt^{a} | Females | Males | Volume/mouse |
| 1 | Matrigel alone | 5 | 0 | 0.5 mL/mouse |
| 2 | Matrigel plus 10 | 5 | 0 | 0.5 mL/mouse |
| | ng/mL bFGF & 100 | | | |
| | ng/mL VEGF | | | |
| 3 | Matrigel plus 10 ng/mL | 5 | 0 | 0.5 mL/mouse |
| | bFGF & 100 ng/mL | | | |
| | VEGF + 1.0 mg/kg, | | | |
| | twice daily, IP. | | | |
| 4 | Matrigel plus 10 ng/mL | 5 | 0 | 0.5 mL/mouse |
| | bFGF & 100 ng/mL | | | |
| | VEGF + 5 mg/kg, twice | | | |
| | daily, IP. | | | |
| 5 | Matrigel plus 10 ng/mL | 5 | 0 | 0.5 mL/mouse |
| | bFGF & 100 ng/mL | | | |
| | VEGF + 10 mg/kg, twice | | | |
| | daily, IP. | | | |

Results and Conclusion

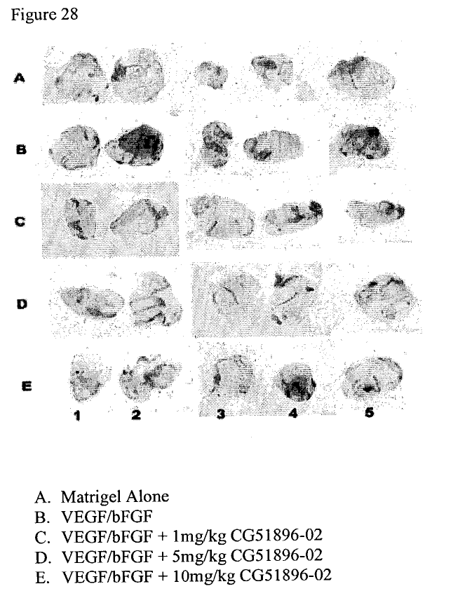

VEGF/bFGF induced significant angiogenesis as evidenced from the distinctly vascularized areas. Gross morphology of the plugs indicate that CG51896-02 treatment (1, 5, 10 mg/kg) inhibited VEGF/bFGF-induced angiogenesis (FIG. 28).

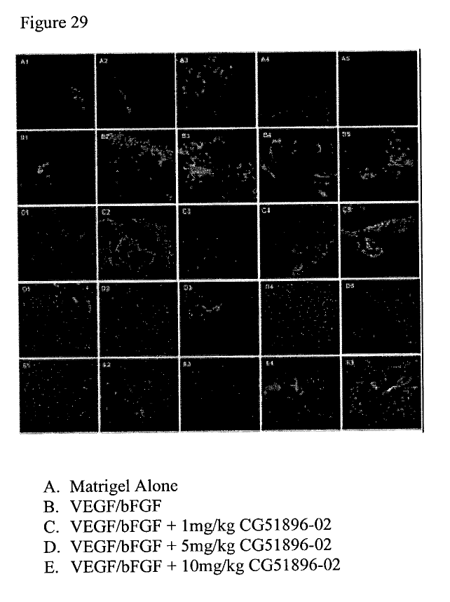

CD31 staining revealed significant reduction in blood vessels with sparse endothelial cells at all three dose levels tested (1, 5, 10 mg/kg, FIG. 29) as compared to the positive control showing higher levels of CD31 staining.

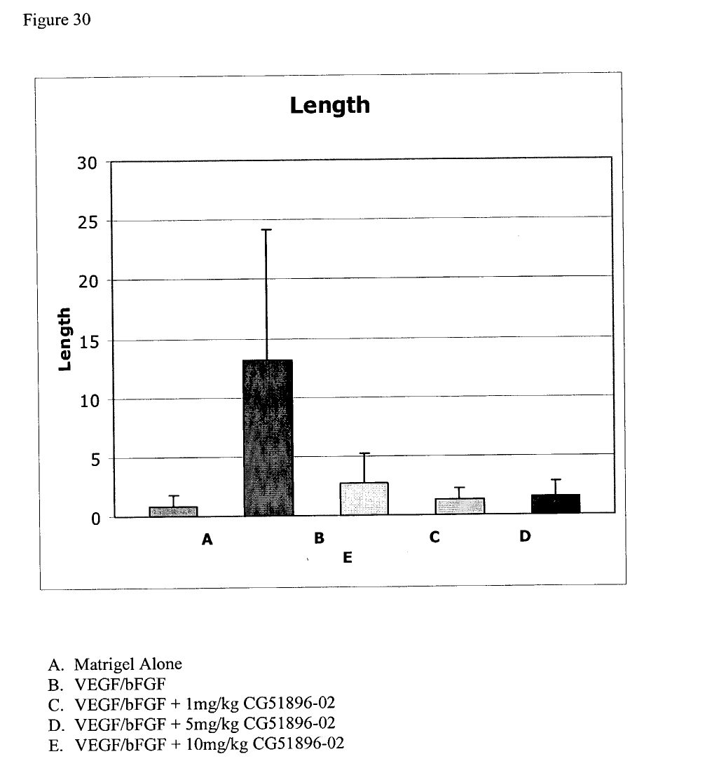

FIG. 30 shows the relative length of blood vessels from each group. Compared to control group, VEGF/bFGF containing gels showed a 16.7-fold increase in total vessel length (0.79 Vs 13.18). Mice treated with CG51896-02 showed marked inhibition in total vessel length. For example, injection of CGS1896-02 at 1.0 mg/kg reduced the vessel length by 85% when compared to the positive control (VEGF/bFGF treated). Higher doses had further decrease in vessel length. Maximum effect was seen at 5.0 mg/kg dose (96% inhibition).

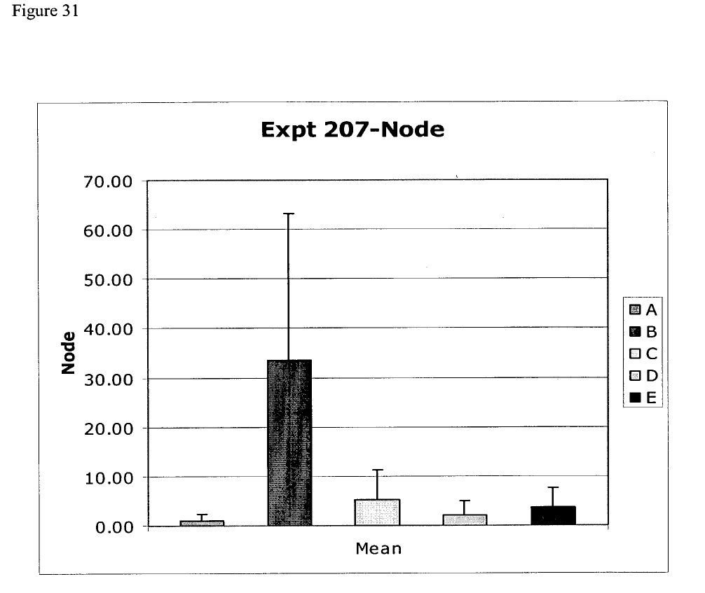

Data in FIG. 31 show comparative angiogenic response (number of nodes) in individual groups. Control group showed mean number of 1.11 nodes per unit area. Inclusion of VEGF/bFGF in the gels stimulated neovascularization as evidenced by a 30-fold increase in the node formation (33.56). When CG51896-02 was administered to mice VEGF/BFGF-induced vascularization was attenuated significantly. At 1.0 mg/kg dose, there was a 87% reduction in the number of nodes. Increasing the dose to 5.0 mg/kg or 10 mg/kg resulted in about 96.5% further decrease in the number of nodes.

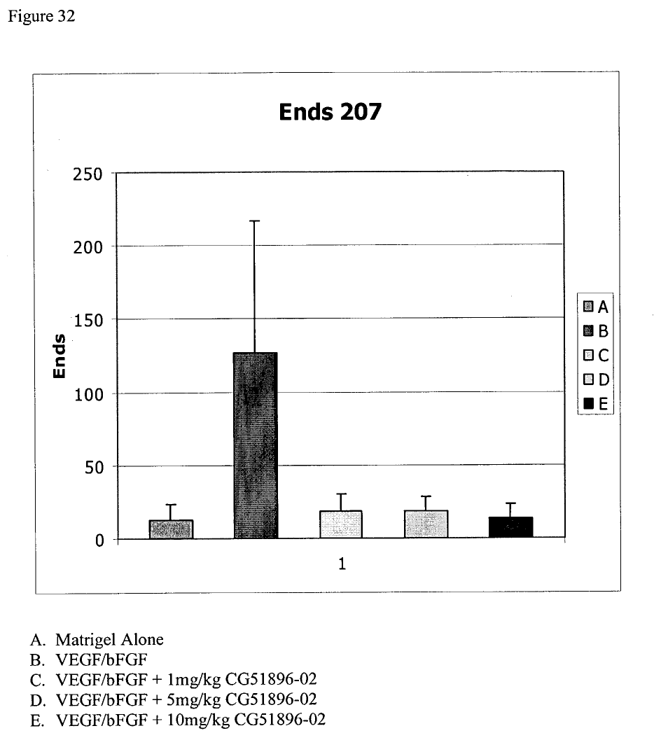

Data in FIG. 32 show the relative number of vessel ends. Control gels (Group A) had a mean number of 12.34 vessel ends. VEGF/bFGF increased the number of vessel ends by 10.3-fold (127.3). Number of vessel ends significantly reduced when mice were treated with CG51896-02 polypeptide. At 1.0 mgfkg dose, vessel ends were reduced to 18.72 and at 5.0 mg/kg dose, 118.2 vessel ends were seen per field. At the highest concentration tested, only 13.26 vessel ends were seen. This corresponds to about 99.2% inhibition of angiogenesis when compared to the positive control group, B, treated with VEGF/bFGF. Inhibition in vessel ends was statistically significant in all the three treatment groups.

### Example 19

Efficacy Evaluation of CG51896-02 Against the U87MG Human Glioma Line Grown as a Xenograft in Nude Mice (N-223)

Human U87MG glioblastomas, implanted subcutaneously in athymic mice, were selected as the tumor model. These tumors are characterized by increased tissue vascularization and expression of angiopoietin-1 and angiopoietin-2 (Audero, E. et al 2001, Arterioscler Thromb Vasc Biol 21, 536-41).

CG51896-02, termed as GU1 in the study, was tested at three dosing levels: 1, 5, and 10 mg/kg administered intraperitoneally (i.p) twice daily for 14 consecutive days (BID×14). Carmustine, a standard chemotherapeutic agent used for the treatment of glioblastomas, was tested as a monotherapy at 15 mg/kg i.p. three doses given once daily on alternate days (QOD×3). The 15 mg/kg carmustine and 5 mg/kg GU1 treatments were used for the combination regimen.

Methods

Female athymic nude mice (nu/nu, Charles River) were 13-14 weeks old on Day 1 of the study. Human U87MG glioblastomas were maintained in athymic nude mice. A tumor fragment (1 mm³) was implanted subcutaneously into the right flank of each test mouse. Tumors were monitored twice weekly and then daily as they approached a size range of 60-100 mg. On Day 1 of the study, the animals were sorted into six groups of ten mice, with tumor sizes of 62.5-126.0 mg and group mean tumor sizes of 70.0-71.4 mg. Tumor weight was estimated with the assumption that 1 mg is equivalent to 1 mm³ of tumor volume. Volume was calculated using the formula:

$$Tumor Volume ( {mm}^{3} ) = \frac{{w}^{2} \times l}{2}$$

where w=width and l=length in mm of a U87MG tumor.

Drugs

Frozen GU1 dosing solutions and the GU1 vehicle were obtained from CuraGen and stored at −20° C. until they were used. Each vial contained sufficient dosing solution for two doses. The thawed solutions were stored at 4° C. between dosing, and discarded after the second dosing.

Treatment

The vehicle for GU1 (CG51896-02) was 20 mM Tris-HCl, pH 7.4, containing 50 mM NaCl. Carmustine (BCNU, 1,3-bis (2-chloroethyl)-1-nitrosourea), Bristol Laboratories) was dissolved in anhydrous ethanol and stored at 4° C. On each day of dosing, an aliquot of the ethanolic solution was diluted tenfold with sterile water, and then diluted to the appropriate dosing concentration with 5% dextrose in water (D5W).

Mice were sorted into six groups containing ten mice each, and treated according to the protocol in Table 13. Control Group 1 mice received the GU1 vehicle i.p. twice daily on Days 1-14 (BID×14). Group 2 was given carmustine i.p. at 15 mg/kg once daily on three alternate days beginning on Day 1 (QOD×3). Groups 3 and 4 received GU1 i.p. BID×14 at 1 and 5 mg/kg, respectively. Group 5 received GU1-carmustine combination therapy, consisting of the treatments administered to both Group 2 and Group 4. Group 6 was given GU1 i.p. b.i.d.×14 at 10 mg/kg. The dosing volume of 0.2 mL/20 g body was scaled to the body weight of each animal.

TABLE 13
Study Design

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 10 | Vehicle | — | i.p. | Bid × 14 | | | | |
| 2 | 10 | BCNU | 15 | i.p. | Qod × 3 | | | | |
| 3 | 10 | GU1 | 1 | i.p. | Bid × 14 | | | | |
| 4 | 10 | GU1 | 5 | i.p. | Bid × 14 | | | | |
| 5 | 30 | GU1 | 5 | i.p. | Bid × 14 | BCNU | 15 | i.p. | Qod × 3 |
| 6 | 10 | GU1 | 10 | i.p. | Bid × 14 | | | | |

Endpoint

Treatment efficacy was determined from tumor growth delay (TGD), which is defined as the increase in the median time to endpoint (TTE) in a treatment group compared to the control group. Each animal was euthanized when its neoplasm reached the predetermined endpoint size (1.5 g). The TTE value was calculated for each animal in each group based on linear regression of a log-transformed tumor growth data set comprised of the first observation that exceeded the study endpoint volume and the three consecutive observations that immediately preceded the attainment of the endpoint volume. The TIE is calculated from the following equations:

y₁=mx₁+b (1)

$${x}_{2} = \frac{{y}_{2} - b}{m} \qquad ( 2 )$$

where:

y1=ordinate, log10 (tumor volume, mm3)

x1=Day of observation

y2=log10 (endpoint volume, mm3)

x2=abscissa=TTE (days)

b=intercept

m=slope,

with the assumptions that errors in tumor volume are substantially greater than errors in days, and deviations of tumor volumes from the fitted regression line are similar. Animals that do not reach the endpoint are assigned a TTE; value equal to the last day of the study. Animals classified as TR (treatment-related) deaths or NTRM (non-treatmentrelated metastasis) deaths are assigned a TTE value equal to the day of death. Animals classified as NTR (non-treatment-related) deaths are excluded from TTE calculations. The median TTE of each group is the basis for determining treatment efficacy. Tumor growth delay (TGD) is calculated as the difference between the median TTE for a treatment group and the median TTE of the control group:

TGD=T−C, (3)

expressed in days, or as a percentage of the median TTE of the control group:

$$\% TGD = \frac{T - C}{C} = \times 100 \qquad ( 4 )$$

where:

T=median TTE for a treatment group,

C=median TTE for the control Group 1.

Treatment may cause partial regression (PR) or complete regression (CR) of the tumor in an animal. In a PR response, the tumor weight is ≦50% of its weight on Day 1, but greater than 0 mg, for three consecutive measurements during the course of the study. In a CR response, there is no measurable tumor mass for three consecutive measurements during the course of the study. Animals, classified as having CR responses (no measurable tumor mass for three consecutive measurements) at the termination of a study are additionally classified as long-term tumor-free survivors (LTTFS).

Toxicity

Animals were weighed daily on Days 1-5, then twice weekly until the completion of a study. The mice were examined frequently for overt signs of any adverse, drug-related side effects. Acceptable toxicity for cancer drugs in mice is defined by the NCI as a group mean body-weight loss of less than 20% during the test, and not more than one toxic death among ten treated animals.

Statistical and Graphical Analyses

The logrank test was employed to analyze differences in the median TIE of treated groups versus the vehicle-treated control group. The logrank test analyzes the data for all animals except those recorded as NTR deaths. The two-tailed statistical analyses were conducted at P=0.05. Results were deemed significant at 0.01≦P <0.05, and highly significant at P<0.01. The group median tumor growth curves show the median tumor volume as a function of time. When an animal exited the study due to tumor size, treatment-related death, or non-treatment-related death, the final tumor volume recorded for the animal was included with the data used to calculate the median volume at subsequent time points. Kaplan-Meier plots were constructed to show the percentage of animals remaining in the study versus time. The Kaplan-Meier plots use the same data set as the logrank test.

Results

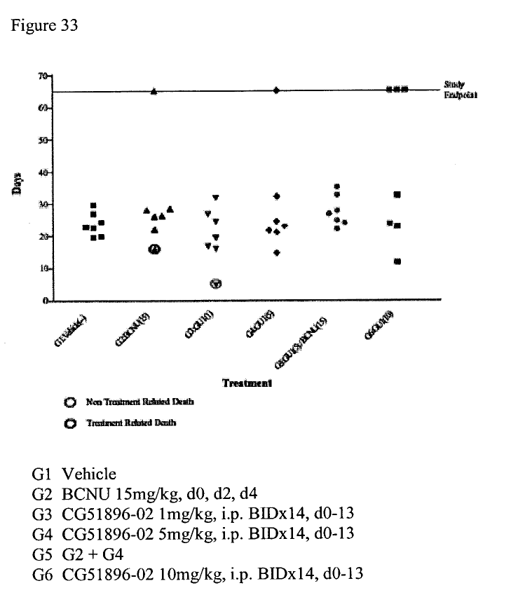

The U87MG-e11 study was performed in accordance with the protocol in Table 13. The 65-day study utilized six groups of ten athymic nude mice bearing well-established (˜71 mg) U87MG glioblastomas on Day 1. On Day 16 of the study, three animals per group were euthanized for tissue sampling. The treatment results are based on the remaining seven mice in each group. Table 14 presents the treatment response summary of median TTE values for the Groups compared. FIG. 33 shows a scatterplot of the TTE values for individual mice in every treatment group. The logrank test was used to determine the significance of any increase in median TTE for a treated group versus the vehicle-treated control group.

TABLE 14
Treatment Response Summary

| | Vehicle | Vehicle | Vehicle | Vehicle | Vehicle |
|---|---|---|---|---|---|
| | Group 1 vs 2 | Group 1 vs 3 | Group 1 vs 4 | Group 1 vs 5 | Group 1 vs 6 |
| | BCNU | GU1 | GU1 | GU1/BCNU | GU1 |
| Groups Compared | 15 mg/kg | 1 mg/kg | 5 mg/kg | 5/15 mg/kg | 10 mg/kg |
| Logrank Test | | | | | |
| Chi square | 0.8228 | 0.02869 | 0.5145 | 2.185 | 3.606 |
| df | 1 | 1 | 1 | 1 | 1 |
| P value | 0.3644 | 0.8655 | 0.4732 | 0.1394 | 0.0576 |
| P value summary | ns | ns | ns | ns | ns |
| Are the survival curves sig different? | No | No | No | No | No |
| Median survival | | | | | |
| Column A | 23 | 23 | 23 | 23 | 23 |
| Column B | 26.3 | 22.05 | 23 | 26.8 | 32.6 |
| Ratio | 0.8745 | 1.043 | 1 | 0.8582 | 0.7055 |
| 95% Cl of ratio | 0.5385 to 1.211 | 0.7070 to 1.379 | 0.6639 to 1.336 | 0.5075 to 1.209 | 0.4128 to 0.9983 |
| Hazard Ratio | | | | | |
| Ratio | 1.634 | 1.09 | 1.456 | 2.081 | 2.911 |
| 95% Cl of ratio | 0.5474 to 5.159 | 0.3368 to 3.647 | 0.4811 to 4.838 | 0.7483 to 7.914 | 0.9603 to 12.85 |

Group 1 mice received the GU1 vehicle i.p. twice daily on Days 1-14 (BID×14).

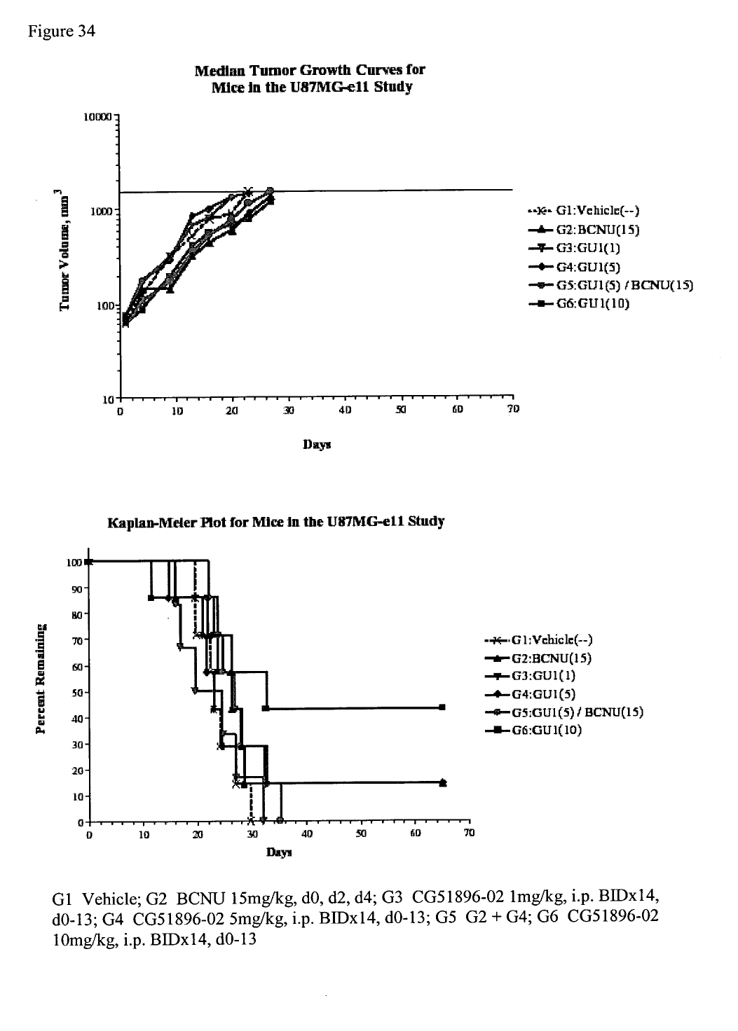

Tumors in all seven vehicle-treated mice grew to the 1.5-g endpoint weight, yielding a median TTE of 23.0 days (Table 14). The absence of 65-day survivors indicates a potential background level of zero unsatisfactory tumor engraftments per group. The median tumor growth curve for the control mice is included in the upper panels of FIG. 34. The percentage of control animals remaining in the study versus time is shown in Kaplan-Meier plots in the lower panels of FIG. 34.

Response of U87MG Xenografts to Intraperitoneal Carmustine

Group 2 mice received carmnustine i.p. at 15 mg/kg. Carmustine was administered, beginning on Day 1, once daily on three alternate days (QOD×3). One treatment-related (TR) death was recorded. Group 2 mice achieved a median TTE of 26.3 days, corresponding to an insignificant 3.3-day TC and 14% tumor growth delay (TGD) relative to control mice (P>0.05). The median tumor burden on Day 65 was 0 mg (n=1 mouse). The treatment yielded one long-term tumor-free survivor (LTTFS). The median tumor growth curve and Kaplan-Meier curve for Group 2 are shifted slightly to the right, compared to the curves for Group 1 (FIG. 34).

Response of U87MG Xenografts to Intraperitoneal CG51896-02 (GU1)

GU1 was administered i.p. to Groups 3, 4, and 6 b.i.d×14 at 1, 5, and 10 mg/kg, respectively. One non-treatment-related (NTR) death was recorded in Group 3. The median TTE for Group 3 mice was 22.0 days. This TTE value is lower than that of vehicle-treated Group 1 mice; however the decrease is not significant (P>0.05). No regression responses were recorded. The median TTE for Group 4 was identical to that of vehicle-treated Group 1 mice (23.0 days). The median tumor burden on Day 65 was 0 mg (n=1). The treatment response in the single 65-day survivor was classified as a PR response, because the tumor first became non-palpable on the last day. Group 6 achieved a median TTE of 32.6 days, corresponding to a 9.6-day T-C and 42% TGD. While the 10 mg/kg GU1 treatment was the most efficacious in this study, the Group 6 median TTE was not significantly greater than that of vehicle-treated Group 1 mice (P=0.0576). The median tumor burden on Day 65 was 0 mg (n=3). One PR response and two LTTFS were recorded. The median tumor growth curves and Kaplan-Meier curves for Groups 3, 4, and 6 do not reflect the regression responses because four of the seven tumors reached the 1.5-g endpoint weight within 32 days (FIGS. 33 and 34).

Response of U87MG Xenografts to GU1-Carmustine Combination Therapy

Group 5 received a combination therapy consisting of GU1 i.p. b.i.d.×14 at 5 mg/kg, and carmustine i.p. qod×3 at 15 mg/kg. The median TTE for Group 5 was 26.8 days, corresponding to a 3.8-day T-C and 17% TGD relative to control mice (P>0.05). There were no regression responses. Comparison of the median tumor growth curve and Kaplan-Meier curve for Group 5 to the curves for Groups 2 and 4 (which received the corresponding monotherapies), does not reveal any enhancement of antitumor efficacy (FIG. 34).

Conclusion

This study evaluated CG51896-02 in athymic mice bearing human U87MG glioblastomas. The tumors in all seven control mice grew at similar rates to the 1.5-g endpoint weight, yielding a median Tm of 23 days. As shown in FIG. 1, the majority of tumors in every treatment group reached the endpoint with TTE values similar to those of the vehicle treated tumors. Hence, none of the test regimens produced a statistically significant increase in median TTE. The highest dose of GU1 produced the greatest TGD, 42%, which was nearly significant (P=0.0576). The number of tumors that did not reach the endpoint provided evidence of some treatment efficacy. Carmustine monotherapy yielded one LTTFS, 5 mg/kg GU1 yielded one PR response (with a 0 mg tumor weight on Day 65), and 10 mg/kg GU1 yielded one PR and two LTTFS. The combination therapy, with 15 mg/kg carmustine and 5 mg/kg GU1, yielded no regression responses, indicating the absence of positive interactions between this GU1 regimen and the alkylator treatment.

In summary, 10 mg/kg dose of GU1 (CG51896-02) yielded three 65-day survivors with a median tumor weight of 0 mg, suggesting that at 10 mg/kg, CG51896-02 could be used as an effective protein therapeutic that could induce the regression of glioblastoma.

Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or protein delivery method is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

We claim

1. A method of inhibiting cell migration comprising contacting a cell with a composition comprising a polypeptide having at least 95% sequence identity to SEQ D NOs: 14, 16, 18, 20, 22, 24, 26, 28, 30, 32, 34, 36, 38, 40, 42, 44, 46, 48, 50, 52, 54, or 56.

2. The method of claim 1, wherein the cell is contacted in vivo, in vitro or ex vivo.

3. The method of claim 1, wherein the cell is selected from the group consisting of an endothelial cell, an epithelial cell, a neuronal cell or a mesenchymal cell.

4. The method of claim 3, wherein the endothelial cell is a microvascular endothelial cell or an umbilical vein endothelial cell.

5. The method of claim 3, wherein the epithelial cell is a renal cell or a pancreatic cell.

6. The method of claim 3, wherein the neuronal cell is selected from the group consisting of a glial cell, an axonal cell, and a dendritic cell.

7. The method of claim 1, wherein the cell is a cancer cell.

8. The method of claim 6, wherein the cancer cell is a neuroblastoma cell, a renal carcinoma cell, a fibrosarcoma cell, a rhabdosarcoma cell or a pancreatic cancer cell.

9. A method of inhibiting cell migration comprising introducing to a cell a composition comprising a nucleic acid having at least 95% sequence identity to SEQ ID NOs: 13, 15, 17, 19, 21, 23, 25, 27, 29, 31, 33, 35, 37, 39, 41, 43, 45,47,49,51,53, or 55.

10. The method of claim 9, wherein the nucleic acid is introduced in vivo, in vitro or ex vivo.

11. The method of claim 9, wherein the cell is selected from the group consisting of an endothelial cell, an epithelial cell, a neuronal cell or a mesenchymal cell.

12. The method of claim 11, wherein the endothelial cell is a microvascular endothelial cell or an umbilical vein endothelial cell.

13. The method of claim 11, wherein the epithelial cell is a renal cell or a pancreatic cell.

14. The method of claim 11, wherein the neuronal cell is selected from the group consisting of a glial cell, an axonal cell, and a dendritic cell.

15. The method of claim 9, wherein the cell is a cancer cell.

16. The method of claim 16, wherein the cancer cell is a neuroblastoma cell, a renal carcinoma cell, a fibrosarcoma cell, a rhabdosarcoma cell or a pancreatic cancer cell.

17. A method of inhibiting angiogenesis of a tissue comprising contacting the tissue with a composition comprising a polypeptide having at least 95% sequence identity to SEQ ID NOs: 14, 16, 18, 20, 22, 24, 26, 28, 30, 32, 34, 36, 38, 40, 42, 44, 46, 48, 50, 52, 54, or 56.

18. The method of claim 17, wherein the tissue is selected from the group consisting of an endothelial tissue, a epithelial tissue a neuronal tissue or a mesenchymal tissue.

19. The method of claim 18 wherein the endothelial tissue is a vein, an artery,or a microvasculature.

20. The method of claim 18, wherein the epithelial tissue is a kidney tissue, a pancreatic tissue or a renal tissue.

21. The method of claim 18, wherein the neuronal tissue is a glial tissue.

22. The method of claim 17, wherein the tissue is a tumor.

23. The method of claim 22, wherein the tumor is cancerous.

24. The method of claim 23, wherein the cancer is neuroblastoma, renal carcinoma, fibrosarcoma, rhabdosarcoma or pancreatic cancer.

25. A method of inhibiting angiogenesis of a tissue introducing to a cell a composition comprising a nucleic acid having at least 95% sequence identity to a SEQ ID NOs: 13, 15, 17, 19, 21, 23, 25, 27, 29, 31, 33, 35, 37, 39, 41, 43, 45, 47, 49, 51, 53, 55, 57, or 59.

26. The method of claim 25, wherein the nucleic acid is introduced in vivo, in vitro or ex vivo.

27. The method of claim 25, wherein the tissue is selected from the group consisting of an endothelial tissue, a epithelial tissue, a neuronal tissue or a mesenchymal tissue.

28. The method of claim 27 wherein the endothelial tissue is a vein, an artery, or a microvasculature.

29. The method of claim 27, wherein the epithelial tissue is a kidney tissue a pancreatic tissue or a renal tissue.

30. The method of claim 27, wherein the neuronal tissue is a glial tissue.

31. The method of claim 25, wherein the tissue is a tumor.

32. The method of claim 31, wherein the tumor is cancerous.

33. The method of claim 32, wherein the cancer is neuroblastoma, a renal carcinoma, fibrosarcoma, rhabdosarcoma or pancreatic cancer.

34. A method of inhibiting actin filament formation in a cell by contacting the cell with a composition comprising a polypeptide having at least 95% sequence identity to SEQ ED NOs: 14, 16, 18, 20, 22, 24, 26, 28, 30, 32, 34, 36, 38, 40, 42, 44, 46, 48, 50, 52, 54, or 56.

35. The method of claim 34, wherein the cell is contacted in vivo, in vitro or ex vivo.

36. The method of claim 34, wherein the cell is selected from the group consisting of an endothelial cell, an epithelial cell, a neuronal cell or a mesenchymal cell.

37. The method of claim 36, wherein the endothelial cell is a microvascular endothelial cell or an umbilical vein endothelial cell

38. The method of claim 36, wherein the epithelial cell is a renal cell or a pancreatic cell.

39. The method of claim 36, wherein the neuronal cell is selected from the group consisting of a glial cell, an axonal cell, and a dendritic cell.

40. The method of claim 34, wherein the cell is a cancer cell.

41. The method of claim 40, wherein the cancer cell is a neuroblastoma cell, a renal carcinoma cell, a fibrosarcoma cell, a rhabdosarcoma cell or a pancreatic cancer cell.

42. A method of inhibiting actin filament formation comprising introducing to a cell a composition comprising a nucleic acid having at least 95% sequence identity to SEQ ID NOs: 13, 15, 17, 19, 21, 23, 25, 27, 29, 31, 33, 35, 37, 39, 41, 43, 45, 47, 49, 51, 53, or 55.

43. The method of claim 42, wherein the nucleic acid is introduced in vivo, in vitro or ex vivo.

44. The method of claim 42, wherein the cell is selected from the group consisting of an endothelial cell, an epithelial cell, a neuronal cell or a mesenchymal cell.

45. The method of claim 44, wherein the endothelial cell is a microvascular endothelial cell or an umbilical vein endothelial cell

46. The method of claim 44, wherein the epithelial cell is a renal cell or a pancreatic cell.

47. The method of claim 44, wherein the neuronal cell is selected from the group consisting of a glial cell, an axonal cell, and a dendritic cell.

48. The method of claim 42, wherein the cell is a cancer cell.

49. The method of claim 48, wherein the cancer cell is a neuroblastoma cell, a renal carcinoma cell, a fibrosarcoma cell, a rhabdosarcoma cell or a pancreatic cancer cell.

50. A method of preventing or alleviating a symptom of an angiogenic related disorder comprising administering to a subject a composition comprising a polypeptide having at least 95% sequence identity to SEQ ID NOs: 14, 16, 18, 20, 22, 24, 26, 28, 30, 32, 34, 36, 38, 40, 42, 44, 46, 48, 50, 52, 54, or 56.

51. The method of claim 50, wherein the angiogenic related disorder is cancer.

52. The method of claim 5 1, wherein the cancer is a pancreatic cancer, a renal cancer or a neuroblastoma.

53. A chimeric protein comprising a first polypeptide comprising a NOVX polypeptide and second polypeptide.

54. The chimeric protein of claim 53, wherein the second polypeptide is at least a portion of an immunoglobulin molecule.

55. The chimeric protein of claim 53, wherein second polypeptide comprises the Fc region of an immunoglobulin molecule.

56. A composition comprising SEQ ID NO: 50 or 54.
